(19) United States
(12) Patent Application Publication
(10) Pub. No.: US 2010/0331397 A1
(43) Pub. Date: Dec. 30, 2010

(54) 2-5A ANALOGS AND THEIR METHODS OF USE

(75) Inventors: Leonid Beigelman, San Mateo, CA (US); Lawrence Blatt, San Francisco, CA (US); Harri Lönnberg, Turku (FI); Roopa Rai, San Carlos, CA (US); Guangyi Wang, Carlsbad, CA (US); Thomas Horn, Berkeley, CA (US); Julian Symons, San Carlos, CA (US); Antitsa Simitrova Stoycheva, Half Moon Bay, CA (US); Dean Ng, San Ramon, CA (US); Jerome Deval, Pacifica, CA (US)

Correspondence Address: KNOBBE MARTENS OLSON & BEAR LLP, 2040 MAIN STREET, FOURTEENTH FLOOR, IRVINE, CA, 92614, US

(73) Assignee: ALIOS BIOPHARMA, INC., South San Francisco, CA (US)

(21) Appl. No.: 12/822,088

(22) Filed: Jun. 23, 2010

Related U.S. Application Data
(60) Provisional application No. 61/219,960, filed on Jun. 24, 2009.
(60) Provisional application No. 61/219,938, filed on Jun. 24, 2009.

(51) Int. Cl. A61K 31/7088; C07H 21/00; A61P 31/04; A61P 31/12; A61P 35/00

(52) U.S. Cl. 514/44 R; 536/25.6

(57) ABSTRACT

Disclosed herein are compounds that activate RNaseL, methods of synthesizing compounds that activate RNaseL and the use of compounds that activate RNaseL for treating and/or ameliorating a disease or a condition, such as a viral infection, a bacterial infection, cancer and/or parasitic disease.

### RELATED APPLICATIONS

This application claims priority to U.S. Provisional Application Nos. 61/219,960, filed Jun. 24, 2009; and 61/219,938, filed Jun. 24, 2009, both of which are hereby incorporated by reference in their entireties.

### BACKGROUND

1. Field

This application relates to the fields of organic chemistry, pharmaceutical chemistry, biochemistry, molecular biology and medicine. In particular, disclosed herein are compounds that activate RNaseL, methods of synthesizing compounds that activate RNaseL, and the use of those compounds for treating and/or ameliorating a disease or a condition, such as a viral infection, a bacterial infection, parasitic infection and/or neoplastic disease.

2. Description

The interferon pathway is induced in mammalian cells in response to various stimuli, including a viral infection. It is believed that this pathway induces the transcription of at least 200 molecules and cytokines, (immuno-regulatory substances that are secreted by cells of the immune system) involved in the defense against viral infections. These molecules and cytokines play a role in the control of cell proliferation, cell differentiation, and modulation of the immune responses.

The 2-5A system is one of the major pathways induced by the interferon pathway and has been implicated in some of its antiviral activities. This system has been described as comprising three enzymatic activities, including 2-5A-synthetases, 2-5A-phosphodiesterase, and RNaseL. 2-5A-synthetases are a family of four interferon-inducible enzymes which, upon activation by double-stranded RNA, convert ATP into the unusual series of oligomers known as 2-5A. The 2-5A-phosphodiesterase is believed to be involved in the catabolism of 2-5A from the longer oligomer. The 2-5A-dependent endoribonuclease L or RNase L is the effector enzyme of this system. RNaseL is normally inactive within the cell, so that it cannot damage the large amount of native RNA essential for normal cell function. Its activation by subnanomolar levels of 2-5A leads to the destruction of viral mRNA within the cell, and at the same time triggers the removal of the infected cell by inducing apoptosis (programmed cell death).

### SUMMARY

Some embodiments disclosed herein relate to a compound of Formula (I) or a pharmaceutically acceptable salt thereof:

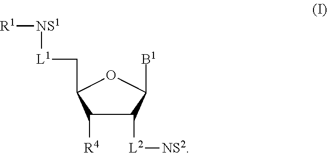

Other embodiments disclosed herein relate to a compound of Formula (Ia) or a pharmaceutically acceptable salt thereof:

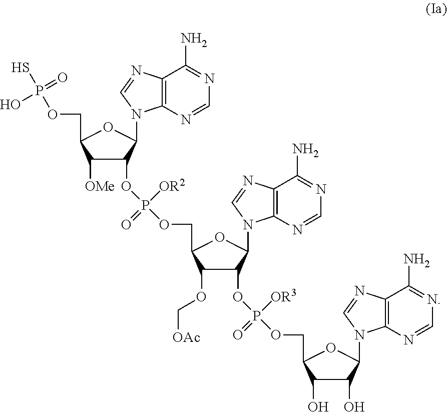

Still other embodiments disclosed herein relate to a compound of Formula (II) or a pharmaceutically acceptable salt thereof:

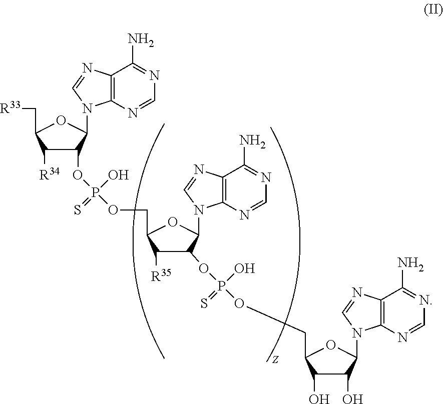

Some embodiments disclosed herein relate to methods of synthesizing a compound of Formula (I), or a pharmaceutically acceptable salt thereof. Other embodiments disclosed herein relate to methods of synthesizing a compound of Formula (Ia), or a pharmaceutically acceptable salt thereof. Still other embodiments disclosed herein relate to methods of synthesizing a compound of Formula (II), or a pharmaceutically acceptable salt thereof.

Some embodiments disclosed herein relate to pharmaceutical compositions that can include one or more compounds of Formulae (I), (Ia) and/or (II), or a pharmaceutically acceptable salt thereof, and a pharmaceutically acceptable carrier, diluent, excipient or combination thereof.

Some embodiments disclosed herein relate to methods of ameliorating or treating a neoplastic disease that can include administering to a subject suffering from a neoplastic disease a therapeutically effective amount of one or more compound of Formulae (I), (Ia) and/or (II), or a pharmaceutically acceptable salt thereof, or a pharmaceutical composition that includes one or more compounds of Formulae (I), (Ia) and/or (II), or a pharmaceutically acceptable salt thereof.

Other embodiments disclosed herein relate to methods of inhibiting the growth of a tumor that can include administering to a subject having a tumor a therapeutically effective amount of one or more compound of Formulae (I), (Ia) and/or (II), or a pharmaceutically acceptable salt thereof, or a pharmaceutical composition that includes one or more compounds of Formulae (I), (Ia) and/or (II), or a pharmaceutically acceptable salt thereof.

Still other embodiments disclosed herein relate to methods of ameliorating or treating a viral infection that can include administering to a subject suffering from a viral infection a therapeutically effective amount of one or more compound of Formulae (I), (Ia) and/or (II), or a pharmaceutically acceptable salt thereof, or a pharmaceutical composition that includes one or more compounds of Formulae (I), (Ia) and/or (II), or a pharmaceutically acceptable salt thereof.

Yet still other embodiments disclosed herein relate to methods of ameliorating or treating a bacterial infection that can include administering to a subject suffering from a bacterial infection a therapeutically effective amount of one or more compound of Formulae (I), (Ia) and/or (II), or a pharmaceutically acceptable salt thereof, or a pharmaceutical composition that includes one or more compounds of Formulae (I), (Ia) and/or (II), or a pharmaceutically acceptable salt thereof.

Some embodiments disclosed herein relate to methods of ameliorating or treating a parasitic disease that can include administering to a subject suffering from a parasitic disease a therapeutically effective amount of one or more compound of Formulae (I), (Ia) and/or (II), or a pharmaceutically acceptable salt thereof, or a pharmaceutical composition that includes one or more compounds of Formulae (I), (Ia) and/or (II), or a pharmaceutically acceptable salt thereof.

### BRIEF DESCRIPTION OF THE DRAWINGS

FIG. 1 shows the 48-well plate after staining with crystal violet in a bovine viral diarrhea virus assay

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R, R¹, R², R³, R⁴, R⁵, R⁶, R⁷, R⁸, R^{a}, R^{b}, R^{A}, R^{B} and R^{C} represent substituents that can be attached to the indicated atom. An R group may be substituted or unsubstituted. If two “R” groups are described as being “taken together” the R groups and the atoms they are attached to can form a cycloalkyl, aryl, heteroaryl or heterocycle. For example, without limitation, if R^{1a} and R^{1b} of an NR^{1a}R^{1b} group are indicated to be “taken together,” it means that they are covalently bonded to one another to form a ring:

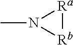

Whenever a group is described as being “optionally substituted” that group may be unsubstituted or substituted with one or more of the indicated substituents Likewise, when a group is described as being “unsubstituted or substituted” if substituted, the substituent may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. The alkyl group may be substituted or unsubstituted.

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl and the like.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

As used herein, “cycloalkynyl” refers to a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

As used herein, “heteroalicyclic” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatoms are independently selected from oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides, cyclic carbamates, and the like. When composed of two or more rings, the rings may be joined together in a fused fashion. Additionally, any nitrogens in a heteroalicyclic may be quaternized. Heteroalicyclyl or heteroalicyclic groups may be unsubstituted or substituted. Examples of such “heteroalicyclic” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylalkyl, 3-phenylalkyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their substituted as well as benzo-fused analogs.

A “(heteroalicyclyl)alkyl” is a heterocyclic or a heteroalicyclylic group connected, as a substituent, via a lower alkylene group. The lower alkylene and heterocyclic or a heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl or a cycloalkynyl is defined as above. A non-limiting list of alkoxys are methoxy, ethoxy, n-propoxy, 1-methylethoxy(isopropoxy), n-butoxy, iso-butoxy, sec-butoxy, tert-butoxy, and the like. An alkoxy may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “aryloxy” and “arylthio” refers to RO- and RS-, in which R is an aryl, such as but not limited to phenyl. Both an aryloxy and arylthio may be substituted or unsubstituted.

A “sulfenyl” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein X is a halogen.

A “trihalomethanesulfonamido” group refers to an “X₃CS(O)₂ N(R_{A})—” group wherein X is a halogen and R_{A} hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An S-sulfonamido may be substituted or unsubstituted.

An “N-sulfonamido” group refers to a “RSO₂N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-sulfonamido may be substituted or unsubstituted.

An “O-carbamyl” group refers to a “—OC(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-carbamyl may be substituted or unsubstituted.

An “N-carbamyl” group refers to an “ROC(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-carbamyl may be substituted or unsubstituted.

An “O-thiocarbamyl” group refers to a “—OC(═S)—N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-thiocarbamyl may be substituted or unsubstituted.

An “N-thiocarbamyl” group refers to an “ROC(═S)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-thiocarbamyl may be substituted or unsubstituted.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A C-amido may be substituted or unsubstituted.

An “N-amido” group refers to a “R^{C}(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-amido may be substituted or unsubstituted.

As used herein, “organylcarbonyl” refers to a group of the formula —C(═O)Rₐ wherein Rₐ can be alkyl, alkenyl, alkynyl, cycloalkyl, cycloalkenyl, cycloalkynyl, aryl, heteroaryl, heteroalicyclyl, aralkyl, or (heteroalicyclyl)alkyl. An organylcarbonyl can be substituted or unsubstituted.

The term “alkoxycarbonyl” as used herein refers to a group of the formula —C(═O)ORₐ wherein Rₐ can be the same as defined with respect to organylcarbonyl. An alkoxycarbonyl can be substituted or unsubstituted.

As used herein, “organylaminocarbonyl” refers to a group of the formula —C(═O)NHRₐ wherein Rₐ can be the same as defined with respect to organylcarbonyl. An organylaminocarbonyl can be substituted or unsubstituted.

As used herein, the term “levulinoyl” refers to a —C(═O)CH₂CH₂C(═O)CH₃ group.

The term “halogen atom,” as used herein, means any one of the radio-stable atoms of column 7 of the Periodic Table of the Elements, i.e., fluorine, chlorine, bromine, or iodine, with bromine and chlorine being preferred.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “nucleoside” is used herein in its ordinary sense as understood by those skilled in the art, and refers to a compound composed of an optionally substituted pentose moiety or modified pentose moiety attached to a heterocyclic base or tautomer thereof via a N-glycosidic bond, such attached via the 9-position of a purine-base or the 1-position of a pyrimidine-base. Examples include, but are not limited to, a ribonucleoside comprising a ribose moiety and a deoxyribonucleoside comprising a deoxyribose moiety. A modified pentose moiety is a pentose moiety in which an oxygen has been replaced with a carbon and/or a carbon has been replaced with a sulfur or an oxygen atom. The compounds described herein are made of monomers that are considered to fall with the definition of “nucleoside,” including all the substitutions in the base and sugar moieties that are disclosed herein. In some instances, the nucleoside can be a nucleoside analog drug.

As used herein, the term “nucleoside analog drug” refers to a compound composed of a nucleoside that has therapeutic activity, such as antiviral, anti-neoplastic, anti-parasitic and/or antibacterial activity. A large number of nucleoside analog drugs are known that can be incorporated into the compounds described herein. For example, a nucleoside analog drug can be in place of NS¹ and/or NS².

The term “nucleotide” is used herein in its ordinary sense as understood by those skilled in the art, and refers to a nucleoside having a phosphate ester bound to the pentose moiety, for example, at the 5′-position.

As used herein, the term “protected nucleoside” refers to a nucleoside in which one or more hydroxy groups attached to the ribose or deoxyribose ring are protected with one or more protecting groups, such as those described herein. An example of protected nucleoside is an adenosine in which the oxygen at the 3′-position is protected with a protecting group such as methyl group or a levulinoyl group.

As used herein, the term “heterocyclic base” refers to an optionally substituted nitrogen-containing heterocyclyl attached to an optionally substituted pentose moiety or modified pentose moiety. In some embodiments, the heterocyclic base can be selected from an optionally substituted purine-base, an optionally substituted pyrimidine-base and an optionally substituted triazole-base (for example, a 1,2,4-triazole). The term “purine-base” is used herein in its ordinary sense as understood by those skilled in the art, and includes its tautomers. Similarly, the term “pyrimidine-base” is used herein in its ordinary sense as understood by those skilled in the art, and includes its tautomers. A non-limiting list of optionally substituted purine-bases includes purine, adenine, guanine, hypoxanthine, xanthine, 7-methylguanine, theobromine, caffeine, uric acid and isoguanine. Examples of pyrimidine-bases include, but are not limited to, cytosine, thymine, uracil, 5,6-dihydrouracil and 5-methylcytosine. An example of an optionally substituted triazole-base is 1,2,4-triazole-3-carboxamide. Other non-limiting examples of heterocyclic bases include diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-fluorouracil, 5-bromouracil, pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases.

The terms “phosphorothioate” and “phosphothioate” refer to a compound of the general formula

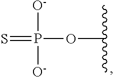

its protonated forms (for example,

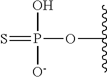

and

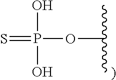

and its tautomers (such as

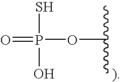

As used herein, the term “phosphate” is used in its ordinary sense as understood by those skilled in the art, and includes its protonated forms (for example,

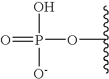

and

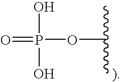

As used herein, the term “protected heterocyclic base” refers to a heterocyclic base in which one or more amino groups attached to the base are protected with one or more suitable protecting groups and/or one or more —NH groups present in a ring of the heterocyclic base are protected with one or more suitable protecting groups. When more than one protecting group is present, the protecting groups can be the same or different.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls (e.g., t-butoxycarbonyl (BOC)); arylalkylcarbonyls (e.g., benzyloxycarbonyl, benzoyl); substituted methyl ether (e.g. methoxymethyl ether); substituted ethyl ether; a substituted benzyl ether; tetrahydropyranyl ether; silyl ethers (e.g., trimethylsilyl, triethylsilyl, triisopropylsilyl, t-butyldimethylsilyl, or t-butyldiphenylsilyl); esters (e.g. benzoate ester); carbonates (e.g. methoxymethylcarbonate); sulfonates (e.g. tosylate, mesylate); acyclic ketal (e.g. dimethyl acetal); cyclic ketals (e.g., 1,3-dioxane or 1,3-dioxolanes); acyclic acetal; cyclic acetal; acyclic hemiacetal; cyclic hemiacetal; and cyclic dithioketals (e.g., 1,3-dithiane or 1,3-dithiolane).

“Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates and halogens. Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference for the limited purpose of disclosing characteristics and examples of leaving groups.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of a phosphorothioate group are intended to be included. Examples of tautomers of a phosphorothioate include the following:

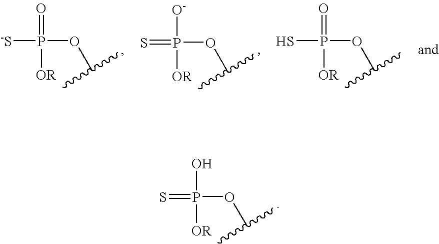

Some embodiments disclosed herein relate to a compound of Formula (I), or a pharmaceutically acceptable salt thereof, wherein: R¹ can be selected from: —(CH₂)ₐ—OR¹⁶, —O—CH₂—COOR¹⁶, —(CH₂)_{b}—COOR¹⁶, —(CH₂)_{c}—C(═S)OR¹⁶, —(CH₂)_{d}—C(═O)NR¹⁷R¹⁸, —(CH₂)ₑ—NH—SO₂—R¹⁶, —(CH₂)_{f}—NH—SO₂—NR¹⁷R¹⁸, —(CH₂)_{g}—NH—CO₂—R¹⁶, —(CH₂)ₕ—NH—C(═O)—R¹⁶, —(CH₂)ᵢ—NH—C(═O)—NR¹⁷R¹⁸, —CH₂—C(R¹⁹)₂—CH₂—OH,

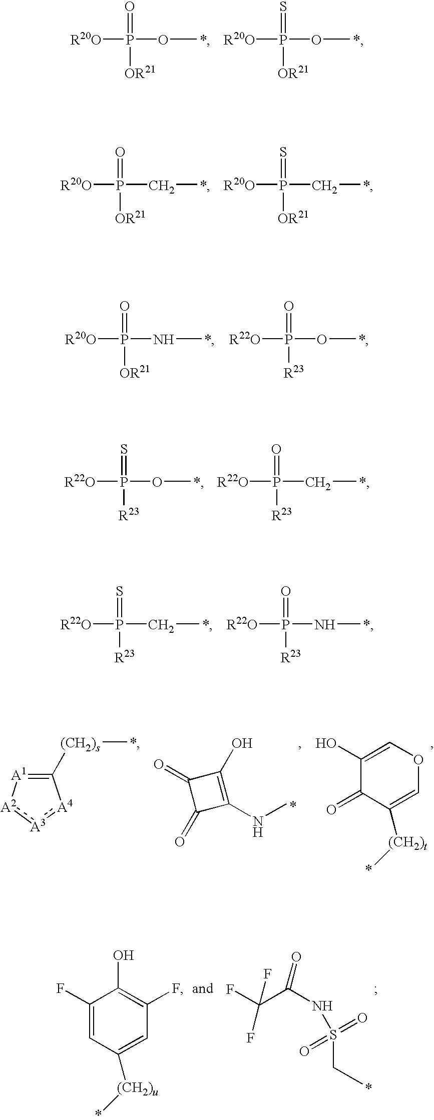

### L¹ can be

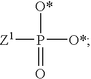

### L² can be

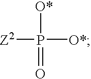

Z¹ can be selected from —OR², S⁻ and —SH; Z² can be selected from —OR³, S⁻ and —SH; R² can be selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl and

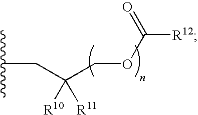

R³ can be selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl and

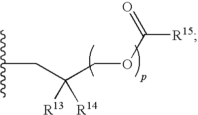

R⁴ can be selected from hydrogen, hydroxy, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R⁵)₂—O—C(═O)R⁶; B¹ can be an optionally substituted heterocyclic base; each R¹⁹ can be independently hydrogen or halogen; R²⁰, R²¹ and R²² can be each independently selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, pivaloyloxymethoxy, isopropyloxycarbonyloxymethoxy and

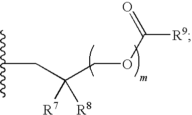

R²³ can be independently selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, and NR²⁴R²⁵; A¹ can be CR²⁶ or N; A² can be C(OH), NH, or O (oxygen); A³ can be C(OH) or N (nitrogen); A⁴ can be C(OH), N (nitrogen), or O (oxygen); R⁷, R⁸, R¹⁰, R¹¹, R¹³ and R¹⁴ can be each independently selected from —C≡N an optionally substituted C₁₋₈ organylcarbonyl, an optionally substituted C₁₋₈ alkoxycarbonyl and an optionally substituted C₁₋₈ organylaminocarbonyl; R⁵, R⁶, R⁹, R¹², R¹⁵, R¹⁶, R¹⁷, R¹⁸, R²⁴, R²⁵ and R²⁶ can be each independently selected from hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl; b, c and d can be each independently selected from 1, 2 and 3; a, e, f, g, h, i, s, t and u can be each independently 0 or 1; m, n and p can be each independently 1 or 2; NS¹ and NS² can be each independently selected from a nucleoside and a protected nucleoside; each [symbol] can be independently a single or double bond, provided that both [symbol] cannot be double bonds; each * represents a point of attachment; and provided that when R¹ is

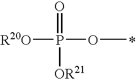

and at least one of R²⁰ and R²¹ is not

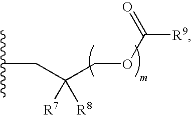

then at least one of Z¹ and Z² is S⁻ or —SH; and provided that if R⁴ is hydroxy, and Z¹ and Z² are both S⁻ or —SH then R¹ cannot be or

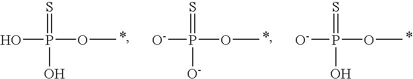

or

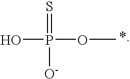

Some embodiments disclosed herein relate to a compound of Formula (I), or a pharmaceutically acceptable salt thereof, wherein R¹ can be selected from: —(CH₂)—OR¹⁶, —O—CH₂—COOR¹⁶, —(CH₂)_{b}—COOR¹⁶, —(CH₂)_{c}—C(═S)OR¹⁶, —(CH₂)_{d}—C(═O)NR¹⁷R¹⁸, —(CH₂)ₑ—NH—SO₂—R¹⁶, —(CH₂)_{f}—NH—SO₂—NR¹⁷R¹⁸, —(CH₂)_{g}—NH—CO₂—R¹⁶, —(CH₂)ₕ—NH—C(═O)—R¹⁶, —(CH₂)ᵢ—NH—C(═O)—NR¹⁷R¹⁸, and —CH₂—C(R¹⁹)₂—CH₂—OH; Z¹ can be selected from —OR², S⁻ and —SH and Z² can be selected from —OR³, S⁻ and —SH. In some of the embodiments of this paragraph, R⁴ can be selected from hydrogen, hydroxy, an optionally substituted —O—C₁₋₆ alkyl and —OC(R⁵)₂—O—C(═O)R⁶. In some of the embodiments of this paragraph, NS¹ can be the structure of Formula (III) or Formula (IIIa). In some of the embodiments of this paragraph, NS² can be the structure of Formula (IV).

Other embodiments disclosed herein relate to a compound of Formula (I), or a pharmaceutically acceptable salt thereof, wherein R¹ can be selected from:

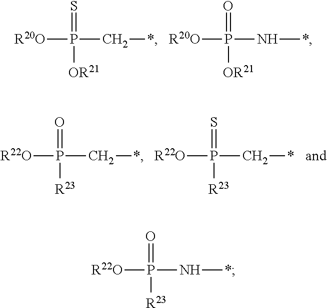

Z¹ can be selected from —OR², S⁻ and —SH and Z² can be selected from —OR³, S⁻ and —SH. In some of the embodiments of this paragraph, R⁴ can be selected from hydrogen, hydroxy, an optionally substituted —O—C₁₋₆ alkyl and —OC(R⁵)₂—O—C(═O)R⁶. In some of the embodiments of this paragraph, NS¹ can be the structure of Formula (III) or Formula (IIIa). In some of the embodiments of this paragraph, NS² can be the structure of Formula (IV).

Still other embodiments disclosed herein relate to a compound of Formula (I), or a pharmaceutically acceptable salt thereof, wherein R¹ can be selected from:

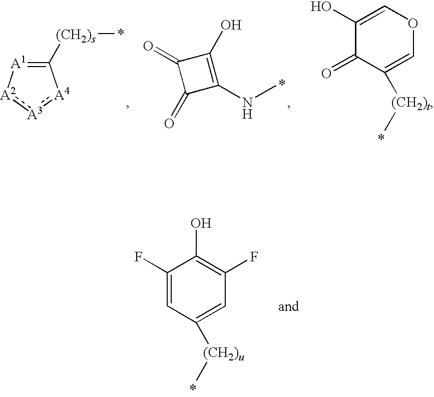

and

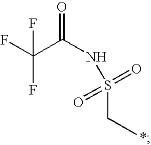

Z¹ can be selected from —OR², S⁻ and —SH and Z² can be selected from —OR³, S⁻ and —SH. In some of the embodiments of this paragraph, R⁴ can be selected from hydrogen, hydroxy, an optionally substituted —O—C₁₋₆ alkyl and —OC(R⁵)₂—O—C(═O)R⁶. In some of the embodiments of this paragraph, NS¹ can be the structure of Formula (III) or Formula (IIIa). In some of the embodiments of this paragraph, NS² can be the structure of Formula (IV).

Yet still other embodiments disclosed herein relate to a compound of Formula (I), or a pharmaceutically acceptable salt thereof, wherein R¹ can be

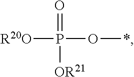

R²⁰ and R²¹ can both be

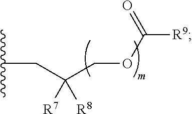

### Z¹ can be —OR²; Z² can be —OR³; R² can be

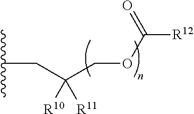

### and R³ can be

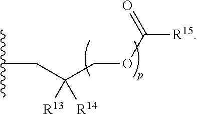

In some of the embodiments of this paragraph, R⁴ can be selected from hydrogen, hydroxy, an optionally substituted —O—C₁₋₆ alkyl and —OC(R⁵)₂—O—C(═O)R⁶. In some of the embodiments of this paragraph, NS¹ can be the structure of Formula (III) or Formula (IIIa). In some of the embodiments of this paragraph, NS² can be the structure of Formula (IV).

Some embodiments disclosed herein relate to a compound of Formula (I), or a pharmaceutically acceptable salt thereof, wherein one of R¹, L¹ and L² are a phosphorothioate. Other embodiments disclosed herein relate to a compound of Formula (I), or a pharmaceutically acceptable salt thereof, wherein two of R¹, L¹ and L² are phosphorothioates. In some of the embodiments of this paragraph, R⁴ can be selected from hydrogen, hydroxy, an optionally substituted —O—C₁₋₆ alkyl and —OC(R⁵)₂—O—C(═O)R⁶. In some of the embodiments of this paragraph, NS¹ can be the structure of Formula (III) or Formula (IIIa). In some of the embodiments of this paragraph, NS² can be the structure of Formula (IV).

Some embodiments disclosed herein relate to a compound of Formula (I), or a pharmaceutically acceptable salt thereof, wherein R¹, L¹ and L² are all phosphorothioates; and R⁴ can be selected from hydrogen, an optionally substituted —O—C₁₋₆ alkyl and —OC(R⁵)₂—O—C(═O)R⁶. In some of the embodiments of this paragraph, NS¹ can be the structure of Formula (III) or Formula (IIIa). In some of the embodiments of this paragraph, NS² can be the structure of Formula (IV).

In some embodiments, a compound of Formula (I) or a pharmaceutically acceptable salt thereof, can have the structure described herein provided that at least one of Z¹ and Z² is S⁻ or —SH when R¹ is

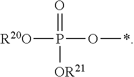

In other embodiments, a compound of Formula (I) or a pharmaceutically acceptable salt thereof, can have the structure described herein provided that at least one of Z¹ and Z² is S⁻ or —SH except when R¹ is —(CH₂)ₐ—OR¹⁶, —O—CH₂—COOR¹⁶, —(CH₂)_{b}—COOR¹⁶, —(CH₂)_{c}—C(═S)OR¹⁶, —(CH₂)_{d}—C(═O)NR¹⁷R¹⁸, —(CH₂)ₑ—NH—SO₂—R¹⁶, —(CH₂)_{f}—NH—SO₂—NR(CH₂)_{g}—NH—CO₂—R¹⁶, —(CH₂)ₕ—NH—C(═O)—R¹⁶, —(CH₂)ᵢ—NH—C(═O)—NR¹⁷R¹⁸, —CH₂—C(R¹⁹)₂—CH₂—OH,

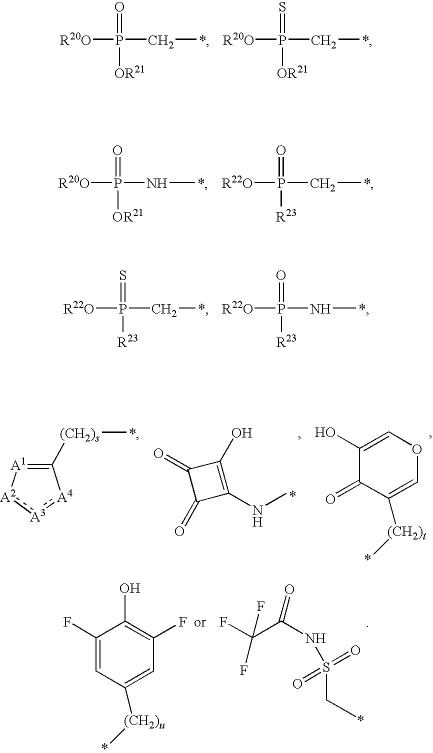

In still other embodiments, a compound of Formula (I) or a pharmaceutically acceptable salt thereof, can have the structure described herein provided that at least one of Z¹ and Z² is S⁻ or —SH except when R¹ is —(CH₂)ₐ—OR¹⁶, —O—CH₂—COOR¹⁶, —(CH₂)_{b}—COOR¹⁶, —(CH₂)_{c}—C(═S)OR¹⁶, —(CH₂)_{d}—C(═O)NR¹⁷R¹⁸, —(CH₂)ₑ—NH—SO₂—R¹⁶, —(CH₂)_{f}—NH—SO₂—NR¹⁷R¹⁸, —(CH₂)_{g}—NH—CO₂—R¹⁶, —(CH₂)ₕ—NH—C(═O)—R¹⁶, —(CH₂)ᵢ—NH—C(═O)—NR¹⁷R¹⁸, —CH₂—C(R¹⁹)₂—CH₂—OH,

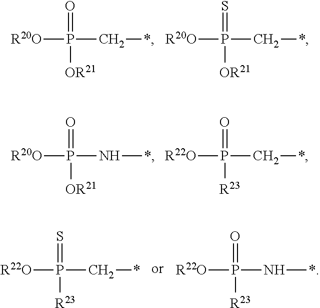

In still other embodiments, a compound of Formula (I) or a pharmaceutically acceptable salt thereof, can have the structure described herein provided that at least one of Z¹ and Z² is S⁻ or —SH except when R¹ is —(CH₂)ₐ13 OR¹⁶, —O—CH₂—COOR¹⁶, —(CH₂)_{b}—COOR¹⁶, —(CH₂)_{c}—C(═S)OR¹⁶, —(CH₂)_{d}—C(═O)NR¹⁷R¹⁸,

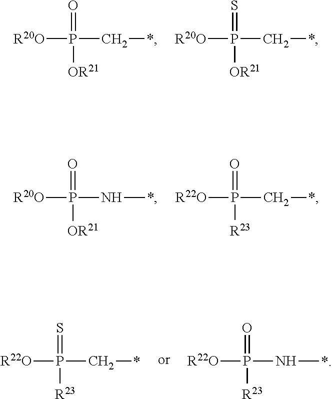

In other embodiments, a compound of Formula (I) or a pharmaceutically acceptable salt thereof, can have the structure described herein provided that at least one of Z¹ and Z² is S⁻ or —SH. In some embodiments, a compound of Formula (I) or a pharmaceutically acceptable salt thereof, can have the structure described herein provided that R¹, L¹ and L² cannot all be phosphorothioates. In other embodiments, a compound of Formula (I) or a pharmaceutically acceptable salt thereof, can have the structure described herein provided that Z¹ and Z² are both S⁻ or SH. In still other embodiments, a compound of Formula (I) or a pharmaceutically acceptable salt thereof, can have the structure described herein provided that R¹ and L¹ are both phosphorothioates. In yet still other embodiments, a compound of Formula (I) or a pharmaceutically acceptable salt thereof, can have the structure described herein provided that R¹ and L² are both phosphorothioates. In some embodiments, a compound of Formula (I) or a pharmaceutically acceptable salt thereof, can have the structure described herein provided that R¹ cannot be

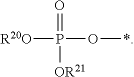

In some embodiments, a compound of Formula (I) or a pharmaceutically acceptable salt thereof, can have the structure described herein provided that R¹ cannot be

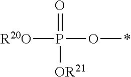

wherein R²⁰ and R²¹ both absent or H. In some embodiments, when R¹ is

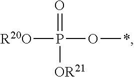

then R²⁰ cannot be a substituted C₁₋₆ alkyl. In some embodiments, when R¹ is

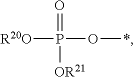

R²¹ cannot be a substituted C₁₋₆ alkyl. In some embodiments, a compound of Formula (I) or a pharmaceutically acceptable salt thereof, can have the structure described herein provided that R¹ cannot be

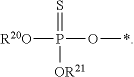

In some embodiments, a compound of Formula (I) or a pharmaceutically acceptable salt thereof, can have the structure described herein provided that R¹ cannot be

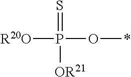

wherein R²⁰ and R²¹ both absent or H. In some embodiments, R¹ cannot be

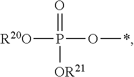

when R⁴ is hydroxy. In other embodiments, R¹ cannot be

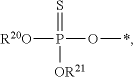

when R⁴ is hydroxy. In some embodiments, a compound of Formula (I) or a pharmaceutically acceptable salt thereof, can have the structure described herein provided that R¹ cannot be —(CH₂)ₐ—OR¹⁶ when L¹ and L² are both phosphorothioates or L¹ and L² are both phosphates. In other embodiments, R¹ cannot be —(CH₂)ₐ—OR¹⁶. In some embodiments, R¹ cannot be —OH. In some embodiments, R⁴ cannot be hydroxy. In some embodiments, R⁴ cannot be hydrogen. In some embodiments, when R¹ is —OH, then R⁴ cannot be hydrogen, hydroxy, an optionally substituted —O—C₁₋₆ alkyl or an optionally substituted —O—C₁₋₆ alkenyl. In some embodiments, when R¹ is —OH and L¹ and L² are both phosphates, then R⁴ cannot be hydrogen, hydroxy or methoxy. In some embodiments, when R¹, L¹ and L² are all phosphates, then R⁴ cannot be hydrogen, hydroxy or methoxy. In some embodiments, the 5-terminal residue cannot be —OH or a phosphate when L² is a phosphorothioate. In other embodiments, the 5-terminal residue cannot be —OH or a phosphate when L¹ is a phosphorothioate. In still other embodiments, the 5-terminal residue cannot be —OH or a phosphate when L¹ and L² are both phosphorothioates.

In some embodiments, L¹ can be

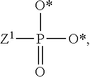

and Z¹ can be selected from S⁻ and —SH. In some embodiments, L² can be

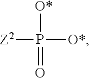

and Z² can be selected from S⁻ and —SH. In an embodiment, L¹ can be

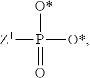

wherein Z¹ can be selected from S⁻ and —SH, and L² can be

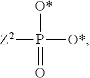

wherein Z² can be selected from S⁻ and —SH

Various substituents can be present at the 5′-terminal position of compounds of Formula (I). In some embodiments, R¹ can be —(CH₂)ₐ—OR¹⁶. In an embodiments, R¹ can be —(CH₂)ₐ—OR¹⁶, wherein R¹⁶ can be hydrogen, and a can be 0. In other embodiments, R¹ can be —(CH₂)_{b}—COOR¹⁶. An example of a —(CH₂)_{b}COOR¹⁶ group is —(CH₂)—COOH. In an embodiment, R¹ can be —(CH₂)_{b}—COOR¹⁶, wherein R¹⁶ can be an optionally substituted C₁₋₆ alkyl, and b is 1. In still other embodiments, R¹ can be —(CH₂)_{c}—C(═S)OR¹⁶. For example, R¹ can be —(CH₂)—C(═S)OR¹⁶, wherein R¹⁶ can be hydrogen or an optionally substituted C₁₋₆ alkyl. In yet still other embodiments, R¹ can be —(CH₂)_{c}—C(═O)NR¹⁷R¹⁸. In an embodiment, R¹ can be —(CH₂)_{c}—C(═O)NR¹⁷R¹⁸, wherein R¹⁷ and R¹⁸ can both be hydrogen or an optionally substituted C₁₋₆ alkyl, and c can be 1. In some embodiments, R¹ can be

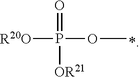

In other embodiments, R¹ can be

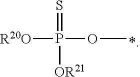

In still other embodiments, R¹ can be

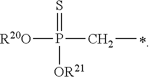

In yet still other embodiments, R¹ can be

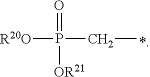

In some embodiments, R¹ can be

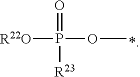

In other embodiments, R¹ can be

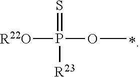

In still other embodiments, R¹ can be

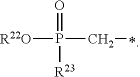

In yet still other embodiments, R¹ can be

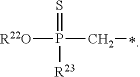

As understood by those skilled in the art, when R²⁰ and/or R²¹ is absent, the oxygen will have an associated negative charge. In some embodiments, when R¹ is

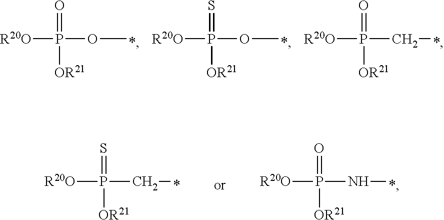

R²⁰ and R²¹ can be both hydrogen. In other embodiments, one of R²⁰ and R²¹ can be hydrogen, and the other of R²⁰ and R²¹ can be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In an embodiment, one of R²⁰ and R²¹ can be hydrogen and the other of R²⁰ and R²¹ can be an optionally substituted C₁₋₆ alkyl. In still other embodiments, both R²⁰ and R²¹ can be independently selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In an embodiment, both R²⁰ and R²¹ can be an optionally substituted C₁₋₆ alkyl. In yet still other embodiments, at least one of R²⁰ and R²¹ can be selected from pivaloyloxymethoxy, isopropyloxycarbonyloxymethoxy and

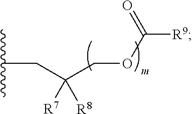

the other of R²⁰ and R²¹ can be selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In some embodiments, both R²⁰ and R²¹ can be independently selected from pivaloyloxymethoxy, isopropyloxycarbonyloxymethoxy and

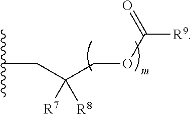

The substituents on

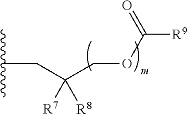

can vary. In an embodiment, R⁷ and R⁸ can be the same. In another embodiment, R⁷ and R⁸ can be different. In some embodiments, R⁷ can be —C≡N and R⁸ can be an optionally substituted C₁₋₈ alkoxycarbonyl such as —C(═O)OCH₃. In other embodiments, R⁷ can be —C≡N and R⁸ can be an optionally substituted C₁₋₈ organylaminocarbonyl, for example, —C(═O)NHCH₂CH₃ and —C(═O)NHCH₂CH₂-phenyl. In some embodiments, R⁷ and R⁸ can be independently C₁₋₈ organylcarbonyl or C₁₋₈ alkoxycarbonyl. In some embodiments, both R⁷ and R⁸ can be an optionally substituted C₁₋₈ organylcarbonyl. In an embodiments, both R⁷ and R⁸ can be —C(═O)CH₃. In other embodiments, both R⁷ and R⁸ can be an optionally substituted C₁₋₈ alkoxycarbonyl. In an embodiment, both R⁷ and R⁸ can be —C(═O)OCH₂CH₃. In another embodiment, both R⁷ and R⁸ can be —C(═O)OCH₃. In some embodiments, including those described in this paragraph, R⁹ can be an optionally substituted C₁₋₄-alkyl. In an embodiment, R⁹ can be methyl or tert-butyl. In some embodiments, m can be 1. In an embodiment, m can be 1 and both R⁷ and R⁸ can be an optionally substituted C₁₋₈ alkoxycarbonyl or an optionally substituted C₁₋₈ organylcarbonyl. In other embodiments, m can be 2. In an embodiment, m can be 2 and both R⁷ and R⁸ can be an optionally substituted C₁₋₈ alkoxycarbonyl. In another embodiment, m can be 2 and both R⁷ and R⁸ can be an optionally substituted C₁₋₈ organylcarbonyl.

Suitable

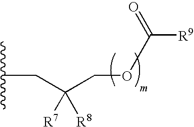

groups include, but are not limited to, the following:

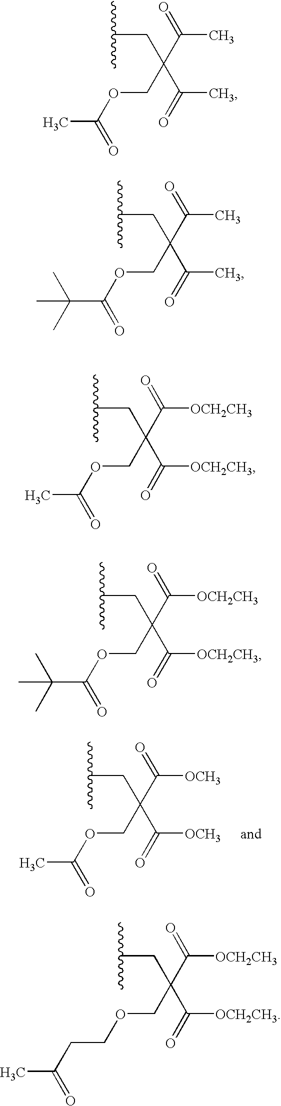

Additionally

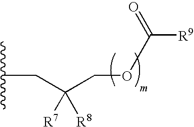

groups include the following.

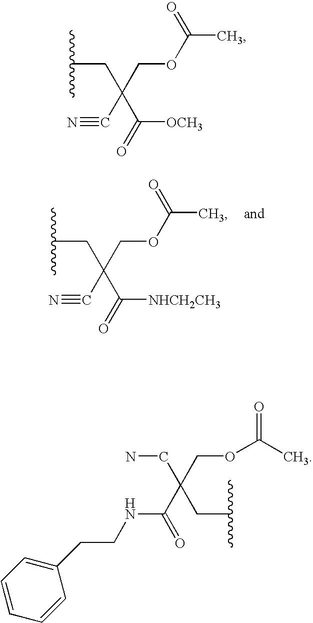

As with

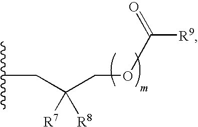

the substituents on

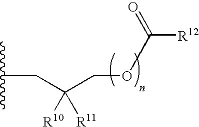

can vary. In some embodiments, R¹⁰ can be —C≡N and R¹¹ can be an optionally substituted C₁₋₈ alkoxycarbonyl such as —C(═O)OCH₃. In other embodiments, R¹⁰ can be —C≡N and R¹¹ can be an optionally substituted C₁₋₈ organylaminocarbonyl, for example, —C(═O)NHCH₂CH₃ and —C(═O)NHCH₂CH₂-phenyl. In some embodiments, R¹⁰ and R¹¹ can be independently C₁₋₈ organylcarbonyl or C₁₋₈ alkoxycarbonyl. In an embodiment, both R¹⁰ and R¹¹ can be an optionally substituted C₁₋₈ organylcarbonyl. In some embodiments, both R¹⁰ and R¹¹ can be —C(═O)CH₃. In other embodiments, both R¹⁰ and R¹¹ can be an optionally substituted C₁₋₈ alkoxycarbonyl. In an embodiment, both R¹⁰ and R¹¹ can be —C(═O)OCH₂CH₃. In another embodiment, both R¹⁰ and R¹¹ can be —C(═O)OCH₃. In some embodiments, including those described in this paragraph, R¹² can be an optionally substituted C₁₋₄-alkyl. In an embodiment, R¹² can be methyl or tert-butyl. In some embodiments, n can be 1. In an embodiment, n can be 1 and both R¹⁰ and R¹¹ can be an optionally substituted C₁₋₈ alkoxycarbonyl or an optionally substituted C₁₋₈ organylcarbonyl. In other embodiments, n can be 2. In an embodiment, n can be 2 and both R¹⁰ and R¹¹ can be an optionally substituted C₁₋₈ alkoxycarbonyl. In another embodiment, n can be 2 and both R¹⁰ and R¹¹ can be an optionally substituted C₁₋₈ organylcarbonyl. In some embodiments R¹⁰ and R¹¹ can be the same. In other embodiments, R¹⁰ and R¹¹ can be different.

The substituents R¹³, R¹⁴ and R¹⁵ on

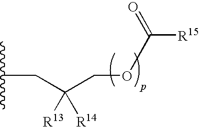

can also vary. In some embodiments R¹³ and R¹⁴ can be the same. In other embodiments, R¹³ and R¹⁴ can be different. In some embodiments, R¹³ can be —C≡N and R¹⁴ can be an optionally substituted C₁₋₈ alkoxycarbonyl such as —C(═O)OCH₃. In other embodiments, R¹³ can be —C≡N and R¹⁴ can be an optionally substituted C₁₋₈ organylaminocarbonyl, for example, —C(═O)NHCH₂CH₃ and —C(═O)NHCH₂CH₂-phenyl. In some embodiments, R¹³ and R¹⁴ can be independently C₁₋₈ organylcarbonyl or C₁₋₈ alkoxycarbonyl. In an embodiment, both R¹³ and R¹⁴ can be an optionally substituted C₁₋₈ organylcarbonyl. In some embodiments, both R¹³ and R¹⁴ can be —C(═O)CH₃. In other embodiments, both R¹³ and R¹⁴ can be an optionally substituted C₁₋₈ alkoxycarbonyl. In an embodiment, both R¹³ and R¹⁴ can be —C(═O)NCH₂CH₃. In another embodiment, both R¹³ and R¹⁴ can be —C(═O)OCH₃. In some embodiments, including those described in this paragraph, R¹⁵ can be an optionally substituted C₁₋₄alkyl. In an embodiment, R¹⁵ can be methyl or tert-butyl. In some embodiments, p can be 1. In an embodiment, p can be 1 and both R¹³ and R¹⁴ can be an optionally substituted C₁₋₈ alkoxycarbonyl or an optionally substituted C₁₋₈ organylcarbonyl. In other embodiments, p can be 2. In an embodiment, p can be 2 and both R¹³ and R¹⁴ can be an optionally substituted C₁₋₈ alkoxycarbonyl. In another embodiment, p can be 2 and both R¹³ and R¹⁴ can be an optionally substituted C₁₋₈ organylcarbonyl.

Examples of suitable R² and R³ groups include, but are not limited to, the following:

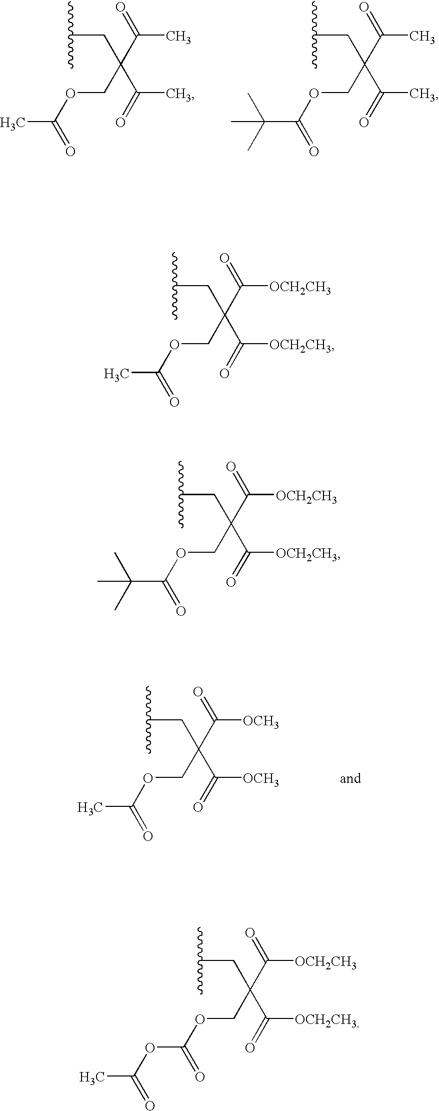

Additional examples of suitable R² and R³ groups include:

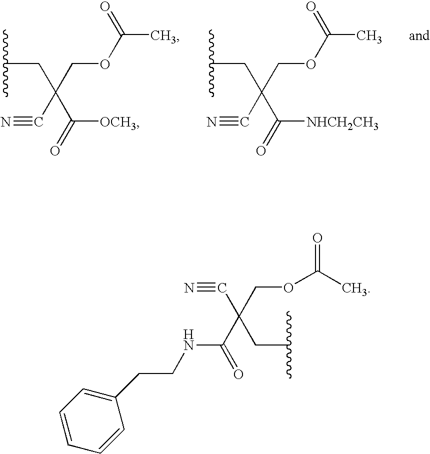

In some embodiments, when R¹ is

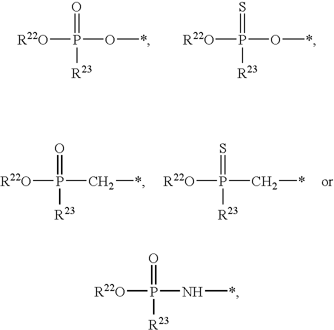

R²² can be selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl; and R²³ can be independently selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. Those skilled in the art understand that when R²² is absent, the oxygen will have an associated negative charge. In other embodiments, R²² can be hydrogen and R²³ can be an optionally substituted C₁₋₆ alkyl. In an embodiment, R²² can be hydrogen and R²³ can be methyl or ethyl. In other embodiments, R²² can be hydrogen, and R²³ can be NR²⁴R²⁵, wherein R²⁴ and R²⁵ can be each independently selected from hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In some embodiments, R²⁴ and R²⁵ can be each independently hydrogen or an optionally substituted C₁₋₆-alkyl.

In some embodiments, the optionally substituted heterocyclic base, B¹, can be selected from:

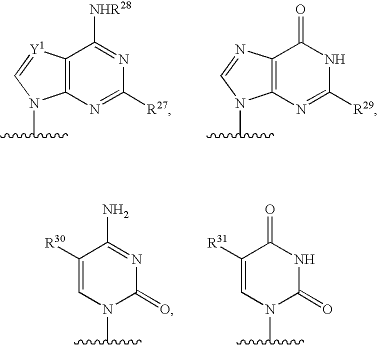

and

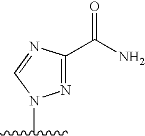

wherein: R²⁷ can be hydrogen or halogen; R²⁸ can be hydrogen, an optionally substituted C₁₋₄ alkyl, or an optionally substituted C₃₋₈ cycloalkyl; R²⁹ can be hydrogen or amino; R³⁰ can be selected from hydrogen, halogen, an optionally substituted C₁₋₄ alkyl, an optionally substituted C₂₋₄ alkenyl and an optionally substituted C₂₋₄ alkynyl; R³¹ can be selected from hydrogen, halogen, an optionally substituted C₁₋₄ alkyl, an optionally substituted C₂₋₄ alkenyl and an optionally substituted C₂₋₄ alkynyl; and Y¹ can be N or CR³², wherein R³² can be selected from hydrogen, halogen, an optionally substituted C₁₋₄-alkyl, an optionally substituted C₂₋₄-alkenyl and an optionally substituted C₂₋₄-alkynyl. In some embodiments, B¹ can be

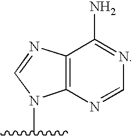

In other embodiments, B¹ can be

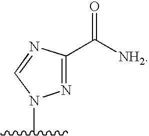

The 3′-position of the middle residue can also vary. For example, in some embodiments, R⁴ can be hydroxy. In other embodiments, R⁴ can be hydrogen. In still other embodiments, R⁴ can be an optionally substituted —O—C₁₋₆ alkyl. In an embodiment, R⁴ can be an unsubstituted or substituted methoxy group. In yet still other embodiments, R⁴ can be —OC(R⁵)₂—O—C(═O)R⁶, for example, —OCH₂—O—C(═O)Me or —OCH₂—O—C(═O)-t-butyl.

An exemplary structure of NS¹ is:

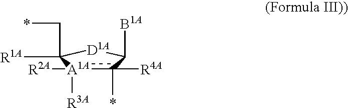

in which [symbol] can be a double or single bond; A^{1A} can be selected from C (carbon), O (oxygen) and S (sulfur); B^{1A} can be an optionally substituted heterocyclic base; D^{1A} can be C═CH₂ or O (oxygen); R^{1A} can be selected from hydrogen, azido, —CN, an optionally substituted C₁₋₄ alkyl and an optionally substituted C₁₋₄ alkoxy; R^{2A} can be absent or selected from hydrogen, halogen, hydroxy and an optionally substituted C₁₋₄ alkyl; R^{3A} can be absent or selected from hydrogen, halogen, azido, amino, hydroxy, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —O—C(R^{5A})₂—O—C(═O)R^{6A}; R^{4A} can be absent or selected from hydrogen, halogen, hydroxy, —CN, —NC, an optionally substituted C₁₋₄ alkyl, an optionally substituted haloalkyl and an optionally substituted hydroxyalkyl; each R^{5A} and R^{6A} can be independently hydrogen or an optionally substituted C₁₋₄-alkyl; and * represents a point of attachment. It is understood by those skilled in the art that when A^{1A}O or S, [symbol] is a single bond. Likewise, those skilled in the art understand that a carbon has four bonds. For example when [symbol] is a double bond, then either R^{2A} or R^{3A} must be absent and R^{4A} must be absent.

In some embodiments, NS¹ can be:

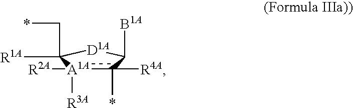

wherein: [symbol] can be a single bond; A^{1A} can be C; B^{1A} can be an optionally substituted heterocyclic base; D^{1A} can be O; R^{1A} can be hydrogen; R^{2A} can be hydrogen; R^{3A} can be selected from hydrogen, hydroxy, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R^{5A})₂—O—C(═O)R^{6A}; each R^{5A} and R^{6A} can be independently hydrogen or an optionally substituted C₁₋₄-alkyl; and * represents a point of attachment.

The substituent R^{3A}, in some embodiments, can be an optionally substituted —O—C₁₋₆ alkyl. In an embodiment, R^{3A} can be —OCH₃. In other embodiments, R^{3A} can be —OC(R^{5A})₂—O—C(═O)R^{6A}. In an embodiment, when R^{3A} can be —OC(R^{5A})₂—O—C(═O)R^{6A}, both R^{5A} groups can be hydrogen and R^{6A} can be an optionally substituted alkyl (e.g., methyl and t-butyl). In some embodiments, R^{3A} cannot be hydroxy. In other embodiments, R^{3A} cannot be hydrogen. In still other embodiments, R^{3A} cannot be an optionally substituted —O—C₁₋₆ alkyl, such as methoxy. In some embodiments, including those in this paragraph, A^{1A} can be carbon, D^{1A} can be oxygen, and [symbol] can be a single bond.

In some embodiments, the heterocyclic base represented by B^{1A} can be selected from:

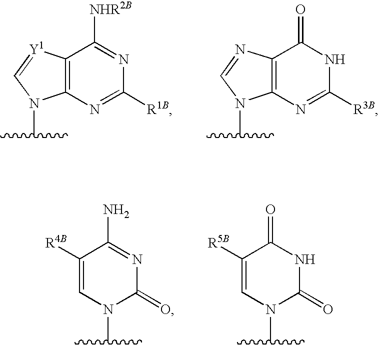

and

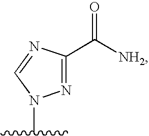

wherein: R^{1B} can be hydrogen or halogen; R^{2B} can be hydrogen, an optionally substituted C₁₋₄ alkyl, or an optionally substituted C₃₋₈ cycloalkyl; R^{3B} can be hydrogen or amino; R^{4B} can be selected from hydrogen, halogen, an optionally substituted C₁₋₄ alkyl, an optionally substituted C₂₋₄ alkenyl and an optionally substituted C₂₋₄ alkynyl; R^{5B} can be selected from hydrogen, halogen, an optionally substituted C₁₋₄ alkyl, an optionally substituted C₂₋₄ alkenyl and an optionally substituted C₂₋₄ alkynyl; and Y^{1B} can be N or CR^{6B}, wherein R^{6B} can be selected from hydrogen, halogen, an optionally substituted C₁₋₄-alkyl, an optionally substituted C₂₋₄ alkenyl and an optionally substituted C₂₋₄ alkynyl. In some embodiments, B^{1A} can be

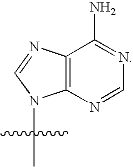

In other embodiments, B^{1A} can be

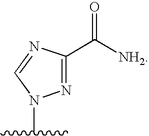

Examples of suitable NS¹ groups include, but are not limited to, the following:

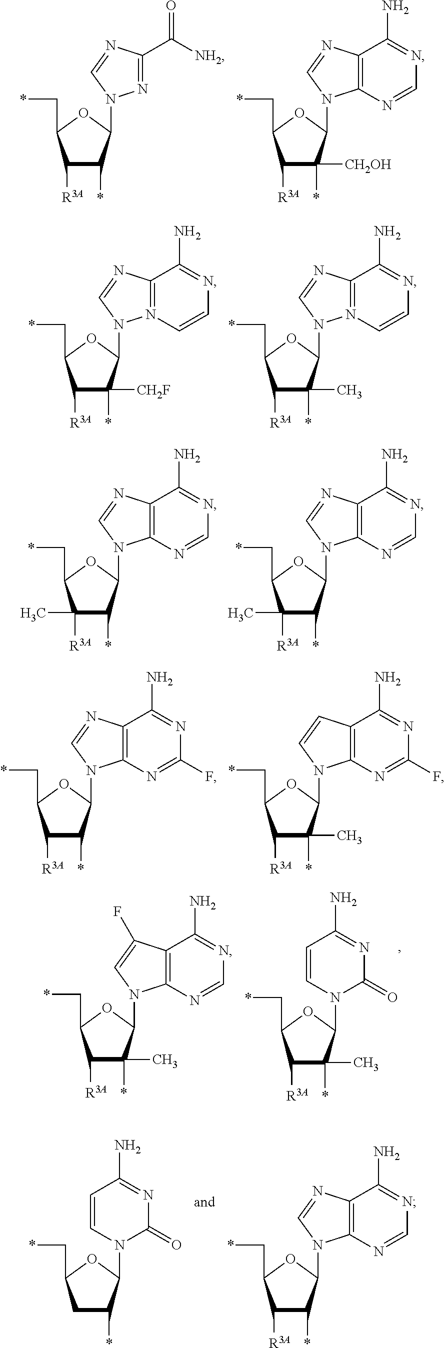

in which * represents a point of attachment; and R^{3A} can be absent or selected from hydrogen, halogen, azido, amino, hydroxy, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R^{16A})₂—O—C(═O)R^{17A}. In some embodiments, R^{3A} can be an optionally substituted —O—C₁₋₆ alkyl, for example, —OCH₃. In other embodiments, R^{3A} can be —OC(R^{5A})₂—O—C(═O)R^{6A}. In an embodiment, when R^{3A} can be —OC(R^{5A})₂—O—C(═O)R^{6A}, both R^{5A} groups can be hydrogen and R^{6A} can be an optionally substituted C₁₋₄ alkyl (e.g., methyl and t-butyl).

An exemplary structure of NS² is:

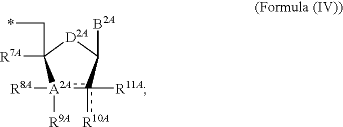

in which each [symbol] can be a double or single bond; A^{2A} can be selected from C (carbon), O (oxygen) and S (sulfur); B^{2A} can be an optionally substituted heterocyclic base; D^{2A} can be C═CH₂ or O (oxygen); R^{7A} can be selected from hydrogen, azido, —CN, an optionally substituted C₁₋₄ alkyl and an optionally substituted C₁₋₄ alkoxy; R^{8A} can be absent or selected from hydrogen, halogen, hydroxy and an optionally substituted C₁₋₄ alkyl; R^{9A} can be absent or selected from hydrogen, halogen, azido, amino and hydroxy; R^{10A} can be absent or selected from hydrogen, halogen, hydroxy, —CN, —NC, an optionally substituted C₁₋₄ alkyl and an optionally substituted C₁₋₄ alkoxy; R^{11A} can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R^{10A} indicated by [symbol] is a double bond, then R^{10A} is a C₁₋₄ alkenyl and R^{11A} is absent; and * represents a point of attachment. Those skilled in the art understand that when A^{2A} O or S, [symbol] is a single bond. Those skilled in the art also understand that when A^{2A} is C, the carbon can have four bonds. Likewise, the 2′-carbon of NS² can have four bonds. Thus, when [symbol] connecting the 2′ and 3′-carbons is a double bond, either R^{8A} or R^{9A} must be absent, and R^{10A} or R^{11A} must be absent. In addition, both [symbol] cannot be simultaneously double bonds. In some embodiments, A^{2A} can be carbon, D^{2A} can be oxygen, and each [symbol] can be a single bond.

In some embodiments, the optionally substituted heterocyclic base, B^{2A}, can be selected from one of the following:

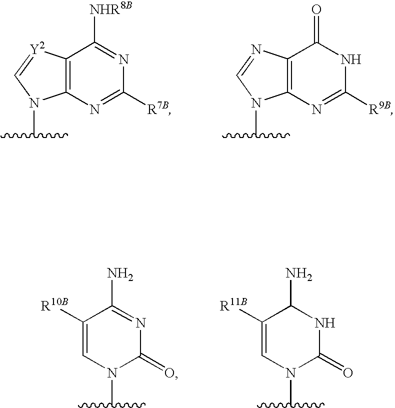

and

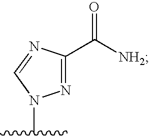

in which R^{7B} can be hydrogen or halogen; R^{8B} can be hydrogen, an optionally substituted C₁₋₄ alkyl, or an optionally substituted C₃₋₈ cycloalkyl; R^{9B} can be hydrogen or amino; R^{10B} can be selected from the group consisting of hydrogen, halogen, an optionally substituted C₁₋₄ alkyl, an optionally substituted C₂₋₄ alkenyl and an optionally substituted C₂₋₄ alkynyl; R^{11B} can be selected from the group consisting of hydrogen, halogen, an optionally substituted C₁₋₄alkyl, an optionally substituted C₂₋₄ alkenyl and an optionally substituted C₂₋₄ alkynyl; and Y^{2B} can be N or CR^{12B}, wherein R^{12B} can be selected from the group consisting of hydrogen, halogen, an optionally substituted C₁₋₄-alkyl, an optionally substituted C₂₋₄-alkenyl and an optionally substituted C₂₋₄-alkynyl. In some embodiments, B^{2A} can be

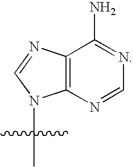

In some embodiments, B^{2A} can be

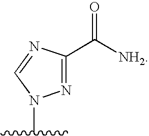

In some embodiments, B^{1A} and B^{2A} can be the same, for example, both B^{1A} and B^{2A} can be

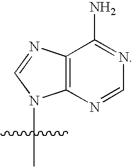

In other embodiments, B^{1A} and B^{2A} can be different. As an example, one of B^{1A} and B^{2A} can be

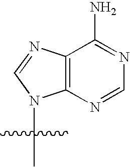

and the other of B^{1A} and B^{2A} can be

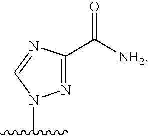

Suitable examples of NS² include, but are not limited to, the following:

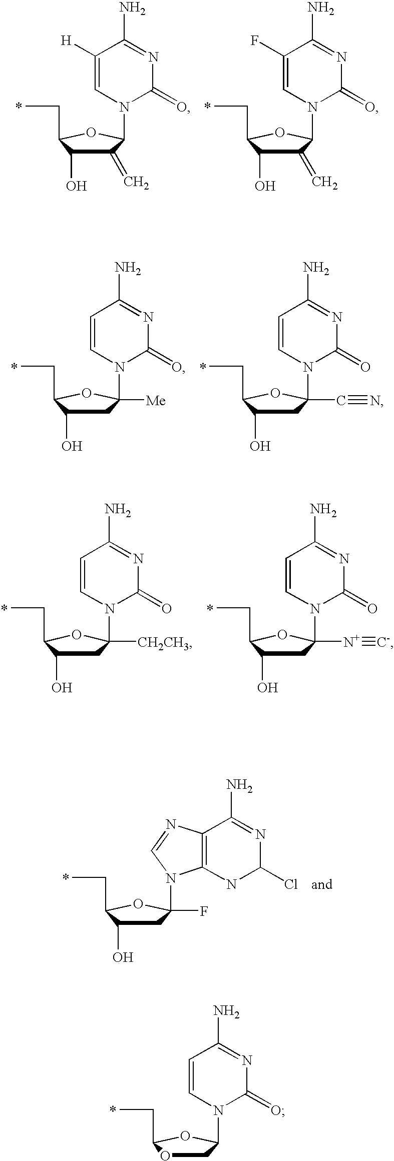

in which * represents a point of attachment.

Additional examples of NS² include the following:

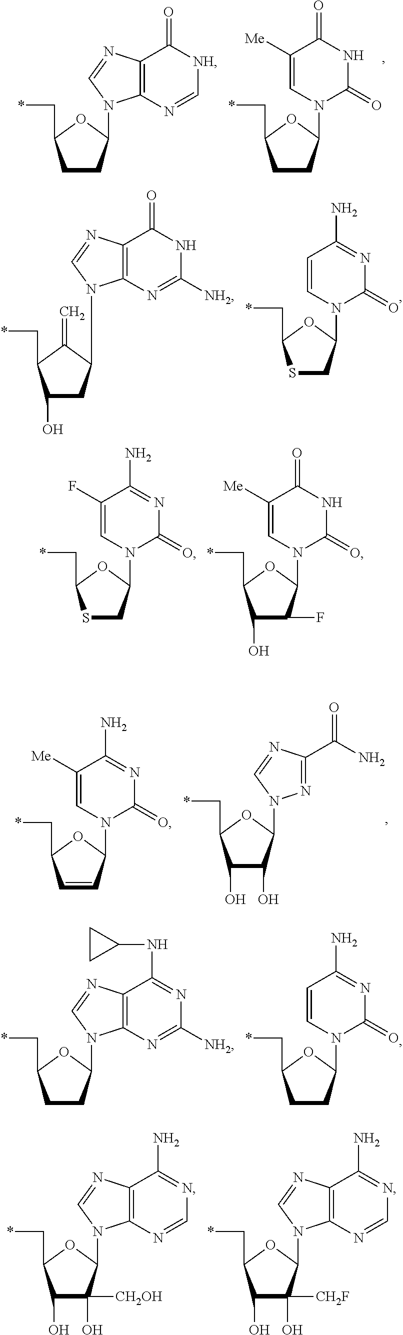

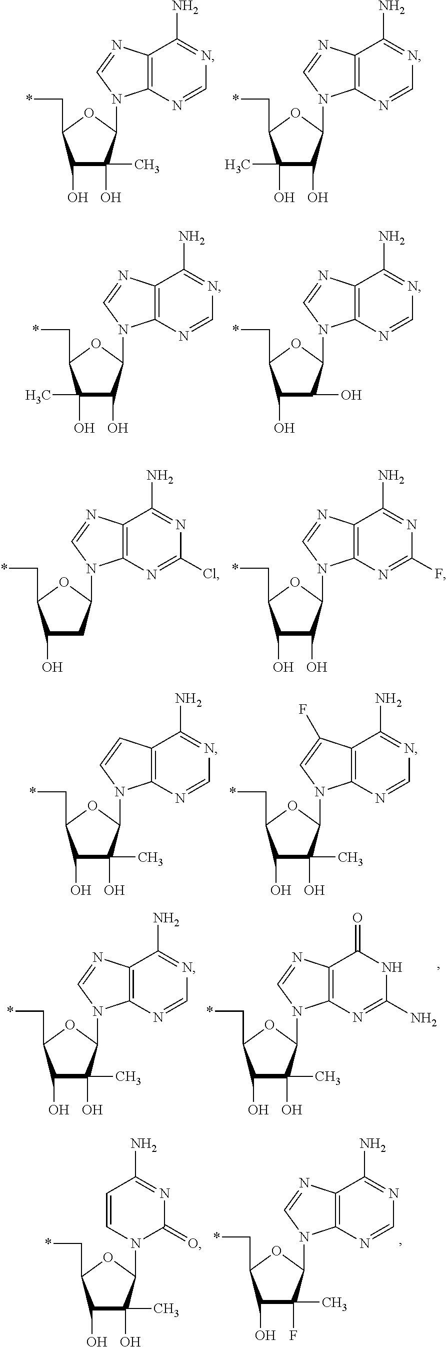

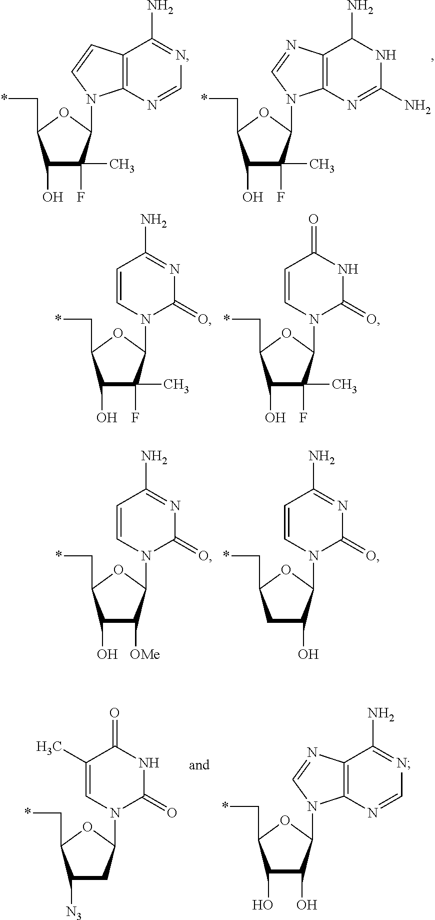

in which * represents a point of attachment.

In some embodiments, the compound of Formula (I) can have NS¹ as

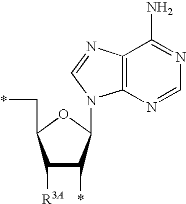

### and NS² as

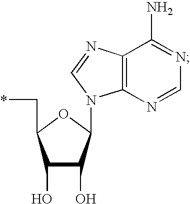

in which R^{3A} can be selected from —OH, an optionally substituted —O—C₁₋₆ alkyl and —OC(R^{5A})₂—O—C(═O)R^{6A}; each R^{5A} and R^{6A} can be independently hydrogen or an optionally substituted C₁₋₄-alkyl; and * represents a point of attachment. In an embodiment, R^{3A} can be —OC(R^{5A})₂—O—C(═O)R^{6A}. In some embodiments when R^{3A} is —OC(R^{5A})₂—O—C(═O)R^{6A}, then both R^{5A} groups can be hydrogen and R^{6A} can be an optionally substituted C₁₋₄-alkyl, such as methyl or t-butyl. In another embodiment, R^{3A} can be an optionally substituted —O—C₁₋₆ alkyl, such as methoxy.

The 5′-terminal residue and the 2′-terminal residue can be various nucleoside residues. In some embodiments, NS¹ can be selected from anti-neoplastic agent, an anti-viral agent, an anti-bacterial agent and an anti-parasitic agent. Similarly, in some embodiments, NS² can be selected from anti-neoplastic agent, an anti-viral agent, an anti-bacterial agent and an anti-parasitic agent. In some embodiments, NS¹ can be a nucleoside analog drug. In some embodiments, NS² can be a nucleoside analog drug. The anti-viral agent can have activity against various viruses, including, but not limited to, one or more of the following: an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae, a Togaviridae, an Arenaviridae and/or a Bornaviridae. In some embodiments, when NS¹ and/or N5² is an anti-neoplastic agent, in some embodiments, the compound of Formula (I) can have activity against cancer, tumors (e.g., solid tumors) and the like. Similarly, in some embodiments, when NS¹ and/or NS² is an anti-parasitic agent, in an embodiment, the compound of Formula (I) can have activity against Chagas' disease. In some embodiments, when NS¹ and/or NS² is an anti-bacterial agent, compound of Formula (I) can have activity against a bacterial infection, for example, an infection caused anthrax and/or E. coli. In some embodiments, NS¹ and NS² can be the same (for example, have the same structure and/or be active against the same disease). In other embodiments, NS¹ and NS² can be different (for example, have the same structure and/or be active against the same disease).

As previously stated, NS¹ and/or NS² can be a nucleoside analog drug, an anti-viral agent, an anti-bacterial agent, an anti-neoplastic agent and/or an anti-parasitic agent. In some embodiments, the nucleoside analog drug can be selected to treat a particular disease and/or condition, thereby providing a dual mode of action Likewise, in some embodiments, the anti-viral agent, an anti-bacterial agent, anti-neoplastic agent and anti-parasitic agent can be selected to target a particular virus, bacteria, tumor or parasite, thereby providing a dual mode of action. Upon administration of one or more compounds of Formula (I) to an animal, such as a human, a non-human mammal, a bird, or another animal, the full molecule can activate RNaseL, producing a general anti-viral response, and upon degradation of the compound in vivo, the nucleoside(s) is released, thus generating the particular (generally more specific) therapeutic action (e.g., anti-viral, anti-bacterial anti-neoplastic and/or anti-parasitic action) of that moiety. Further, upon release of the nucleoside(s), the intracellular cleavage releases not a nucleoside, but its active, phosphorylated form. This not only makes the nucleoside(s) more immediately available in the intracellular environment, but also bypasses some potential resistance mechanisms such as those described herein. One mechanism that is bypassed is the need for kinase-mediated phosphorylation that both reduces the efficacy of nucleosides in general, but also provides a potential resistance mechanism. This dual-mode of action can provide a powerful benefit in addressing difficult diseases, conditions, neoplasms, viral infections, bacterial infections and/or parasitic infections.

A non-limiting list of examples of compounds of Formula (I) are shown herein in Table 1.

TABLE 1

| R_{¹} | L_{¹} | L_{²} | R_{⁴} |
|---|---|---|---|
| —(CH^{₂})^{ₐ}—OR_{¹⁶} | PHO | PHO | —H. —OH, —OMe or |
| | | | —C(R_{⁵})^{₂}—O—C(═O)R_{⁶} |
| —(CH^{₂})^{ₐ}—OR_{¹⁶} | PHO | PHS | —H. —OH, —OMe or |
| | | | —C(R_{⁵})^{₂}—O—C(═O)R_{⁶} |
| —(CH^{₂})^{ₐ}—OR_{¹⁶} | PHS | PHO | —H. —OH, —OMe or |
| | | | —C(R_{⁵})^{₂}—O—C(═O)R_{⁶} |
| —(CH^{₂})^{ₐ}—OR_{¹⁶} | PHS | PHS | —H. —OH, —OMe or |
| | | | —C(R_{⁵})^{₂}—O—C(═O)R_{⁶} |
| —(CH^{₂})^{ₐ}—OR_{¹⁶} | PHOP | PHOP | —H. —OH, —OMe or |
| | | | —C(R_{⁵})^{₂}—O—C(═O)R_{⁶} |
| —(CH^{₂})^{ₐ}—OR_{¹⁶} | PHOP | PHS | —H. —OH, —OMe or |
| | | | —C(R_{⁵})^{₂}—O—C(═O)R_{⁶} |
| —(CH^{₂})^{ₐ}—OR_{¹⁶} | PHS | PHOP | —H. —OH, —OMe or |
| | | | —C(R_{⁵})^{₂}—O—C(═O)R_{⁶} |
| —(CH^{₂})^{_{b}}—COOR_{¹⁶} | PHO | PHO | —H. —OH, —OMe or |
| | | | —C(R_{⁵})^{₂}—O—C(═O)R_{⁶} |
| —(CH^{₂})^{_{b}}—COOR_{¹⁶} | PHO | PHS | —H. —OH, —OMe or |
| | | | —C(R_{⁵})^{₂}—O—C(═O)R_{⁶} |
| —(CH^{₂})^{_{b}}—COOR_{¹⁶} | PHS | PHO | —H. —OH, —OMe or |
| | | | —C(R_{⁵})^{₂}—O—C(═O)R_{⁶} |
| —(CH^{₂})^{_{b}}—COOR_{¹⁶} | PHS | PHS | —H. —OH, —OMe or |
| | | | —C(R_{⁵})^{₂}—O—C(═O)R_{⁶} |
| —(CH^{₂})^{_{b}}—COOR_{¹⁶} | PHOP | PHOP | —H. —OH, —OMe or |
| | | | —C(R_{⁵})^{₂}—O—C(═O)R_{⁶} |
| —(CH^{₂})^{_{b}}—COOR_{¹⁶} | PHOP | PHS | —H. —OH, —OMe or |
| | | | —C(R_{⁵})^{₂}—O—C(═O)R_{⁶} |
| —(CH^{₂})^{_{b}}—COOR_{¹⁶} | PHS | PHOP | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHO | PHO | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHO | PHS | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHS | PHO | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHS | PHS | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHOP | PHOP | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHOP | PHS | —H. —OH, —OMe or |
| | | | —C(R_{⁵})^{₂}—O—C(═O)R_{⁶} |
| —(CH^{₂})^{_{c}}—C(═O)NR_{¹⁷}R_{¹⁸} | PHS | PHOP | —H. —OH, —OMe or |
| | | | —C(R_{⁵})^{₂}—O—C(═O)R_{⁶} |
| A-A | PHO | PHO | —H. —OMe or |
| | | | —C(R_{⁵})^{₂}—O—C(═O)R_{⁶} |
| A-A | PHO | PHS | —H. —OH, —OMe or |
| | | | —C(R_{⁵})^{₂}—O—C(═O)R_{⁶} |
| A-A | PHS | PHO | —H. —OH, —OMe or |
| | | | —C(R_{⁵})^{₂}—O—C(═O)R_{⁶} |
| A-A | PHS | PHS | —H. —OH, —OMe or |
| | | | —C(R_{⁵})^{₂}—O—C(═O)R_{⁶} |
| A-A | PHOP | PHOP | —H. —OH, —OMe or |
| | | | —C(R_{⁵})^{₂}—O—C(═O)R_{⁶} |
| A-A | PHOP | PHS | —H. —OH, —OMe or |
| | | | —C(R_{⁵})^{₂}—O—C(═O)R_{⁶} |
| A-A | PHS | PHOP | —H. —OH, —OMe or |
| | | | —C(R_{⁵})^{₂}—O—C(═O)R_{⁶} |
| B-B | PHO | PHO | —H. —OH, —OMe or |
| | | | —C(R_{⁵})^{₂}—O—C(═O)R_{⁶} |
| B-B | PHO | PHS | —H. —OH, —OMe or |
| | | | —C(R_{⁵})^{₂}—O—C(═O)R_{⁶} |
| B-B | PHS | PHO | —H. —OH, —OMe or |
| | | | —C(R_{⁵})^{₂}—O—C(═O)R_{⁶} |
| B-B | PHS | PHS | —H. —OMe or |
| | | | —C(R_{⁵})^{₂}—O—C(═O)R_{⁶} |
| B-B | PHOP | PHOP | —H. —OH, —OMe or |
| | | | —C(R_{⁵})^{₂}—O—C(═O)R_{⁶} |
| B-B | PHOP | PHS | —H. —OH, —OMe or |
| | | | —C(R_{⁵})^{₂}—O—C(═O)R_{⁶} |
| B-B | PHS | PHOP | —H. —OH, —OMe or |
| | | | —C(R_{⁵})^{₂}—O—C(═O)R_{⁶} |
| C-C | PHO | PHO | —H. —OH, —OMe or |
| | | | —C(R_{⁵})^{₂}—O—C(═O)R_{⁶} |
| C-C | PHO | PHS | —H. —OH, —OMe or |
| | | | —C(R_{⁵})^{₂}—O—C(═O)R_{⁶} |
| C-C | PHS | PHO | —H. —OH, —OMe or |
| | | | —C(R_{⁵})^{₂}—O—C(═O)R_{⁶} |
| C-C | PHS | PHS | —H. —OH, —OMe or |
| | | | —C(R_{⁵})^{₂}—O—C(═O)R_{⁶} |
| C-C | PHOP | PHOP | —H. —OH, —OMe or |
| | | | —C(R_{⁵})^{₂}—O—C(═O)R_{⁶} |
| C-C | PHOP | PHS | —H. —OH, —OMe or |
| | | | —C(R_{⁵})^{₂}—O—C(═O)R_{⁶} |
| C-C | PHS | PHOP | —H. —OH, —OMe or |
| | | | —C(R_{⁵})^{₂}—O—C(═O)R_{⁶} |
| D-D | PHO | PHO | —H. —OH, —OMe or |
| | | | —C(R_{⁵})^{₂}—O—C(═O)R_{⁶} |
| D-D | PHO | PHS | —H. —OH, —OMe or |
| | | | —C(R_{⁵})^{₂}—O—C(═O)R_{⁶} |
| D-D | PHS | PHO | —H. —OH, —OMe or |
| | | | —C(R_{⁵})^{₂}—O—C(═O)R_{⁶} |
| D-D | PHS | PHS | —H. —OH, —OMe or |
| | | | —C(R_{⁵})^{₂}—O—C(═O)R_{⁶} |
| D-D | PHOP | PHOP | —H. —OH, —OMe or |
| | | | —C(R_{⁵})^{₂}—O—C(═O)R_{⁶} |
| D-D | PHOP | PHS | —H. —OH, —OMe or |
| | | | —C(R_{⁵})^{₂}—O—C(═O)R_{⁶} |
| D-D | PHS | PHOP | —H. —OH, —OMe or |
| | | | —C(R_{⁵})^{₂}—O—C(═O)R_{⁶} |
| E-E | PHO | PHO | —H. —OH, —OMe or |
| | | | —C(R_{⁵})^{₂}—O—C(═O)R_{⁶} |
| E-E | PHO | PHS | —H. —OH, —OMe or |
| | | | —C(R_{⁵})^{₂}—O—C(═O)R_{⁶} |
| E-E | PHS | PHO | —H. —OH, —OMe or |
| | | | —C(R_{⁵})^{₂}—O—C(═O)R_{⁶} |
| E-E | PHS | PHS | —H. —OH, —OMe or |
| | | | —C(R_{⁵})^{₂}—O—C(═O)R_{⁶} |
| E-E | PHOP | PHOP | —H. —OH, —OMe or |
| | | | —C(R_{⁵})^{₂}—O—C(═O)R_{⁶} |
| E-E | PHOP | PHS | —H. —OH, —OMe or |
| | | | —C(R_{⁵})^{₂}—O—C(═O)R_{⁶} |
| E-E | PHS | PHOP | —H. —OH, —OMe or |
| | | | —C(R_{⁵})^{₂}—O—C(═O)R_{⁶} |
| F-F | PHO | PHO | —H. —OH, —OMe or |
| | | | —C(R_{⁵})^{₂}—O—C(═O)R_{⁶} |
| F-F | PHO | PHS | —H. —OH, —OMe or |
| | | | —C(R_{⁵})^{₂}—O—C(═O)R_{⁶} |
| F-F | PHS | PHO | —H. —OH, —OMe or |
| | | | —C(R_{⁵})^{₂}—O—C(═O)R_{⁶} |
| F-F | PHS | PHS | —H. —OH, —OMe or |
| | | | —C(R_{⁵})^{₂}—O—C(═O)R_{⁶} |
| F-F | PHOP | PHOP | —H. —OH, —OMe or |
| | | | —C(R_{⁵})^{₂}—O—C(═O)R_{⁶} |
| F-F | PHOP | PHS | —H. —OH, —OMe or |
| | | | —C(R_{⁵})^{₂}—O—C(═O)R_{⁶} |
| F-F | PHS | PHOP | —H. —OH, —OMe or |
| | | | —C(R_{⁵})^{₂}—O—C(═O)R_{⁶} |
| G-G | PHO | PHO | —H. —OH, —OMe or |
| | | | —C(R_{⁵})^{₂}—O—C(═O)R_{⁶} |
| G-G | PHO | PHS | —H. —OH, —OMe or |
| | | | —C(R_{⁵})^{₂}—O—C(═O)R_{⁶} |
| G-G | PHS | PHO | —H. —OH, —OMe or |
| | | | —C(R_{⁵})^{₂}—O—C(═O)R_{⁶} |
| G-G | PHS | PHS | —H. —OH, —OMe or |
| | | | —C(R_{⁵})^{₂}—O—C(═O)R_{⁶} |
| G-G | PHOP | PHOP | —H. —OH, —OMe or |
| | | | —C(R_{⁵})^{₂}—O—C(═O)R_{⁶} |
| G-G | PHOP | PHS | —H. —OH, —OMe or |
| | | | —C(R_{⁵})^{₂}—O—C(═O)R_{⁶} |
| G-G | PHS | PHOP | —H. —OH, —OMe or |
| | | | —C(R_{⁵})^{₂}—O—C(═O)R_{⁶} |

### In Table 1:

A-A denotes

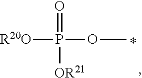

B-B, denotes

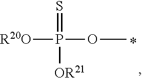

C-C denotes

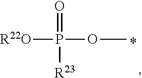

D-D denotes

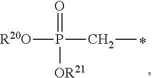

E-E denotes

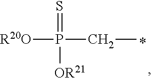

F-F denotes

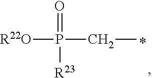

G-G denotes

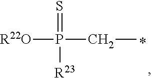

PHO denotes

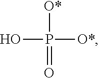

PHS denotes

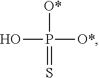

and its tautomer

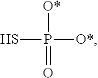

PHOP denotes

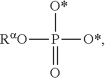

wherein R^{α} is pivaloyloxymethoxy, isopropyloxycarbonyloxymethoxy,

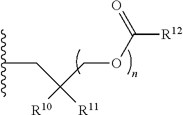

### if L¹ and

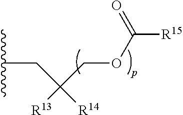

### if L².

Additional examples of compounds of Formula (I) are shown below.

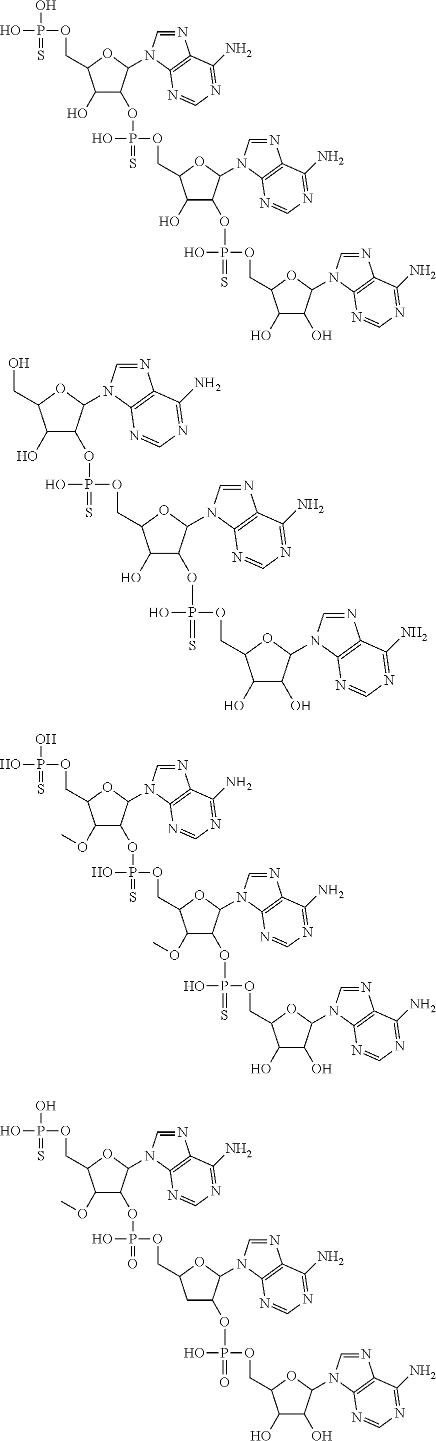

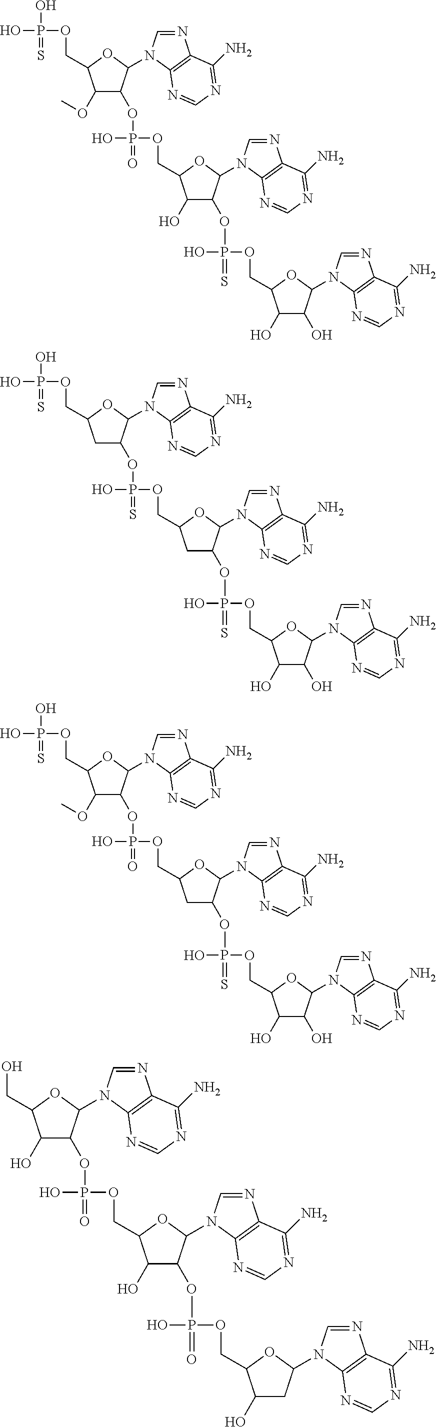

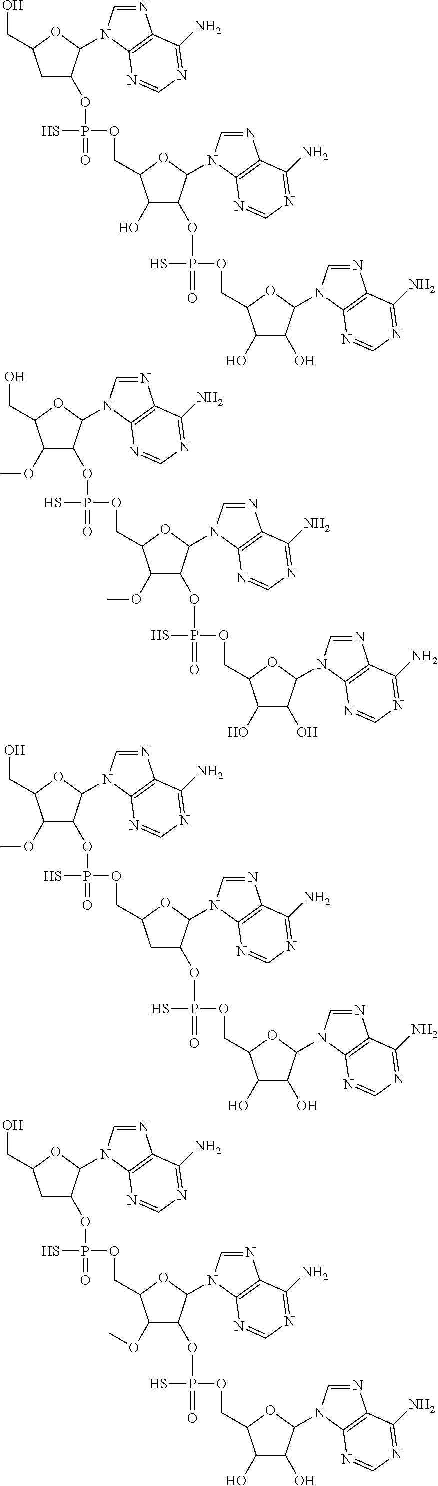

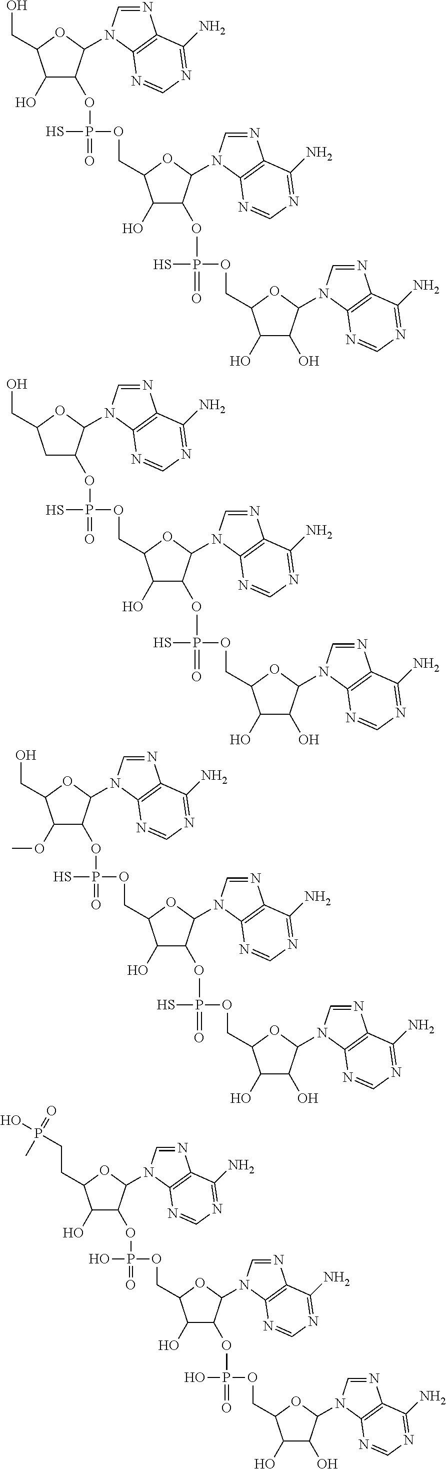

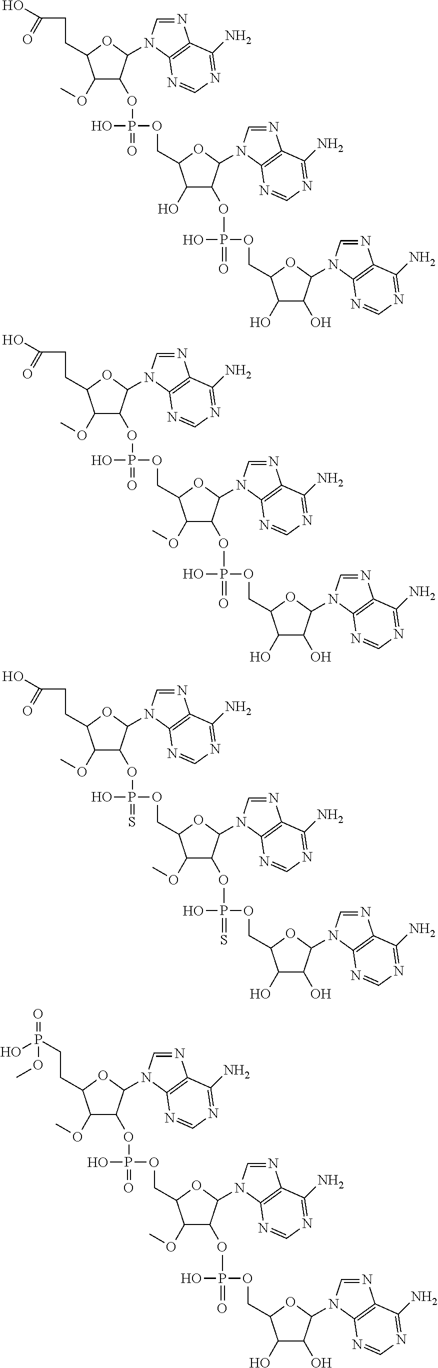

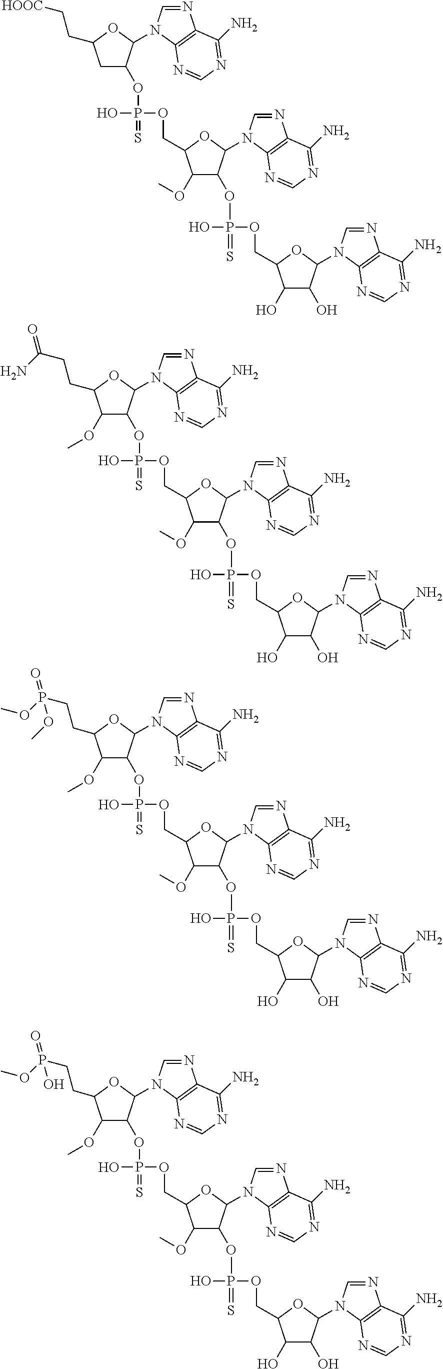

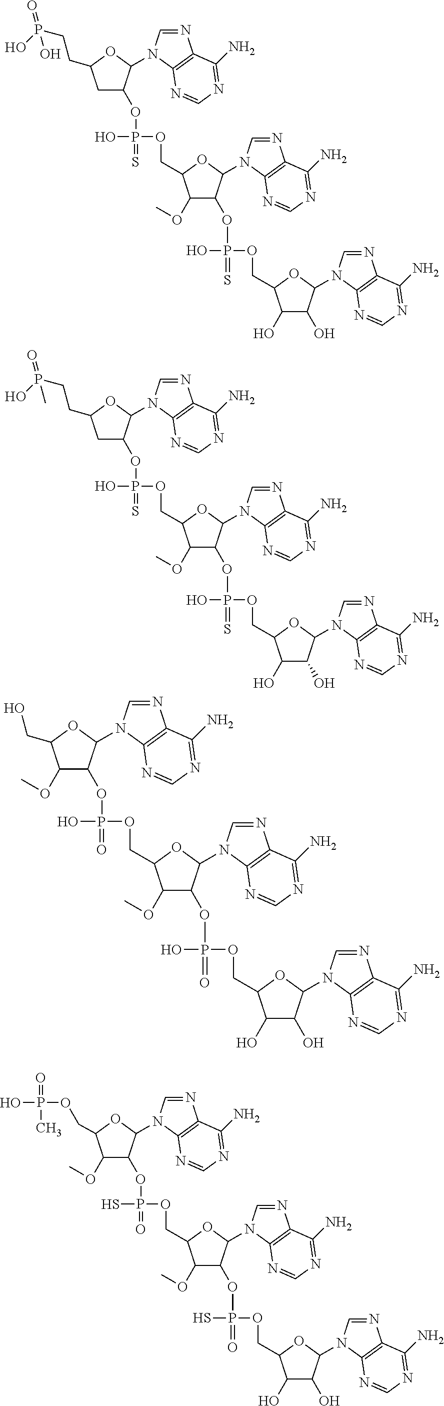

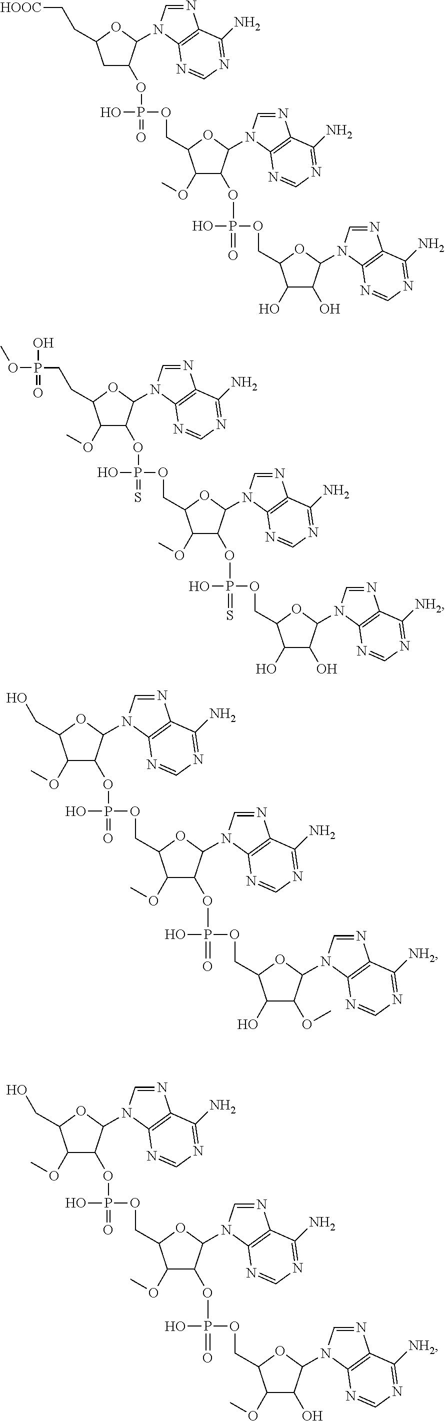

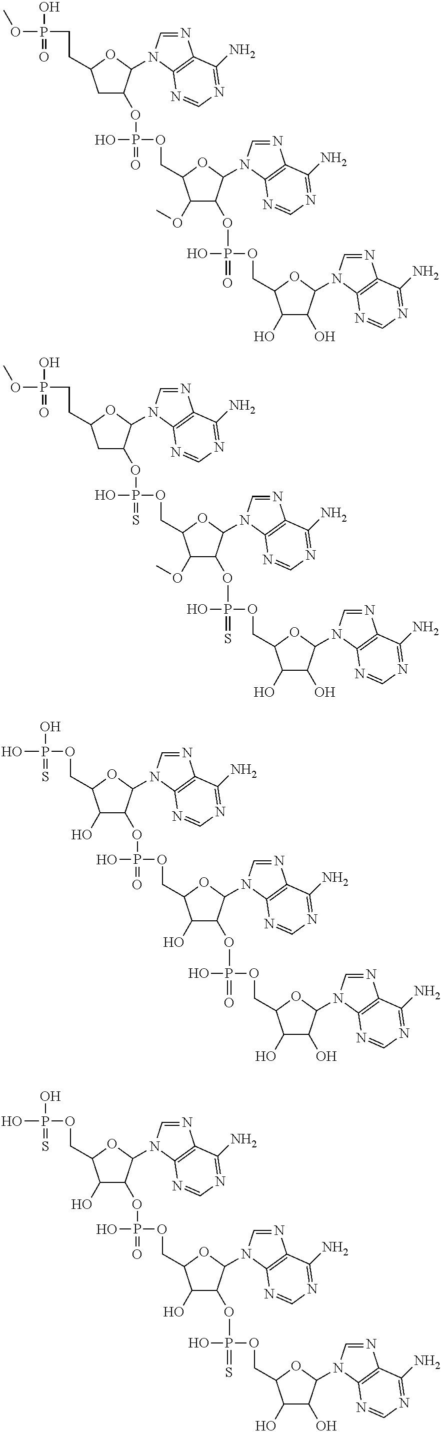

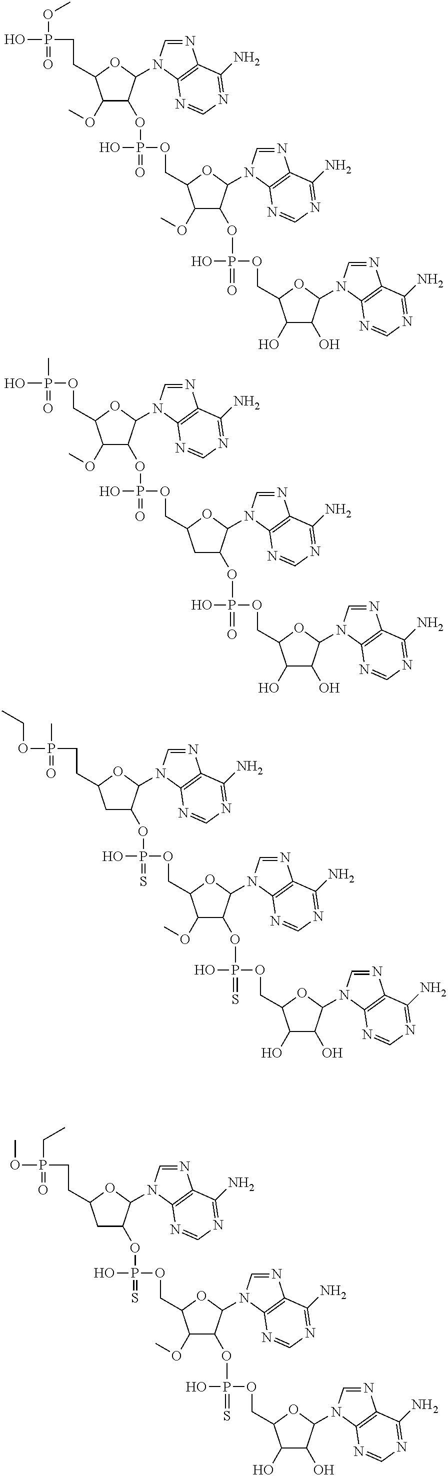

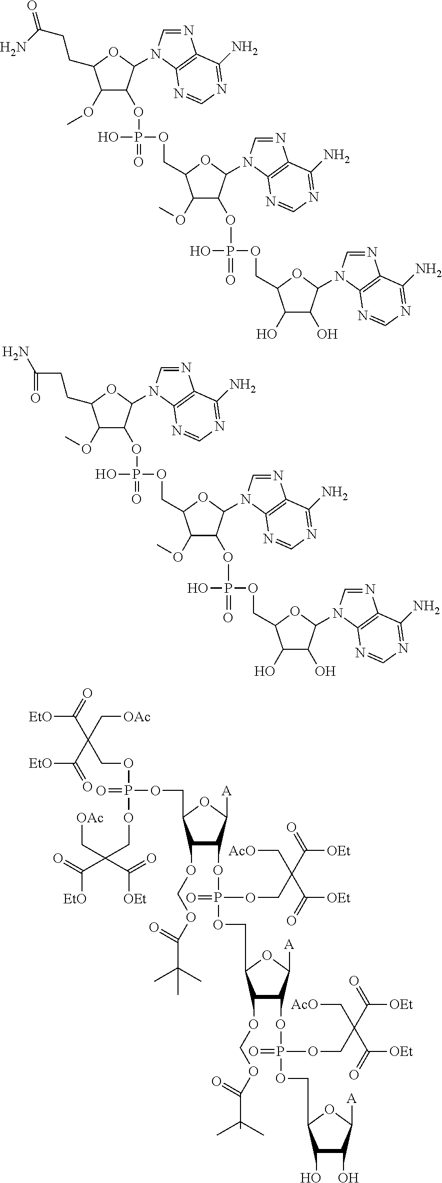

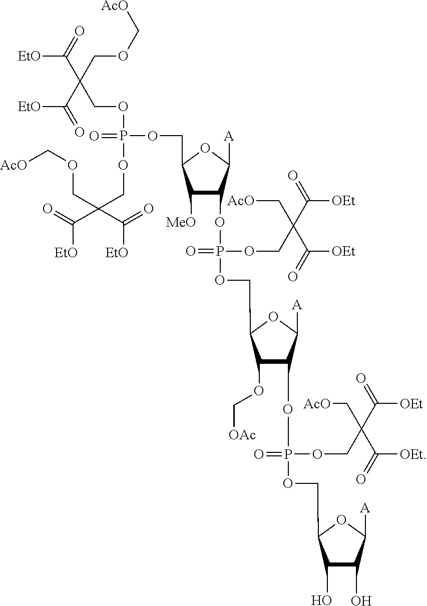

Other embodiments disclosed herein relate to a compound of Formula (Ia) as shown herein, or a pharmaceutically acceptable salt thereof, in which both R² and R³ can be

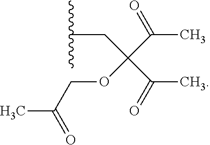

Still other embodiments disclosed herein relate to a compound of Formula (II) or a pharmaceutically acceptable salt thereof, wherein: R³³ can be selected from: —(CH₂)_{A}—OR³⁶, —O—CH₂—COOR³⁶, —(CH₂)_{B}—COOR³⁶, —(CH₂)_{C}—C(═S)OR³⁶, —(CH₂)_{D}—C(═O)NR³⁷R³⁸, —(CH₂)_{E}—NH—SO₂—R³⁶, —(CH₂)_{F}—NH—SO₂—NR³⁷R³⁸, —(CH₂)_{G}—NH—CO₂—R³⁶, —(CH₂)_{H}—NH—C(═O)—R³⁶, —(CH₂)_{I}—NH—C(═O)—NR³⁷R³⁸, —CH₂—C(R³⁹)₂—CH₂OH,

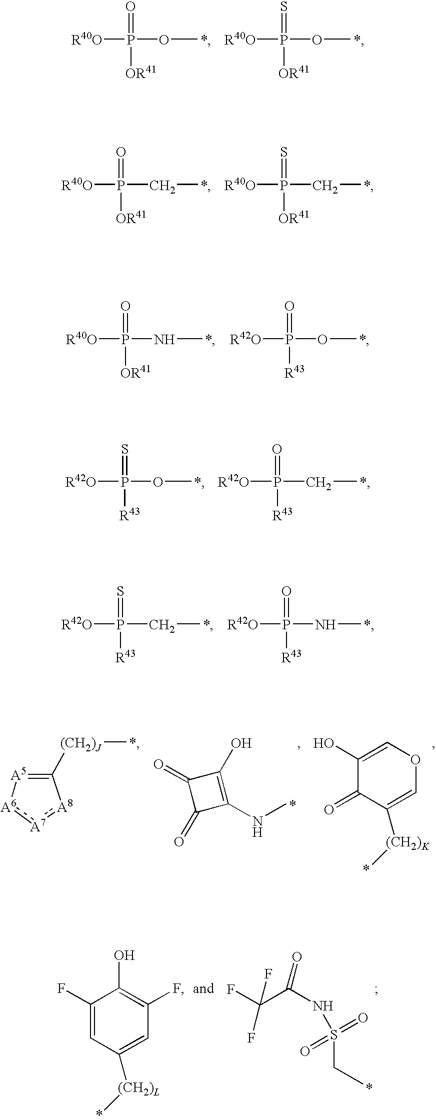

R³⁴ and each R³⁵ can be each independently selected from hydrogen, hydroxy, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R⁵⁰)₂—O—C(═)R⁵¹; R³⁶, R³⁷, R³⁸, R⁵⁰ and R⁵¹ can be each independently selected from hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl; each R³⁹ can be independently hydrogen or halogen; R⁴⁰, R⁴¹ and R⁴² can be each independently selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, pivaloyloxymethoxy, isopropyloxycarbonyloxymethoxy and

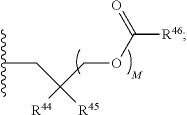

R⁴³ can be independently selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, and NR⁴⁷R⁴⁸; R⁴⁴ and R⁴⁵ can be each independently —C≡N or selected from of an optionally substituted C₁₋₈ organylcarbonyl, an optionally substituted C₁₋₈ alkoxycarbonyl and an optionally substituted C₁₋₈ organylaminocarbonyl; R⁴⁶, R⁴⁷, R⁴⁸ and R⁴⁹ can be each independently selected from hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl; A⁵ can be CR⁴⁹ or N; A⁶ can be C(OH), NH, or O (oxygen); A⁷ can be C(OH) or N (nitrogen); A⁸ can be C(OH), N (nitrogen), or O (oxygen); B, C and D can be each independently selected from 1, 2 and 3; A, E, F, G, H and I can be each independently 0 or 1; J, K and L can be each independently 0 or 1; M can be 1 or 2; each [symbol] can be a single or double bond, provided that both [symbol] cannot be double bonds; and Z can be an integer in the range of 2-10.

In some embodiments, R³³ can be —(CH₂)_{A}—OR³⁶. In an embodiments, R³³ can be —(CH₂)_{A}—OR³⁶, wherein R³⁶ can be hydrogen, and A can be 0. In other embodiments, R³³ can be —(CH₂)_{B}—COOR³⁶. An example of a —(CH₂)_{B}—COOR³⁶ group is —(CH₂)—COOH. In an embodiment, R³³ can be —(CH₂)_{B}—COOR³⁶, wherein R³⁶ can be an optionally substituted C₁₋₆ alkyl, and B is 1. In still other embodiments, R³³ can be —(CH₂)_{C}—C(═S)OR³⁶. For example, R³³ can be —(CH₂)—C(═S)OR³⁶, wherein R³⁶ can be hydrogen or an optionally substituted C₁₋₆ alkyl. In yet still other embodiments, R³³ can be —(CH₂)_{C}—C(═O)NR³⁷R³⁸. In an embodiment, R³³ can be —(CH₂)_{C}—C(═O)NR³⁷R³⁸, wherein R³⁷ and R³⁸ can both be hydrogen or an optionally substituted C₁₋₆ alkyl, and c can be 1. In some embodiments, R³³ can be

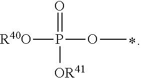

In other embodiments, R³³ can be

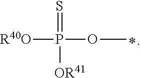

In still other embodiments, R³³ can be

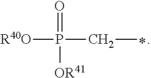

In yet still other embodiments, R³³ can be

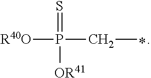

In some embodiments, R³³ can be

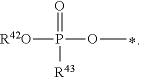

In other embodiments, R³³ can be

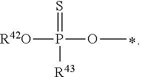

In still other embodiments, R³³ can be

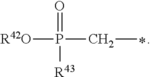

In yet still other embodiments, R³³ can be

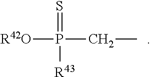

In some embodiments, R³³ cannot be

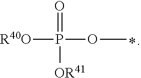

In some embodiments, R³³ cannot be

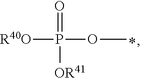

wherein R⁴⁰ and R⁴¹ are both either absent or H. In some embodiments, R³³ cannot be

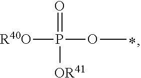

when R³⁴ and R³⁵ are hydroxy or hydrogen. In other embodiments, R³³ cannot be —(CH₂)_{A}—OR³⁶. In some embodiments, R³³ cannot be —OH. In other embodiments, when R³³ is

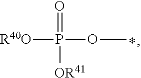

then R⁴⁰ cannot be a substituted C₁₋₆ alkyl. In other embodiments, when R³³ is

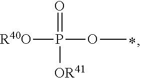

then R⁴¹ cannot be a substituted C₁₋₆ alkyl. In some embodiments, when R³³ is —OH, then R³⁴ cannot be hydrogen, hydroxy, an optionally substituted —O—C₁₋₆ alkyl or an optionally substituted —O—C₁₋₆ alkenyl. In some embodiments, when R³³ is —OH, then R³⁵ cannot be hydrogen, hydroxy, an optionally substituted —O—C₁₋₆ alkyl or an optionally substituted —O—C₁₋₆ alkenyl. In some embodiments, the 5′-terminal residue cannot be —OH or a phosphate when the internal nucleoside linkages are all phosphorothioates.

It is understood by those skilled in the art that when R⁴⁰ and/or R⁴¹ is absent, the oxygen will have an associated negative charge. In some embodiments, when R³³ is

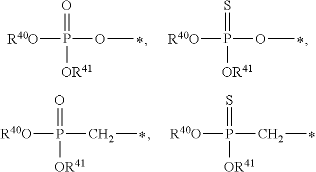

or

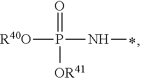

R⁴⁰ and R⁴¹ can be both hydrogen. In other embodiments, one of R⁴⁰ and R⁴¹ can be hydrogen, and the other of R⁴⁰ and R⁴¹ can be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In an embodiment, one of R⁴⁰ and R⁴¹ can be hydrogen and the other of R⁴⁰ and R⁴¹ can be an optionally substituted C₁₋₆ alkyl. In still other embodiments, both R⁴⁰ and R⁴¹ can be independently selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In an embodiment, both R⁴⁰ and R⁴¹ can be an optionally substituted C₁₋₆ alkyl. In yet still other embodiments, at least one of R⁴⁰ and R⁴¹ can be selected from pivaloyloxymethoxy, isopropyloxycarbonyloxymethoxy and

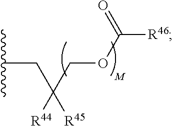

and the other of R⁴⁰ and R⁴¹ can be selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In some embodiments, both R⁴⁰ and R⁴¹ can be independently selected from pivaloyloxymethoxy, isopropyloxycarbonyloxymethoxy and

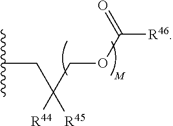

The substituents on

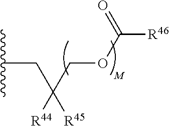

can vary. In an embodiment, R⁴⁴ and R⁴⁵ can be the same. In another embodiment, R⁴⁴ and R⁴⁵ can be different. In some embodiments, R⁴⁴ can be —C≡N and R⁴⁵ can be an optionally substituted C₁₋₈ alkoxycarbonyl such as —C(═O)OCH₃. In other embodiments, R⁴⁴ can be —C≡N and R⁴⁵ can be an optionally substituted C₁₋₈ organylaminocarbonyl, for example, —C(═O)NHCH₂CH₃ and —C(═O)NHCH₂CH₂-phenyl. In some embodiments, R⁴⁴ and R⁴⁵ can be independently C₁₋₈ organylcarbonyl or C₁₋₈ alkoxycarbonyl. In some embodiments, both R⁴⁴ and R⁴⁵ can be an optionally substituted C₁₋₈ organylcarbonyl. In an embodiments, both R⁴⁴ and R⁴⁵ can be —C(═O)CH₃. In other embodiments, both R⁴⁴ and R⁴⁵ can be an optionally substituted C₁₋₈ alkoxycarbonyl. In an embodiment, both R⁴⁴ and R⁴⁵ can be —C(═O)OCH₂CH₃. In another embodiment, both R⁴⁴ and R⁴⁵ can be —C(═O)OCH₃. In some embodiments, including those described in this paragraph, R⁴⁶ can be an optionally substituted C₁₋₄-alkyl. In an embodiment, R⁴⁶ can be methyl or tert-butyl. In some embodiments, M can be 1. In an embodiment, M can be 1 and both R⁴⁴ and R⁴⁵ can be an optionally substituted C₁₋₈ alkoxycarbonyl or an optionally substituted C₁₋₈ organylcarbonyl. In other embodiments, M can be 2. In an embodiment, M can be 2 and both R⁴⁴ and R⁴⁵ can be an optionally substituted C₁₋₈ alkoxycarbonyl. In another embodiment, M can be 2 and both R⁴⁴ and R⁴⁵ can be an optionally substituted C₁₋₈ organylcarbonyl.

Suitable

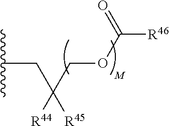

groups include, but are not limited to, the following:

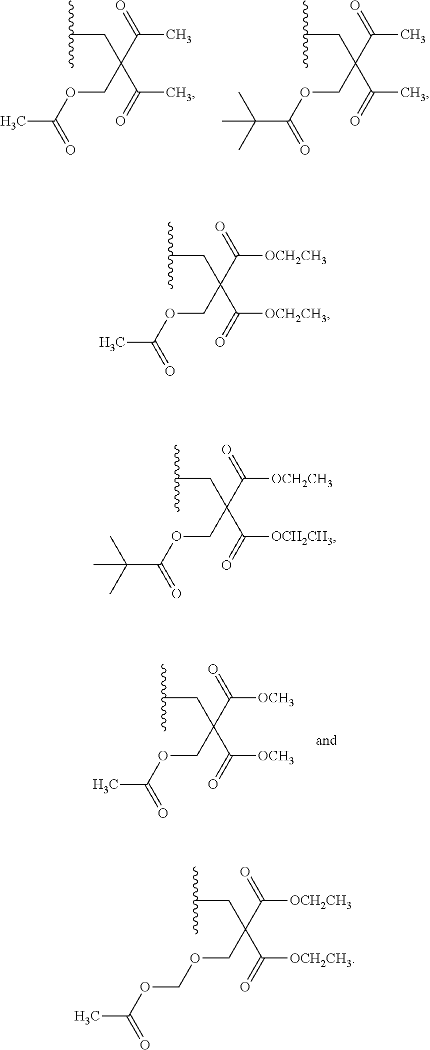

Additional

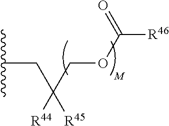

groups include the following.

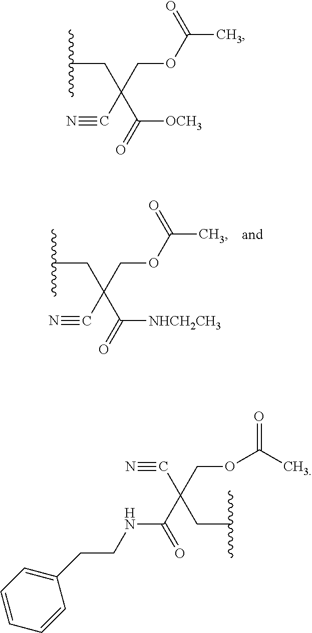

In some embodiments, when R³³ is

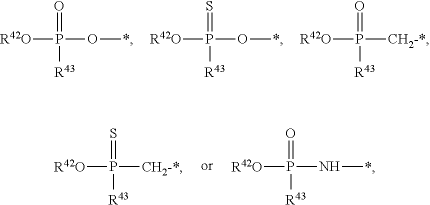

R⁴² can be selected from absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl; and R⁴³ can be independently selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. Those skilled in the art understand that when R⁴² is absent, the oxygen will have an associated negative charge. In other embodiments, R⁴² can be hydrogen and R⁴³ can be an optionally substituted C₁₋₆ alkyl. In an embodiment, R⁴² can be hydrogen and R⁴³ can be methyl or ethyl. In other embodiments, R⁴² can be hydrogen, and R⁴³ can be NR⁴⁷R⁴⁸, wherein R⁴⁷ and R⁴⁸ can be each independently selected from hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl. In some embodiments, R⁴⁷ and R⁴⁸ can be each independently hydrogen or an optionally substituted C₁₋₆-alkyl.

In some embodiments, R³⁴ can be hydrogen, hydroxy, an optionally substituted —O—C₁₋₆ alkyl and —OC(R⁵⁰)₂—O—C(═O)R⁵¹. In some embodiments, each R³⁵ can be independently selected from hydrogen, hydroxy, an optionally substituted —O—C₁₋₆ alkyl and —OC(R⁵⁰)₂—O—C(═O)R⁵¹. In some embodiments, R³⁴ and each R³⁵ can be independently selected from an optionally substituted —O—C₁₋₆ alkyl and —OC(R⁵⁰)₂—O—C(═O)R⁵¹. In some embodiments of this paragraph, R⁵⁰ can be hydrogen and R⁵¹ can be an optionally substituted C₁₋₆ alkyl (for example, methyl and t-butyl). In some embodiments, R³⁴ cannot be hydroxy. In some embodiments, each R³⁵ cannot be hydroxy. In some embodiments, R³⁴ cannot be hydrogen. In some embodiments, each R³⁵ cannot be hydrogen. In some embodiments, R³⁴ cannot be methoxy. In some embodiments, each R³⁵ cannot be methoxy. In some embodiments, R³⁴ cannot be hydrogen, hydroxy, an optionally substituted —O—C₁₋₆ alkyl or an optionally substituted —O—C₁₋₆ alkenyl. In some embodiments, R³⁵ cannot be hydrogen, hydroxy, an optionally substituted —O—C₁₋₆ alkyl or an optionally substituted —O—C₁₋₆ alkenyl.

In some embodiments, Z can be 2, 3 or 4. In other embodiments, Z can be 2, 3, 4, 5 or 6. In yet still other embodiments, Z can be 3, 4, 5, 6 or 7. In some embodiments, Z can be 4 or 5.

Examples of compounds of Formula (II) are shown below.

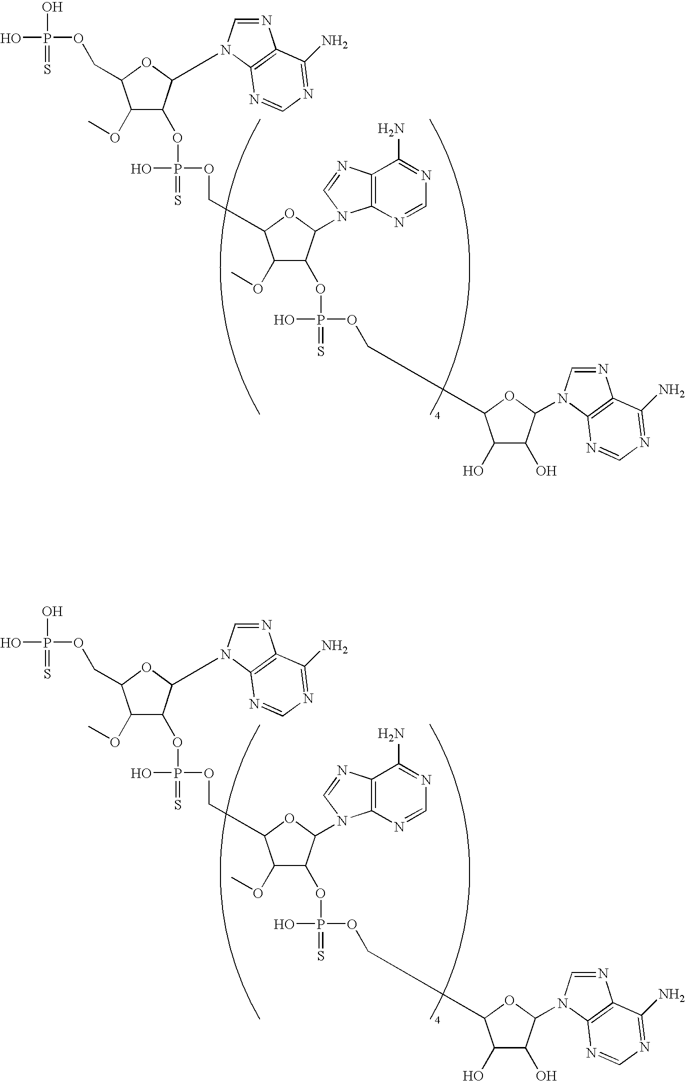

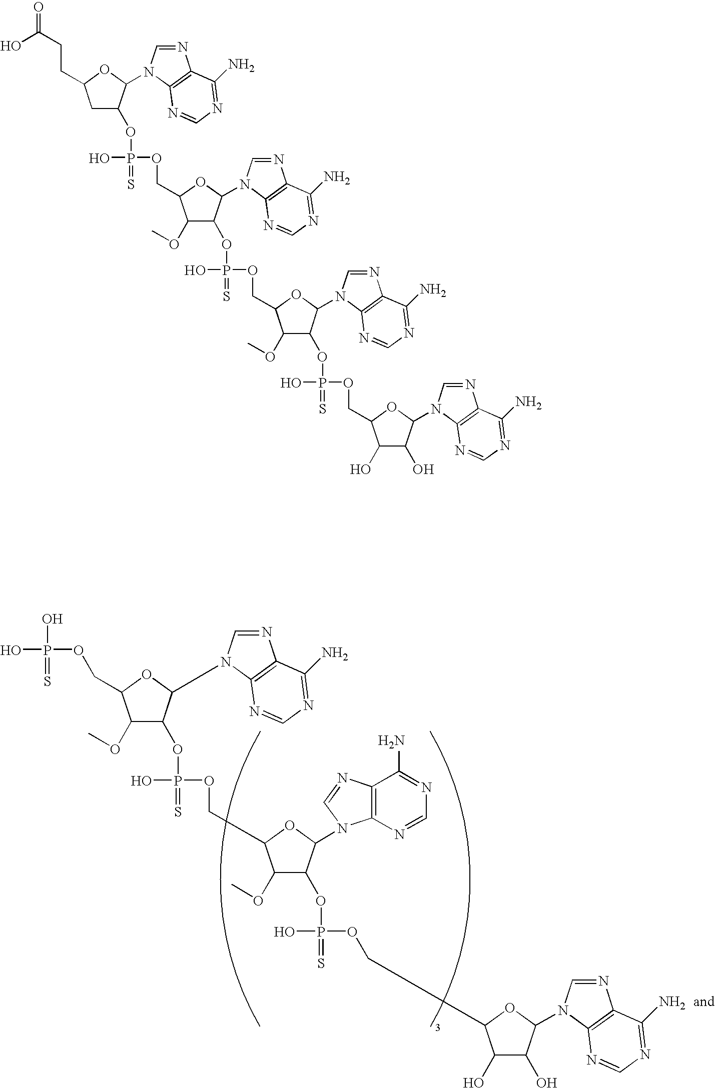

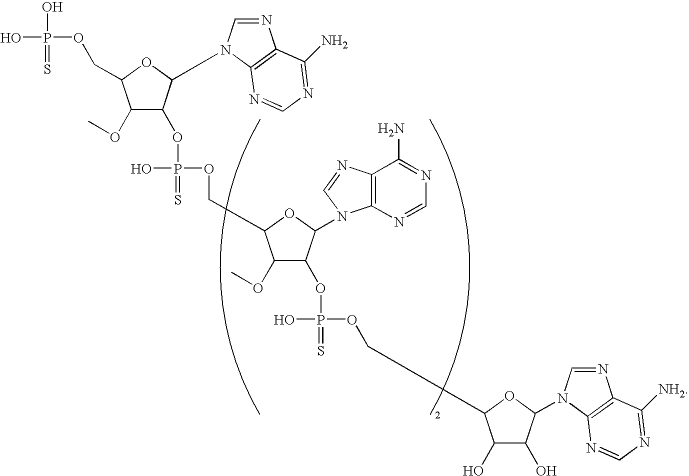

Without asking to be bound by any particular theory, it is believed that neutralizing the charge on one or more of the phosphate groups facilitates the penetration of the cell membrane by compounds of Formulae (I), (Ia) and (II) by making the compound more lipophilic. Furthermore, it is believed that the 2,2-disubstituted-acyl(oxyalkyl) groups; for example

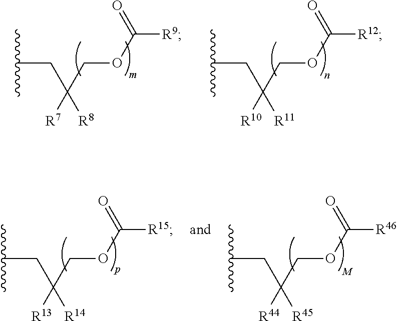

attached to the phosphate impart increased plasma stability to the compounds of Formulae (I), (Ia) and (II) by inhibiting the degradation of the compound. Once inside the cell, the 2,2-disubstituted-acyl(oxyalkyl) groups attached to the phosphate can be easily removed by esterases via enzymatic hydrolysis of the acyl group. The remaining portions of the group on the phosphate can then be removed by elimination. The general reaction scheme is shown below in Scheme 1. Upon removal of the 2,2-disubstituted-acyl(oxyalkyl) group, the resulting trinucleotide analog possesses a monophosphate. Thus, in contrast to use of trinucleoside compounds, the necessity of an initial intracellular phosphorylation is no longer a prerequisite to obtaining the biologically active phosphorylated form.

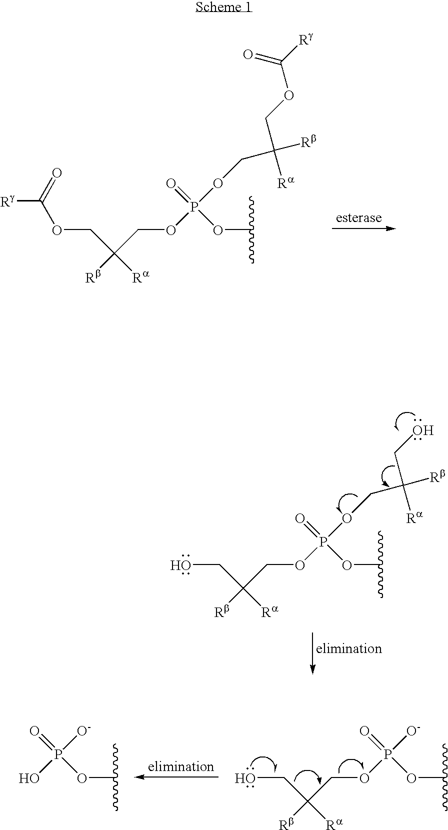

A further advantage of the 2,2-disubstituted-acyl(oxyalkyl) groups described herein is the rate of elimination of the remaining portion of the 2,2-disubstituted-acyl(oxyalkyl) group is modifiable. Depending upon the identity of the groups attached to the 2-carbon, shown in Scheme 1 as R^{α} and R^{β}, the rate of elimination may be adjusted from several seconds to several hours. As a result, the removal of the remaining portion of the 2,2-disubstituted-acyl(oxyalkyl) group can be retarded, if necessary, to enhance cellular uptake but, readily eliminated upon entry into the cell.

Additionally, when groups on the 2-carbon are identical, the 2,2-disubstituted-acyl(oxyalkyl) group is achiral, thus, markedly reducing the number of stereoisomers in the final compound (e.g., compounds of Formulae (I), (Ia) and/or (II)). Having achiral 2,2-disubstituted-acyl(oxyalkyl) group also can simplify separation and characterization of the trimers.

The 3′-positions of the middle and 5′-terminal residues can be protected with various protecting groups. Examples of suitable protecting groups are an optionally substituted —O—C₁₋₆ alkyl and an acyloxyalkyl group. When the group on the 3′-position is protected with an acyloxyalkyl group, it can also be removed by esterases via enzymatic hydrolysis of the acyl group followed by elimination of the remaining portion of the group. By varying the group at the 3′-position, the rate of elimination can be modified. It is believed that protecting the 3′-position minimizes and/or inhibits the isomerization of the phosphate on the 2′-position to the 3′-position. Additionally, protection of the 3′-position can reduce the likelihood that the phosphate will be prematurely cleaved off before entry into the cell.

As noted above, the rate of elimination of the groups on the 3′-positions and the phosphates can be adjusted; thus, in some embodiments, the identity of the groups on the phosphates and the 3′-positions can be chosen such that one or more groups on the phosphates are removed before the groups on the 3′-positions. In other embodiments, the identity of the groups on the phosphates and the 3′-positions can be chosen such that at least one group on the phosphates is removed after the groups on the 3′-positions. In an embodiment, the identity of the groups on the phosphates and the 3′-positions can be chosen such that the groups on the internal phosphates attached to the middle and 2′-terminal residues are removed before the groups on the 3′-positions of the middle and 5′-terminal residues. In another embodiment, the identity of the groups on the phosphates and the 3′-positions can be chosen such that the groups on the internal phosphates attached to the middle and 2′-terminal residues are removed before at least one group on the 5′-terminal phosphate and at least one group on the 5′-terminal residue is removed before the groups on the 3′-positions of the middle and 5′-terminal residues. In still another embodiment, the identity of the groups on the phosphates and the 3′-positions can be chosen such that the groups on the internal phosphates attached to the middle and 2′-terminal residues are removed before the groups on the 5′-terminal phosphate which in turn are removed before the groups on the 3′-positions of the middle and 5′-terminal residues. In some embodiments, identity of the groups on the phosphates and the 3′-positions can be chosen such that at least one group on the 5′-terminal residue is removed before the groups on the internal phosphates attached to the middle and 2′-terminal residues and the group on the 3′-position of the middle residue. In other embodiments, identity of the groups on the phosphates and the 3′-positions can be chosen such that both groups on the 5′-terminal residue is removed before the groups on the internal phosphates attached to the middle and 2′-terminal residues and the group on the 3′-position of the middle residue. In still other embodiments, all the groups on the phosphates are removed before the group on the 3′-position on the middle residue. In yet still other embodiments, the groups on the phosphates of the 5′-terminal residue and the phosphate group of the middle residue are removed before the group on the 3′-position on the middle residue, and the group on the 3′-position of the middle residue is removed before the group on the phosphate of the 2′-terminal residue.

In some embodiments, the breakdown of the trimer can be adjusted by protecting the phosphate groups, the 3′-positions of the middle and/or 5′-terminal residues. This in turn can enhance cellular uptake and assist in maintaining the balance between unwanted viral RNA and native cellular RNA.

Additionally, in some embodiments, the presence of one or more phosphorothioate groups in a compound of Formulae (I), (Ia) and/or (II) can increase the stability of the compound by inhibiting its degradation. Also, in some embodiments, the presence of one or more phosphorothioate groups can make the compound more resistant to cleavage in vivo and provide sustained, extended efficacy. In an embodiment, the phosphorothioate groups can facilitate the penetration of the cell membrane by a compound of Formulae (I), (Ia) and/or (II) by making the compound more lipophilic.

In some embodiments, 5′-terminal residue can facilitate transmittal of the compound across a cell membrane. Moreover, in some embodiments, the substituent attached to the 5′-position of the 5′-terminal residue can be cleaved in vivo to give a biologically active compound. In some embodiments, 5′-terminal residue can provide in vivo stability which can lead to improved pharmacokinetic properties.

### Synthesis

Compounds of Formulae (I), (Ia) and (II), and those described herein may be prepared in various ways. General synthetic routes to the compounds of Formulae (I), (Ia) and (II), and the starting materials used to synthesize the compounds of Formula (I), (Ia) and (II) are shown in Schemes 2a-2k. Various synthetic routes that may be used to synthesize compounds of Formula (I) and (Ia) are disclosed in U.S. Patent Publication Nos. 2008/0207554 and 2009/0181921, both of which are hereby incorporated by reference in their entirety. The routes shown are illustrative only and are not intended, nor are they to be construed, to limit the scope of the claims in any manner whatsoever. Those skilled in the art will be able to recognize modifications of the disclosed synthesis and to devise alternate routes based on the disclosures herein; all such modifications and alternate routes are within the scope of the claims.

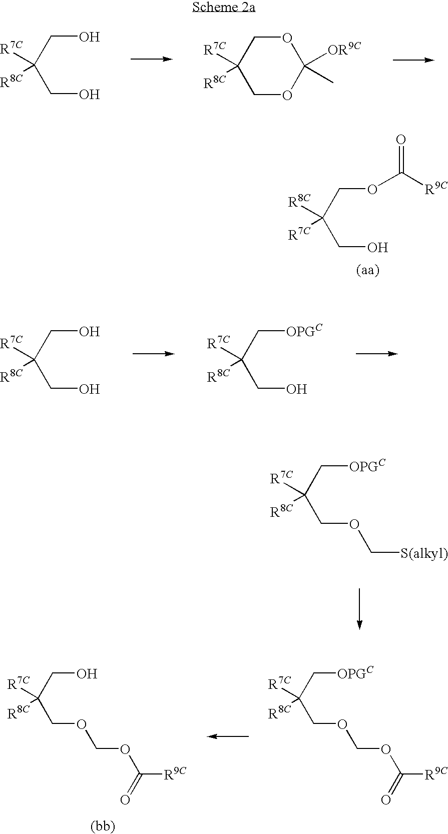

A compound of Formula aa can be synthesized as shown in above in Scheme 2a. The compound of Formula aa can be synthesized starting with an appropriate 2,2-bis(hydroxymethyl). An orthoester can be formed from the 2,2-bis(hydroxymethyl), followed by a ring-opening reaction to give a compound of Formula aa.

A compounds of Formulae bb can be synthesized also starting with an appropriate 2,2-bis(hydroxymethyl). One of the hydroxy groups can be protected with a suitable protecting group, such as a silyl ether group. Suitable silyl ether groups are described herein. A alkylthiomethyl ether can be formed at the position occupied by the remaining hydroxyl group using acetic anhydride and dimethylsulfoxide (DMSO). The newly formed alkylthiomethyl ether can undergo to oxidative-halogenation reaction using a suitable reagent such as sulfuryl chloride. An ester salt, such as potassium acetate, can then be added to form the terminal ester group. The protecting group on the initially protected hydroxyl group can be removed using a suitable reagent known to those skilled in the art, for example, an acid or tetraalkylammonium halide. The following articles provide exemplary methods for synthesizing the hydroxy precursors: Ora, et al., J. Chem. Soc. Perkin Trans. 2, 2001, 6, 881-5; Poijärvi, P. et al., Helv. Chim. Acta. 2002, 85, 1859-76; Poijärvi, P. et al., Lett. Org. Chem., 2004, 1, 183-88; and Poijárvi, P. et al., Bioconjugate Chem., 2005 16(6), 1564-71, all of which are hereby incorporated by reference in their entireties. In Scheme 2a, R^{7C}, R^{8C} and R^{9C} can be the same as R⁷, R⁸ and R⁹ as described herein with respect to a compound of Formula (I), and PG^{C} can be an appropriate protecting group. The hydroxy precursors of

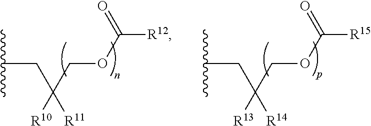

and

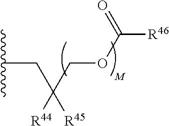

can also be obtained in a similar manner as described in Scheme 2a.

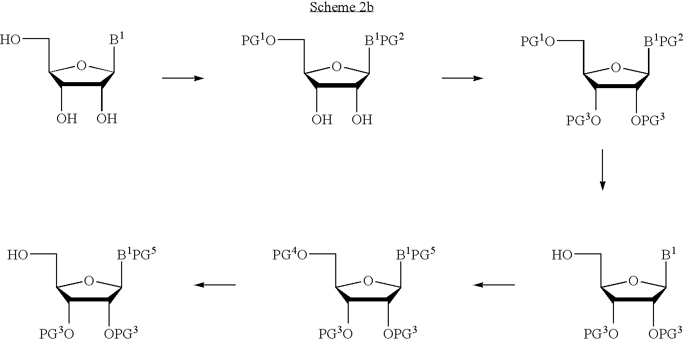

One example for synthesizing a compound that can be used to form the 2′-terminal residue is shown in Scheme 2b. The oxygen attached to the 5′-carbon and one or more amino groups attached to B¹ and/or a NH group(s) present in a ring of the heterocyclic base, represented by B¹, can be protected using appropriate protecting group moieties represented by PG¹ and PG², respectively. If more then one amino group is attached to a heterocyclic base, more than one protecting group can be used. If more than one protecting group is used, the protecting groups can be the same or different. In some embodiments, PG¹ and PG² can be the same or different. In an embodiment, PG¹ and PG² can be triarylmethyl protecting groups. A non-limiting list of triarylmethyl protecting groups are trityl, monomethoxytrityl (MMTr), 4,4′-dimethoxytrityl (DMTr), 4,4′,4″-trimethoxytrityl (TMTr), 4,4′,4″-tris-(benzoyloxy)trityl (TBTr), 4,4′,4″-tris(4,5-dichlorophthalimido) trityl (CPTr), 4,4′,4″-tris(levulinyloxy)trityl (TLTr), p-anisyl-1-naphthylphenylmethyl, di-o-anisyl-1-naphthylmethyl, p-tolyldipheylmethyl, 3-(imidazolylmethyl)-4,4′-dimethoxytrityl, 9-phenylxanthen-9-yl (Pixyl), 9-(p-methoxyphenyl)xanthen-9-yl (Mox), 4-decyloxytrityl, 4-hexadecyloxytrityl, 4,4′-dioctadecyltrityl, 9-(4-octadecyloxyphenyl)xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl.

Any oxygens attached as hydroxy groups to the 2′ and 3′-positions can also be protected using appropriate protecting groups. In some embodiments, the protecting groups on the 2′ and 3′-positions, represented by PG³, can be the same or different. In an embodiment, the PG³ groups are the same. In some embodiments, one or both PG³ groups can be silyl ether groups. Exemplary silyl ethers include, but are not limited to, trimethylsilyl (TMS), tert-butyldimethylsilyl (TBDMS), triisopropylsilyl (TIPS) and tert-butyldiphenylsilyl (TBDPS). In other embodiments, one or both PG³ groups can be levulinoyl groups.

After protecting any oxygens at the 2′ and 3′-positions, the protecting group on the oxygen attached to the 5′-carbon and any protecting groups on the heterocyclic base can be removed. In some embodiments, the protecting groups on the oxygen attached to the 5′-carbon and any protecting groups on the heterocyclic base can be removed using an acid (e.g., acetic acid). In an embodiment, the protecting group on the oxygen attached to the 5′-carbon can be removed before deprotecting one or more amino groups attached to B¹ and/or a NH group(s) present in a ring of B¹. In another embodiment, the protecting group on the oxygen attached to the 5′-carbon can be removed after deprotecting one or more amino groups attached to B¹ and/or a NH group(s) present in a ring of B¹. In still another embodiment, the protecting group on the oxygen attached to the 5′-carbon can be removed almost simultaneously with the removal of any protecting groups on the heterocyclic base.

The oxygen attached to the 5′-carbon and one or more amino groups attached to B¹ and/or a NH group(s) present in a ring of the heterocyclic base can then be reprotected using appropriate protecting groups represented by PG⁴ and PG⁵. The protecting groups PG⁴ and PG⁵ can be the same or different from the protecting groups used previously. In some embodiments, PG⁴ can be different from PG¹. In some embodiments, PG⁵ can be the same as PG². In an embodiment, the oxygen attached to the 5′-carbon can be protected with a silyl ether protecting group. As noted above, PG³, PG⁴ and PG⁵ can be different, thus, in some embodiments, PG³, PG⁴ and PG⁵ can be chosen such that conditions that would remove one of the group of PG³, PG⁴ and PG⁵ would not remove the remaining two protecting groups. As an example, PG³, PG⁴ and PG⁵ can be chosen such that PG⁵ can be removed without removing PG³ and/or PG⁴. In some embodiments, one or more amino groups attached to B¹ and/or a NH group(s) present in a ring of the heterocyclic base can be protected with a triarylmethyl protecting group(s). In an embodiment, the oxygen attached to the 5′-carbon can be reprotected before reprotecting any amino groups attached to B¹ and/or a NH group(s) present in a ring of B¹. In other embodiments, any amino groups attached to B¹ and/or a NH group(s) present in a ring of B¹ can be reprotected before protecting the oxygen attached to the 5′-carbon.

In some embodiments, the oxygen attached to the 5′-carbon can then be selectively deprotected using methods known to those skilled in the art. For example, the protecting group on the oxygen attached to the 5′-carbon can be selectively deprotected without removing any protecting groups on the heterocyclic base and/or any protecting groups on the oxygens attached to the 2′ and 3′-positions. In an embodiment, the protecting group on the oxygen attached to the 5′-carbon can be removed with a tetraalkylammonium halide, such as tetra(t-butyl)ammonium fluoride, or an acid.

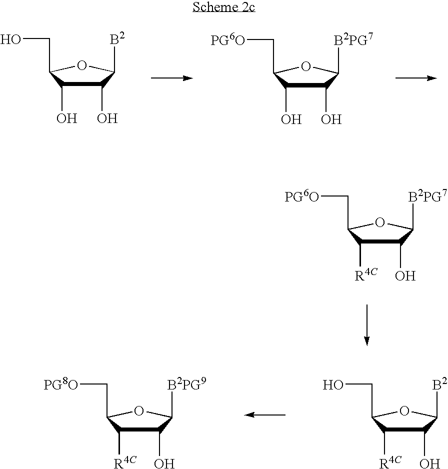

One example for synthesizing a nucleoside in which the 3′-position has R^{4C} being —OC(R^{16C})₂—O—C(═O)R^{17C}, wherein each R^{16C} and R^{17C} are each independently hydrogen or an optionally substituted C₁₋₄ alkyl is shown in Scheme 2c. The oxygen attached to the 5′-carbon and one or more amino groups attached to B² and/or a NH group(s) present in a ring of the heterocyclic base represented B² can be protected using appropriate protecting groups represented by PG⁶ and PG⁷, respectively. In some embodiments, PG⁶ and PG⁷ can be the same or different. In an embodiment, PG⁶ and PG⁷ can be triarylmethyl protecting groups. R^{4C} can be added by removing the hydrogen on the oxygen attached to the 3′-position using an appropriate reagent such as sodium hydride and adding the —C(R^{16C})₂—O—C(═O)R^{17C} group. In an embodiment, the —C(R^{16C})²—O—C(═O)R^{17C} group can be added using an appropriate alkylating reagent, such as sodium iodide, and X¹—C(R^{16C})₂—O—C(═O)R^{17C}, wherein R^{16C} and R^{17C} are described herein and X¹ can be a halide. The protecting groups on the oxygen attached to the 5′-carbon and any protecting groups on the heterocyclic base can then be removed using methods known to those in the art. For example, when PG⁶ and PG⁷ are triarylmethyl groups, both can be removed using an appropriate acid or a zinc dihalide (e.g., ZnBr₂). In some embodiments, the protecting groups on the oxygen attached to the 5′-carbon and any protecting groups on the heterocyclic base can be removed using acetic acid. In an embodiment, the protecting group on the oxygen attached to the 5′-carbon can be removed before deprotecting one or more amino groups attached to B² and/or a NH group(s) present in a ring of B². In another embodiment, the protecting group on the oxygen attached to the 5′-carbon can be removed after deprotecting one or more amino groups attached to B² and/or a NH group(s) present in a ring of B². In still another embodiment, the protecting group on the oxygen attached to the 5′-carbon can be removed almost simultaneously with the removal of any protecting groups on the heterocyclic base.

The oxygen attached to the 5′-carbon can then be reprotected with PG⁸, which can be the same or different protecting group as used previously. Similarly, any amino groups attached B² and/or a NH group(s) present in a ring of B² can be reprotected with PG⁹, which can be the same or different protecting groups as used previously. In some embodiments, PG⁸ and PG⁹ can be different. In some embodiments, PG⁸ can be different from PG⁶. In some embodiments, PG⁷ can be the same as PG⁹. In some embodiments, the oxygen attached to the 5′-carbon can be protected with a triarylmethyl group. In some embodiments, one or more amino groups attached to B² and/or a NH group(s) present in a ring of B² can be protected with a silyl ether group(s). In an embodiment, the oxygen attached to the 5′-carbon can be reprotected before reprotecting any amino groups attached to B² and/or a NH group(s) present in a ring of B². In other embodiments, any amino groups attached to B² and/or a NH group(s) present in a ring of B² can be reprotected before protecting the oxygen attached to the 5′-carbon. In an embodiment, PG⁸ can be a protecting group that cannot be removed under the same conditions as PG⁹. For example, PG⁹ can be a protecting group that can be removed by an acid that cannot remove PG⁸.

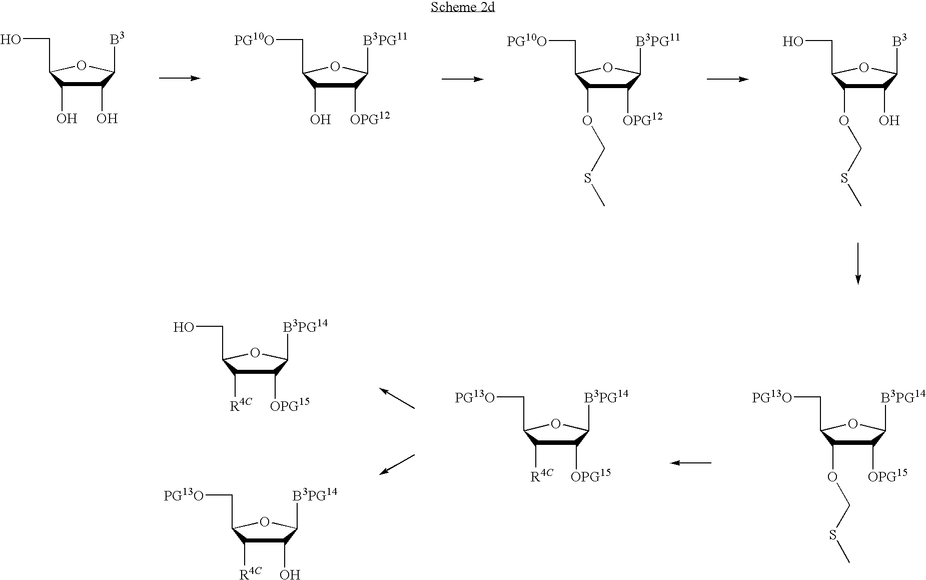

Another example for synthesizing a nucleoside in which the 3′-position has R^{4C} being —OC(R^{16C})₂—O—C(═O)R^{17C}, wherein each R^{16C} and R^{17C} are each independently hydrogen or an optionally substituted C₁₋₄-alkyl is shown in Scheme 2d. The oxygen attached to the 5′-carbon, any amino groups attached to B³ and/or a NH group(s) present in a ring of the heterocyclic base represented by B³ and any oxygens attached as hydroxy groups to the 2′-position can be protected using appropriate protecting groups represented by PG10, PG¹¹ and PG¹². In some embodiments, one, two or all of PG¹⁰, PG¹¹ and PG¹² can be the same or different. In an embodiment, PG¹⁰, PG¹¹ and PG¹² can be triarylmethyl protecting groups. The hydrogen of the —OH group attached to the 3′-position can then be removed using methods known to those skilled in the art, such as sodium hydride, followed by alkylation with a (halomethyl)(alkyl)sulfane. Any protecting groups represented by PG¹⁰, PG¹¹ and PG¹² can be then removed using methods known to those skilled in the art. For example, when PG¹⁰, PG¹¹ and PG¹² are triarylmethyl groups, PG¹⁰, PG¹¹ and PG¹² can be removed using an acid such as acetic acid or a zinc dihalide such as zinc dibromide. In an embodiment, PG¹⁰, PG¹¹ and PG¹² can be removed with acetic acid.

The oxygen attached to the 5′-carbon, any amino groups attached to B³ and/or a NH group(s) present in a ring of B³ and any oxygens attached as hydroxy groups to the 2′-position can be reprotected using appropriate protecting groups which can be the same or different from those used previously. In some embodiments, PG¹³ can be different from PG¹⁰. In an embodiment, PG¹⁴ can be the same as PG¹¹. In some embodiments, PG¹⁵ can be different from PG¹². In other embodiments, PG¹⁵ can be the same as PG¹². In some embodiments, the oxygen attached to the 5′-carbon can be protected using a triarylmethyl protecting group. In an embodiment, any amino groups attached to B³ and/or a NH group(s) present in a ring of B³ can be protected with a silyl ether group(s). In some embodiments, any oxygens attached as hydroxy groups at the 2′-position can be protected using levulinoyl group(s). In other embodiments, any oxygens attached as hydroxy groups to the 2′-position can be protected using silyl ether group(s). In an embodiment, PG¹³, PG¹⁴ and PG¹⁵ can be different from each other. In an embodiment, the oxygen attached to the 5′-carbon can be reprotected before reprotecting any amino groups attached to B³ and/or a NH group(s) present in a ring of B³ and/or any oxygens attached as hydroxy groups to the 2′-position. In some embodiments, any amino groups attached to B³ and/or a NH group(s) present in a ring of B³ can be reprotected after protecting the oxygen attached to the 5′-carbon but before reprotecting any oxygens attached as hydroxy groups to the 2′-position. In an embodiment, any oxygens attached as hydroxy groups to the 2′-position can be reprotected after reprotecting the oxygen attached to the 5′-carbon and any amino groups attached to B³ and/or a NH group(s) present in a ring of B³. In some embodiments, PG¹³ can be a protecting group that can be selectively removed without removing PG¹⁴ and/or PG¹⁵. As example, PG¹³ can be a protecting group that can be removed using a tetraalkylammonium halide that cannot remove PG¹⁴ and/or PG¹⁵. In an embodiment, PG¹⁴ can be a protecting group that cannot be removed under the same conditions as PG¹³ and/or PG¹⁵. For example, PG¹⁴ can be a protecting group that cannot be removed by a tetraalkylammonium halide or hydrazinium acetate when one or either condition can remove PG¹³ and/or PG¹⁵. In some embodiments, PG¹⁵ can be a protecting group than cannot be removed under the same conditions as PG¹³ and/or PG¹⁴. For example, PG¹⁵ can be levulinoyl group that can be removed using hydrazinium acetate which cannot remove PG¹³ and/or PG¹⁴. In other embodiments, PG¹⁴ and PG¹⁵ can be removed under the same conditions, but those conditions cannot remove PG¹³.

The methyl(alkyl)sulfane added to the oxygen attached to the 2′-position can under go an oxidative-halogenation reaction using an appropriate reagent such as sulfuryl chloride. An ester in the form of an ester salt can then be added to form R^{4C}. The protecting groups, PG¹³ can then be selectively removed. For example, as described above PG¹³ can be removed without removing PG¹⁴ and/or PG¹⁵. In an embodiment, PG¹³ can be removed using a tetraalkylammonium halide such as tetrabutylammonium fluoride. In another embodiment, PG¹⁵ can be selectively removed such that PG¹⁵ is removed without removing PG¹³ and/or PG¹⁴. In an embodiment, PG¹⁵ can be removed with hydrazinium acetate.

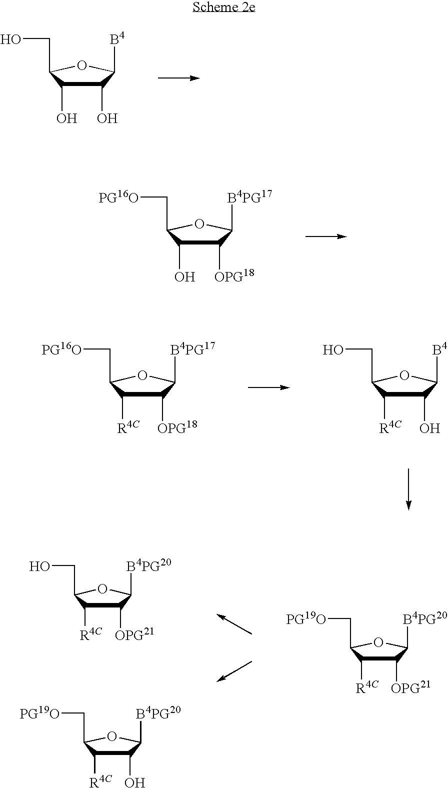

An example for synthesizing a nucleoside in which the substituent attached to the 3′-position has R^{4C} being an optionally substituted —O—C₁₋₆ alkyl is shown in Scheme 2e. The oxygen attached to the 5′-carbon, any amino groups attached to the heterocyclic base represented by B⁴ and any oxygens attached as hydroxy groups to the 2′-position can be protected using appropriate protecting groups represented by PG¹⁶, PG¹⁷ and PG¹⁸. In some embodiments, one, two or all of PG¹⁶, PG¹⁷ and PG¹⁸ can be the same or different. In an embodiment, PG¹⁶, PG¹⁷ and PG¹⁸ can be triarylmethyl protecting groups. The hydrogen of the —OH attached to the 3′-position can then be removed using methods known to those skilled in the art such as sodium hydride followed by alkylation with a haloalkyl, which can be optionally substituted. Any protecting groups represented by PG¹⁶, PG¹⁷ and PG¹⁸ can be then removed using the appropriate reagent and conditions known to those skilled in the art. For example, when PG¹⁶, PG¹⁷ and PG¹⁸ can be removed using an acid or a zinc dihalide. In an embodiment, PG¹⁶, PG¹⁷ and PG¹⁸ can be removing using acetic acid.

The oxygen attached to the 5′-carbon, any amino groups attached to B⁴ and/or a NH group(s) present in a ring of B⁴ and any oxygens attached as hydroxy groups to the 2′-position can be reprotected using appropriate protecting groups which can be the same or different from those protecting groups used previously. In some embodiments, PG¹⁹ can be different from PG¹⁶. In an embodiment PG²⁰ can be different from PG¹⁷. In some embodiments, PG²¹ can be different from PG¹⁸. In other embodiments, PG²¹ can be the same as PG¹⁸. In some embodiments, the oxygen attached to the 5′-carbon can be protected using a triarylmethyl protecting group. In an embodiment, any amino groups attached to the heterocyclic base can be protected with a silyl ether group(s). In some embodiments, any oxygens attached as hydroxy groups to the 2′-position can be protected using levulinoyl group(s). In other embodiments, any oxygens attached as hydroxy groups to the 2′-position can be protected using silyl group(s). In an embodiment, PG¹⁹, PG²⁰ and PG²¹ can be different from each other. In an embodiment, the oxygen attached to the 5′-carbon can be reprotected before reprotecting any amino groups attached to B⁴ and/or a NH group(s) present in a ring of B⁴ and/or any oxygens attached as hydroxy groups to the 2′-position. In some embodiments, any amino groups attached to B⁴ and/or a NH group(s) present in a ring of B⁴ can be reprotected after protecting the oxygen attached to the 5′-carbon but before reprotecting any oxygens attached as hydroxy groups to the 2′-position. In an embodiment, any oxygens attached as hydroxy groups to the 2′-position can be reprotected after reprotecting the oxygen attached to the 5′-carbon and any amino groups attached to B⁴ and/or a NH group(s) present in a ring of B⁴. In an embodiment, PG¹⁹ can be a protecting group that can be selectively removed without removing PG²⁰ and/or PG²¹. As an example, PG¹⁹ can be a protecting group that can be removed using a tetraalkylammonium halide that cannot remove PG²⁰ and/or PG²¹. In an embodiment, PG²⁰ can be a protecting group that cannot be removed under the same conditions as PG¹⁹ and/or PG²¹. For example, PG²⁰ can be a protecting group that cannot be removed by a tetraalkylammonium halide or hydrazinium acetate when one or either condition can remove PG¹⁹ and/or PG²¹. In some embodiments, PG²¹ can be a protecting group than cannot be removed under the same conditions as PG¹⁹ and/or PG²⁰. For example, PG²¹ can be levulinoyl group that can be removed using hydrazinium acetate which cannot remove PG²⁰ and/or PG²¹.

In some embodiments, PG¹⁹ can be selectively removed. As described above, PG¹⁹ can be chosen such that it can be removed without removing PG²⁰ and/or PG²¹. In an embodiment, PG¹⁹ can be removed using a tetraalkylammonium halide such as tetrabutylammonium fluoride. Alternatively, in other embodiments, PG²¹ can be removed without removing PG¹⁹ and PG²⁰ using, for example, hydrazinium acetate.

Compounds that can be used to form the 5′-terminal residue are known to those skilled in the art. Some of the compounds that can be used to the 5′-terminal residue are commercially available. Other compounds that can be used to obtain the 5′-terminal residue can be synthesized using methods known to those skilled in the art.

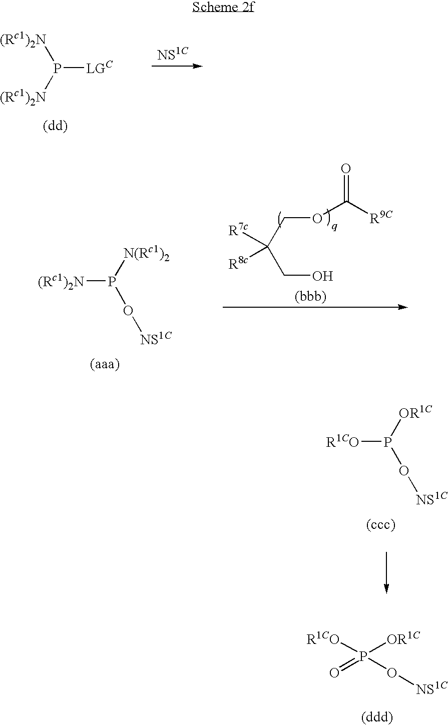

When the 5′-terminal residue is protected by one or more 2,2-disubstituted-acyl(oxyalkyl) groups, the 5′-terminal residue can be obtained by various methods. An example of a suitable method is shown in Scheme 2f. In Scheme 2f, R^{7C}, R^{8C}, R^{9C}, NS^{1C} and q can be the same as R⁷, R⁸, R⁹, NS¹ and m, respectively, as described above with respect to Formula (I). In Scheme 2f, R^{1C} indicate a 2,2-disubstituted-acyl(oxyalkyl) group. A phosphoamidite can be formed at the 5′-position or equivalent position of a nucleoside or a protected nucleoside by reacting a compound of Formula dd with NS^{1C} to form a compound of Formula aaa. For a compound of Formula dd, in an embodiment, each R^{c1} can be independently an optionally substituted C₁₋₄ alkyl, and LG^{1C} can be a suitable leaving group. In an embodiment, the leaving group on a compound of Formula dd can be a halogen.

One or more R^{1C} moieties can be added to a compound of Formula aaa by reacting a compound of Formula aaa with a compound of Formula bbb to form a compound of Formula ccc. An activator can be used to assist the addition. An example of a suitable activator is a tetrazole such as benzylthiotetrazole. Additional activators that can be used are disclosed in Nurminen, et al., J. Phys. Org. Chem., 2004, 17, 1-17 and Michalski, J. et al., State of the Art. Chemical Synthesis of Biophosphates and their Analogues via P^{III} Derivatives, Springer Berlin (2004) vol. 232, pages 43-47; which is hereby incorporated by reference for the limited purpose of their disclosure of additional activators. In some embodiments, one R^{1C} moiety can be added to a compound of Formula aaa. In other embodiments, two R^{1C} moieties can be added to a compound of Formula aaa. When two R^{1C} moieties are added, in some embodiments, both R^{1C} can be the same. In other embodiments, when two R^{1C} moieties are added, the two R^{1C} moieties can be different. In an embodiment, both R^{1C} can be

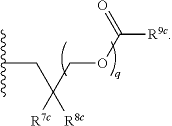

In some embodiments, the nucleoside, or the protected nucleoside can have the structure of a compound of Formula pp,

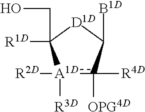

in which [symbol] can be a double or single bond; A^{1D} can be selected from C (carbon), O (oxygen) and S (sulfur); B^{1D} can be selected from an optionally substituted heterocyclic base and an optionally substituted protected heterocyclic base; D^{1D} can be C═CH₂ or O (oxygen); R^{1D} can be selected from hydrogen, azido, —CN, an optionally substituted C₁₋₄ alkyl and an optionally substituted C₁₋₄ alkoxy; R^{2D} can be absent or selected from hydrogen, halogen, hydroxy and an optionally substituted C₁₋₄ alkyl; R^{3D} can be absent or selected from hydrogen, halogen, azido, amino, hydroxy, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl, —OC(R^{5D})₂—O—C(═O)R^{6D} and —OPG^{5D}; R^{4D} can be absent or selected from hydrogen, halogen, hydroxy, —CN, —NC, an optionally substituted C₁₋₄ alkyl, an optionally substituted haloalkyl and an optionally substituted hydroxyalkyl; each R^{5D} and R^{6D} can be independently hydrogen or an optionally substituted C₁₋₄-alkyl; and PG^{4D} can be a protecting group. In some embodiments, PG^{4D} can be a levulinoyl group. In other embodiments, PG^{4D} can be a silyl ether group. In some embodiments, PG^{5D} can be a levulinoyl group. In other embodiments, PG^{5D} can be a silyl ether group.

A compound of Formula ddd can be obtained by oxidizing the phosphite to a phosphate using an appropriate oxidizing agent and oxygen donor. In an embodiment, the oxidizing agent can be iodine and the oxygen donor can be water. Where a phosphorothioate is present at the 5′-carbon of NS^{1C}, the phosphorothioate can be formed using suitable methods known to those skilled in the art. For example, a compound of Formula of aaa can be oxidized with elemental sulfur to obtain a phosphorothioate (not shown). Alternatively, the double-bonded oxygen on the phosphorus can be exchanged with a sulfur using a suitable reagent, such as cyclooctasulfur, Lawesson's reagent and 3-[dimethylaminomethylidene]amino-3H-1,2,4-dithiazole-3-thione (DDTT).

In some embodiments, various protecting groups may be present on NS^{1C}. For example, any hydroxy groups attached to the 2′-position and 3′-position may be protected using one or more appropriate protecting groups, such as a levulinoyl group. Similarly, any amino groups and/or any —NH groups present in the ring of the heterocyclic base may be protected using suitable one or more suitable protecting groups. Suitable protecting groups include, but are not limited to, silyl ethers and triarylmethyl groups. The protecting groups can promote the addition of a dimer containing the middle and 2′-terminal residue (for example, a compound of Formula kk or a compound of Formula xx) to the 5′-position or equivalent position of NS^{1C}. Thus, the presence of protecting groups on NS^{1C} can be advantageous for minimizing unwanted side reactions. Additionally, by minimizing the number and/or amount of side products, the separation and isolation of the desired product can be made easier.

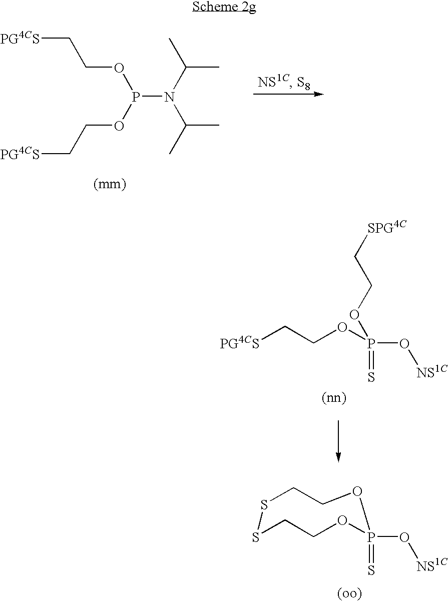

Various methods known to those skilled in the art can also be used to form a 5′-terminal residue with R¹ being a phosphorothioate. One example of a suitable method is shown in Scheme 2g. A phosphitylating reagent such as a compound of Formula mm can be coupled to a nucleoside or a protected nucleoside. In Scheme 2g, the nucleoside or the protected nucleoside is denoted by NS^{1C}. In some embodiments, NS^{1C} can have the structure of a compound of Formula pp as described herein. To facilitate the reaction between the nucleoside, or the protected nucleoside and a compound of Formula mm, an activator can be used. In some embodiments, the activator can be a tetrazole. The phosphorus can undergo sulfurization to form a compound of Formula nn. Various reagents that can be used for the sulfurization of the phosphorus are known to those skilled in the art. In some embodiments, the sulfurization reagent can be cyclooctasulfur. As shown in Scheme 2g, the phosphorus can undergo oxidation from phosphorus(III) to phosphorus(V). In some embodiments, a compound of Formula nn can be oxidized and a disulfide bond can be formed as shown in a compound of Formula oo. Suitable oxidizing agents are known to those skilled in the art. A non-limiting list of suitable oxidizing agents include, but are not limited to, iodine, dimethyl sulfoxide, glutathione, potassium ferricyanide, thallium trifluoroacetate or silver triflate. In an embodiment, the compound of Formula nn can be oxidized using iodine.

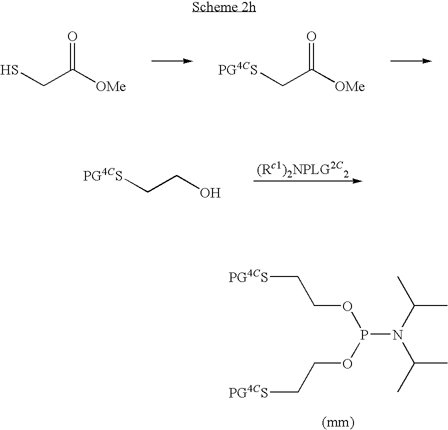

A phosphitylating reagent can be prepared as described in Austin, C.; Grajkowski, A.; Cieslak, J.; Beaucage, S. L. Org. Lett. 2005, 7, 4201-4204, which is hereby incorporated by reference in its entirety. As shown in Scheme 2h, the sulfur of methyl 2-mercaptoacetate can be protected using a suitable protecting group denoted by PG^{4D}. Suitable protecting groups that can be used to protect the sulfur are known to those skilled in the art. In some embodiments, PG^{4C} can be a triarylmethyl protecting group, such as those described herein. In an embodiment, PG^{4C} can be 4,4′-dimethoxytrityl. The ester of the sulfur protected methyl 2-mercaptoacetate can be reduced to an alcohol using an appropriate reducing agent. Appropriate reducing agents are known to those skilled in the art. Examples of suitable reducing agents include lithium aluminum hydride (LiAlH₄), diisobutylaluminum hydride (DIBAH), lithium triethylborohydride, BH₃—SMe₂ in refluxing THF and triethoxysilane (HSi(OEt)₃). In some embodiments, the reducing agent can be lithium aluminum hydride (LiAlH₄). A phosphitylating reagent, such as a compound of Formula mm, can be formed by reacting PG^{4C}S(CH₂)₂OH with a compound of formula (LG^{2C})₂P(N(R^{c1})₂))) wherein each LG^{2C} can be an appropriate leaving group and each R^{c1} can be independently an optionally substituted C₁₋₄ alkyl. In an embodiment, each LG^{2C} can be a halogen.

The substituent R^{3D}, in some embodiments, can be an optionally substituted —O—C₁₋₆ alkyl. In an embodiment, R^{3D} can be —OCH₃. In other embodiments, R^{3D} can be —OC(R_{5D})₂—O—C(═O)R^{6D}. In an embodiment, when R^{3D} can be —OC(R^{5D})₂—O—C(═O)R^{6D}, both R^{5D} groups can be hydrogen and R^{6D} can be an optionally substituted alkyl (e.g., methyl). In another embodiment, R^{3D} can be —OPG^{5D}. In some embodiments, including those in this paragraph, A^{1D} can be carbon, D^{1D} can be oxygen, and [symbol] can be a single bond.

In an embodiment, B^{1D} can each be independently selected from:

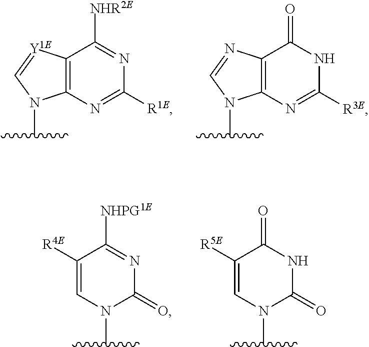

and

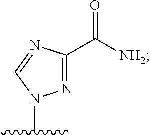

wherein: R^{1E} can be hydrogen or halogen; R^{2E} can be hydrogen, an optionally substituted C₁₋₄ alkyl, an optionally substituted C₃₋₈ cycloalkyl or PG^{2E}; R^{3E} can be hydrogen or amino; R^{4E} can be selected from hydrogen, halogen, an optionally substituted C₁₋₄ alkyl, an optionally substituted C₂₋₄ alkenyl and an optionally substituted C₂₋₄ alkynyl; R^{5E} can be selected from hydrogen, halogen, an optionally substituted C₁₋₄alkyl, an optionally substituted C₂₋₄ alkenyl and an optionally substituted C₂₋₄ alkynyl; and Y^{1E} can be N or CR^{6E}, wherein R^{6E} can be selected from hydrogen, halogen, an optionally substituted C₁₋₄-alkyl, an optionally substituted C₂₋₄-alkenyl, an optionally substituted C₂₋₄-alkynyl; and PG^{1E} and PG^{2E} can be each independently hydrogen or a protecting group. In an embodiment, one or both of PG^{2E} and PG^{1E} can be a triarylmethyl protecting group such as those described herein. In an embodiment, B^{1D} can be

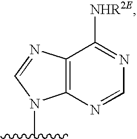

where R^{2E} can be hydrogen or PG^{2E}.

Another suitable method for preparing a 5′-terminal residue is described as follows. The 5′-hydroxy group of a nucleoside, or a protected nucleoside (such as a compound of Formula pp) can be oxidized with a suitable oxidizing agent to form an aldehyde. Various oxidizing agents that can be used are known to those skilled in the art. An example of a suitable oxidizing agent is Dess Martin reagent. The aldehyde can be converted to an alkenyl via an olefination reaction. In some embodiments, the olefination reaction can be conducted in the presence of a base (for example, triethylamine or sodium hydride) or an acid. The alkenyl bond can be hydrogenated to form a R¹ group, such as those described herein. An example of agents that be used to hydrogenate that double bond is H₂ and Pd/C, H₂ and Pd(OH)₂. In some embodiments, any oxygens attached to the 2′-position and/or the 3′-position of a nucleoside, or a protected nucleoside can be protected (for example, by levulinoyl groups). In some embodiments, any amino groups attached to the heterocyclic base and/or a NH group(s) present in a ring of the heterocyclic base of the 5′-terminal residue can be protected with one or more appropriate protecting groups. Any protecting moieties present can be removed using methods known to those skilled in the art.

Some embodiments disclosed herein relate to a method of preparing a compound of Formula (I) and/or a compound of Formula (Ia). Other embodiments disclosed herein relate to a method of preparing a compound of Formula (II).

In some embodiments, a compound of Formulae (I) and/or (Ia) can be obtained through the following steps: (1) forming a phosphoroamidite at the 2′-position of the middle residue, (2) coupling the middle residue with the phosphoroamidite to the substituent at the 5′-position of the 2′-terminal residue, (3) optionally, adding a protecting group to the phosphoroamidite (for example, R³), (4) oxidizing the phosphorus to a phosphate, (5) optionally, transforming the phosphate to a phosphorothioate, (6) forming a phosphoroamidite at the 2′-position of the 5′-terminal residue, (7) coupling the 5′-terminal residue to the substituent at the 5′-position of the dimer of the middle and 2′-terminal residue, (8) optionally, adding a protecting group to the phosphoroamidite (for example, R²), (9) oxidizing the phosphorus to a phosphate and (10) optionally, transforming the phosphate to a phosphorothioate. Protecting groups can be added, exchanged and/or removed from the 5′,2′ and middle residues before and/or after any of the aforementioned steps. Similarly, the internal phosphates and/or phosphorothioates can be modified after the 5′,2′ and middle residues have been linked together. The group attached to the 5′-position of the 5′-terminal residue can also be modified after the 5′,2′ and middle residues have been linked together. Further details of the aforementioned steps are provided herein.

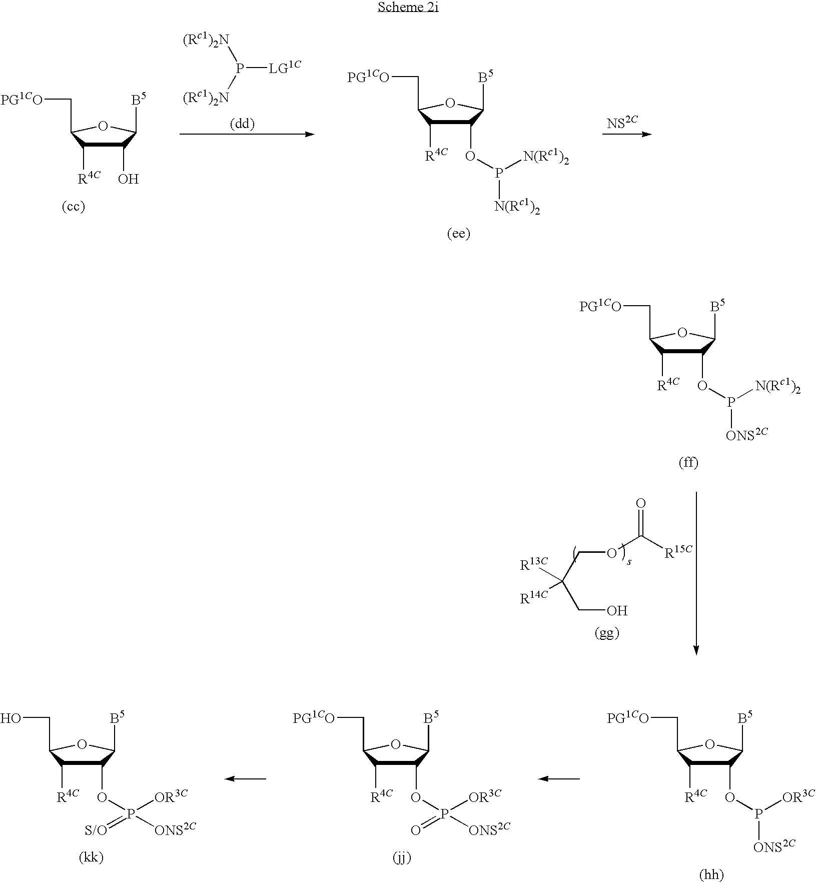

One embodiment disclosed herein relates to a method of synthesizing a compound of Formula kk that includes the transformations shown in Scheme 2i. In Scheme 2i, R^{3C}, R^{13C}, R^{14C}, R^{15C} and s can be the same as R³, R¹³, R¹⁴, R¹⁵ and p, respectively, as described above with respect to the compound of Formula (I). NS^{2C} represents a nucleoside, or a protected nucleoside, and PG^{1C} represents an appropriate protecting group. R^{4C} can be one of the substituents of R⁴ or —OPG^{3C}, wherein PG^{3C} represented a protecting group, and B⁵ can be an optionally substituted heterocyclic base, for example, those described with respect to B¹, or an optionally substituted heterocyclic base where any amino groups attached to the heterocyclic base and/or a NH group(s) present in a ring of the heterocyclic base are protected with an appropriate protecting group.

The protecting groups, PG^{1C} and PG^{3C} can be the same of different. In some embodiments PG¹ can be a triarylmethyl group. Exemplary triarylmethyl protecting group include, but are not limited to, trityl, monomethoxytrityl (MMTr), 4,4′-dimethoxytrityl (DMTr), 4,4′,4″-trimethoxytrityl (TMTr), 4,4′,4″-tris-(benzoyloxy)trityl (TBTr), 4,4′,4″-tris (4,5-dichlorophthalimido) trityl (CPTr), 4,4′,4″-tris(levulinyloxy)trityl (TLTr), p-anisyl-1-naphthylphenylmethyl, di-o-anisyl-1-naphthylmethyl, p-tolyldipheylmethyl, 3-(imidazolylmethyl)-4,4′-dimethoxytrityl, 9-phenylxanthen-9-yl (Pixyl), 9-(p-methoxyphenyl)xanthen-9-yl (Mox), 4-decyloxytrityl, 4-hexadecyloxytrityl, 4,4′-dioctadecyltrityl, 9-(4-octadecyloxyphenyl)xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl. In an embodiment, PG^{3C} can be a silyl ether group. A non-limiting list of silyl ether groups include trimethylsilyl (TMS), tert-butyldimethylsilyl (TBDMS), triisopropylsilyl (TIPS) and tert-butyldiphenylsilyl (TBDPS). When one or more protecting groups are present on B⁵, in some embodiments, at least one protecting group can be a benzoyl group. In other embodiments, at least one protecting group on B⁵ can be a triarylmethyl group, such as those described herein. The protecting groups can be selected such that the protecting group on the 5′-oxygen is more labile than the protecting group on the 3′-oxygen. By having a more labile protecting group on the 5′-oxygen, the 5′-oxygen protecting group can be selectively removed. For example, the 5′-oxygen protecting group, PG^{1C}, can be removed without removing the 3′-oxygen's protecting group, PG^{3C}. In some embodiments, PG^{1C} can be selectively removed without removing any protecting groups present on B⁵.

A compound of Formula ee can be obtained by forming a phosphoamidite at the 2′-position of a compound of Formula cc by reacting a compound of Formula dd with the —OH attached to the 2′-position of a compound of Formula cc to form a compound of Formula ee. In an embodiment, each R^{c1} can be independently an optionally substituted C₁₋₄ alkyl, and LG^{1C} can be a suitable leaving group. In an embodiment, the leaving group on a compound of Formula dd can be a halogen. One benefit of having the other hydroxy groups and any amino groups attached to the heterocyclic base and/or a NH group(s) present in a ring of the heterocyclic base protected is that the addition of a compound of Formula dd can be directed to the 2′-position of a compound of Formula cc. Furthermore, the protecting groups on the hydroxy groups and any amino groups attached to the heterocyclic base and/or a NH group(s) present in a ring of the heterocyclic base can block undesirable side reactions that may occur during later synthetic transformations. Minimization of unwanted side compound can assist in the separation and isolation of the desired compound(s).

A nucleoside, or a protected nucleoside can be added to a compound of Formula ee in which the —OH attached to the 5′-carbon group of the nucleoside or a protected nucleoside reacts with the phosphoamidite of a compound of Formula ee to form a compound of Formula ff. In some embodiments, the nucleoside or the protected nucleoside can have the structure of a compound of Formula II,

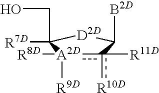

in which each [symbol] can be a double or single bond, provided that both [symbol] cannot be double bonds; A^{2D} can be selected from C (carbon), O (oxygen) and S (sulfur); B^{2D} can be selected from an optionally substituted heterocyclic base, and an optionally substituted protected heterocyclic base; D^{2D} can be C═CH₂ or O (oxygen); R^{7D} can be selected from hydrogen, azido, —CN, an optionally substituted C₁₋₄ alkyl and an optionally substituted C₁₋₄ alkoxy; R^{8D} can be absent or selected from hydrogen, halogen, hydroxy and an optionally substituted C₁₋₄ alkyl; R^{9D} can be absent or selected from hydrogen, halogen, azido, amino, hydroxy and —OPG^{1D}; R^{10D} can be absent or selected from hydrogen, halogen, hydroxy, —CN, —NC, an optionally substituted C₁₋₄ alkyl, an optionally substituted C₁₋₄ alkoxy and —OPG^{2D}; R^{11D} can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R^{10D} indicated by [symbol] is a double bond, then R^{10D} is a C₁₋₄ alkenyl and R^{11D} is absent; and PG^{1D} and PG^{2D} can each be a protecting group. In some embodiments, PG^{1D} can be a levulinoyl group. In some embodiments, PG^{2D} can be a levulinoyl group. In other embodiments, PG^{1D} can be a silyl ether group. In other embodiments, PG^{2D} can be a silyl ether group. In some embodiments, A^{2D} can be carbon, D^{2D} can be oxygen, and each [symbol] can be a single bond.

In an embodiment, B^{2D} can each be independently selected from:

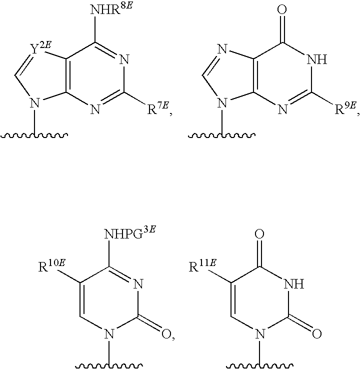

and

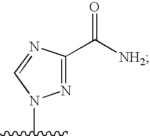

R^{7E} can be hydrogen or halogen; R^{8E} can be hydrogen, an optionally substituted C₁₋₄ alkyl, an optionally substituted C₃₋₈ cycloalkyl or PG^{4E}; R^{9E} can be hydrogen or amino; R^{10E} can be selected from hydrogen, halogen, an optionally substituted C₁₋₄ alkyl, an optionally substituted C₂₋₄ alkenyl and an optionally substituted C₂₋₄ alkynyl; R^{11E} can be selected from hydrogen, halogen, an optionally substituted C₁₋₄alkyl, an optionally substituted C₂₋₄ alkenyl and an optionally substituted C₂₋₄ alkynyl; and Y^{2E} can be N or CR^{12E}, wherein R^{12E} can be selected from hydrogen, halogen, an optionally substituted C₁₋₄-alkyl, an optionally substituted C₂₋₄-alkenyl, an optionally substituted C₂₋₄-alkynyl and PG^{3E} and PG^{4E} can be each independently hydrogen or a protecting group. In an embodiment, one or both of PG^{4E} and PG^{3E} can be a triarylmethyl protecting group such as those described herein. In an embodiment, B^{2D} can be

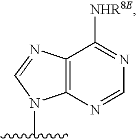

where R^{8E} can be hydrogen or PG^{4E}.

To facilitate the reaction between the nucleoside or the protected nucleoside (for example, a compound of Formula II) and a compound of Formula ee, an activator, such as a tetrazole, can be used. The tetrazole can protonate the nitrogen of the phosphoamidite making it susceptible to nucleophilic attack by the nucleoside or the protected nucleoside.

Optionally, a R^{3C} moiety can be added to a compound of Formula ff by reacting a compound of Formula ff with a compound of Formula gg to form a compound of Formula hh. An activator can also be used to promote this reaction as described above. As mentioned previously, having protecting group(s) on the hydroxy groups and any amino groups attached to the heterocyclic base and/or a NH group(s) present in a ring of the heterocyclic base can direct the addition of compounds such as a compound of Formula gg. As a result, undesirable side reactions that may occur during later synthetic transformations can be minimized, thus, making the separation and isolation of the desired compound(s) more facile.

The phosphite of a compound of Formula hh can be oxidized to a phosphate moiety to form a compound of Formula jj. In an embodiment, the oxidation can be carried out using iodine as the oxidizing agent and water as the oxygen donor. The phosphate moiety can be transformed to a phosphorothioate by using an appropriate sulfurization agent. Suitable sulfurization agents include, but are not limited to, elemental sulfur, Lawesson's reagent, cyclooctasulfur, 3H-1,2-Benzodithiole-3-one-1,1-dioxide (Beaucage's reagent) and 3-((N,N-dimethylaminomethylidene)amino)-3H-1,2,4-dithiazole-5-thione (DDTT).

The protecting group moiety, PG^{1C}, can be removed to form a compound of Formula kk. In an embodiment, PG^{1C} can be removed with a tetra(alkyl)ammonium halide such as tetra(t-butyl)ammonium fluoride. In some embodiments, PG^{1C} can be selectively removed such that PG^{1C} is removed without removing PG^{2C}, PG^{3C}, and/or any protecting groups on B^{2D}. For example, PG^{1C} can be removed using a reagent such as a tetra(alkyl)ammonium halide that does not remove PG^{2C}, PG^{3C}, and/or any protecting groups on B^{2D}.

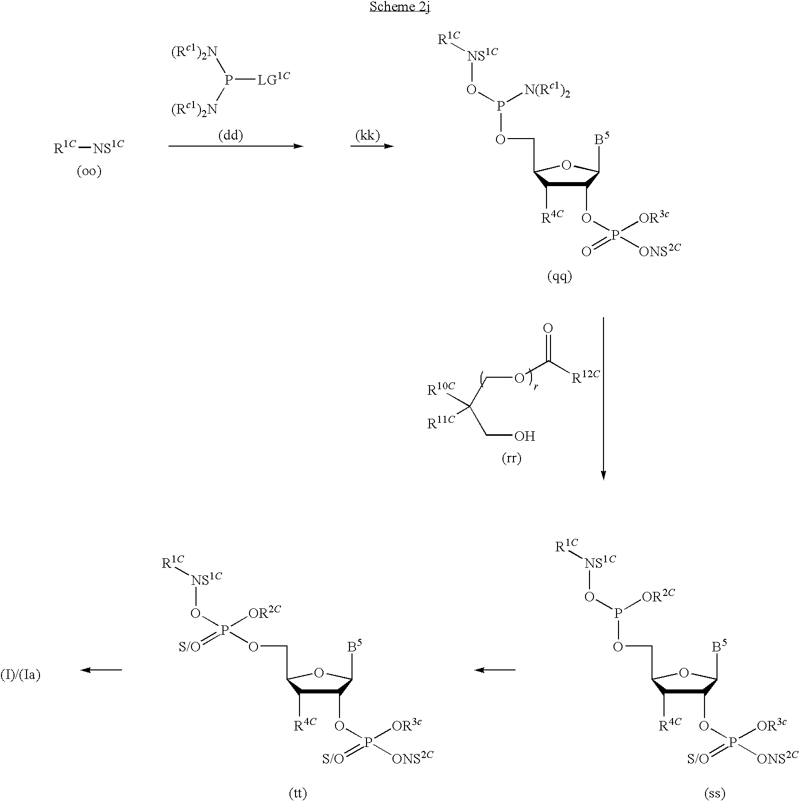

One method for obtaining compounds of Formulae (I) and (Ia) is shown in Scheme 2j. As shown in Scheme 2j, R^{1C}, R^{2C}, R^{3C}, R^{10C}, R^{11C}, R^{12C}, NS^{1C}, NS^{2C} and r can be the same as R¹, R², R³, R¹⁰, R¹¹, R¹², NS¹, NS² and n, respectively, as disclosed above for the compound of Formula (I), and R^{4C} and B⁵ can be same as described with reference to Scheme 2i. In some embodiments, NS^{2C} can be a compound of Formula II. If desired, any protecting groups attached to the 2′-position of NS^{1C} of a compound of Formula oo can be removed using one or more suitable reagents. For example, if the oxygen attached to the 2′-position of NS^{1C} is a levulinoyl group, the levulinoyl group can be removed using hydrazinium acetate. Likewise if the oxygen attached to the 2′-position is a silyl group, the silyl group can be removed using a tetraalkylammonium halide. In an embodiment, the tetraalkylammonium halide can be tetrabutylammonium fluoride.

A phosphoamidite at the 2′-position of a compound of Formula oo can be formed by reacting a compound of Formula oo with a compound of Formula dd. In an embodiment, each R^{c1} of a compound of Formula dd can be independently an optionally substituted C₁₋₄ alkyl, and LG^{1C} can be a suitable leaving group. In an embodiment, the leaving group on a compound of Formula dd can be a halogen. An advantage of having any hydroxy groups and any amino groups attached to the heterocyclic base and/or a NH group(s) present in a ring of the heterocyclic base protected on NS^{1C} is that the addition of a compound of Formula dd can be directed to the 2′-position of a compound of Formula oo. In some embodiments, the protecting groups on any hydroxy groups and any amino groups attached to the heterocyclic base and/or a NH group(s) present in a ring of the heterocyclic base thereof on NS^{1C} can block undesirable side reactions that may occur during later synthetic transformations. By minimizing any unwanted side reactions, separation and isolation of the desired compound(s) can be simplified.

A compound of Formula kk can be reacted with the phosphoamidite to form a compound of Formula qq. If desired, a R^{2c} moiety can be added to a compound of Formula qq by reacting a compound of Formula qq with a compound of Formula rr to form a compound of Formula ss. In some embodiments, the reaction between a compound of Formula rr and a compound of Formula qq can be facilitated using a suitable activator, such as those described herein. As mentioned previously, having protecting group(s) on the hydroxy groups and any amino groups attached to the heterocyclic base and/or a NH group(s) present in a ring of the heterocyclic base can direct the addition of compounds such as a compound of Formula rr. As a result, undesirable side reactions that may occur during later synthetic transformations can be minimized, thus, making the separation and isolation of the desired compound(s) more facile.

The phosphite of a compound of Formula ss can be oxidized to a phosphate moiety to form a compound of Formula tt. In some embodiments, the oxidation of the phosphorus can be carried out using one or more suitable oxidizing agents. In some embodiments, the oxidizing agent can be iodine and the oxygen donor can be water. In some embodiments, the phosphate can further be transformed to a phosphorothioate using an appropriate sulfurization agent, such as those described herein.

Any protecting groups on a compound of Formula tt can be removed using suitable reagents to yield a compound of Formulae (I) or (Ia). Any protecting groups present on B⁵, any additional protecting groups present attached to the heterocyclic bases of NS^{1C} and NS^{2C}, any protecting groups represented by R^{4C}, and any protecting group on the oxygens attached as hydroxy groups to the 2′ and 3′-positions of NS^{1C} and NS^{2C} can be removed using methods known to those skilled in the art to form a compound of Formula (I) or (Ia). In an embodiment, when a protecting group (such as benzoyl or a triarylmethyl group) is present on B⁵, it can be removed with an acid such as acetic acid or a zinc dihalide, such as ZnBr₂. In some embodiments, the heterocyclic bases of NS^{1C} and NS^{2C} are protected with triarylmethyl protecting groups which can removed with an acid (e.g., acetic acid). In some embodiments, levulinoyl protecting groups can be attached to one or more oxygens of NS^{2C}. In an embodiment, the levulinoyl protecting groups can be removed with hydrazinium acetate. In other embodiment, silyl ether protecting groups can be attached to one or more oxygens of NS^{2C}. In an embodiment, the silyl ether groups can be removed using a tetraalkylammonium halide (e.g., tetrabutylammonium fluoride). In some embodiments, the protecting groups on a compound of Formula tt can be removed selectively. In some embodiments, the protecting groups on the oxygens attached to the 2′ and 3′-positions of NS^{2C}, if present, can be removed selectively. For example, the groups on the oxygens attached to the 2′ and 3′-positions of NS^{2C} can be removed without removing any protecting groups attached to the heterocyclic bases of NS^{1C} and NS^{2C}. Alternatively, any protecting groups on the heterocyclic bases of NS^{1C} and NS^{2C} can be selectively removed such that the protecting groups on the heterocyclic bases of NS^{1C} and NS^{2C} can be removed without removing any protecting groups on the oxygens attached to the 2′ and 3′-positions of NS^{2C}. In an embodiment, the protecting groups on the oxygens attached to the 2′ and 3′-positions of NS^{2C}, if present, can be removed before removing any protecting groups on the heterocyclic bases of NS^{1C} and NS^{2C}. In another embodiment, the protecting groups on the oxygens attached to the 2′ and 3′-positions of NS^{2C}, if present, can be removed after removing any protecting groups on the heterocyclic bases of NS^{1C} and NS^{2C}.

Another method for forming compounds of Formula (I) and/or (Ia) having one or more phosphorothioates is provided below.

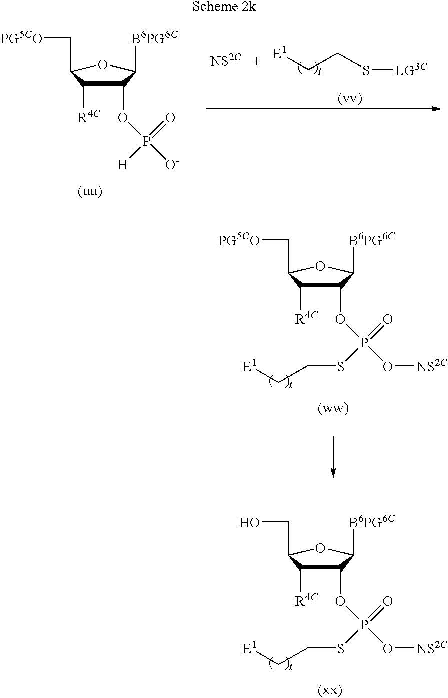

In Scheme 2k, NS^{2C} can be the same as NS² as described herein for a compound of Formula (I) or a compound of Formula II. The compound of Formula uu can be coupled with a compound of Formula vv and NS^{2C} to form a compound of Formula ww. In an embodiment, R^{4C} can be one of the substituents of R⁴ as described with respect to the compound of Formula (I) or —OPG^{7C}, where PG^{7C} represents a protecting group. In some embodiments, PG^{7C} can be a silyl ether group. In Scheme 2k, PG^{5C} and PG^{6C} represent appropriate protecting groups for the 5′-OH and heterocyclic base, respectively. A compound of Formula vv can be the following substituents: E¹ can be an electron-withdrawing group, LG^{3C} can be an appropriate leaving group and t can be 0, 1 or 2. One example of a suitable electron-withdrawing group is a cyano group. In an embodiment, the compound of Formula vv can be N-[(2-cyanoethyl)sulfanyl]phthalimide. In some embodiments, the reaction of NS^{2C} and compounds of Formulae uu and vv can be facilitated by using an activating reagent such as bis-(2-chlorophenyl)phosphorochloridate. As described herein, NS^{2C} can be a nucleoside, or a protected nucleoside, such as a compound of Formula pp. The PG^{6C} protecting group can be removed using methods known to those skilled in the art to form a compound of Formula xx. In some embodiments, when PG^{5C} is a silyl ether group, then PG^{5C} can be removed with tetraalkylammonium halide or hydrazinium acetate.

The 5′-terminal residue can be added to a compound of Formula xx using one or more methods described herein. In some embodiments, a 5′-terminal moiety can be coupled to a compound of Formula xx using similar transformations and condition as those described with respect to Scheme 2j. In other embodiments, a compound of formula uu can be coupled to a compound of formula xx using similar transformations and conditions for forming a compound of formula xx.

To form the phosphorothioate, the

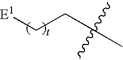

moiety can be cleaved using one or more methods known to those skilled in the art. For example, the moiety may be cleaved using an appropriate base. In some embodiments, the base can be an amidine or an amine base. Examples of suitable bases include, but are not limited to 1,8-diazabicyclo[5.4.0]undec-7-ene (DBU), 1,5-diazabicyclo[4.3.0]non-5-ene (DBN), or 1,4-diazabicyclo[2.2.2]octane (DABCO). In an embodiment, the

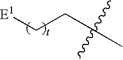

moiety can be cleaved with 1,8-diazabicyclo[5.4.0]undec-7-ene. In another embodiment, the

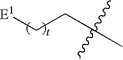

moiety can be cleaved with 1,8-diazabicyclo[5.4.0]undec-7-ene and chlorotrimethylsilane. The phosphorothioate(s) can be formed at any appropriate time during the synthesis. For example, the phosphorothioate(s) can be formed after formation of a dimer (for example, a compound of Formula xx). The phosphorothioate(s) can also be formed after the trimer has been fully assembled. In some embodiments, one or more phosphorothioates can be formed after one or more protecting groups on the heterocyclic bases have been removed. In other embodiments, one or more phosphorothioates can be formed before one or more protecting groups on the heterocyclic bases have been removed.

The compounds of Formula dd used form the phosphoamidites can be the same or different. In some embodiments, the compound

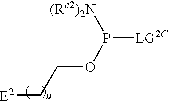

wherein R^{c2} can be each independently an optionally substituted C₁₋₄ alkyl, E² can be an electron-withdrawing group, LG^{2C} can be a leaving group and t can be 0, 1 or 2, can be substituted in place of a compound of Formula dd.

Compounds of Formulae (I), (Ia) and/or (II) can also be prepared utilizing a solid-phase method. In some embodiments, the monomeric residues can be attached to an appropriate solid-support loaded with a 3′-terminal residue. Various solid-supports and 3′-terminal residues are known to those skilled in the art. In some embodiments, the solid-support with a 3′-terminal residue can be 5′-O-DMT-A(NH-Bz)-2′-O-acetyl-3′-succinyl-CPG (controlled pore glass). In some embodiments, compounds of Formulae (I), (Ia) and/or (II) can be prepared through a method that includes the following steps: (1) removal of the protecting group on the oxygen attached to the 5′-position of the 3′-terminal residue, (2) coupling of a phosphoramidite to the 3′-terminal residue, (3) oxidation of the phosphorus to a phosphate and/or sulfurization to form a phosphorothioate (4) coupling of another phosphoramidite (5) oxidation of the phosphorus to a phosphate and/or sulfurization to form a phosphorothioate and (6) cleavage of the compound from the solid support. Additional steps that can be included include removal of any protecting groups present on the 2′-positions, 3′-positions, 5′-positions and/or heterocyclic bases (for example, any amino groups attached to the heterocyclic base and/or a NH group(s) present in a ring of the heterocyclic base). Suitable reagents for removing one or more of the protecting groups are known to those skilled in the art, and described herein. A non-limiting list of example reagents for removing the silyl protecting group(s) include tetrabutylammonium fluoride (TBAF) and triethylamine/triethylamine-3HF.

Various reagents know to those skilled in the art can be used to oxidize the phosphorus from phosphorus(III) to phosphorus(V). Suitable oxidizing agents are described herein. In some embodiments, the oxidizing agent can be iodine and the oxygen donor can be water. Likewise, various sulfurization agents are known to those skilled in the art. Examples of suitable sulfurization agents are described herein. In some embodiments, the sulfurization agent can be 3-[dimethylaminomethylidene]amino-3H-1,2,4-dithiazole-3-thione (DDTT). The solid support can be cleaved using suitable reagents known to those skilled in the art. An example of a suitable cleaving reagent is ammonium hydroxide.

### Pharmaceutical Compositions

Some embodiments described herein relates to a pharmaceutical composition, that can include a therapeutically effective amount of one or more compounds described herein (e.g., a compound of Formula (I), a compound of Formula (Ia) and/or a compound of Formula (II), or a pharmaceutically acceptable salt thereof) and a pharmaceutically acceptable carrier, diluent, excipient or combination thereof.

The term “pharmaceutical composition” refers to a mixture of a compound disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

One embodiment disclosed herein relates to a method of treating and/or ameliorating a disease or condition that can include administering to a subject a therapeutically effective amount of one or more compounds described herein, such as a compound of Formulae (I), (Ia) and/or (II), or a pharmaceutically acceptable salt thereof, or a pharmaceutical composition that includes a compound described herein.

Some embodiments disclosed herein relate to a method of ameliorating or treating a neoplastic disease that can include administering to a subject suffering from a neoplastic disease a therapeutically effective amount of one or more compounds described herein (e.g., a compound of Formulae (I), (Ia) and/or (II), or a pharmaceutically acceptable salt thereof, or a pharmaceutical composition that includes a compound described herein). In an embodiment, the neoplastic disease can be cancer. In some embodiments, the neoplastic disease can be a tumor such as a solid tumor. In an embodiment, the neoplastic disease can be leukemia. Exemplary leukemias include, but are not limited to, acute lymphoblastic leukemia (ALL), acute myeloid leukemia (AML) and juvenile myelomonocytic leukemia (JMML).

An embodiment disclosed herein relates to a method of inhibiting the growth of a tumor that can include administering to a subject having a tumor a therapeutically effective amount of one or more compounds described herein or a pharmaceutical composition that includes one or more compounds described herein.

Other embodiments disclosed herein relates to a method of ameliorating or treating a viral infection that can include administering to a subject suffering from a viral infection a therapeutically effective amount of one or more compounds described herein or a pharmaceutical composition that includes one or more compounds described herein. In an embodiment, the viral infection can be caused by a virus selected from an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae, a Togaviridae an Arenaviridae and/or a Bornaviridae. In some embodiments, the viral infection can be a hepatitis C viral infection. In other embodiments, the viral infection can be influenza. In still other embodiments, the viral infection can be HIV.

Still other embodiments disclosed herein relates to a method of ameliorating or treating a bacterial infection that can include administering to a subject suffering from a bacterial infection a therapeutically effective amount of one or more compounds described herein or a pharmaceutical composition that includes one or more compounds described herein. See Li et al. PNAS (2008) 105(52):20816-20821. In some embodiments, the bacterial infection can be a Gram-positive bacteria, such as Bacillus anthracis. In other embodiments, the bacterial infection can be a Gram-negative bacteria, for example, Escherichia coli.

Yet still other embodiments disclosed herein relates to a method of ameliorating or treating a parasitic disease that can include administering to a subject suffering from a parasitic disease a therapeutically effective amount of one or more compounds described herein or a pharmaceutical composition that includes one or more compounds described herein. In an embodiment, the parasite disease can be Chagas' disease.

In some embodiments, compounds disclosed herein, such as a compound of Formulae (I), (Ia) and/or (II), or a pharmaceutically acceptable salt thereof, or a pharmaceutical composition that includes a compound described herein, can be administered in combination with an agent(s) currently used in a conventional standard of care. For example, for the treatment of HCV, a compound disclosed herein can be used in combination with Pegylated interferon-alpha-2a or Pegylated interferon-alpha-2b (brand names Pegasys or PEG-Intron) and ribavirin. As another example, a compound disclosed herein can be used in combination with oseltamivir (Tamiflu) or zanamivir (Relenza). In other embodiments, compounds disclosed herein, such as a compound of Formulae (I), (Ia) and/or (II), or a pharmaceutically acceptable thereof, or a pharmaceutical composition that includes a compound described herein, can be substituted for an agent currently used in a conventional standard of care therapy. As an example, for the treatment of HCV, a compound disclosed herein can be used in place of ribavirin.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount needed to prevent, alleviate or ameliorate symptoms of disease or prolong the survival of the subject being treated This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages my be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### 2-acetyl-2-(hydroxymethyl)-3-oxobutyl acetate (1)

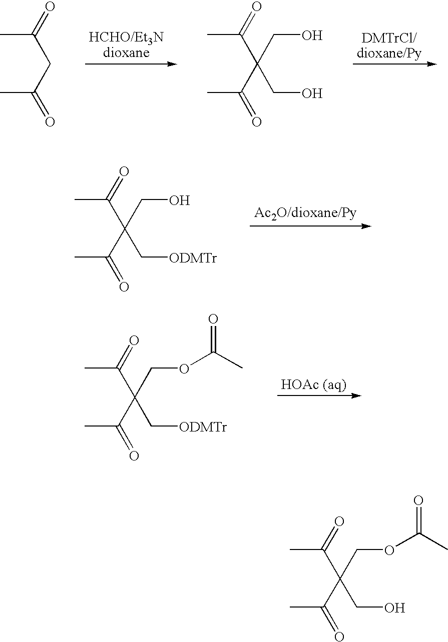

### 2-acetyl-2-hydroxymethyl-3-oxobutyl acetate (2)

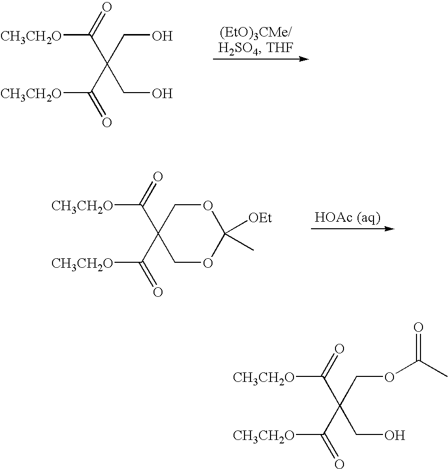

Diethyl 2-ethoxy-2-methyl-1,3-dioxane-5,5-dicarboxylate. Concentrated H₂SO₄ (1.3 mmol; 71 μL) was added to a mixture of diethyl 2,2-bis(hydroxymethyl)malonate (43.5 mmol, 9.6 g) and triethyl orthoacetate (65.2 mmol; 11.9 mL) in dry THF (15 mL). The reaction was allowed to proceed overnight and the mixture was the poured into an ice-cold solution of 5% NaHCO₃ (50 mL). The product was extracted with diethyl ether (2×50 mL), washed with saturated aqueous NaCl (2×50 mL) and dried over Na₂SO₄. The solvent was evaporated and the crude product was purified on a silica gel column eluting with a mixture of dichloromethane and methanol (95:5, v/v). The product was obtained as clear oil in 89% yield (11.3 g). ¹H NMR δ_{H} (500 MHz, CDCl₃): 4.30-4.36 (m, 6H, 4-CH₂, 6-CH₂ and 5-COOCH₂Me), 4.18 (q, J=7.1 Hz, 5-COOCH₂Me), 3.54 (q, J=7.10 Hz, 2H, 2-OCH₂Me), 1.46 (s, 3H, 2-CH₃), 1.32 (t, J=7.10 Hz, 3H, 2-OCH₂Me), 1.27 (t, J=7.1 Hz 3H, 5-COOCH₂Me), 1.26 (t, J=7.1 Hz 3H, 5-COOCH₂Me). ¹³C NMR (500 MHz, CDCl₃): δ=168.0 and 167.0 (5-COOEt), 111.1 (C2), 62.0 and 61.9 (5-COOCH₂Me), 61.6 (C4 and C6), 58.7 (2-OCH₂Me), 52.3 (C5), 22.5 (2-Me), 15.1 (2-OCH₂CH₃), 14.0 and 13.9 (5-COOCH₂CH₃).

Diethyl 2-(acetyloxymethyl)-2-(hydroxymethyl)malonate. Diethyl 2-ethoxy-2-methyl-1,3-dioxane-5,5-dicarboxylate (17.9 mmol; 5.2 g) was dissolved in 80% aqueous acetic acid (30 mL) and left for 2 h at room temperature. The solution was evaporated to dryness and the residue was co-evaporated three times with water. The product was purified by silica gel column chromatography eluting with ethyl acetate in dichloromethane (8:92, v/v). The product was obtained as yellowish oil in 75% yield (3.6 g). ¹H NMR δ_{H} (500 MHz, CDCl₃): 4.76 (s, 2H, CH₂OAc), 4.26 (q, J=7.10 Hz, 4H, OCH₂Me), 4.05 (d, J=7.10 Hz, 2H, CH₂OH), 2.72 (t, J=7.1 Hz, 1H, CH₂OH), 2.08 (s, 3H, Ac), 1.27 (t, J=7.10 Hz, 6H, OCH₂CH₃). ¹³C NMR (500 MHz, CDCl₃): δ=170.9 (C═O Ac), 168.1 (2×C═O malonate), 62.3 and 62.2 (CH₂OH and CH₂OAc), 61.9 (2×OCH₂CH₃) 59.6 (spiro C), 20.7 (CH₃ Ac), 14.0 (2×OCH₂CH₃).

### 2-acetyl-2-(hydroxymethyl)-3-oxobutyl pivalate (3)

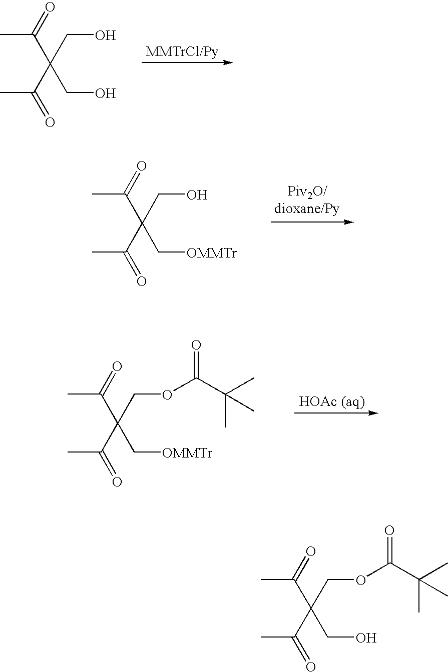

### 2,2-bis(ethoxycarbonyl)-3-hydroxypropyl pivalate (4)

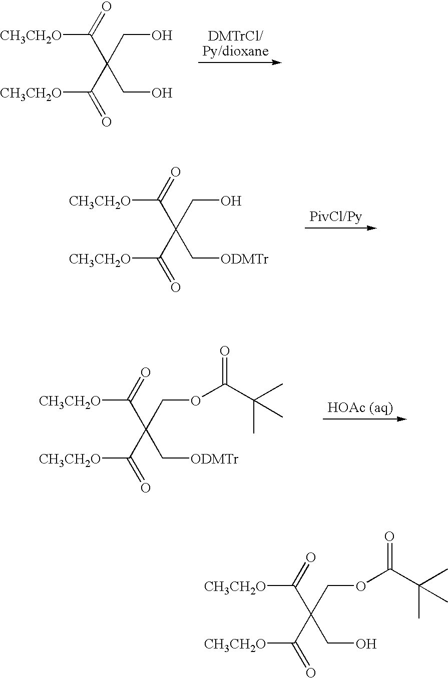

2,2-Bis(ethoxycarbonyl)-3-(4,4′-dimethoxytrityloxy)propyl pivalate. Diethyl 2,2-bis(hydroxymethyl)malonate was reacted with 1 equiv. of 4,4′-dimethoxytrityl chloride in 1,4-dioxane containing 1 equiv. of pyridine. Diethyl 2-(4,4′-dimethoxytrityloxymethyl)-2-(hydroxymethyl)malonate obtained (2.35 g, 4.50 mmol) was acylated with pivaloyl chloride (0.83 mL, 6.75 mmol) in dry MeCN (10 mL) containing 3 equiv. pyridine (1.09 mL, 13.5 mmol). After 3 days at room temperature, the reaction was quenched with MeOH (20 mL) and a conventional CH₂Cl₂/aq HCO₃⁻—workup was carried out. Silica gel chromatography (EtOAc/hexane 1:1, v/v) gave 2.47 g (90%) of the desired product as yellowish syrup. ¹H NMR (CDCl₃, 200 MHz): 7.13-7.39 [m, 9H, (MeO)₂ Tr]; 6.81 (d, 4H, [MeO]₂ Tr); 4.71 (s, 2H, CH₂OPiv); 4.15 (q, J=7.1, 4H, OCH₂CH₃); 3.78 [s, 6H, (CH₃O)₂Tr]; 3.67 (s, 2H, CH₂ODMTr); 1.27 (t, J=7.1, 6H, OCH₂CH₃); 1.02 [s, 9H, COC(CH₃)₃].

2,2-Bis(ethoxycarbonyl)-3-hydroxypropyl pivalate. 2,2-Bis(ethoxycarbonyl)-3-(4,4′-dimethoxytrityloxy)propyl pivalate (2.47 g, 4.07 mmol) in a 4:1 mixture of CH₂Cl₂ and MeOH (20 mL) was treated for 4 hours at room temperature with TFA (2.00 mL, 26.0 mmol) to remove the dimethoxytrityl group. The mixture was neutralized with pyridine (2.30 mL, 28.6 mmol), subjected to CH₂Cl₂/aq workup and purified by Silica gel chromatography (EtOAc/hexane 3:7, v/v) to obtain 1.15 g (93%) of the desired product. ¹H NMR (CDCl₃, 200 MHz): 4.59 (s, 2H, CH₂OPiv); 4.25 (q, J=7.1, 4H, OCH₂CH₃); 4.01 (s, 2H, CH₂OH); 1.28 (t, J=7.1, 6H, OCH₂CH₃); 1.18 [s, 9H, COC(CH₃)₃]. ESI-MS⁺: m/z 305.4 ([MH]⁺), 322.6 ([MNH₄]⁺), 327.6 ([MNa]⁺), 343.5 ([MK]⁺).

### Diethyl 2-acetyloxymethyl-2-hydroxymethylmalonate (5)

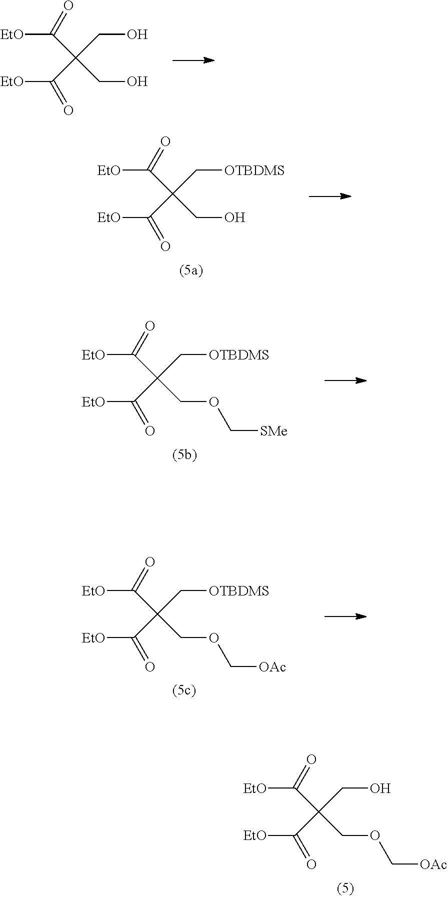

Diethyl 2-(tert-butyldimethylsilyloxymethyl)-2-hydroxymethylmalonate (5a). Diethyl 2,2-bis(hydroxymethyl)malonate (28.3 mmol; 6.23 g) was co-evaporated twice from dry pyridine and dissolved in the same solvent (20 mL). tert-Butyldimethylsilyl chloride (25.5 mmol; 3.85 g) in dry pyridine (10 mL) was added portionwise. The reaction was allowed to proceed for 4 days. The mixture was evaporated to a solid foam, which was then equilibrated between water (200 mL) and DCM (4×100 mL). The organic phase was dried on Na₂SO₄. The product was purified by silica gel chromatography eluting with 10% ethyl acetate in DCM. The yield was 78%. ¹H NMR (CDCl₃) δ 4.18-4.25 (m, 4H, OCH₂Me), 4.10 (s, 2H, CH₂OSi), 4.06 (s, 2H, CH₂OH), 2.63 (br s, 1H, OH), 1.26 (t, J=7.0 Hz, 6H, OCH₂CH₃), 0.85 (s, 9H, Si—SMe₃), 0.05 (s, 6H, Me-Si). ¹³C NMR (CDCl₃) δ 169.2 (C═O), 63.3 (CH₂OH), 62.8 (CH₂OSi), 61.6 (spiro C), 61.4 (OCH₂Me), 25.6 [C(CH₃)₃], 18.0 (Si—CMe₃), 14.0 (OCH₂CH₃), −3.6 (Si—CH₃). MS [M+H]⁺ obsd. 335.7, calcd. 335.2; [M+Na] obsd. 357.6, calcd. 357.2.

Diethyl 2-(tert-butyldimethylsilyloxymethyl)-2-methylthiomethylmalonate (5b). Compound 5a (19.7 mmol; 6.59 g) was dissolved into a mixture of acetic anhydride (40 mL), acetic acid (12.5 mL) and DMSO (61 mL) and the mixture was stirred overnight. The reaction was stopped by dilution with cold aqueous Na₂CO₃ (290 mL 10% aqueous solution) and the product was extracted in diethyl ether (4×120 mL). The combined organic phase was dried on Na₂SO₄. The product was purified by silica gel chromatography using DCM as an eluent. The yield was 91%. ¹H NMR (CDCl₃) δ 4.61 (s, 2H, OCH₂S), 4.14-4.19 (m, 4H, OCH₂Me), 4.06 (s, 2H, CH₂OSi), 4.00 (s, 2H, CH₂OCH₂SMe), 2.06 (SCH₃), 1.22 (t, J=7.0 Hz, 6H, OCH₂CH₃), 0.83 (s, 9H, Si—SMe₃), 0.02 (s, 6H, Me-Si). ¹³C NMR (CDCl₃) δ 168.3 (C═O), 75.6 (CH₂S), 65.7 (CH₂OCH₂SMe), 61.4 (CH₂OSi), 61.2 (spiro C), 60.9 (OCH₂Me), 25.6 [C(CH₃)₃], 18.0 (Si—CMe₃), 14.0 (OCH₂CH₃), 13.7 (SCH₃), −3.6 (Si—CH₃). MS [M+H]⁺ obsd. 395.4, calcd. 395.2; [M+Na]⁺ obsd. 417.6, calcd. 417.2.

Diethyl 2-acetyloxymethyl-2-(tert-butyldimethylsilyloxymethyl)malonate (5c). Compound 5b (17.9 mmol; 7.08 g) was dissolved in dry DCM (96 mL) under nitrogen. Sulfurylchloride (21.5 mmol; 1.74 mL of 1.0 mol L⁻¹ solution in DCM) was added in three portions and the mixture was stirred for 70 min under nitrogen. The solvent was removed under reduced pressure and the residue was dissolved into dry DCM (53 mL). Potassium acetate (30.9 mmol; 3.03 g) and dibenzo-18-crown-6 (13.5 mmol; 4.85 g) in DCM (50 mL) were added and the mixture was stirred for one hour and a half. Ethyl acetate (140 mL) was added, the organic phase was washed with water (2×190 mL) and dried on Na₂SO₄. The product was purified by silica gel chromatography using DCM as an eluent. The yield was 71%. ¹H NMR (CDCl₃) δ 5.24 (s, 2H, OCH₂O), 4.15-4.22 (m, 4H, OCH₂Me), 4.13 (s, 2H, CH₂OSi), 4.08 (s, 2H, CH₂OAc), 2.08 (Ac), 1.26 (t, J=8.0 Hz, 6H, OCH₂CH₃), 0.85 (s, 9H, Si—SMe₃), 0.04 (s, 6H, Me-Si). ¹³C NMR (CDCl₃) δ 170.2 (Ac), 168.0 (C═O), 89.3 (OCH₂O), 67.5 (CH₂OAc), 61.4 (OCH₂Me), 61.1 (CH₂OSi), 60.2 (spiro C), 25.6 [C(CH₃)₃], 21.0 (Ac), 18.1 (Si—CMe₃), 14.0 (OCH₂CH₃), −5.7 (Si—CH₃). MS [M+Na]⁺ obsd. 429.6, calcd. 429.2.

Diethyl 2-acetyloxymethyl-2-hydroxymethylmalonate (5). Compound 5c (7.2 mmol; 2.93 g) was dissolved in dry THF (23 mL) and trietylamine trihydrogenfluoride (8.64 mmol; 1.42 mL) was added. The mixture was stirred for one week. Aqueous triethylammonium acetate (13 mL of 2.0 mol L⁻¹ solution) was added. The mixture was evaporated to dryness and the residue was purified by silica gel chromatography using DCM containing 2-5% MeOH as an eluent. The yield was 74%. ¹H NMR (CDCl₃) δ 5.25 (s, 2H, OCH₂O), 4.16-4.29 (m, 6H, OCH₂Me and CH₂OAc), 4.13 (s, 2H, CH₂OH), 2.10 (Ac), 1.81 (br s, 1H, OH), 1.26 (t, J=9.0 Hz, 6H, OCH₂CH₃). MS [M+Na]⁺ obsd. 315.3, calcd. 315.1.

### 1-methyl 3-acetoxy-2-cyano-2-(hydroxymethyl)propanoate (6)

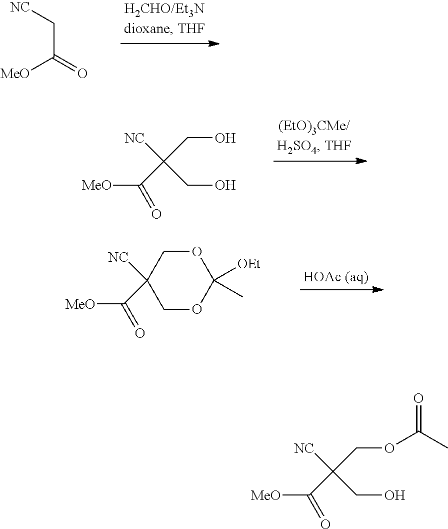

### 2-cyano-3-(ethylamino)-2-(hydroxymethyl)-3-oxopropyl acetate (7)

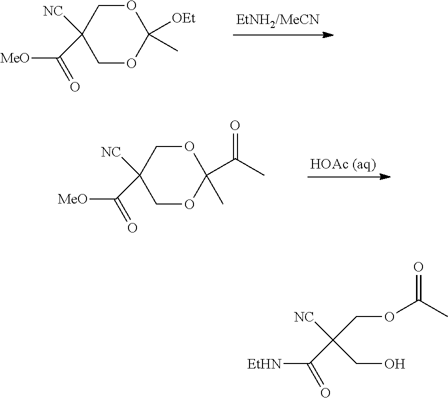

### 5′-O-(4,4′-dimethoxytrityl)-2′-O-(tert-butyldimethylsilyl)-3′-deoxyadenostne

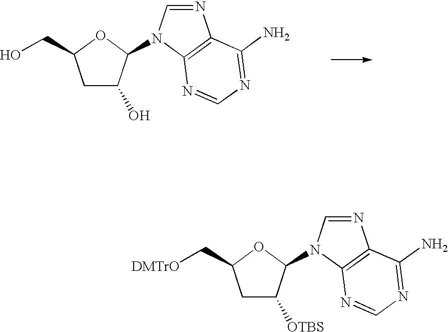

To a solution of 3′-deoxyadenosine (1.16 g, 4.62 mmol) in anhydrous pyridine (40 mL) under N₂, triethylamine (1.0 g, 6.93 mmol), DMAP (31 mg, 0.25 mmol), and DMTr-Cl (2.19 g, 6.47 mmol) were added. The reaction mixture was stirred at room temperature overnight. Completion of the reaction was verified using TLC. The reaction mixture was cooled to 5° C., and poured into 200 mL of water. The aqueous layer was extracted with ethyl acetate (3×100 mL), dried over anhydrous Na₂SO₄ and concentrated in-vacuo to dryness. The product, 5′-O-(4,4′-dimethoxytrityl)-3′-deoxyadenosine, was an off white foam, which was used in the next step without further purification.

To a solution of 5′-O-(4′,4′-dimethoxytrityl)-3′-deoxyadenosine in anhydrous DMF (25 mL) at 0° C. under N₂, imidazole (1.25 g, 18.5 mmol), DMAP (56 mg, 0.46 mmol) and TBDMSCl (1.4 g, 9.24 mmol) were added. The reaction mixture was stirred at room temperature overnight. Completion of the reaction was verified using TLC. The reaction mixture was cooled to 0° C., and saturated NaHCO₃ (100 mL) was added. The reaction mixture was diluted with ethyl acetate, dried over anhydrous Na₂SO₄ and concentrated. Chromatography on silica gel with MeOH:DCM:TEA (2:96:2 v/v) gave 5′-O-(4,4′-dimethoxytrityl)-2′-O-(tert-butyldimethylsilyl)-3′-deoxyadenosine as a white foam (1.7 g, 55%).

### 5′-O-(4,4′-dimethoxytrityl)-2′-O-(tert-butyldimethylsilyl)-N-6-phenoxyacetyl -3′-deoxyadenosine

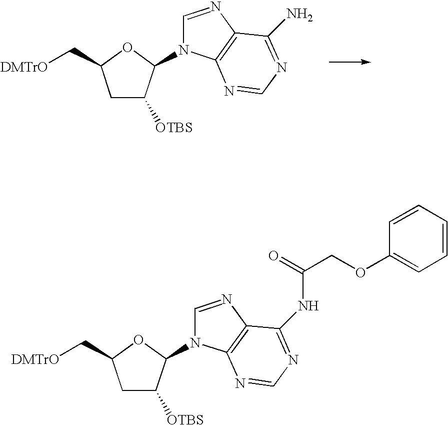

Phenoxyacetyl chloride (168 μl, 1.18 mmol) was added to a solution of 1-hydroxy-benzotriazole (1-HOBT) (0.16 g, 1.18 mmol) in CH₃CN:pyridine (1:1, 2 mL) under N₂. The reaction mixture was stirred at room temperature for 5 minutes. The reaction mixture was cooled to 0° C. (ice/water bath), and a pre cooled solution of 5′-O-(4,4′-dimethoxytrityl)-2′-O-(tert-butyldimethylsilyl)-3′-deoxyadenosine (495 mg, 0.74 mmol) in 25 mL of pyridine was added. The resulting mixture was stirred at room temperature overnight. The reaction mixture was then cooled to 0° C., and saturated NaHCO₃ (50 mL) was added. The reaction mixture was diluted with ethyl acetate, dried over anhydrous Na₂SO₄ and concentrated. Chromatography on silica gel with MeOH:DCM:TEA (1:97:2 to 2:96:2 v/v) gave 5′-O-(4,4′-dimethoxytrityl)-2′-O-(tert-butyldimethylsilyl)-N⁶-phenoxyacetyl -3′-deoxyadenosine (0.45 g, 76%).

### 5′-O-(4,4′-dimethoxytrityl)-N6-phenoxyacetyl-3′-deoxyadenosine

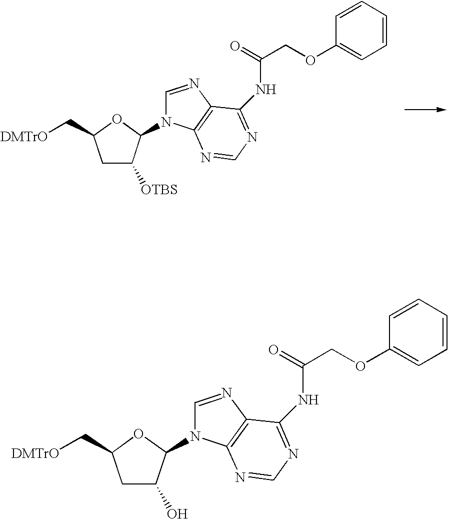

Tetrabutylammonium fluoride (TBAF) (2.39 mL, 1M in THF, 2.39 mmol) was added dropwise into a solution of 5′-O-(4,4′-dimethoxytrityl)-2′-O-(tert-butyldimethylsilyl)-N⁶-phenoxyacetyl-3′-deoxyadenosine (1.16 g, 2.0 mmol) in anhydrous THF (35 mL) at 0° C. After addition was complete, the reaction mixture was stirred at room temperature for 1 hour. Completion of the reaction was verified using TLC. The reaction mixture was cooled down to 0° C. EtOH (0.5 mL) was added followed by ethyl acetate (50 mL). The reaction mixture was then washed with brine three times, dried over anhydrous Na₂SO₄ and concentrated. Chromatography on silica gel with MeOH:DCM:TEA (0:99:1 to 2:96:2 v/v) gave 5′-O-(4,4′-dimethoxytrityl)-N⁶-phenoxyacetyl-3′-deoxyadenosine as a white foam (1.13 g, 82%).

### 5′-O-(4,4′-dimethoxytrityl)-N6-phenoxyacetyl-3′-deoxyadenosine-2′-O-[RP/SP]-(N,N,-diisopropylamino)methylphosphine

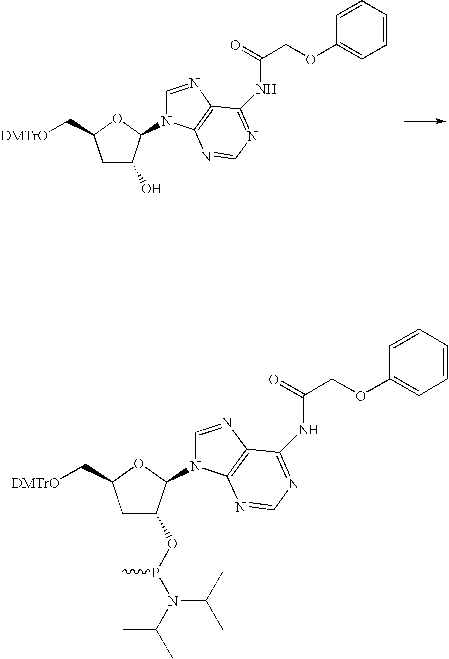

Chloro-N,N-diisopropylamino-methyl phosphine (410 μL, 2.26 mmol) and N,N-diisopropylethylamine (1.0 mL, 5.66 mmol) was added to a solution of 5′-O-(4,4′-dimethoxytrityl)-N⁶-phenoxyacetyl-3′-deoxyadenosine (0.62 g, 0.91 mmol) in anhydrous THF (4.4 mL) under N₂. The reaction mixture was stirred at room temperature for 2 hours. Completion of the reaction was verified using TLC. The reaction mixture was cooled to 5° C., and cold saturated aqueous NaHCO₃ was added. The reaction mixture was then extracted with ethyl acetate, dried over anhydrous Na₂SO₄ and concentrated. Chromatography on silica gel with ethyl acetate:hexane:triethanolamine (0:98:2 to 10:88:2 v/v) gave 5′-O-(4,4′-dimethoxytrityl)-N⁶-phenoxyacetyl-3′-deoxyadenosine-2′-O-[Rp/Sp]-(N,N,-diisopropylamino)methylphosphine as a white foam (430 mg, 57%) (Rp:Sp; ˜1:1 by ¹H NMR & ³¹P NMR). ¹H NMR (CDCl₃) δ 9.39 (br s, 1H, —NHCO), 8.79 & 8.72 (each s, 1H, H-8), 8.29 & 8.27 (each s, 1H, H-2), 7.45-6.19 (m, 18H, aromatic protons of DMTr and O—C₆H₅), 6.19 & 6.13 (each s, 1H, H1′), 4.86 (s, 2H, —O—CH₂-Ph), 4.79-4.59 (m, 2H, H2′, H4′), 3.79 & 3.78 (2s, 6H, 2×OCH₃), 3.52-3.31 (m, 4H, H5′, 2×CH iPr), 2.37-2.01 (m, 2H, H3′), 1.32-0.85 (m, 15H, P—CH₃, 2×CH(CH₃)₂); ³¹P NMR (CDCl₃) δ 124.9 and 119.9.

### 2-cyanoethyl-(N,N-diisopropylamino)methylphosphoramidite

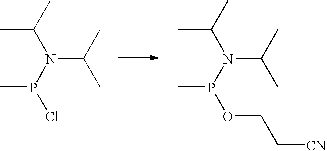

To a stirred solution of (N,N-diisopropylamino)methylphosphochloridate (1.82 g, 10 mmol) in anhydrous DCM at 0° C. was added 3-hydroxypropionitrile (0.71 mL, 10.5 mmol), followed by addition of N,N-diisopropylethylamine (3.65 mL, 21 mmol). The solution was then stirred at room temperature for 4 hours, cooled with ice, quenched with 2% NaHCO₃, diluted with EtOAc, washed with cold 2% NaHCO₃ three times, dried over sodium sulfate, and concentrated at room temperature to dryness. The syrup was dried under vacuum for 2 days. ¹H NMR (CDCl₃) δ 1.10 (d, 6H, J=6.8 Hz, iPr), 1.19 (d, 6H, J=6.4 Hz, iPr), 1.23 (d, 3H, J=8.0 Hz, P-Me), 2.59 (t, 2H, J=6.4 Hz, CH₂CN), 3.54 (m, 2H, iPr), 3.77 (m, 2H, CH₂OP); ³¹P NMR (CDCl₃) δ 124.98 (s).

### Dimethyl (2-((2R,3S,4R,5R)-5-(6-((bib(4-methoxyphenyl)(phenyl)methyl)amino)-9H-purin-9-yl)-4-hydroxy-3-methoxytetrahydrofuran-2-yl)ethyl)phosphonate (28)

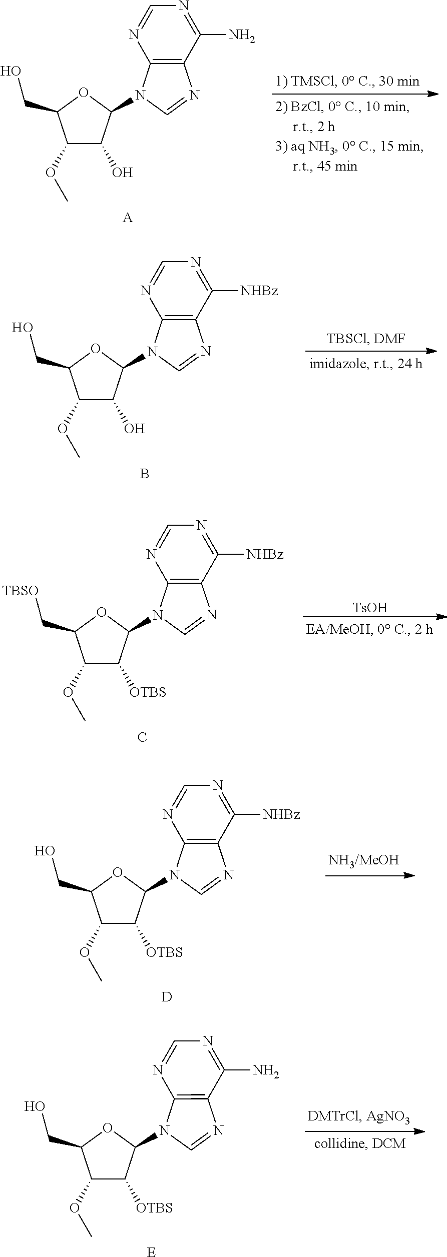

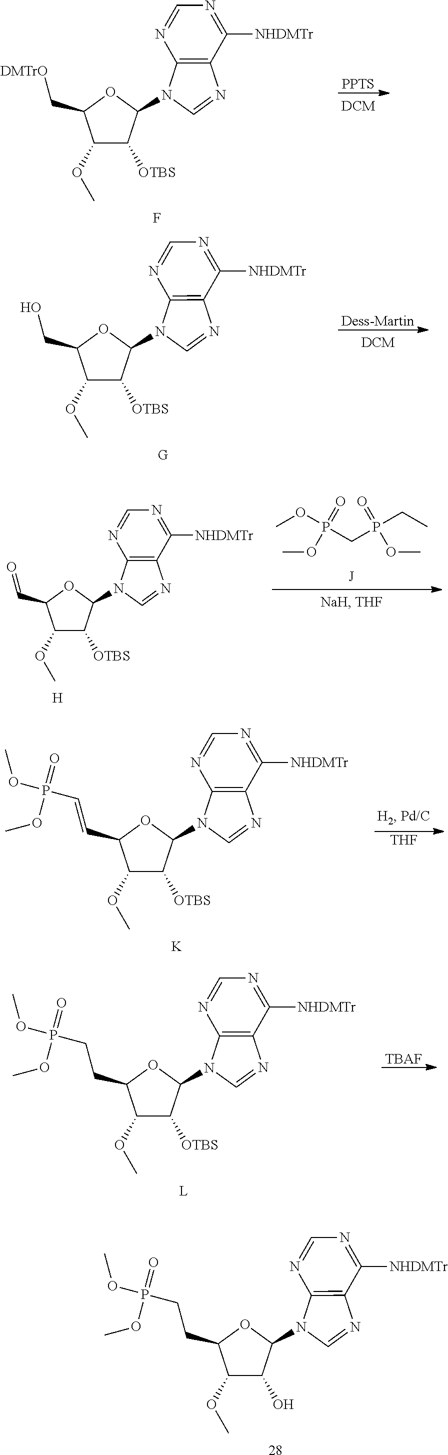

Compound A (9.5 g, 33.2 mmol) was co-evaporated with 100 mL anhydrous pyridine three times, re-dissolved in anhydrous pyridine (300 mL) and cooled to 0° C. under nitrogen. TMSCl (18.4 g, 170 mmol) was added dropwise, and the mixture was stirred at 0° C. for 30 minutes. BzCl (19.6 mL, 170 mmol) was then added dropwise over 10 minutes. After addition was complete, the reaction mixture stirred at 0° C. for 10 minutes and then stirred for 2 hours at ambient temperature. The reaction mixture was cooled to 0° C. and water (76 mL) was added. The reaction mixture was further stirred for 15 minutes. Aqueous ammonium hydroxide (18M, 76 mL) was then added. The reaction mixture was stirred for 15 minutes at 0° C. and then 45 minutes at ambient temperature. The reaction mixture was concentrated, and a white solid was recovered. The solid was filtered and washed with water and ethyl acetate to afford purified compound B (11.8, 92.2%) as a white solid. ¹H NMR (DMSO-d6, 400 MHz): δ 11.2 (s, 1H), 8.74 (s, 1H), 8.71 (s, 1H), 8.02 (d, J=7.2 Hz, 2H), 7.61-7.65 (m, 1H), 8.02 (t, J=7.6 Hz, 2H), 6.01 (d, J=6.0 Hz, 1H), 5.63 (d, J=6.0 Hz, 1H), 5.19 (t, J=4.8 Hz, 1H), 4.80 (q, J=5.6 Hz, 1H), 4.06 (q, J=4.0 Hz, 1H), 3.89 (q, J=3.6 Hz, 1H), 3.71-3.66 (m, 1H), 3.59-3.53 (m, 1H), 3.40 (s, 3H).

A solution of TBSCl (11.6 g, 78.0 mmol) in anhydrous DMF (20 mL) was added dropwise under nitrogen to an ice-cold mixture of compound B (12.0 g, 31.2 mmol) and imidazole (7.4 g, 190 mmol) in anhydrous DMF (80 mL). The reaction mixture was stirred at room temperature overnight. Ethyl acetate (300 mL) was added to the mixture. The mixture was then washed with brine (3×100 mL). The ethyl acetate layer was separated, dried over anhydrous Na₂SO₄ and filtered. The filtrate was concentrated under vacuum to give compound C (19.1 g, crude) as a white solid, which was used without further purification. LCMS calculated 613.31, observed 614.6 (M+1).

To an ice-cold solution of compound C (25.0 g, 40 mmol) in 300 mL ethyl acetate/methanol (1/1, v/v) was added dropwise a solution of TsOH.H₂O (15.5 g, 82 mmol) in 80 mL ethyl acetate/methanol (1/1, v/v). The reaction mixture was stirred at 0° C. for 6 hours. Completion of the reaction was verified using TLC. Solid K₂CO₃ (16 g, 120 mmol) was added, and the mixture was stirred at 0° C. for 1 hour. The reaction mixture was filtered. The filtrate was concentrated, re-dissolved in ethyl acetate (200 mL), washed with water and brine. The ethyl acetate layer was separated, dried over anhydrous Na₂SO₄ and concentrated. The residue was purified by chromatography on silica gel (CH₂Cl₂/CH₃OH=100:1 to 40:1) to give compound D (13.2 g, 69%). ¹H NMR (DMSO-d6, 400 MHz): δ 11.21 (brs, 1H), 8.73 (s, 1H), 8.72 (s, 1H), 8.05 (d, J=7.2 Hz, 2H), 7.64 (t, J=7.2 Hz, 1H), 7.55 (t, J=7.6 Hz, 2H), 6.06 (d, J=5.6 Hz, 1H), 5.30 (t, J=6.4 Hz, 1H), 4.94 (t, J=4.8 Hz, 1H), 4.13 (q, J=3.6 Hz , 1H), 3.92 (m, 1H), 3.76 (m, 1H), 3.63 (m, 1H), 3.43 (s, 3H), 0.76 (s, 9H), −0.03 (s, 3H), −0.2 (s, 3H).

Compound D (10.0 g, 20 mmol) was dissolved in 5M NH₃ in CH₃OH (100 mL) at 0° C. The mixture was stirred overnight at ambient temperature. The mixture was then concentrated under vacuum to give compound E (7.9 g, crude) as a white solid, which was used without further purification. LCMS calculated 395.20, observed 396.3 (M+1).

Collidine (24.2 g, 200 mmol) was added dropwise under nitrogen to an ice-cold mixture of compound E (7.9 g, 20 mmol), AgNO₃ (13.5 g, 80 mmol) and DMTrCl (27.1 g, 80 mmol) in anhydrous CH₂Cl₂ (80 mL). The reaction mixture was stirred at room temperature overnight. The mixture was then filtered through a pad of celite, and the filtrate was concentrated. The recovered residue was purified by chromatography on silica gel (PE/EA=10:1 to 5:1) to give compound F (18.3 g, 91%) as yellow foam solid.

A solution of PPTS (0.13 g, 0.5 mmol) in dry CH₂Cl₂ was added dropwise to an ice-cold solution of compound F (0.5 g, 0.5 mmol) in dry CH₂Cl₂ (5 mL). The reaction mixture was stirred at the room temperature overnight. The reaction was monitored by TLC. The reaction mixture was washed with brine (100 mL×3). The organic layer was separated, dried over anhydrous Na₂SO₄ and filtered. The filtrate was concentrated, and the residue was purified by chromatography on silica gel (PE/EA=10:1 to 2:1) to give compound G (0.25 g, 73%) as a white foam solid. ¹H NMR (DMSO-d6, 400 MHz): δ 8.45 (s, 1H), 7.90 (s, 1H), 7.25 (m, 5H), 7.16 (d, J=7.2 Hz , 4H), 6.82 (d, J=7.2 Hz, 4H), 5.87 (d, J=6 Hz, 1H), 5.42 (q, J=4.8 Hz, 1H), 4.86 (t, J=6.4 Hz, 1H), 4.07 (q, J=4 Hz, 1H), 3.84 (q, J=2.8 Hz , 1H), 3.70 (s, 6H), 3.39 (s, 3H), 3.63 (m, 1H), 0.68 (s, 9H), −0.11 (s, 3H), −0.29 (s, 3H).

To an ice-cold suspension of Dess-Martin (1.67 g, 3.95 mmol) in dry CH₂Cl₂ (30 mL) was added a solution of compound G (2.3 g, 3.3 mmol) in dry CH₂Cl₂ (10 mL) under nitrogen. The reaction mixture was stirred at the ambient temperature for 4 hours. Completion of the reaction was verified using TLC. The solution was washed with aqueous Na₂S₂O₃, aqueous Na₂HCO₃ and brine (30 mL×3). The organic layer was separated, dried over anhydrous Na₂SO₄ and concentrated under vacuum to give compound H (2.0 g, crude) as a yellow foam solid, which was used without further purification.

To an ice cold solution of compound J (1.2 g, 5.18 mmol) in dry THF (20 mL) was added NaH (0.26 g, 6.45 mmol) in one portion under nitrogen. The mixture was stirred at 0° C. for 30 min. A solution of compound H (3.3 g, 4.3 mmol) in dry THF (5 mL) was added dropwise under nitrogen to the solution containing compound J. The reaction mixture was stirred at the ambient temperature for 1 hour and then quenched with aqueous NH₄Cl. The reaction mixture was then extracted with ethyl acetate. The ethyl acetate layer was separated, dried over anhydrous Na₂SO₄ and concentrated under vacuum to give compound K (2.6 g, crude) as a yellow foam solid, which was used without further purification. ¹H NMR (MeOD, 400 MHz): δ 8.26 (s, 1H), 7.90 (s, 1H), 7.30 (m, 2H), 7.21 (m, 7H), 7.01 (m, 1H), 6.79-4.75 (m, 1H), (d, J=8.4 Hz, 4H), 6.25 (m, 1H), 5.98 (d, J=6 Hz, 1H), 5.17 (dd, J₁=4.8 Hz, J₂=1.6 Hz, 1H), 3.90 (m, 1H), 3.74 (s, 6H), 3.72 (d, J=6.8 Hz, 3H), 3.70 (d, J=6.8 Hz 3H), 3.52 (s, 3H), 0.74 (s, 9H), −0.04 (s, 3H), −0.26 (s, 3H).

To a suspension of PtO₂ (2.0 g, 2.5 mmol) in CH₃OH (20 mL) was added compound K (0.2 g) at ambient temperature under nitrogen. The reaction mixture was stirred under hydrogen (50 psi) for 1 hour. The reaction mixture was then filtered through a pad of celite. The filtrate was concentrated under vacuum to give compound L (2.0 g, crude) as a yellow foam solid, which was used without further purification. ¹H NMR (MeOD, 400 MHz): δ 8.26 (s, 1H), 7.90 (s, 1H), 7.30 (m, 2H), 7.21 (m, 7H), 6.78 (d, J=2 Hz, 4H), 5.88 (d, J=6 Hz, 1H), 5.10 (q, J=5.4 Hz, 1H), 4.13 (m, 1H), 3.76 (s, 6H), 3.74 (s , 1H), 3.70 (s, 3H), 3.67 (s, 3H), 3.48 (s, 3H), 1.98 (m, 4H), 0.76 (s, 9H), −0.02 (s, 3H), −0.24 (s, 3H).

Compound L (2.0 g, 2.5 mmol) and TBAF (0.97 g, 3.7 mmol) were dissolved in THF (20 mL) at ambient temperature. The reaction mixture was stirred overnight and then concentrated under vacuum. The residue was purified by chromatography on silica gel (CH₂Cl₂/CH₃OH=100:1 to 30:1) to give compound 28 (1.3 g, 76.5% for 3 steps) as a white foam solid. ¹H NMR (MeOD, 400 MHz): δ 8.22 (s, 1H), 7.78 (s, 1H), 7.29 (m, 2H), 7.20 (m, 7H), 6.78 (d, J=2.4 Hz, 4H), 5.88 (d, J=5.2 Hz, 1H), 4.94 (t, J=5.2 Hz, 1H), 4.09 (m, 1H), 3.87 (s, 1H), 3.73 (s , 6H), 3.67 (s, 3H), 3.64 (s, 3H), 3.47 (s, 3H), 1.95 (m, 4H).

### {2-[(2R,3S,4R,5R)-5-(6-benzoylamino-purin-9-yl)-4-hydroxy-3-methoxy-tetrahydro-furan-2-yl]-ethyl}-phosphonic acid dimethyl ester (29)

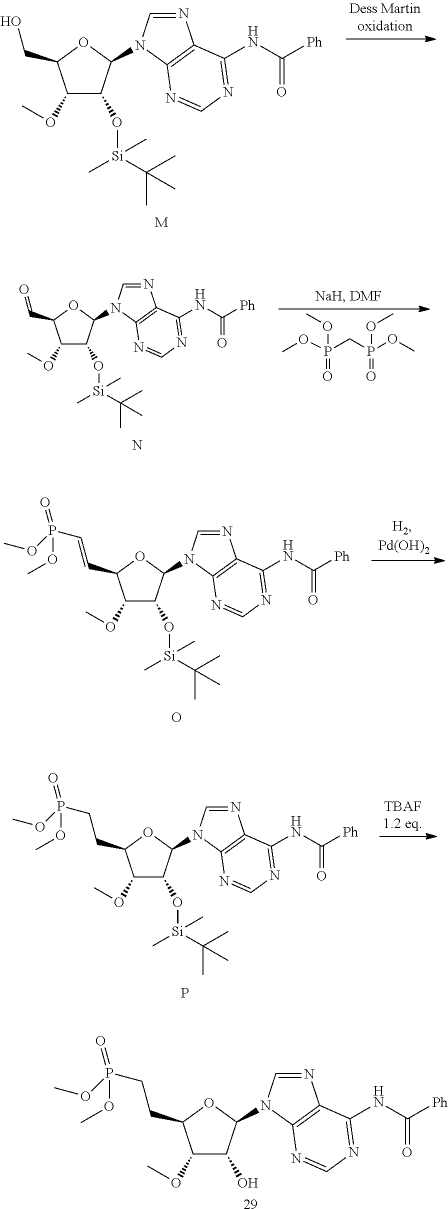

{(E)-2-[(2R,3R,4R,5R)-5-(6-Benzoylamino-purin-9-yl)-4-(tert-butyl-dimethyl-silanyloxy)-3-methoxy-tetrahydro-furan-2-yl]-vinyl}-phosphonic acid dimethyl ester (O). 3′-methoxy-2′-tert-butyl-dimethylsilyl-N-benzoyl adenosine (compound M, 1.03 g, 2.06 mmol) was co-evaporated twice with anhydrous DCM (20 mL). Compound M then dissolved in DCM (20 mL) and pyridine (0.17 mL, 2.06 mmol). Dess-Martin periodinane (1.05 g, 2.5 mmol) was added, and the reaction mixture stirred for 4 hours under Ar. DCM was evaporated off, and the mixture was taken up in ethyl acetate and saturated NaHCO₃. An extractive work-up afforded the crude aldehyde product (compound N) (0.92 g) as a foam. (Dimethoxy-phosphorylmethyl)-phosphonic acid dimethyl ester (0.64 g, 2.77 mmol) was dissolved in DMF (12 mL) and sodium hydride (60%, 0.08 g, 2.03 mmol) was added portion-wise. The mixture was stirred at room temperature for 0.5 hours. Compound N was dissolved in DMF (12 mL), and then added dropwise to the (Dimethoxy-phosphorylmethyl)-phosphonic acid dimethyl ester solution. The reaction mixture was stirred at ambient temperature under Ar for 5 hours. DMF was evaporated off, and the crude product was obtained after an extractive work-up with ethyl acetate and saturated NaHCO₃. The crude product was purified using silica gel and a gradient of methanol in DCM. Compound 5 was obtained as a white solid (0.62 g, 55%). LCMS: calculated 603.7, observed 604.7 (M+1), 626.4 (M+23).

{2-[(2R,3R,4R,5R)-5-(6-Benzoylamino-purin-9-yl)-4-(tert-butyl-dimethyl-silanyloxy)-3-methoxy-tetrahydrofuran-2-yl]-ethyl}-phosphonic acid dimethyl ester (P). Compound O (0.62 g, 1.03 mmol) was dissolved in (100 mL) and Pearlman's catalyst (0.1 g) was added. The reaction mixture was subjected to hydrogenation under a balloon for 18 hours. The reaction mixture was then filtered through a pad of Celite, and the filtrate was dried to afford compound P as a foam (0.48 g, 73%), which was used without further purification. LCMS calculated 605.7, observed 606.8 (M+1), 628.3 (M+23).

Compound 29. Compound P (0.48 g, 0.79 mmol) was dissolved in THF (10 mL). TBAF (1M in THF, 0.95 mL) was then added dropwise under Ar. After stiffing at ambient temperature >2 hours, the reaction mixture was evaporated and quenched with saturated aqueous NaHCO₃. Following an extractive workup with ethyl acetate, the crude product was purified (silica gel using a gradient of MeOH in DCM) to afford compound 29 as a white solid (0.18 g, 46%). LCMS calculated 491.4, observed 492.7 (M+1), 514.3 (M+23). ¹H NMR (DMSO-d6, 400 MHz) δ 11.22 (s, 1H), 8.76 (s, 1H), 8.69 (s, 1H), 8.04 (d, 2H, J=7.2 Hz), 7.65 (t, 1H, J=7.6 Hz), 7.55 (t, 2H, J=8 Hz), 5.98 (d, 1H, J=6.4 Hz), 5.62 (d, 1H, J=6.4 Hz), 4.97 (q, 1H, J=6 Hz), 4.06 (m, 1H), 3.87 (t, 1H, J=4.4 Hz), 3.61 (d, 3H, J=1.6 Hz), 3.59 (d, 3H, J=1.2 Hz), 3.44 (s, 3H), 2.0-1.7 (m, 4H).

### 3-[(2R,3S,4R,5R)-5-(6-benzoylamino-purin-9-yl)-4-hydroxy-3-methoxy-tetrahydro-furan-2-yl]-propionic acid methyl ester (30)

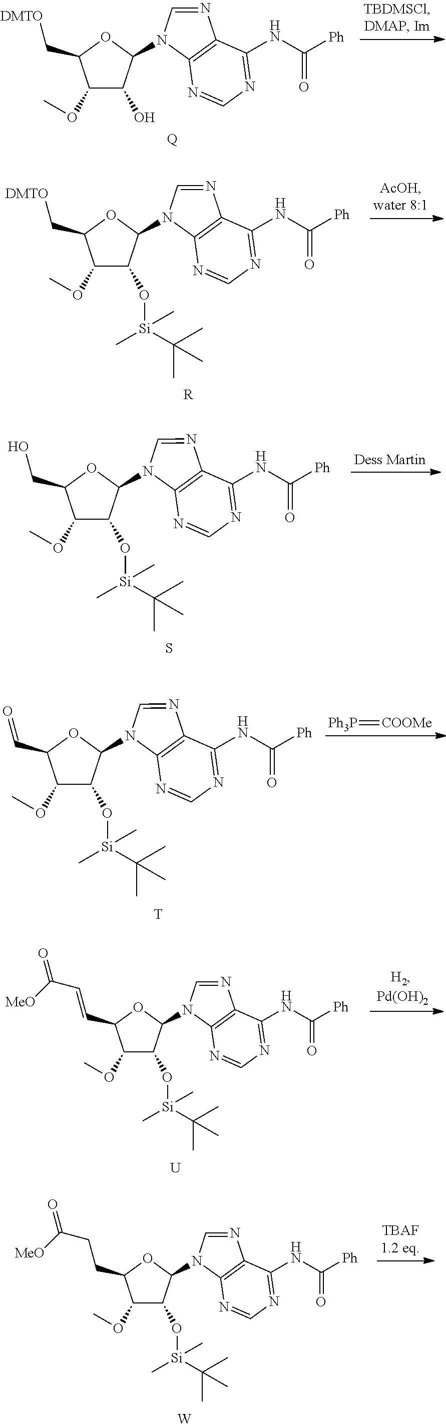

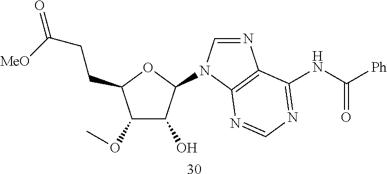

3′-Methoxy-2′-tert-butyl-dimethylsilyl-N-benzoyl Adenosine (S). 3′-Methoxy-5′-dimethoxytrityl-N-benzoyl Adenosine, compound Q, (purchased from ChemGenes, 2.12 g, 3.1 mmol), DMAP (0.036 g, 0.3 mmol) and imidazole (0.84 g, 12.4 mm01) were dissolved in DMF (18 mL). The reaction mixture was cooled to 0° C. and TBDMS-chloride (0.93 g, 6.2 mmol) was added portion-wise under a stream of Ar. The reaction mixture was allowed to warm to ambient temperature and stirred for 18 hours. The reaction mixture was concentrated using a rotovap and then quenched with a saturated sodium bicarbonate solution. After a normal extractive workup with ethyl acetate, the organic layers were pooled and dried to afford the crude 3′-methoxy-2′-tert-butyl-dimethylsilyl 5′-dimethoxytrityl N-benzoyl adenosine, compound R, as an oil. The oil was dissolved in a mixture of acetic acid (80 mL) and water (20 mL). The reaction mixture was then stirred at ambient temperature for 18 hours. The reaction was quenched by addition of water (50 mL). The reaction mixture was then concentrated using a rotovap and neutralized by addition of saturated aqueous NaHCO₃. After a normal extractive work-up with ethyl acetate, the crude product was purified (silica gel using a gradient of MeOH in DCM) to afford compound S as a white solid (1.21 g, 78%). LCMS calculated 499.63, observed 500.5 (M+1). ¹H NMR (DMSO-d6, 400 MHz) δ 11.3 (s, 1H), 8.76 (s, 1H), 8.75 (s, 1H), 8.04 (d, 2H, J=6.8 Hz), 7.64 (t, 1H, J=7 Hz), 7.55 (t, 2H, J=8 Hz), 6.05 (d, 1H, J=6 Hz), 5.29 (t, 1H, J=6 Hz), 4.94 (t, 1H, J=5 Hz), 4.11 (q, 1H, J=4 Hz), 3.92 (t, 1H, J=4 Hz), 3.76 (m, 1H), 3.64 (m, 1H), 3.43 (s, 3H), 0.76 (s, 9H), −0.03 (s, 3H), −0.20 (s, 3H).

(E)-3-[(2R,3R,4R,5R)-5-(6-Benzoylamino-purin-9-yl)-4-(tert-butyl-dimethyl-silanyloxy)-3-methoxy-tetrahydro-furan-2-yl]-acrylic acid methyl ester (U). 3′-Methoxy-2′-tert-butyl-dimethylsilyl-N-benzoyl adenosine (compound S, 0.48 g, 0.96 mmol) was dissolved in DCM (5 mL) and pyridine (0.08 mL, 0.96 mmol). To this solution was added Dess-Martin periodinane (0.8 g, 1.92 mmol). The reaction was then stirred for 4 hours under Ar. Methyl(triphenylphosphoranylidene)acetate (0.48 g, 1.44 mmol) was added, and the reaction mixture stirred for 18 hours. DCM was evaporated, and the reaction mixture was subjected to an extractive work-up with NaHCO₃ and ethyl acetate. The crude product was purified using silica gel and a gradient of ethyl acetate in hexanes to afford compound U as a white solid (0.37 g, 70%). LCMS calculated 553.7, observed 554.7 (M+1). ¹H NMR (DMSO-d6, 400 MHz) δ 11.24 (br s, 1H), 8.76 (br s, 2H), 8.05 (d, 2H, J=5 Hz), 7.65 (t, 1H, J=8 Hz), 7.56 (t, 2H, J=8 Hz), 7.17 (dd, 1H, J=6, 16 Hz), 6.19 (dd, 1H, J=2, 16 Hz), 6.12 (d, 1H, J=6 Hz), 5.17 (t, 1H, J=5 Hz), 4.79 (m, 1H), 4.04 (br m, 1H), 3.70 (s, 3H), 3.48 (s, 3H), 0.07 (s, 9H), 0.00 (s, 3H), −0.21 (s, 3H).

3-[(2R,3R,4R,5R)-5-(6-Benzoylamino-purin-9-yl)-4-(tert-butyl-dimethyl-silanyloxy)-3-methoxy-tetrahydro-furan-2-yl]-propionic acid methyl ester (W). (E)-3-[(2R,3R,4R,5R)-5-(6-Benzoylamino-purin-9-yl)-4-(tert-butyl-dimethyl-silanyloxy)-3-methoxy-tetrahydro-furan-2-yl]-acrylic acid methyl ester (compound U, 0.37 g, 0.67 mmol) was dissolved in CHCl₃ (5 mL) and ethanol (100 mL). Pearlman's catalyst (0.2 g) was added, and the reaction mixture was subjected to hydrogenation under a balloon for 18 hours. The reaction mixture was then filtered through a pad of Celite, and the filtrate was dried to afford the desired product as a foam (0.37 g), which was used without further purification. LCMS calculated 555.7, observed 556.6 (M+1)

3-[(2R,3S,4R,5R)-5-(6-Benzoylamino-purin-9-yl)-4-hydroxy-3-methoxy-tetrahydro-furan-2-yl]-propionic acid methyl ester (30). 3-[(2R,3R,4R,5R)-5-(6-Benzoylamino-purin-9-yl)-4-(tert-butyl-dimethyl-silanyloxy)-3-methoxy-tetrahydro-furan-2-yl]-propionic acid methyl ester (compound W, 0.37 g, 0.67 mmol) was dissolved in THF (9 mL) and TBAF (1M in THF, 0.8 mL) was added dropwise under Ar. After stirring at ambient temperature for 2 hours, the reaction mixture was evaporated and quenched with saturated aqueous NaHCO₃. After an extractive workup with ethyl acetate, the crude product was purified (silica gel using a gradient of MeOH in DCM) to afford compound 30 as a white solid (0.2 g, 69%). LCMS calculated 441.4, observed 442.6 (M+1). ¹H NMR (DMSO-d6, 400 MHz) δ 11.21 (s, 1H), 8.75 (s, 1H), 8.67 (s, 1H), 8.04 (d, 2H, J=7 Hz), 7.65 (t, 1H, J=8 Hz), 7.55 (t, 2H, J=8 Hz), 5.98 (d, 1H, J=6 Hz), 5.62 (d, 1H, J=6 Hz), 4.92 (q, 1H, J=5 Hz), 4.03 (m, 1H), 3.86 (t, 1H, J=5 Hz), 3.55 (s, 3H), 3.43 (s, 1H), 2.41 (t, 2H, J=8 Hz), 1.98 (dd, 2H, J=3, 8 Hz).

### Ethyl(2-((2S,4R,5R)-5-(6-((bis(4-methoxyphenyl)(phenyl)methyl)amino)-9H-purin-9-yl)-4-hydroxytetrahydrofuran-2-yl)ethyl)(methyl)phosphinate (31)

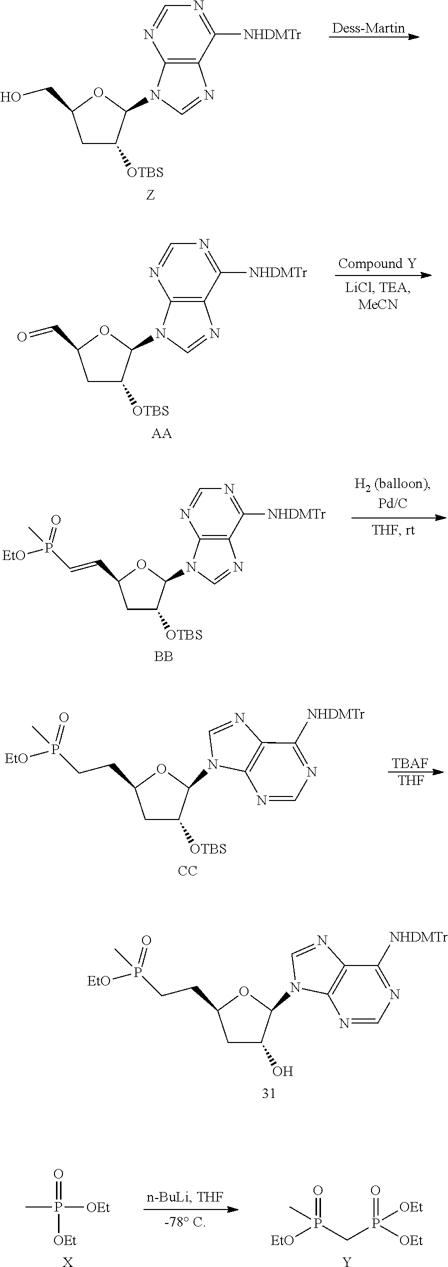

A solution of compound X (7.6 g, 50 mmol) in anhydrous THF (15 mL) was cooled to −78° C. under nitrogen. n-BuLi (32 mL of 1.6 M solution in hexane) was added dropwise to the solution of compound X with rapid stiffing. After the addition was complete, the solution was warmed to room temperature, and stirred for an additional 2 hours. The reaction mixture was quenched with 3N HCl and extracted with CH₂Cl₂. The combined organic layer was dried over anhydrous Na₂SO₄ and filtered. The filtrate was concentrated in vacuo to afford crude compound Y (4.5 g, 70%) as a colorless liquid. ¹H NMR (CDCl₃, 400 MHz): δ 4.09 (m, 6H), 1.61 (d, J=15.2 Hz, 3H), 1.30 (m, 9H).

To an ice-cold suspension of Des s-Martin (8.8 g, 20.8 mmol) in anhydrous CH₂Cl₂ (80 mL) was added dropwise a solution of compound Z (11.6 g, 17.4 mmol) in anhydrous CH₂Cl₂ (20 mL) under nitrogen. The resulting mixture was stirred for 3 hours at room temperature. The mixture was washed with aqueous Na₂S₂O₃, aqueous NaHCO₃ and brine. The organic layer was separated and dried over anhydrous Na₂SO₄. The mixture was then filtered. The filtrate was concentrated under vacuum to give compound AA (19.1 g, crude) as a foam solid, which was used without further purification.

To an ice-cold suspension of LiCl (0.36 g, 9.0 mmol) in anhydrous CH₃CN (50 mL) was added a solution of compound Y (2.1 g, 8.3 mmol) in anhydrous CH₃CN (10 mL) under nitrogen. The reaction mixture was then stirred for 30 minutes. Et₃N (0.9 g, 9.0 mmol) was added, and the solution was stirred for an additional 30 minutes. A solution of the compound AA (5.0 g, 7.5 mmol) in anhydrous CH₃CN was then added. The mixture was stirred overnight. The solution was concentrated under vacuum, and the residue was purified by silica gel chromatography to give compound BB (1.4 g, 13.8% for 2 steps) as a pale white solid, R_{f}=0.25 (PE/EA=0:1). ¹H NMR (CDCl₃, 400 MHz): δ 7.95 (s, 1H), 7.73 (s, 1H), 7.24 (d, J=1.2 Hz, 2H), 7.16 (m, 7H), 6.71 (m, 1H), 5.80 (d, J=1.2 Hz, 2H), 4.97 (m, 1H), 4.80 (m, 1H), 4.05 (m, 2H), 3.86 (m, 1H), 3.70 (s, 6H), 2.12 (m, 2H), 1.43 (d, J=14.8 Hz , 3H), 1.20 (m, 3H), 0.740 (s, 9H), −0.03 (s, 6H).

Compound BB (1.4 g, 1.8 mmol) and Pd/C (0.5 g) were added to 50 mL of THF. The resulting mixture was stirred under a hydrogen (balloon) at room temperature for 12 hours. The solution was filtered. The filtrate was concentrated under vacuum to give compound CC (1.3 g, crude) as a foam solid, which was used without further purification. LCMS calculated 771.4, observed 303.1 (DMTr), 772.6 (M+1).

Compound CC (1.4 g, 1.82 mmol) was dissolved in THF (20 mL). TBAF (0.71 g, 2.72 mmol) was added to the solution of compound CC at room temperature. The resulting mixture was stirred overnight. The reaction mixture was then concentrated under vacuum, and the residue was purified by silica gel chromatography to give compound 31 (0.8 g, 67.2% for two steps) as a pale white foam solid, R_{f}=0.3 (DCM/MeOH=20:1). ¹H NMR (MeOD, 400 MHz): δ 8.19 (s, 1H), 7.88 (s, 1H), 7.29 (d, J=5.6 Hz, 2H), 7.20 (m, 7H), 6.77 (d, J=8.8 Hz, 4H), 5.90 (s, 1H), 4.72 (m, 1H), 4.48 (m, 1H), 3.99 (m, 2H), 3.73 (s, 6H), 2.13 (m, 2H), 1.94 (m, 4H), 1.47 (d, J=13.6 Hz, 3H), 1.30 (m, 3H). LCMS calculated 657.3, observed 303.1 (DMTr), 658.3 (M+1).

### Dimethyl(2-((2S,4R,5R)-5-(6-((bis(4-methoxyphenyl)(phenyl)methyl)amino)-9H-purin-9-yl)-4-hydroxytetrahydrofuran-2-yl)ethyl)phosphonate (32)

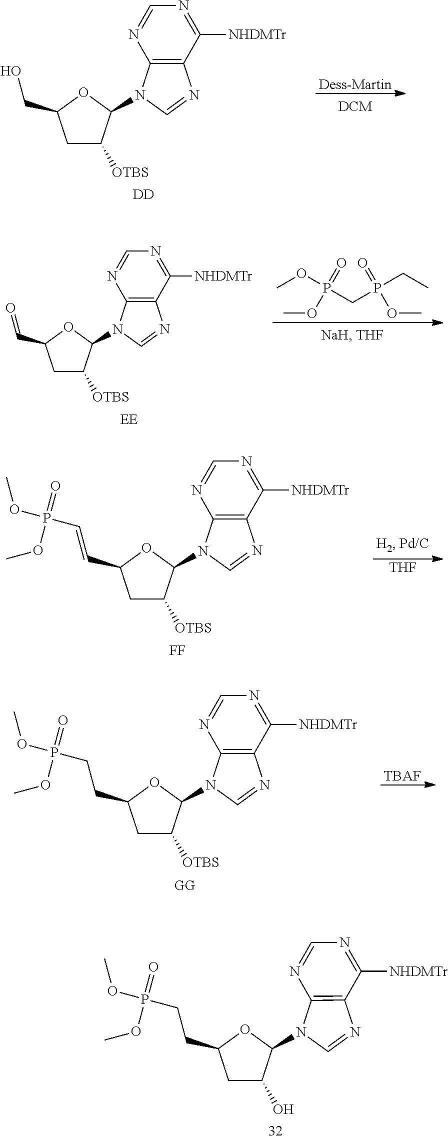

To an ice-cold mixture of Dess-Martin (3.8 g, 9.0 mmol) and pyridine (3.5 g, 44.3 mmol) in 60 mL CH₂Cl₂ was added a solution of compound DD (5.0 g, 7.5 mmol) in 30 mL CH₂Cl₂ under nitrogen. The reaction mixture was warmed to room temperature, and stirred for 3 hours. The reaction mixture was then diluted with CH₂Cl₂ (100 mL) and washed with saturated aqueous Na₂S₂O₃ (2×100 mL), saturated aqueous NaHCO₃ (2×100 mL) and brine (2×100 mL). The organic layer was separated, dried over anhydrous Na₂SO₄ and filtered. The filtrate was concentrated under vacuum to give crude compound EE (5.0 g), which was used without further purification.

To an ice-cold solution of (dimethoxy-phosphorylmethyl)-phosphonic acid dimethyl ester (2.6 g, 11.2 mmol) in 50 mL THF was added NaH (60% in mineral oil, 0.45 g, 11.2 mmol) in small portions under nitrogen. The resulting mixture was stirred for 30 minutes. A solution of compound EE (5.0 g, 7.5 mmol) in 20 mL anhydrous THF was then added dropwise at 0° C. under nitrogen. The reaction mixture was stirred at 0° C. for 2 hours. The mixture was quenched by water (5 mL). The reaction mixture was then diluted with brine (200 mL) and extracted with ethyl acetate (2×200 mL). The combined organic layers was dried over Na₂SO₄ and filtered. The filtrate was concentrated under reduced pressure, and the residue was purified by silica gel chromatography (ethyl acetate/petroleum ether) to give compound FF (2.1 g, 36%) as a white solid. ¹H-NMR: (CD₃OD, 400 MHz): δ 8.20 (s, 1H), 7.90 (s, 1H), 7.19-7.33 (m, 9H), 6.90-7.00 (m, 1H), 6.78-6.80 (dd, J₁=2.0 Hz, J₂=6.8 Hz, 4H), 6.00-6.10 (m, 1H), 5.91 (d, J=1.6 Hz, 1H), 4.95-5.04 (m, 2H), 3.74 (s, 6H), 3.70 (d, J=1.2 Hz, 3H), 3.67 (d, J=1.2 Hz, 3H), 2.25-2.39 (m, 2H), 0.86 (s, 9H), 0.04 (s, 3H), 0.00 (s, 3H).

A mixture of compound FF (2.1 g, 2.7 mmol) and 10% Pd/C (0.5 g) in THF (20 mL) was stirred for 1 hour under a hydrogen balloon. The reaction mixture was filtered through celite, and the filtrate was concentrate in vacuo to give compound GG (2.0 g, 95%) as a white solid. LCMS calculated 773.34, observed: 303.1 ((CH₃OPh)₂CPh⁺), 774.4 (M+1).

To an ice-cold solution of compound GG (2.0 g, 2.6 mmol) in THF (20 mL) was added a solution of TBAF (2.0 g, 7.7 mmol) in THF (20 mL). The reaction mixture was warmed to room temperature and stirred overnight. The solvent was removed under reduced pressure, and the residue was purified by silica gel chromatography (CH₂Cl₂/CH₃OH=100:1 to 30:1) to give compound 32 (1.1 g, 65%) as a white solid. ¹H-NMR (CD₃OD, 400 MHz): δ 8.19 (s, 1H), 7.90 (s, 1H), 7.17-7.30 (m, 9H), 6.79-6.82 (dd, J₁=2.0 Hz, J₂=6.8 Hz, 4H), 5.91 (d, J=1.6 Hz, 1H), 4.74 (m, 1H), 4.43 (m, 1H), 3.77 (s, 6H), 3.75 (s, 3H), 3.72 (s, 3H), 2.13-2.16 (m, 2H), 1.92-1.98 (m, 4H).

### (2′,3′-O-lev)-N⁶-(4-methoxytrityl)-adenosine (8)

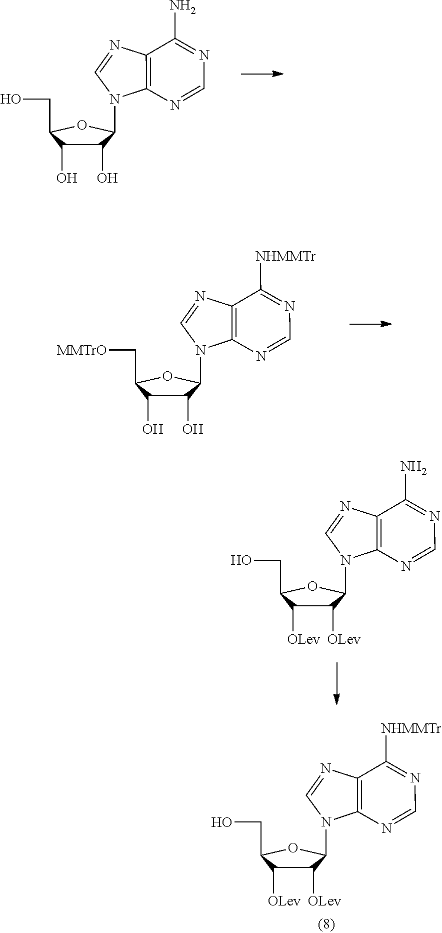

5′-O,⁶-Bis(4-methoxytrityl)adenosine. Adenosine (26.2 mmol, 7.00 g) was coevaporated twice from dry pyridine and dissolved in the same solvent (40 mL). A solution of 4-methoxytrityl chloride (57.7 mmol, 17.S g) in pyridine (60 mL) was added, and the mixture was stirred overnight at 35° C. MeOH (25 mL) was added, and the stirring was continued for 0.5 hours. The solvent was removed by evaporation under reduced pressure and the residual oil was partitioned between water and chloroform. The organic layer was washed with saturated aqueous NaHCO₃ and saturated aqueous NaCl, dried over Na₂SO₄ and evaporated to dryness. The residue was coevaporated with toluene and purified by Silica gel chromatography using dichloromethane containing 1-2% MeOH as eluent. The product was obtained as yellowish foam in 71% yield (15.2 g). ¹H NMR (500 MHz, CDCl₃) δ: S.07 (s, I H, HS), S.02 (s, 1H, H2), 7.17-7.39 (m, 24H, MMTr), 7.09 (hr s, 1H, N⁶H), 6.79-6.84 (m, 4H, MMTr), 6.64 (br s, 1H, 2′-OH), 5.95 (d, J=6.0 Hz, 1H. HI′), 4.79 (m, 1H, H2′), 4.42-4.44 (m, 1H. H4′). 4.37 (dd, J=5.5 and 2.0 Hz, 1H, H3′). 3.80 (s, 6H, MeO MMTr), 3.70 (br s. 1H 3′-OH), 3.49 (dd, J=10.5 and 3.5 Hz, 1H. H5′). 3.25 (dd, J=10.5 and 3.5 Hz. 1H. H5″). ¹³C NMR (126 MHz, CDCl₃) δ: 158.7 (MMTr), 158.4 (MMTr), 154.3 (C6), 151.6 (C2), 147.9 (C4), 145.0, 143.9 (MMTr), 138.1 (C8), 137.1, 134.8, 130.4, 130.2, 128.9, 128.2, 127.9, 127.0 (MMTr), 121.2 (C5), 113.2 (MMTr). 91.0 (C1′). 86.8 (C4′), 86.5 (MMTr), 76.1 (C2′), 72.9 (C3′), 71.1 (MMTr), 63.7 (C5′). 55.2 (OCH₃ MMTr). ESI′-MS: m/z obsd 812.3420, calcd (M+H) 812.3443.

2′,3′-Di-O-levulinoyladenosine. Levulinic anhydride was prepared by dissolving levulinic acid (29.6 mmol, 3.43 g) in dry 1,4-dioxane (40 ml) on an ice bath and adding dicyclohexylcarbodiimide (14.8 mmol, 3.05 g) into the solution in small portions within an hour. The ice bath was removed, and the reaction was allowed to proceed at room temperature for 2 hours. Precipitated dicyclohexylurea was filtered off, and the precipitate washed with 10 mL of dry dioxane. The filtrate was added to a solution of compound 5′-O,⁶-Bis(4-methoxytrityl)adenosine (7.4 mmol, 6.00 g) in dry pyridine (30 mL) and a catalytic amount of 4-dimethylaminopyridine was added. After two hours at room temperature, the mixture was evaporated to dryness. The residue was dissolved in DCM, washed with saturated aqueous NaHCO₃ and saturated aqueous NaCl. The organic phase was dried over Na₂SO₄ and evaporated to dryness. The compound was subjected to detritylation without purification. The crude product was dissolved in 80% (v/v) aqueous AcOH (80 mL). After stiffing over night at room temperature, the reaction mixture was evaporated to dryness. The product was purified by Silica gel chromatography, eluting with dichloromethane containing 5-10% MeOH. 2′,3′-Di-O-levulinoyladenosine was obtained as white foam in 81% yield (2.79 g). 1H NMR (500 MHz,

2′,3′-Di-O-levulinoyl-N⁶-(4-methoxytrityl)adenosine (8). 2′,3′-Di-O-levulinoyladenosine (3.2 mmol, 1.5 g) was evaporated twice from dry pyridine and dissolved in the same solvent (25 mL). Trimethylsilyl chloride (8.1 mmol, 1.03 mL) was added, and the mixture was stirred for 1.5 hours. Another portion of trimethylsilyl chloride (8.1 mmol, 1.03 mL) was added, and the stiffing was continued for 1 hour. 4-Methoxytrityl chloride (3.6 mmol, 1.1 g) was added, and the mixture was stirred overnight at 35° C. The mixture was then further stirred at 45° C. for 7 hours. 4-methoxytrityl chloride (0.7 mmol, 0.2 g) was added, and the mixture was stirred at 40° C. overnight. Saturated aqueous NaHCO₃ was added, and the mixture was stirred for 10 minutes and extracted with ethyl acetate. The organic phase was washed with saturated aqueous NaHCO₃, dried over Na₂SO₄ and evaporated to dryness. The product was purified by Silica gel chromatography, eluting with DCM containing 3% MeOH, and then ethyl acetate containing 5% MeOH. Two products were obtained: compound 8 (0.67 g) and its 5′-O-trimethylsilyl ether (0.96 g). The trimethylsilyl group was removed from the latter by treatment with Bu₄NF in THF under acidic conditions. Bu₄NF (2.5 mmol, 0.65 g) was dissolved in dry THF (16 mL) and AcOH (3 mL) was added. The nucleoside was added, and the mixture was stirred at room temperature for 15 minutes. Saturated aqueous NaHCO₃ was added, and the mixture was extracted with dichloromethane. The organic phase was washed with saturated aqueous NaCl and dried over Na₂SO₄ and evaporated to dryness. Compound 8 was obtained as yellowish foam. The overall yield was 61% (0.67 g+0.78 g). ¹H NMR (500 MHz, CDCl₃) δ: 8.03 (s. 1H, H2), 7.83 (s, 1H, H8), 7.23-7.36 (m, 12H, MMTr), 7.04 (s, 1H, N⁶H), 6.83-6.84 (m, 3H, MMTr and 5′OH), 6.06 (d, J=8.0 Hz, 1H, H1′), 5.94 (dd, J=8.0 and 5.5 Hz, 1H, H2′), 5.71 (dd, J=5.5 and 0.5 Hz, 1H, H3′), 4.36-4.37 (m, 1H, H4′), 3.95 (dd, J=13.0 and 1.0 Hz, 1H, H5′), 3.79-3.83 (m, 4H, MeO MMTr and H5″), 2.53-2.80 (m, 8H, CH₂ Lev), 2.24 (s, 3H, CH₃ Lev), 2.19 (s, 3H, CH₃ Lev). ¹³C NMR (126 MHz, CDCl₃) δ: 206.4 (OC═O), 206.2 (OC═O), 171.7 (CC═O), 171.2 (CC═O), 158.3 (MMTr), 154.6 (C6), 151.9 (C2), 147.2 (C4), 144.9 (MMTr), 139.8 (C8), 136.8 (MMTr), 130.1, 128.8, 127.9, 126.9 (MMTr), 122.4 (e5), 113.2 (MMTr). 88.7 (C1′), 86.6 (C4′), 73.1 (C2′), 72.9 (C3′), 71.1 (MMTr), 62.6 (C5′), 55.2 (OCH₃ MMTr), 37.7 (CH₂C═O Lev), 37.6 (CH₂C═O Lev), 29.8 (CH₃ Lev), 29.8 (CH₃ Lev), 27.6 (CH₂C=00 Lev), 27.4 (CH₂C=00 Lev). ESI⁺-MS: m/z obsd (M+H)⁺ 736.2944, calcd (M+H)⁺ 736.2977.

### (3′-O-pivoxymethyl)-(5′-O-TBDMSO)-N⁶-(4-methoxytrityl)-adenosine (9a) and 2′-O-levulinoyl-N⁶-(4-methoxytrityl)-3′-O-pivaloyloxymethyladenosine (9b)

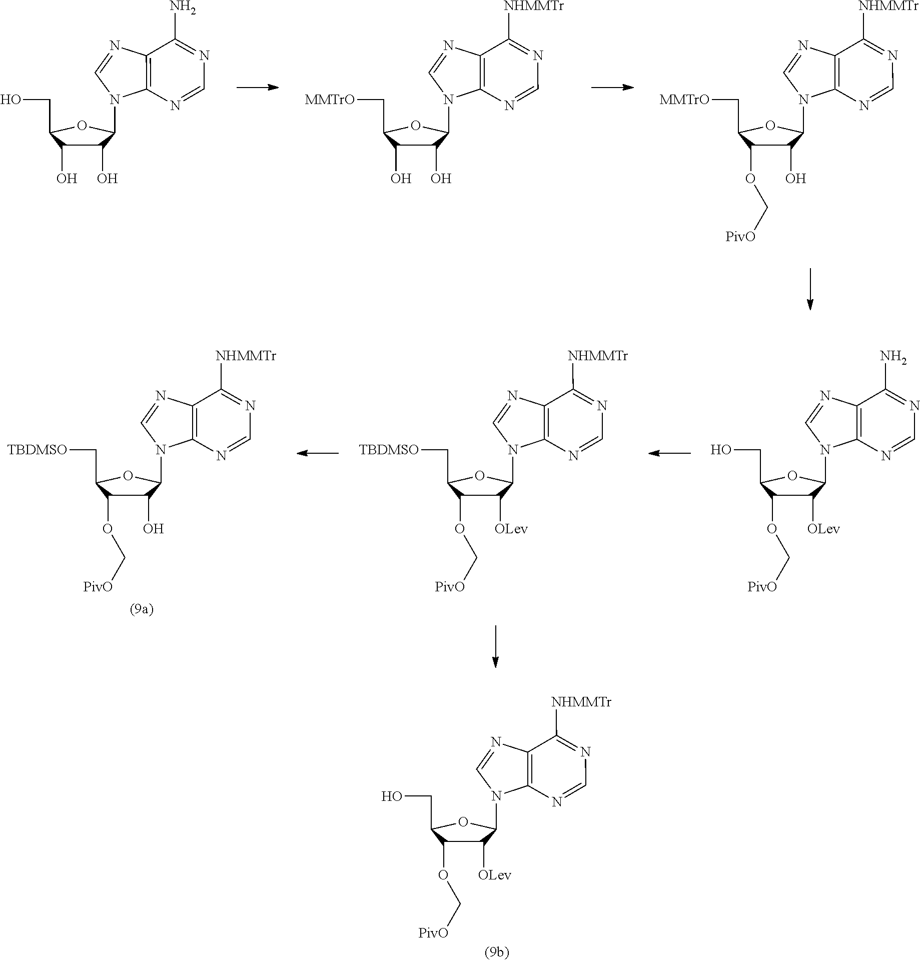

5′-O,N⁶-Bis(4-methoxytrityl)-3′-O-pivaloyloxymethyladenosine . 5′-O,⁶-Bis(4-methoxytrityl)adenosine (0.60 mmol, 0.49 g), dried over P₂O₅ overnight, was dissolved in dry THF (5 mL) and 1 equiv. of sodium hydride (24 mg of 60% dispersion, 0.60 mmol) was added. After stirring for 1 hour at room temperature, the mixture was added into a mixture of pivaloyloxymethyl chloride (0.66 mmol, 94 μL) and NaI (9 mg). The reaction was allowed to proceed for 5 hours. The reaction was quenched by adding water, and the mixture was extracted three times with Et₂O. The ether layer was dried over Na₂SO₄ and evaporated to dryness. The products were separated by Silica gel chromatography, eluting with a 1:1 (v/v) mixture of ethyl acetate and petroleum ether. Three products were obtained, 5′-O,N⁶-Bis(4-methoxytrityl)-3′-O-pivaloyloxymethyladenosine (0.12 g, 21% yield), its 2′-O-isomer (0.04 g) and, 5′-O,N⁶-bis(4-methoxytrityl)-3′-O-pivaloyladenosine (0.20 g). ¹H NMR (500 MHz, CDCl³) δ: 8.02 (s, 1H, H2), 8.01 (s, 1H, H8), 7.22-7.38 (m, 24H, MMTr), 7.02 (s, 1H, N⁶H), 6.80-6.84 (m, 4H, MMTr), 5.93 (d, J=6.5 Hz, 1H, H1′), 5.43 (d, J=6.5 Hz, 1H, OCH₂O), 5.39 (d, J=6.5 Hz, 1H, OCH₂O), 4.93 (dd, J=6.5 and 5.5 Hz, 1H, H2′), 4.74 (d, J=5.5 Hz, 2′-OH), 4.51 (dd, J=5.5 and 3.0 Hz, 1H, H3′), 4.37-4.38 (m, 1H, H4′), 3.80 (s, 6H, MeO MMTr), 3.48 (dd, J=10.5 and 4.0 Hz, 1H, H5′), 3.28 (dd, J=10.5 and 4.0 Hz, 1H, H5″), 1.17 (s, 9H, (CH₃)₃C Piv). ¹³C NMR (126 MHz, CDCl₃) δ: 177.8 (C═O Piv), 158.7 (C6), 158.3 (MMTr), 152.0 (C2), 148.3 (C4), 145.13 (MMTr) 138.6 (C8), 126.9, 127.9, 128.2, 128.9, 130.2, 130.3, 134.9 (MMTr), 121.3 (C5), 113.2 (MMTr), 89.7 (C1′), 88.7 (OCH₂O), 86.8 (MMTr), 83.6 (C4′), 79.3 (C3′). 74.7 (C2′), 71.1 (MMTr), 63.2 (C5′), 55.2 (OCH₃ MMTr), 38.7 (CMe₃ Piv), 27.0 (CH₃ Piv). ESI-MS: m/z obsd 926.4115, calcd (M+H)⁺ 926.4123.

2′-O-Levulinoyl-3′-O-pivaloyloxymethyladenosine. Levulinic anhydride was prepared by dissolving levulinic acid (5.6 mmol, 0.65 g) in dry 1,4-dioxane (10 mL) on an ice bath and adding dicyclohexylcarbodiimide (2.8 mmol, 0.58 g) into the solution in small portions within an hour. The ice bath was removed, and the reaction was allowed to proceed at room temperature for 2 hours. Precipitated dicyclohexylurea was filtered off and washed with 5 mL of dry dioxane. The filtrate was added to a solution of 5′-O,N⁶-Bis(4-methoxytrityl)-3′-O-pivaloyloxymethyladenosine (2.3 mmol, 2.1 g, dried over P₂O₅, over night) in dry pyridine (9 mL) and a catalytic amount of 4-dimethylaminopyridine was added. After stiffing overnight at room temperature, the mixture was evaporated to dryness. The residue was dissolved in dichloromethane, washed with saturated aqueous NaHCO₃ and saturated aqueous NaCl and dried over Na₂SO₄. The organic phase was evaporated to dryness and co evaporated with toluene. The compound was subjected to detritylation without purification. The crude product was dissolved in 80% (v/v) aqueous AcOH (50 mL), and after stirring for 5 hours at 40° C., the reaction mixture was evaporated to dryness. The residue was dissolved in dichloromethane and washed three times with water. The organic phase was dried over Na₂SO₄ and evaporated to dryness. The product was purified by Silica gel chromatography, eluting with dichloromethane containing 5% MeOH. 2′-O-Levulinoyl-3′-O-pivaloyloxymethyladenosine was obtained as white foam in 84% yield (0.91 g). ¹H NMR (500 MHz, CDCl₃) δ: 8.33 (s, 1H, H2), 7.83 (s, 1H, H8), 6.54 (m, 1H, 5′-OH), 6.01 (d, J=7.5 Hz, 1H, H1′), 5.74 (dd, J=7.5 and 5.5 Hz, 1H, H2′), 5.64 (m, 1H, N⁶H), 5.54 (d, J=6.5 Hz, 1H, OCH₂O), 5.12 (d, J=6.5 Hz, 1H, OCH₂O), 4.80 (m 1H, H3′), 4.35 (m, 1H, H4′), 3.98 (m, 1H, H5′), 3.77 (m, 1H, H5″), 2.49-2.75 (m, 4H, CH₂CH₂ Lev), 2.16 (s, 3H, CH₃, Lev), 1.23 (s, 9H, (CH₃)₃C Piv). ¹³C NMR (126 MHz, CDCl₃) δ: 206.1 (C═O, Lev), 177.8 (C═O, Piv), 171.5 (C═O, Lev), 155.9 (C6), 152.7 (C2), 148.7 (C4), 140.6 (C8), 121.3 (C5), 88.9 (OCH₂O), 88.9 (C1′), 87.3 (C4′), 78.3 (C3′), 74.3 (C2′), 62.7 (C5′), 38.9 (CMe₃ Piv), 37.7 (CH₂ Lev), 29.8 (CH₃ Lev), 27.5 (CH₂ Lev), 27.0 (CH₃ Piv). ESI-MS: m/z obsd 480.2072, calcd (M+H) 480.2089.

5′-O-(tert-Butyldimethylsilyl)-2′-O-levulinoyl-N⁶-(4-methoxytrityl)-3′-O-pivaloyloxymethyladenosine. 2′-O-Levulinoyl-3′-O-pivaloyloxymethyladenosine (1.7 mmol, 0.82 g) was coevaporated twice from dry pyridine and dissolved in the same solvent (5 mL). tert-Butyldimethylsilyl chloride (2.1 mmol; 0.31 g) was added, and the mixture was stirred overnight at room temperature. The reaction was quenched with MeOH, and the mixture was evaporated to dryness. The residue was dissolved in dichloromethane and washed with saturated aqueous NaHCO₃ and saturated aqueous NaCl. The organic phase was dried over Na₂SO₄ and evaporated to dryness. The product was purified by Silica gel chromatography, eluting with dichloromethane containing 10% MeOH and subjected directly to tritylation. The product (1.6 mmol, 0.94 g) was coevaporated twice from dry pyridine and dissolved in dry pyridine (6 mL). 4-methoxytrityl chloride (1.9 mmol, 0.59 g) was added, and the mixture was stirred over two nights at 40° C. The reaction was quenched with MeOH, and the mixture was evaporated to dryness. The residue was dissolved in dichloromethane and washed with water and saturated aqueous NaCl. The organic phase was dried over Na₂SO₄ and evaporated to dryness. The product was purified by Silica gel chromatography, eluting with dichloromethane containing 1-3% MeOH. 5′-O-(tert-Butyldimethylsilyl)-2′-O-levulinoyl-N⁶-(4-methoxytrityl)-3′-O-pivaloyloxymethyladenosine was obtained as yellowish foam in 85% yield (1.27 g). ¹H NMR (500 MHz, CDCl₃) δ: 8.06 (s. 1H, H2). 8.03 (s, 1H, H8), 7.20-7.35 (m, 12H, MMTr), 6.89 (s, 1H, N⁶H), 6.79 (m, 2H, MMTr), 6.18 (d, J=5.0 Hz, 1H, HI′). 5.68 (m, 1H. H2′). 5.37 (d, J=6.5 Hz, 1H, OCH₂O), 5.18 (d, J=6.5 Hz, 1H, OCH₂O), 4.78 (m, 1H, H3′), 4.20 (dd. J=7.5 and 3.0 Hz, 1H, H4′). 3.92 (dd, J=11.3 and 3.3 Hz, 1H. H5′), 3.80 (dd, J=11.3 and 3.3 Hz. 1H. H5″), 3.78 (s. 3H, MeO MMTr), 2.56-2.75 (m, 4H, CH₂CH₂ Lev), 2.14 (s, 3H, CH₃, Lev), 1.21 (s, 9H, (CH₃)₃C Piv), 0.89 (s, 9H, (CH₃)₃CSi), 0.07 (s, 3H, CH₃Si), 0.05 (s, 3H, CH₃Si). ¹³C NMR (126 MHz, CDCl₃) δ: 206.1 (C═O, Lev), 177.8 (C═O, Piv), 171.7 (C═O, Lev), 158.3 (MMTr), 154.1 (C6), 152.5 (C2), 148.8 (C4), 145.2 (MMTr), 138.5 (C8), 137.2, 130.2, 128.9, 127.9, 126.9 (MMTr), 121.2 (C5), 113.2 (MMTr), 88.3 (OCH₂O), 85.9 (C1′), 83.9 (C4′), 76.2 (C3′). 74.9 (C2′), 71.0 (MMTr), 62.6 (C5′), 55.2 (OMe MMTr), 38.8 (CMe₃ Piv), 37.8 (CH₂ Lev), 29.7 (CH₃ Lev), 27.7 (CH₂ Lev), 27.0 (CH₃ Piv), 25.9 (CH₃)₃CSi), 18.4 ((CH₃)₃CSi), −5.4, −5.5 (CH₃)₃CSi). ESI-MS: m/z obsd 866.4145, calcd (M+H)⁺ 866.4155.

5′-O-(tert-Butyldimethylsilyl)-N⁶-(4-methoxytrityl)-3′-O-pivaloyloxymethyladenosine (9a). 5′-O-(tert-Butyldimethylsilyl)-2′-O-levulinoyl-N⁶-(4-methoxytrityl)-3′-O-pivaloyloxymethyladeno sine was dissolved in a solution of hydrazine hydrate (6.0 mmol, 0.29 mL) in pyridine (10 mL) and acetic acid (2 mL) on an ice bath, and the mixture was stirred for 1.5 hours. The ice bath was removed and the reaction was allowed to proceed at room temperature for 5 hours. The reaction was quenched with saturated aqueous NaHCO₃ and the mixture was extracted with DCM. The organic phase was washed with saturated aqueous NaCl and dried over Na₂SO₄ and evaporated to dryness. The product was purified by Silica gel chromatography, eluting with DCM containing 3% MeOH. Compound 9a was obtained as yellowish foam in 92% yield (1.04 g). ¹H NMR (500 MHz, CDCl₃) δ: 8.03 (s. 1H, H2), 8.01 (s. 1H. H8), 7.23-7.37 (m, 12H, MMTr), 6.97 (s, 1H, N⁶H), 6.81 (d, J=9 Hz, 2H, MMTr), 5.95 (d, J=5.5 Hz, 1H, H1′). 5.44-5.49 (m, 2H, OCH₂O), 4.75 (m, 1H, H2′). 4.51 (dd, J=5.3 and 2.8 Hz. 1H, H3′). 4.41 (d, J=5.0 Hz. 2′-OH), 4.30 (m, 1H. H4′), 3.88 (dd. J=11.3 and 3.8 Hz. 1H. H5′). 3.80-3.83 (m. 4H, H5″ and MeO MMTr). 1.25 (s. 9H. (CH₃)₃C Piv), 0.86 (s, 9H, (CH₃)₃CSi), 0.08 (s, 3H, CH₃Si), 0.04 (s, 3H, CH₃Si). ¹³C NMR (126 MHz. CDCl₃) δ: 177.9 (C═O, Piv), 158.3 (MMTr), 154.2 (C6), 152.1 (C2), 148.3 (C4), 145.2 (MMTr) 138.4 (C8), 137.2, 130.2, 128.9, 127.9, 126.9 (MMTr), 121.2 (C5), 113.2 (MMTr), 89.3 (C1′), 88.5 (OCH₂O), 84.4 (C4′), 79.0 (C3′), 74.9 (C2′), 71.0 (MMTr), 62.8 (C5′), 55.2 (MeO MMTr), 38.7 (Cme₃ Piv), 27.0 (CH₃ Piv), 25.8 ((CH₃)₃CSi), 18.3 ((CH₃)₃CSi), −5.5 (CH₃Si).

2′-O-Levulinoyl-N⁶-(4-methoxytrityl)-3′-O-pivaloyloxymethyladenosine (9b). 2′-O-levulinoyl-3′-O-pivaloyloxymethyladenosine (1.9 mmol, 0.9 g), dried over P₂O₅ overnight, was dissolved in dry pyridine (12 mL). The solution was cooled on an ice-bath and trimethylsilyl chloride (9.4 mmol, 1.19 mL) was added. The mixture was stirred for 2.5 hours at room temperature. 4-Methoxytrityl chloride (2.3 mmol, 0.70 g) was added, and the mixture was stirred over three nights at 37° C. The solvent was removed by evaporation under reduced pressure, and the residual oil was partitioned between water and ethyl acetate. The organic layer was washed with saturated aqueous NaHCO₃ and dried over Na₂SO₄ and evaporated to dryness. The trimethylsilyl group was removed by treatment with tetrabutylammonium fluoride in THF under acidic conditions. Bu₄NF (2.8 mmol, 0.74 g) was dissolved in dry THF (16 mL) and AcOH (3 mL) was added. The nucleoside was added, and the mixture was stirred at room temperature for 2 hours. Saturated aqueous NaHCO₃ was added, and the mixture was extracted with dichloromethane. The organic phase was dried over Na₂SO₄ and evaporated to dryness. The product was purified by Silica gel chromatography, eluting with dichloromethane containing 3% MeOH. Compound 9b was obtained as yellowish foam in 83% yield (1.17 g). ¹H NMR (500 MHz, CDCl₃) δ: 8.02 (s, 1H, H2), 7.80 (s, 1H, H8), 7.24-7.36 (m, 12H, MMTr), 7.05 (s, 1H, N⁶H), 6.81-6.84 (m, 2H, MMTr), 6.62 (dd, J=12 and 2 Hz, 1H, 5′-OH) 6.03 (d, J=7.5 Hz, 1H, H1′), 5.68 (dd, J=7.5 and 5.3 Hz, 1H, H2′), 5.55 (d, J=6.5 Hz, 1H, OCH₂O), 5.12 (d, J=6.5 Hz, 1H, OCH₂O), 4.82 (dd, J=5.3 and 1 Hz, 1H, H3′), 4.35 (m, 1H, H4′), 3.93-3.97 (m, 1H, H5′), 3.81 (s, 3H, MeO MMTr), 3.72-3.78 (m, 1H, H5″), 2.53-2.78 (m, 4H, CH₂CH₂ Lev), 2.20 (s, 3H, CH₃ Lev), 1.25 (s, 9H, (CH₃)₃C Piv). ¹³C NMR (126 MHz, CDCl₃) δ: 206.1 (C═O Lev) 177.7 (C═O Piv), 171.5 (C═O Lev), 158.4 (MMTr), 154.6 (C6), 151.9 (C2), 147.3 (C4), 144.9 (MMTr), 139.8 (C8), 136.9, 130.2, 128.9, 128.0, 127.0 (MMTr), 122.5 (C5), 113.2 (MMTr), 88.9 (OCH₂O), 88.9 (C1′), 87.3 (C4′), 78.2 (C3′), 74.6 (C2′), 71.1 (MMTr), 62.7 (C5′), 55.2 (OMe MMTr), 38.8 (CMe₃ Piv), 37.7 (CH₂C═O Lev), 29.8 (CH₃ Lev), 27.5 (—CH₂-000 Lev) 27.0 (CH₃, Piv). ESI⁺-MS: m/z obsd 752.3312, calcd (M+H)⁺ 752.3290.

### (2′-O-lev)-(3′-O-methyl)-N⁶-(4-methoxytrityl)-adenosine (10)

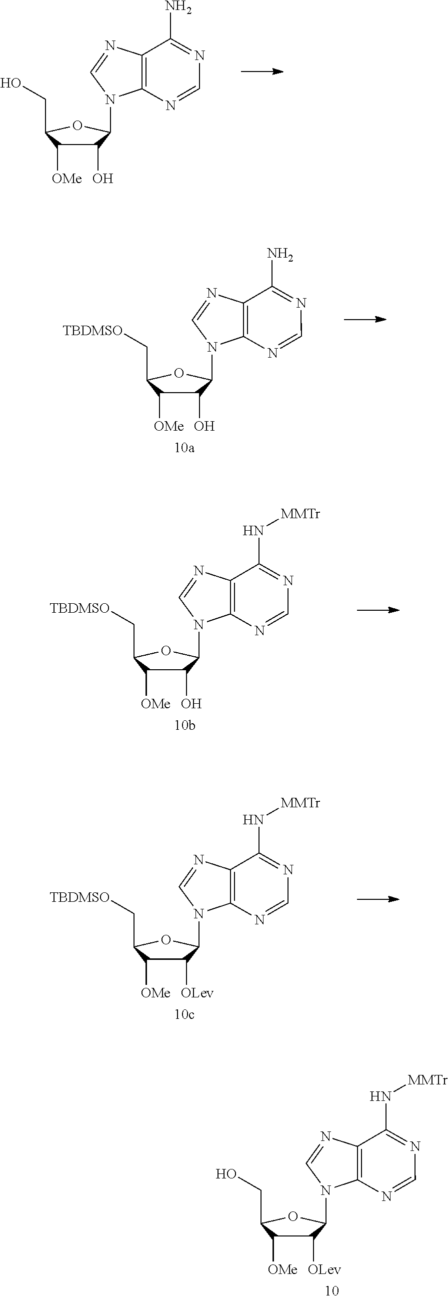

5′-O-tert-Butyldimethylsilyl-N⁶-3′-O-methyladenosine (10a). 3′-O-Methyladenosine (3.6 mmol, 1.01 g) was coevaporated twice from anhydrous pyridine, and the residue was dissolved in pyridine (7 mL). 1.1 equiv. of tert-butyl-dimethylsilylchloride (4.0 mmol, 0.60 g) was added, and the mixture was stirred over night at room temperature. The reaction was quenched with MeOH and evaporated to dryness. The residue was purified by Silica gel chromatography using DCM containing 10% MeOH as the eluent. ¹H NMR (500 MHz, MeOD) δ 8.41 (s, 1H, H2), 8.23 (s, 1H, H8), 6.06 (d, J=4.2 Hz, 1H, H1′), 4.77 (dd, J=4.2 and 4.6 Hz, 1H, H2′), 4.22 (m, 1H, H4′), 4.06 (dd, J=4.6 and 5.0 Hz, 1H, H3′), 4.03 (dd, J=11.5 and 3.4 Hz, 1H, H5′), 3.87 (dd, J=11.5 and 3.0 Hz, 1H, H5″), 3.50 (s, 3H, 3′-OMe), 0.96 (s, 9H, ^{t}Bu), 0.14 (s, 6H, ^{t}Bu). ¹³C NMR (126 MHz, MeOD) 155.9 (C6), 152.5 (C2), 149.1 (C4), 139.3 (C8), 119.0 (C5), 88.8 (C1′), 82.7 (C4′), 78.9 (C3′), 73.3 (C2′), 62.3 (C5′), 57.0 (OMe), 25.0 (C-Me₃), 17.9 (CMe₃), −6.7 (SiMe₂).

5′-O-tert-Butyldimethylsilyl-N⁶-(4-methoxytrityl)-3′-O-methyladenosine (10b). Compound 10a was coevaporated twice from anhydrous pyridine, and the residue was dissolved in dry pyridine (6 mL). 4-methoxytrityl chloride was added, and the mixture was stirred over three nights at room temperature. The reaction was quenched with MeOH, and the mixture was evaporated to dryness. The residue was dissolved in DCM and washed with saturated aqueous NaHCO₃ and saturated aqueous NaCl. The organic phase was dried over Na₂SO₄ and evaporated to dryness. The product was purified by Silica gel chromatography using DCM containing 2-3% MeOH as eluent. Compound 10b was obtained as white foam in 75% yield from 3′-O-methyladenosine (1.80 g). ¹H NMR (500 MHz, CDCl₃) δ 8.06 (br s, 2H, H2&8), 7.34-7.37 (m, 4H, MMTr), 7.23-7.30 (m, 8H, MMTr), 6.96 (br s, 1H, NH), 6.81 (d, J=8.8 Hz, 2H, MMTr), 5.98 (d, J=5.6 Hz, 1H, H1′), 4.73 (m, 1H, H2′), 4.27 (m, 1H, H4′), 4.16 (d, J=6.5 Hz, 1H, OH), 4.05 (m, 1H, H3′), 3.91 (dd, J=11.2 and 4.2 Hz, 1H, H5′), 3.78-3.81 (m, 4H, OMe&H5″), 3.52 (s, 3H, 3′-OMe), 0.90 (s, 9H, ^{t}Bu), 0.09&0.10 (2×s, 6H, ^{t}Bu). ¹³C NMR (126 MHz, CDCl₃) 158.3 (MMTr), 154.1 (C6), 152.1 (C2), 148.5 (C4), 145.2 (MMTr), 138.3 (C8), 137.2 (MMTr), 130.2 (MMTr), 128.9 (MMTr), 127.9 (MMTr), 126.9 (MMTr), 121.3 (C5), 113.1 (MMTr), 89.3 (C1′), 83.1 (C4′), 80.0 (C3′), 74.3 (C2′), 71.0 (MMTr), 63.0 (C5′), 58.1 (3′-OMe), 55.2 (MMTr), 25.9 (C-Me₃), 18.3 (CMe₃), −5.5 (SiMe₂). HRMS (ESI) Calcd for

5′-O-tert-butyldimethylsilyl-2′-O-levulinoyl-N⁶-(4-methoxytrityl)-3′-O-methyladenosine (10c). Levulinic anhydride was prepared by dissolving levulinic acid (6.7 mmol, 0.73 g) in dry 1,4-dioxane (10 mL) on an ice bath and adding DCC (3.4 mmol, 0.70 g) in small portions within an hour. The solution was stirred at room temperature for two hours. Precipitated dicyclohexylurea was filtered off and washed with 5 mL of dry dioxane. The filtrate was added to a solution of compound 10b (2.7 mmol, 1.80 g, dried over P₂O₅ over night) in dry pyridine (9 mL) and a catalytic amount of 4-dimethylaminopyridine was added. After stiffing over night at room temperature, the mixture was evaporated to dryness. The residue was dissolved in DCM and washed with saturated aqueous NaHCO₃ and saturated aqueous NaCl. The organic phase was dried over Na₂SO₄ and evaporated to dryness. The product was purified by Silica gel chromatography using DCM containing 1-2% MeOH as eluent. Compound 10c was obtained as white foam in 89% yield (1.84 g). ¹H NMR (500 MHz, CDCl₃) δ: 8.10 (s, 1H, H8), 8.06 (s, 1H, H8), 7.23-7.38 (m, 12H, MMTr), 6.92 (s, 1H, NH), 6.82 (m, 2H, MMTr), 6.19 (d, J=4.0 Hz, 1H, H1′), 5.74 (dd, J=4.5 and 4.0 Hz, 1H, H2′), 4.30 (m, 1H, H3′), 4.18 (m, 1H, H4′), 4.01 (dd, J=11.5 and 3.0 Hz, 1H, H5′), 3.83 (dd, J=11.5 and 3.0 Hz, 1H, H5″), 3.80 (s, 3H, MMTr), 3.42 (s, 3H, OMe), 2.62-2.81 (m, 4H, Lev), 2.18 (s, 3H, Lev), 0.94 (s, 9H, SiCMe₃), 0.12 (s, 3H, ), 0.11 (s, 3H, Si-Me). ¹³C NMR (126 MHz, CDCl₃) δ: 206.1 (C═O Lev), 171.7 (C═O Lev), 158.3 (MMTr), 154.1 (C6), 152.4 (C2), 148.5 (C4), 145.2 (MMTr), 138.4 (C8), 137.2 (MMTr), 130.2 (MMTr), 128.9 (MMTr), 127.9 (MMTr), 126.8 (MMTr), 121.2 (C5), 113.1 (MMTr), 86.5 (C1′), 82.9 (C4′), 77.7 (C3′), 74.5 (C2′), 71.0 (MMTr), 62.2 (C5′), 58.8 (OMe), 55.2 (MMTr), 37.8 (Lev), 29.8 (Lev), 27.8 (Lev), 26.0 (TBDMS), 18.4 (TBDMS), −5.3 (TBDMS), −5.5 (TBDMS). HRMS (ESI) Calcd for

2′-O-levulinoyl-N⁶-(4-methoxytrityl)-3′-O-methyladenosine (10). The trimethylsilyl group was removed by treatment with Bu₄NF in THF under acidic conditions. Bu₄NF (3.6 mmol, 0.94 g) was dissolved in dry THF (30 mL) and AcOH (6 mL) was added. Compound 10c was added, and the mixture was stirred over two nights at room temperature. Saturated aqueous NaHCO₃ was added, and the mixture was extracted with DCM. The organic phase was washed with saturated aqueous NaHCO₃ and saturated aqueous NaCl, and dried over Na₂SO₄ and evaporated to dryness. The product was purified by Silica gel chromatography using DCM containing 1-3% MeOH as eluent. Compound 10 was obtained as white foam in 80% yield (1.25 g). ¹H NMR (500 MHz, CDCl₃) δ: 7.99 (s, 1H, H2), 7.77 (s, 1H, H8), 7.21-7.34 (m, 12H, MMTr), 7.00 (s, 1H, NH), 6.80 (m, 2H, MMTr), 6.59 (dd, J=12.0 and 2.0 Hz, 5′OH), 6.00 (d, J=7.5 Hz, 1H, H1′), 5.71 (dd, J=7.5 and 5.0 Hz, 1H, H2′), 4.33-4.36 (m, 2H, H3′ and H4′), 3.98 (m, 1H, H5′), 3.78 (s, 3H, MMTr), 3.69 (m, 1H, H5″), 3.44 (s, 3H, OMe), 2.53-2.75 (m, 4H, Lev), 2.17 (s, 3H, Lev). ¹³C NMR (126 MHz, CDCl₃) δ: 206.1 (C═O Lev), 171.6 (C═O Lev), 158.4 (MMTr), 154.6 (C6), 151.9 (C2), 147.3 (C4), 145.0 (MMTr), 139.8 (C8), 136.9 (MMTr), 130.2 (MMTr), 128.9 (MMTr), 128.0 (MMTr), 127.0 (MMTr), 122.5 (C5), 113.2 (MMTr), 89.1 (C1′), 86.1 (C4′), 79.4 (C3′), 75.0 (C2′), 71.1 (MMTr), 63.2 (C5′), 58.5 (OMe), 55.2 (MMTr), 37.7 (Lev), 29.8 (Lev), 27.6 (Lev). HRMS (ESI) Calcd for C31H38N7O9, 652.2726; found 652.2718.

### (3′-O-acetyloxymethyl)-(2′-O-lev)-N⁶-(4-methoxytrityl)-adenosine (11) and (3′-O-acetyloxymethyl)-N⁶-(4-methoxytrityl)-adenosine (12)

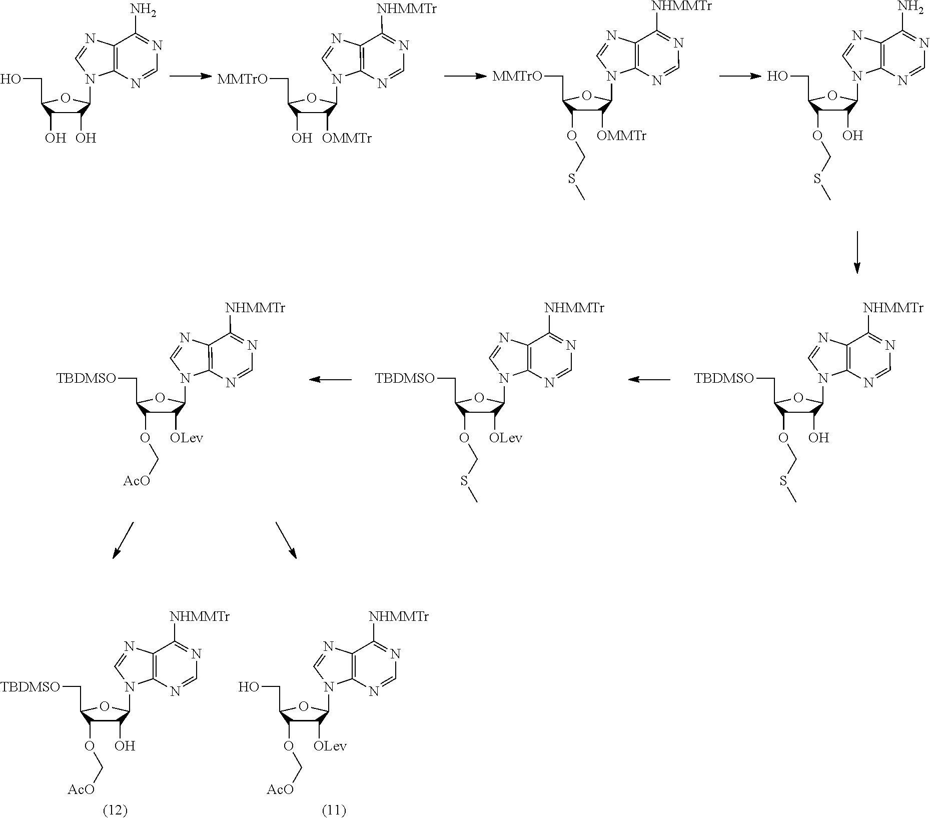

2′-O,5′-O,N⁶-Tris-(4-methoxytrityl)adenosine. Adenosine (18.7 mmol, 5.00 g) was dried on P₂O₅ overnight. The nucleoside was coevaporated from dry pyridine and dissolved in the same solvent (50 mL). 4-Methoxytrityl chloride (59.9 mmol, 18.5 g) was added, and the mixture was stirred at 60° C. overnight. The reaction was quenched by adding MeOH (50 mL), and the volatiles were removed under reduced pressure. The residue was dissolved in DCM, washed with water and brine and the organic layer was dried on Na₂SO₄, and evaporated to dryness. The residue was purified in three portions by silica gel chromatography using DCM containing 0-2% MeOH as eluent. The isolated yield of 2′-O,5′-O,N⁶-Tris-(4-methoxytrityl)adenosine was 50% (10.3 g). ¹H NMR (500 MHz, CDCl₃) δ 7.98 (s, 1H, H2), 7.97 (s, 1H, H8), 7.00-7.43 (m, 36H, MMTr), 6.84, 6.73 and 6.66 (3×d, J=8.5 Hz, 2H, MMTr), 6.38 (d, J=7.5 Hz, 1H, H1′), 5.09 (dd, J=7.5 and 4.5 Hz, 1H, H2′), 4.08 (dd, J=3.0 and 3.5 Hz, 1H, H4′), 3.77, 3.78 and 3.79 (3×s, 3H, MMTr), 3.31 (dd, J=11.0 and 3.5 Hz, 1H, H5′), 3.00 (dd, J=11.0 and 3.0 Hz, 1H, H5″), 2.92 (d, J=4.5 Hz, 1H, H3′), 2.32 (s, 1H, 3′-OH). ¹³C NMR (126 MHz, CDCl₃) δ: 159.2. 158.6. 158.3 (MMTr), 154.1 (C6). 152.4 (C2), 149.4 (C4), 145.3. 144.4, 143.9, 143.8, 143.0 (MMTr), 139.1 (C8), 137.3, 135.2, 134.5, 130.4, 130.2, 130.2, 128.9, 128.4, 128.3, 128.1, 128.0, 127.9, 127.8, 127.5, 127.4, 127.2, 127.0, 126.8 (MMTr), 121.2 (C5), 113.5, 113.1, 113.1 (MMTr), 87.5 (C1′), 86.8. 86.1 (MMTr) 84.3 (C4′). 77.0 (C2′, under CDCl₃), 71.0 (MMTr), 70.6 (C3′), 63.8 (C5′), 55.2 (OCH₃ MMTr). ES⁴-MS: m/z obsd (M+H)⁺ 1084.4649, calcd (M+H)⁺ 1084, 4649; obsd (M+Na) 1106.4432, calcd (M+Na) 1106.4469; obsd (M+K)⁺ 1122.4191, calcd (M+K)′ 1122, 4208.

2′-O,5′-O,N⁶-Tris(4-methoxytrityl)-3′-O-methylthiomethyladenosine. 2′-O,5′-O,N⁶-Tris-(4-methoxytrityl)adenosine (1.72 mmol, 1.86 g) was dried by coevaporation from dry pyridine and twice from dry MeCN. The residue was dissolved in dry DMF (4.0 mL), and the solution was cooled to 0° C. on an ice-bath. NaH (3.4 mmol, 0.137 g of 60% dispersion in oil) and NaI (1.5 mmol, 0.230 g) were added. The mixture was stirred for half an hour on an ice-bath. Methylthiomethyl chloride (2.1 mmol, 170 μL) was added, and the stiffing was continued for 4 hours at room temperature. Another portion of methylthiomethyl chloride (1.0 mmol, 85 μL) was then added, and the stiffing was continued for half an hour. The reaction was quenched by water. The mixture was extracted 3 times with diethyl ether. The combined organic phase was washed with brine and dried on Na₂SO₄ and evaporated to dryness. Silica gel chromatography with 30-40% ethyl acetate in petroleum ether gave 2′-O,5′-O,N⁶-Tris(4-methoxytrityl)-3′-O-methyltiomethyladenosine in 43% (0.86 g) yield. ¹H NMR (500 MHz, CDCl₃) δ 7.77 (s, 1H, H2), 7.70 (s, 1H, H8), 6.95-7.43 (m, 36H, MMTr), 6.85 (s, 1H, NH), 6,83, 6.77 and 6.64 (3×d, J=9.0 Hz, 2H, MMTr), 6.05 (d, J=6.5 Hz, 1H, H1′), 5.33 (dd, J=6.5 and 5.0 Hz, 1H, H2′), 4.58 (d, J=11.5 Hz, 1H, OCH₂S), 4.20 (m, 1H, H4′), 4.18 (d, J=11.5 Hz, 1H, OCH₂S), 3.71, 3.78 and 3.81 (3×s, 3H, MMTr), 3.57 (dd, J=4.5 and 2.0 Hz, 1H, H3′), 3.37 (dd, J=10.5 and 5.5 Hz, 1H, H5′), 3.17 (dd, J=10.5 and 4.5 Hz, 1H, H5′), 2.08 (s, 3H, MeS). ESI⁺-MS: m/z obsd (M+H)⁺ 1144.4692, calcd (M+H)⁻ 1144, 4683; obsd (M+Na)⁻ 1166.4479, calcd (M+Na)⁺ 1106.4502; obsd (M+K)⁻ 1182.4248, calcd (M+K)⁺ 1182, 4242.

3′-O-Methylhtiomethyladenosine. 2′-O,5′-O,N⁶-Tris(4-methoxytrityl)-3′-O-methyltiomethyladenosine (2.15 mmol, 2.46 g) was dissolved in 80% AcOH, and the mixture was stirred overnight at room temperature. The mixture was evaporated to dryness and coevaporated twice from water. The product was purified by silica gel chromatography eluting with 10-20% MeOH in DCM. 3′-O-Methyltiomethyladenosine was obtained in 70% (0.49 g) yield. ¹H NMR (500 MHz, CD₃OD) δ 8.33 (s, 1H, H2), 8.21 (s, 1H, H8), 5.98 (d, J=6.5 Hz, 1H, H1′), 4.92 (d, J=12.0 Hz, 1H, OCH₂S), 4.87-4.89 (m, 2H, OCH₂S and H2′), 4.52 (dd, J=5.5 and 2.5 Hz, 1H, H3′), 4.29 (m, 1H, H4′), 3.91 (dd, J=12.5 and 2.5 Hz, 1H, HS′), 3.78 (dd, J=12.5 and 2.5 Hz, 1H, H5″), 2.22 (s, 3H, MeS). ¹³C NMR (126 MHz, CD₃OD) δ: 156.1 (C6), 152.2 (C2), 149.0 (C4), 140.5 (C8), 121.2 (C5), 89.8 (C1′), 84.9 (C4′). 75.6 (C3′). 74.6 (OCH₂S). 73.6 (C2′). 61.9 (C5′), 12.4 (SMe). ESI-MS: m/z obsd (M+H) 328.1067, calcd (M+H)⁺ 328.1080; obsd (M+Na)⁻350.0882, calcd (M+Na) 350.0899.

5′-O-(tert-Butyldimethylsilyl)-3′-O-methyltiomethyl-N⁶-(4-methoxytrityl)adenosine. 3′-O-Methyltiomethyladenosine (2.5 mmol, 0.81 g) dried on P₂O₅ overnight was dissolved in dry pyridine (9.0 mL). tert-Butyldimethylsilyl chloride (3.0 mmol, 0.45 g) was added, and the mixture was stirred overnight at room temperature. The reaction was quenched with MeOH, and the mixture was evaporated to dryness. The product was purified by eluting through a thin layer of silica gel with 10% MeOH in DCM. The volatiles were removed under reduced pressure. The residue was dried by coevaporating twice with anhydrous pyridine and dissolved in the same solvent (10 mL). 4-Methoxytrityl chloride (2.7 mmol, 0.84 g) was added, and the mixture was stirred overnight at 40° C. The reaction was quenched with MeOH, and the mixture was evaporated to dryness. The residue was dissolved in DCM and washed with water, saturated aqueous NaHCO₃ and saturated aqueous NaCl. The organic layer was dried on Na₂SO₄ and evaporated to dryness. The product was purified by silica gel chromatography eluting with 1-2% MeOH in DCM. 5′-O-(tert-Butyldimethylsilyl)-3′-O-methyltiomethyl-N⁶-(4-methoxytrityl)adenosine was obtained as yellowish foam in 88% yield (1.56 g). ¹H NMR (500 MHz, CDCl₃) δ 8.05 (s, 1H, H2). 8.04 (s, 1H. H8), 7.23-7.38 (m, 12H, MMTr), 6.97 (s, 1H, NH), 6.81 (d, J=7.0 Hz, 2H, MMTr), 5.99 (d, J=5.5 Hz, 1H, H1′), 4.88 (d, J=11.5 Hz, 1H, OCH₂S), 4.83 (d, J=11.5 Hz, OCH₂S), 4.71 (m, 1H, H2′), 4.52 (dd, J=5.0 and 3.0 Hz, 1H, H3′), 4.37 (d, J=5.5 Hz, 1H, 2′-OH), 4.31 (m, 1H, H4′), 3.89 (dd, J=11.5 and 4.0 Hz, 1H, H5′), 3.83 (dd, J=11.5 and 3.0 Hz, 1H, H5″), 3.80 (s, 3H, MMTr), 2.23 (s, 3H, MeS), 0.87 (s, 9H, SiCMe₃), 0.10 (s, 3H, Si-Me), 0.06 (s, 3H, Si-Me). ¹³C NMR (126 MHz, CDCl₃) δ: 158.3 (MMTr), 154.2 (C6), 152.0 (C2), 148.4 (C4), 145.2 (MMTr) 138.2 (C8), 137.2, 130.2, 128.9, 127.9. 126.9 (MMTr), 121.2 (C5), 113.2 (MMTr), 89.5 (C1′), 84.3 (C4′), 76.2 (C3′), 75.3 (OCH₂S), 75.0 (C2′), 71.0 (MMTr). 62.8 (C5′), 55.2 (MeO MMTr). 25.9 (CH₃)₃CSi), 18.3 ((CH₃)₃CSi), 14.2 (SMe), −5.5, −5.6 (CH₃Si). ESI-MS: m/z obsd (M+H)⁺ 714.3140, calcd (M+H)⁺ 714.3145; obsd (M+H)⁻ 736.2947, calcd (M+H)⁺ 736.2965.

5′-O-(tert-Butyldimethylsilyl)-2′-O-levulinoyl-3′-O-methyltiomethyl-,v6-(4-methoxytrityl)adenosine . Levulinic acid (5.5 mmol, 0.63 g) was dissolved in dry dioxane (10.0 mL). The mixture was stirred on an ice-bath and dicyclohexylcarbodiimide (2.7 mmol, 0.56 g) was added portion wise within one hour, and the stiffing was continued at room temperature for 2 hours. Dicyclohexylurea was filtered off and washed with dioxane (5.0 mL). The washing was combined to the filtrate and 5′-O-(tert-Butyldimethylsilyl)-3′-O-methyltiomethyl-N⁶-(4-methoxytrityl)adenosine (2.2 mmol, 1.56 g; dried on P₂O₅) in dry pyridine (9.0 mL) was added. A catalytic amount of 4-diaminopyridine was added, and the mixture was stirred overnight at room temperature. The mixture was evaporated to dryness, the residue was dissolved in DCM, washed with saturated aqueous NaHCO₃ and saturated aqueous NaCl and the organic phase was dried on Na₂SO₄. The product was purified by silica gel chromatography eluting with 1% MeOH in DCM. The yield of 5′-O-(tert-Butyldimethylsilyl)-2′-O-levulinoyl-3′-O-methyltiomethyl-,v6-(4-methoxytrityl)adenosine was 88% (1.57 g). ¹H NMR (500 MHz, CDCl₃) δ: 8.11 (s, 1H, H2), 8.06 (s, 1H, H8), 7.23-7.37 (m, 12H, MMTr), 6.94 (s, 1H, NH), 6.81 (d, J=9.0 Hz, 2H, MMTr), 6.23 (d, J=4.5 Hz, 1H, H1′), 5.69 (dd, J=4.5 and 5.0 Hz, 1H, H2′), 4.79 (dd, J=5.0 and 5.5 Hz, 1H, H3′), 4.75 (d, J=11.5 Hz, 1H, OCH₂S), 4.60 (d, J=11.5 Hz, OCH₂S), 4.22 (m, 1H, H4′), 4.01 (dd, J=11.5 and 2.5 Hz, 1H, H5′), 3.86 (dd, J=11.5 and 2.5 Hz, 1H, H5″), 3.80 (s, 3H, MMTr), 2.61-2.80 (m, 4H, Lev), 2.18 (s, 3H, MeS), 2.17 (s, 3H, Lev), 0.94 (s, 9H, SiCMe₃), 0.13 (s, 3H, Si-Me), 0.11 (s, 3H, Si-Me). ESI-MS: m/z obsd (M+H) 812.3474, calcd (M+H)⁺ 812.35 13; obsd (M+Na)⁻ 834.3277, calcd (M+Na)⁻ 812.3333; obsd (M+K)⁺ 850.3009, calcd (M+K)⁺ 850.3072.

3′-O-Acetyloxymethyl-5′-O-(tert-butyldimethylsilyl)-2′-O-levulinoyl-N⁶-(4-methoxytrityl)adenosine. 5′-O-(tert-Butyldimethylsilyl)-2′-O-levulinoyl-3′-O-methyltiomethyl-,v6-(4-methoxytrityl)adenosine (1.04 mmol, 0.84 g) was dried on P₂O ; overnight and dissolved in dry DCM (7.0 mL) under nitrogen. Sulfuryl chloride in DCM (1.24 mmol, 1.14 mL of 1 mol L⁻¹ solution) was added dropwise, and the mixture was stirred for 1 h at room temperature. The volatiles were removed under reduced pressure. The residue was dissolved in DCM (3.0 ml) and added dropwise to a mixture of potassium acetate (1.78 mmol, 0.175 g) and dibenzo-18-crown-6 (0.77 mol, 0.28 g) in dry DCM. After 2 hours stirring at room temperature, the mixture was diluted with ethyl acetate and washed with water and brine. The organic phase was dried on Na₂SO₄. The mixture was concentrated under reduced pressure and the crown ether precipitate was removed by filtration. The product was purified on a silica gel column eluting with DCM containing 1% MeOH. The yield of 3′-O-Acetyloxymethyl-5′-O-(tert-butyldimethylsilyl)-2′-O-levulinoyl-N⁶-(4-methoxytrityl)adenosine was 91% (0.78 g). ¹H NMR (500 MHz, CDCl₃) δ 8.06 (s, 1H, H2), 8.04 (s, 1H, H8), 7.23-7.37 (m, 12H, MMTr), 6.93 (s, 1H, NH), 6.81 (d, J=9.0 Hz, 2H, MMTr), 6.19 (d, J=4.5 Hz, 1H, H1′), 5.71 (dd, J=4.5 and 5.0 Hz, 1H, H2′), 5.30 (d, J=6.5 Hz, 1H, OCH₂O), 5.27 (d , J=6.5 Hz, 1H, OCH₂O), 4.80 (dd, J=5.0 and 5.5 Hz, 1H, H3′), 4.21 (m, 1H, H4′), 3.97 (dd, J=11.5 and 3.0 Hz, 1H, H5′), 3.83 (dd, J=11.5 and 3.0 Hz, 1H, H5″), 3.81 (s, 3H, MMTr), 2.61-2.78 (m, 4H, Lev), 2.17 (s, 3H, Lev), 2.11 (s, 3H, Ac), 0.92 (s, 9H, SiCMe₃), 0.11 (s, 3H, Si-Me), 0.09 (s, 3H, SiMe) ¹³C NMR (126 MHz, CDCl₃) Ii 206.1 (C═O lev), 171.7 (C═O lev), 170.4 (C═O Ac), 158.3 (MMTr), 154.1 (C6), 152.5 (C2), 148.6 (C4), 145.2 (MMTr), 138.4 (C8), 137.2 (MMTr), 130.2 (MMTr), 128.9 (MMTr), 127.9 (MMTr), 126.9 (MMTr), 121.2 (C5), 113.2 (MMTr), 88.3 (OCH₂O), 86.2 (C1′), 83.5 (C4′), 76.2 (C3′), 74.8 (C2′), 71.0 (MMTr), 62.1 (C5′), 55.2 (MMTr), 37.8 (lev), 29.8 (lev), 27.7 (lev), 25.9 (TBDMS), 21.0 (CH₃COOCH₂), 18.4 (TBDMS), −5.5 (TBDMS), −5.4 (TBDMS). ESI-MS: m/z obsd (M+H)⁺ 824.3641, calcd (M+H) 824.3685; obsd (M+Na)⁺ 846.3485, calcd (M+Na)″ 846.3 505.

3′-O-Acetyloxymethyl-5′-O-(tert-butyldimethylsilyl)-N⁶-(4-methoxytrityl)adenosine (12). 3′-O-Acetyloxymethyl-5′-O-(tert-butyldimethylsilyl)-2′-O-levulinoyl-N⁶-(4-methoxytrityl)adenosine (0.78 mmol, 0.64 g) was dissolved in dry DCM (18 ml). Hydrazine acetate (1.17 mmol; 0.107 g) in dry MeOH (2.0 mL) was added, and the mixture was stirred at room temperature for 1 hour. The reaction was quenched with 1.5 equiv, of acetone, stirred for 15 min and evaporated to dryness. The product was purified on a silica gel column eluting with DCM containing 1-2% MeOH. The yield of 12 was 78% (0.44 g). ¹H NMR (500 MHz, CDCl₃) δ 8.03 (5, 1H, H2), 8.01 (2.1H, H8), 7.23-7.37 (m, 12H, MMTr), 6.96 (s, 1H, NH), 6.81 (d, J=9.0 Hz, 2H, MMTr), 5.94 (d, J=6.0 Hz, 1H, H1′), 5.46 (d, J=6.5 Hz, 1H, OCH₂O), 5.44 (d, J=6.5 Hz, 1H, OCH₂O), 4.71 (m, 1H, H2′), 4.51 (m, 1H, H3′), 4.46 (d, J=5.5 Hz, 1H, 2′-OH), 4.32 (m, 1H, H4′), 3.87 (dd, J=11.5 and 4.0 Hz, 1H, H5′), 3.81-3.83 (m, 4H, MMTr and H5′), 3.81 (s, 3H, MMTr), 2.14 (5, 3H, Ac), 0.85 (5, 9H, SiCMe₃), 0.08 (s, 3H, Si-Me), 0.03 (s, 3H, Si-Me). ¹³C NMR (126 MHz, CDCl₃) δ: 170.5 (C═O), 158.3 (MMTr), 154.2 (C6), 152.0 (C2), 148.0 (C4), 145.1 (MMTr) 138.3 (C8), 137.2, 130.2, 128.9, 127.9, 126.9 (MMTr), 121.2 (C5), 113.2 (MMTr), 89.5 (C1′),88.5 (OCH₂O), 84.6 (C4′). 79.3 (C3′), 75.2 (C2′), 71.0 (MMTr), 62.8 (C5′), 55.2 (MeO MMTr), 25.8 ((CH₃)₃CSi), 21.1 (Ac), 18.2 ((CH₃)₃CSi), −5.5, −5.6 (CH₃Si). ESI⁺-MS: m/z obsd (M+H)⁺ 726.3295, calcd (M+H)⁺ 726.3318; obsd (M+Na)⁻ 748.3106, calcd (M+Na)⁻ 748,3137; obsd (M+K) 764.2834, calcd (M+K)⁺ 764.2876.

2′-O-Levulinoyl-N⁶-(4-methoxytrityl)-3′-O-acetyloxymethyladenosine (11). The trimethylsilyl group is removed by treatment with tetrabutylammonium fluoride in THF under acidic conditions. Bu₄NF is dissolved in dry THF and AcOH is added. The nucleoside is added, and the mixture is stirred at room temperature for several hours. Saturated aqueous NaHCO₃ is added, and the mixture is extracted with dichloromethane. The organic phase is dried over Na₂SO₄ and evaporated to dryness. The product is purified by Silica gel chromatography, eluting with dichloromethane containing 3% MeOH.

### Bis[2-(4,4-dimethoxytritylthio)ethyl]-N,N-diisopropylphosphoramidite (13)

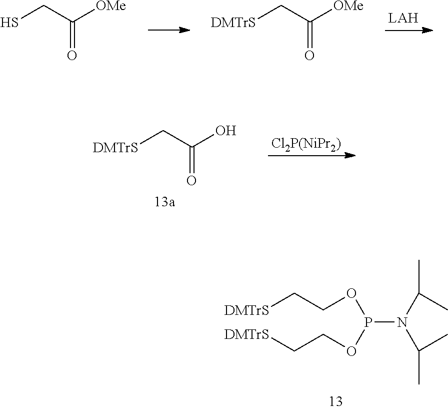

The phosphitylating reagent 13 was prepared as described in Austin, C.; Grajkowski, A.; Cieslak, J.; Beaucage, S. L. Org. Lett. 2005, 7, 4201-4204, which is hereby incorporated by reference in its entirety.

2-(4,4′-Dimethoxytrityltio)ethanol (13a). ¹H NMR (CDCl₃) 7.44-7.46 (m, 2H, DMTr), 7.25-7.38 (m, 7H, DMTr), 6.83-6.86 (m, 4H, DMTr), 3.82 (s, 6H, DMTr), 3.45 (q, J=5.8 Hz, 1H, CH₂OH), 2.53 (t, J=6.2 Hz, 2H, SCH₂), 1.74 (t, J=5.1 Hz, 1H, OH). ¹³C NMR (CDCl₃) δ 158.1 (DMTr), 145.4 (DMTr), 137.2 (DMTr), 130.7 (DMTr), 129.4 (DMTr), 128.3 (DMTr), 126.7 (DMTr), 113.2 (DMTr), 65.8 (DMTr), 61.0 (CH₂O), 55.3 (OCH₃), 35.4 (CH₂S).

Bis[2-(4,4′-dimethoxytritylthio)ethyl]-N,N-diisopropylphosphoramidite (13). ¹H NMR (CDCl₃) δ 7.41-7.43 (m, 4H, DMTr), 7.26-7.34 (m, 14H, DMTr), 6.81-6.83 (m, 8H, DMTr), 3.82 (s, 12H, DMTr), 3.36-3.54 (m, 6H, 2×CH₂O and 2×CHMe₂), 2.45-2.50 (q, J=7.8 Hz, 4H, CH₂S), 1.12 (d, J=6.8 Hz, 12H, CH₃). ³¹P NMR (CDCl₃) 146.4.

### 2-[2′-O-levulinoyl-3′-O-methyladenosin-5′-yl]-2-oxo-6,7-dithia-1,3,2-dioxaphosphonane (14)

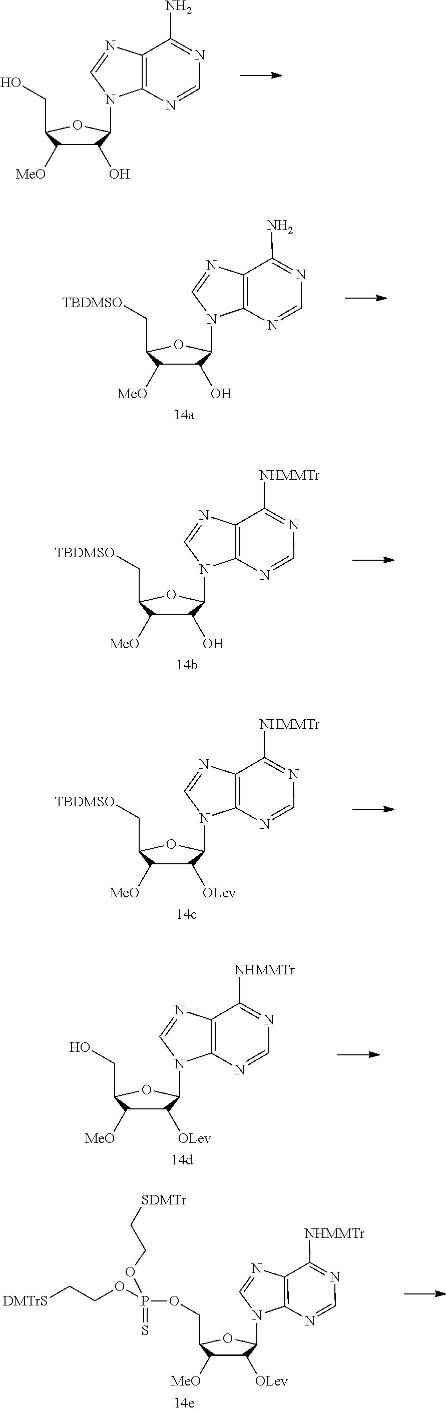

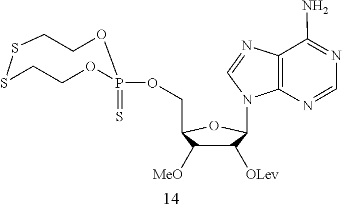

5′-O-(tert-Butyldimethylsilyl)-3′-O-methyladenosine (14a). Commercial (Chem Genes corporation) 3′-O-methyladenosine (2.8 mmol; 0.80 g) was co-evaporated twice from dry pyridine and dissolved in pyridine (5.0 mL). tert-Butyldimethylsilyl chloride (3.1 mmol; 0.47 g) was added, and the reaction was allowed to proceed overnight. The mixture was diluted with MeOH (8.0 mL) and then evaporated to dryness. The residue was dissolved in chloroform, washed with water, aqueous NaHCO₃ and brine. The organic phase was then dried with Na₂SO₄. The product was purified by silica gel chromatography eluting with 5% MeOH in DCM. (Yield 72%). ¹H NMR (CD₃OD) δ 8.41 (s, 1H, H2), 8.23 (s, 1H, H8), 6.06 (d, J=4.2 Hz, 1H, H1′), 4.77 (dd, J=4.2 and 4.9 Hz, 1H H2′), 4.21-4.23 (m, 1H, H4′), 4.06 (dd, J=4.9 and 5.1 Hz, 1H, H3′), 4.02 (dd, J=11.5 and 3.4 Hz, 1H, H5′), 3.87 (dd, J=11.5 and 3.0 Hz, 1H, H5″), 3.04 (s, 3H, 3′-OMe), 0.96 (s, 9H, TBDMS), 0.14 (2×s, 6H, TBDMS). ¹³C NMR (CD₃OD) δ 155.9 (C6), 152.5 (C2), 149.1 (C4), 139.3 (C8), 119.0 (C5), 88.8 (C1′), 82.7 (C4′), 78.9 (C3′), 73.3 (C2′), 62.3 (C5′), 57.1 (OMe), 25.0 (TBDMS), 17.9 (TBDMS), −6.7 (TBDMS).

5′-O-(tert-Butyldimethylsilyl)-N⁶-(4-methoxytrityl)-3′-O-methyladenosine (14b). Compound 14a (3.0 mmol, 1.17 g) was dissolved in dry pyridine (7.0 mL) and 4-methoxytrityl chloride (3.2 mmol; 1.00 g) was then added, and the reaction was allowed to proceed overnight at 54° C. The mixture was diluted with MeOH and evaporated to dryness. The product was purified by silica gel chromatography eluting with a 1:1 mixture of petroleum ether and ethyl acetate. (Yield 74%). ¹H NMR (CDCl₃) δ 8.05 (s, 2H, H2 and H8), 7.35-7.37 (m, 4H, MMTr), 7.23-7.30 (m, 8H, MMTr), 6.96 (s, 1H, NH), 6.81-6.82 (m, 2H, MMTr), 5.98 (d, J=5.6 Hz, 1H, H1′), 4.71-4.75 (m, 1H, H2′), 4.26-4.29 (m, H, H4′), 4.16 (d, J=6.5 Hz, 1H, OH), 4.03-4.06 (m, 1H, H3′), 3.91 (dd, J=11.2 and 4.2 Hz, 1H, H5′), 3.78-3.81 (m, 4H, MMTr and H5″), 3.52 (s, 3H, 3′-OMe), 0.90 (s, 9H, TBDMS), 0.10 (s, 3H, TBDMS), 0.08 (s, 3H, TBDMS). ¹³C NMR (CDCl₃) δ 158.3 (MMTr), 154.1 (C6), 152.1 (C2), 148.5 (C4), 145.2 (MMTr), 138.3 (C8), 137.2 (MMTr), 130.2 (MMTr), 128.9 (MMTr), 127.9 (MMTr), 126.9 (MMTr), 121.3 (C5), 113.2 (MMTr), 88.8 (C1′), 83.1 (C4′), 80.0 (C3′), 74.4 (C2′), 71.0 (MMTr), 63.0 (C5′), 58.1 (3′-OMe), 55.2 (MMTr), 25.9 (TBDMS), 18.3 (TBDMS), −5.4 (TBDMS).

5′-O-(tert-Butyldimethylsilyl)-2′-O-levulinoyl-N⁶-(4-methoxytrityl)-3′-O-methyladenosine (14c). Levulinic acid (4.1 mmol; 0.48 g) was dissolved in dry dioxane (4.0 mL) and the solution was cooled on an ice-bath. Dicyclohexylcarbodiimide (DCC) (2.0 mmol; 0.42 g) was added during 50 min in 3 portions. One hour after the first addition, dicyclohexylurea byproduct formed, and was removed by filtration. The filtrate was then added into a solution of compound 14b (1.3 mmol; 0.90 g) in pyridine (5.0 mL). The reaction was allowed to proceed overnight. The mixture was evaporated to dryness and the residue was subjected to DCM/aq NaHCO₃ workup. The organic phase was dried on Na₂SO₄. The crude product was subjected without purification to removal of the TBDMS group. ¹H NMR (CDCl₃) δ 8.08 (s, 1H, H2), 8.03 (s, 1H, H8), 7.33-7.35 (m, 4H, MMTr), 7.22-7.28 (m, 8H, MMTr), 6.89 (s, 1H, NH), 6.78-6.80 (m, 2H, MMTr), 6.16 (d, J=4.0 Hz, 1H, H1′), 5.71 (dd, J=4.0 and 5.0 Hz, 1H, H2′), 4.26 (dd, J=5.0 and 5.3, 1H, H3′), 4.13-4.16 (m, 1H, H4′), 3.99 (dd, J=11.5 and 3.1 Hz, 1H, H5′), 3.79 (dd, J=11.5 and 3.0 Hz, H5″), 3.78 (s, 3H, MMTr), 3.39 (s, 3H, 3′-OMe), 2.59-2.82 (m, 4H, Lev), 2.15 (s, 3H, Lev), 0.91 (s, 9H, TBDMS), 0.10 (s, 3H, TBDMS), 0.09 (s, 3H, TBDMS).

2′-O-Levulinoyl-N⁶-(4-methoxytrityl)-3′-O-methyladenosine (14d). Compound 14c (1.4 mmol; 1.06 g) was dissolved in a mixture of acetic acid (3.0 mL) and THF (16 mL). Tetrabutylammonium fluoride (2.7 mmol; 0.72 g) was then added, and the reaction was allowed to proceed 2 days at room temperature. The volatiles were removed at reduced pressure, and the residue was co-evaporated from water. The crude product was subjected to ethyl acetate/aqueous NaHCO₃ workup. The crude product was purified on a silica gel column eluting with 5% MeOH in DCM. (Yield 92%). ¹H NMR (CDCl₃) δ 7.99 (s, 1H, H2), 7.77 (s, 1H, H8), 7.32-7.35 (m, 4H, MMTr), 7.22-7.28 (m, 8H, MMTr), 7.00 (s, 1H, NH), 6.78-6.80 (m, 2H, MMTr), 6.00 (d, J=7.2 Hz, 1H, H1′), 5.71 (dd, J=4.9 and 7.2 Hz, 1H, H2′), 4.34-4.36 (m, 2H, H3′ and H4′), 3.97 (d, J=13.0 Hz, 1H, H5′), 3.78 (s, 3H, MMTr), 3.67 (d, J=13.0 Hz, H5″), 3.44 (s, 3H, 3′-OMe), 2.53-2.78 (m, 4H, Lev), 2.17 (s, 3H, Lev). ¹³C NMR (CDCl₃) δ 206.1 (C═O Lev), 171.6 (C═O Lev), 158.4 (MMTr), 154.6 (C6), 151.9 (C2), 147.3 (C4), 144.9 (MMTr), 139.8 (C8), 136.9 (MMTr), 130.2 (MMTr), 128.8 (MMTr), 128.0 (MMTr), 127.0 (MMTr), 122.5 (C5), 113.2 (MMTr), 89.1 (C1′), 86.1 (C4′), 79.4 (C2′), 75.0 (C3′), 71.1 (MMTr), 63.2 (C5′), 58.5 (3′-OMe), 55.2 (MMTr), 37.7 (Lev), 29.8 (Lev), 27.6 (Lev).

2′-O-Levulinoyl-N⁶-(4-methoxytrityl)-3′-O-methyladenosine 5′-bis[2-(4,4′-dimethoxytritylthio)ethyl]phosphorothioate (14e). Compound 14d (1.3 mmol; 0.87 g) and compound 2 (1.6 mmol; 1.44 g) were dried for 2 days on P₂O₅. Compound 14d was dissolved in dry MeCN (3.0 mL) under nitrogen. Compound 13 was dissolved in MeCN (3.0 mL). The two solutions were mixed, and tetrazole (2.0 mmol; 4.45 mL of 0.45 mol L⁻¹ solution in MeCN) was added under nitrogen. After 30 minutes, elemental sulfur (S₈) (9.0 mmol; 0.29 g) in 10 mL of dry pyridine was added, and the reaction was allowed to proceed for 40 minutes. The reaction was quenched by adding aqueous triethylammonium acetate (18.7 mL of 1.0 mol L⁻¹ aq solution) and the mixture was stirred for 30 minutes. The product was extracted using DCM (40 mL), and the organic phase was washed with water (4×15 mL) and dried on Na₂SO₄. The product was purified by silica gel chromatography using a 7:3 mixture of ethyl acetate and petroleum ether containing 1% triethylamine as an eluent. (Yield was 57%). ¹H NMR (CDCl₃) δ 8.00 (s, 1H, H2), 7.93 (s, 1H, H8), 7.19-7.37 (m, 30H, DMTr and MMTr), 6.91 (br s, 1H, NH), 6.73-6.78 (m, 10H, DMTr and MMTr), 6.07 (d, J=5.6 Hz, 1H, H1′), 5.68 (dd, J=5.6 and 3.8 Hz, 1H H2′), 4.13-4.30 (m, 4H, H3′, H4′, H5′ and H5″), 3.69-3.76 (m, 19H, 5×OMe and 2×CH₂O), 3.32 (s, 3H, 3′-OMe), 2.57-2.78 (m, 4H, Lev), 2.46-2.51 (m, 4H, 2×CH₂S), 2.15 (s, 3H, Lev). ³¹P NMR (CDCl₃) δ7.6.

2-[2′-O-Levulinoyl-3′-O-methyladenosin-5′-yl]-2-oxo-6,7-dithia-1,3,2-dioxaphosphonane (14). Compound 14e (1.0 mmol; 1.39 g) was dissolved in DCM (5.0 mL), and 1% methanolic solution of iodine (20 mL) was than added. After 1 hour, the reaction was quenched with 10% aqueous NaHSO₃. The organic phase was separated and dried on Na₂SO₄. The crude product was purified on a silica gel column eluting with 1-20% MeOH in DCM. (Yield 12%). ¹H NMR (CDCl₃) δ 8.34 (s, 1H, H2), 8.01 (s, 1H, H8), 6.14 (d, J=3.4 Hz, 1H, H1′), 5.98 (br s, 2H, NH₂), 5.93 (dd, J=3.4 and 5.1 Hz, 1H H2′), 4.30-4.48 (m, 8H, H3′, H4′, H5′, H5″ and 2×CH₂O), 3.46 (s, 3H, 3′-OMe), 2.90-2.98 (br s, 4H, 2×CH₂S), 2.57-2.78 (m, 4H, Lev), 2.18 (s, 3H, Lev). ¹³C NMR (CDCl₃) δ 206.1 (C═O Lev), 171.7 (C═O Lev), 155.6 (C6), 153.2 (C2), 149.4 (C4), 139.4 (C8), 120.1 (C5), 87.4 (C1′), 80.6 (C3′), 77.9 (C4′), 73.8 (C2′), 66.3 (C5′), 63.5 (2×OCH₂), 59.2 (3′-OMe), 37.8 (Lev), 37.6 (2×SCH₂) 29.7 (Lev), 27.8 (Lev).

### 5′-dimethoxytrityl-3′-tert-butyldimethylsilyl-6-N-benzoyladenosine-2′-H-phosphonate DMT-A-P(H) (15)

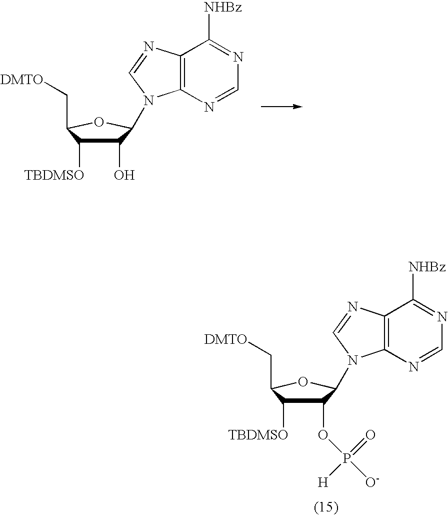

5′-Dimethoxytrityl-3′-tert-butyldimethylsilyl-6-N-benzoyladenosine-2′-H-phosphonate DMT-A-P(H) (15). 5′-DMT-3′-TBDMS-6-N-Benzoyladenosine (10 g) was added to a solution of diphenyl phosphate (10 g) in pyridine (50 mL). The mixture was stirred at room temperature for 2 hours. Water (5 mL) and triethylamine (5 mL) were added, and the mixture was stirred for an additional 15 minutes. The mixture was then diluted with water (200 mL) and extracted with dichloromethane (200 mL). The organic layer was dried and evaporated. The residue was purified by silica gel chromatography using methanol:dichloromethane (0:100-5:95 v/v). The appropriate fractions were combined and evaporated to give the title compound as its triethylammonium salt (9 g), a solid.

### 3′,6-dibenzoyl-2′-C-methyladenosine HO-A[2′-C-methyl] (16)

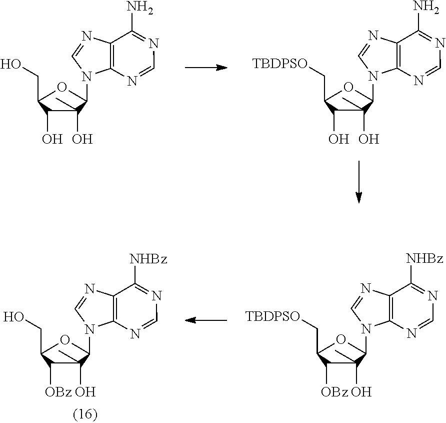

3′,6-Dibenoyl-2′-C-methyladenosine HO-A[2′-C-methyl] (16). 2′-C-Methyladenosine (562 mg), imidazole (2 g) and t-butyldiphenylsilyl chloride (0.8 mL) was stirred in anhydrous pyridine (20 mL) for 24 hours. The mixture was then poured into an aqueous sodium bicarbonate solution (100 mL) and extracted with dichloromethane (50 mL). The organic layer was evaporated and co-evaporated with pyridine (3×10 mL). The residue was then dissolved in anhydrous pyridine (20 mL) followed by the addition of benzoyl chloride (1.2 mL) and 4-dimethylaminopyridine (0.5 g). After addition was complete, the mixture was stirred at room temperature for 24 hours. The mixture was then poured into aqueous sodium bicarbonate (50 mL) and then extracted with dichloromethane (50 mL). The organic layer was evaporated, and the residue was dissolved in THF (20 mL). Tetrabutylammonium fluoride (5 mL, 1 M in THF) was then added and the mixture was stirred at room temperature for 24 hours. The mixture was then poured into aqueous sodium bicarbonate (50 mL) and extracted with dichloromethane (50 mL). The organic layer was evaporated, and the residue was purified by column chromatography using methanol:dichloromethane (4:96) as the eluent. The appropriate fractions were evaporated to give the title compound as a colorless solid (280 mg).

### 2′,3′,6-triacetyl-3′-C-methyladenosine, HO-A[3′-C-methyl] (17)

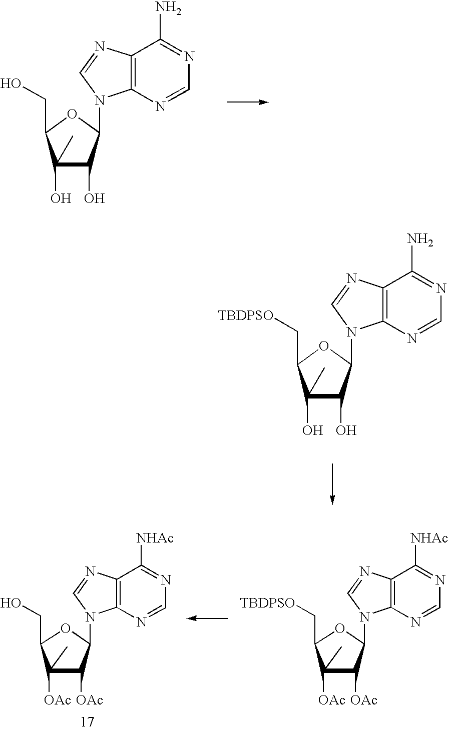

2′,3′,6-triacetyl-3′-C-methyladenosine, HO-A[3′-C-methyl] (17). 3′-C-Methyladenosine (281 mg), imidazole (1 g) and t-butyldiphenylsilyl chloride (0.4 mL) was stirred in anhydrous pyridine (10 mL) for 24 hours. The mixture was then poured into aqueous sodium bicarbonate (50 mL) and extracted with dichloromethane (50 mL). The organic layer was evaporated and co-evaporated with pyridine (3×10 mL). The residue was dissolved in anhydrous pyridine (10 mL) followed by the addition of acetic anhydride (0.5 mL) and 4-dimethylaminopyridine (0.1 g). The mixture was stirred at room temperature for 24 hours. The mixture was then poured into aqueous sodium bicarbonate (50 mL) and extracted with dichloromethane (50 mL). The organic layer was evaporated, and the residue was dissolved in THF (20 mL). Tetrabutylammonium fluoride (2 mL, 1 M in THF) was then added, and the mixture was stirred at room temperature for 24 hours. The mixture was poured into aqueous sodium bicarbonate (50 mL) and extracted with dichloromethane (50 mL). The organic layer was evaporated, and the residue was purified by column chromatography using methanol:dichloromethane (5:95) as the eluent. The appropriate fractions were evaporated to give the title compound as a colorless solid (215 mg).

### 2′,3′,6-triacetyl-2′-C-methyl-7-deaza-adenosine HO-A[2′C-methy-7-deazal] (18)

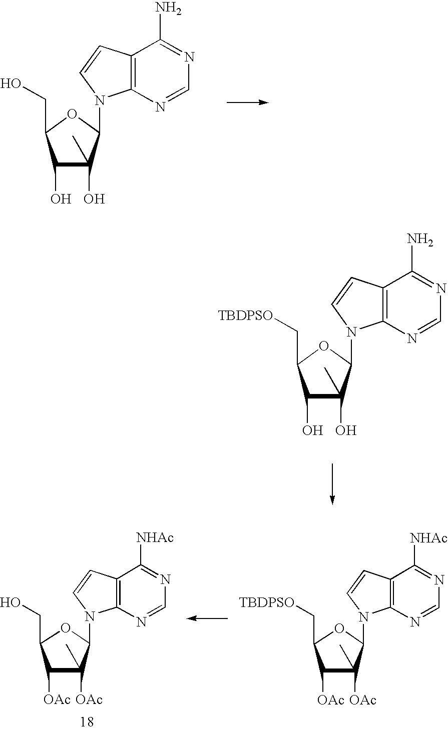

2′,3′,6-triacetyl-2′-C-methyl-7-deaza-adenosine HO-A[2′-C-methyl-7-deazal] (18). 3′-C-Methyladenosine (280 mg), imidazole (1 g) and t-butyldiphenylsilyl chloride (0.4 mL) was stirred in anhydrous pyridine (10 mL) for 24 hours. The mixture was then poured into aqueous sodium bicarbonate (50 mL) and extracted with dichloromethane (50 mL). The organic layer was evaporated and co-evaporated with pyridine (3×10 mL). The residue was dissolved in anhydrous pyridine (10 mL) followed by the addition of acetic anhydride (0.5 mL) and 4-dimethylaminopyridine (0.1 g). The mixture was stirred at room temperature for 24 hours. The mixture was then poured into aqueous sodium bicarbonate (50 mL) and extracted with dichloromethane (50 mL). The organic layer was evaporated, and the residue was dissolved in THF (20 mL). Tetrabutylammonium fluoride (2 mL, 1 M in THF) was then added. After addition was complete, the mixture was stirred at room temperature for 24 hours. The mixture was then poured into aqueous sodium bicarbonate (50 mL) and extracted with dichloromethane (50 mL). The organic layer was evaporated, and the residue was purified by column chromatography using methanol:dichloromethane (5:95) as the eluent. The appropriate fractions were evaporated to give the title compound as a colorless solid (225 mg).

### DMT-APA[2′-C-methyl] (19)

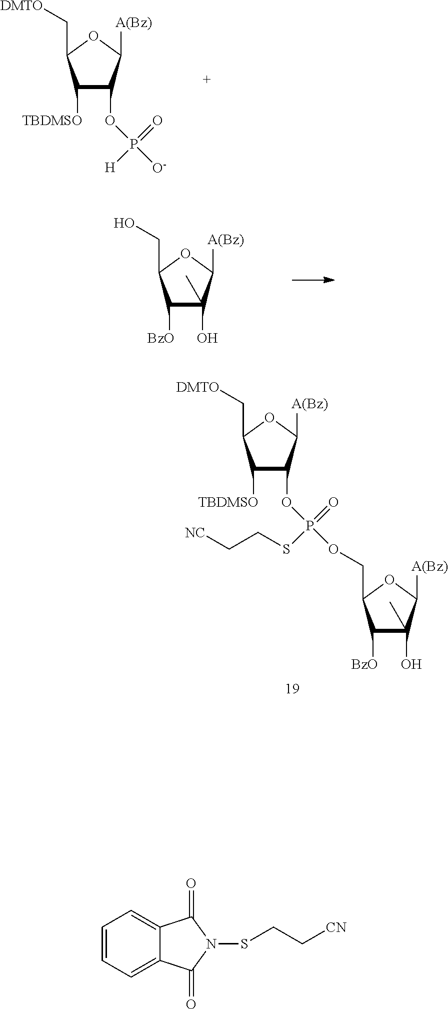

Compound 15 (250 mg) and compound 16 (98 mg) were co-evaporated with anhydrous pyridine (2×2 mL) and then dissolved in anhydrous pyridine (5 mL). The solution was cooled to −40° C. using an acetone-dry ice bath. Bis-(2-chlorophenyl)phosphorochloridate (0.2 mL) in dry dichloromethane (1 mL) was then added over 5 min, and the mixture was stirred for 5 minutes. N-[(2-cyanoethyl)sulfanyl]phthalimide (100 mg) was then added, and the mixture was stirred for an additional 15 minutes at −40° C. A solution of water:pyridine (1 mL, 1:1 v/v) was added. The reaction mixture was poured into saturated aqueous sodium bicarbonate (20 mL) and extracted with dichloromethane (2×20 mL). The combined organic layers were washed with saturated aqueous sodium bicarbonate (3×20 mL), dried with MgSO₄ and concentrated. The residue was purified by silica gel chromatography using methanol:dichloromethane (0:100-4:96 v/v) as the eluent. The appropriate fractions were combined and evaporated to give the title compound as a colorless solid (230 mg).

### HO-APA[2′-C-methyl] (20)

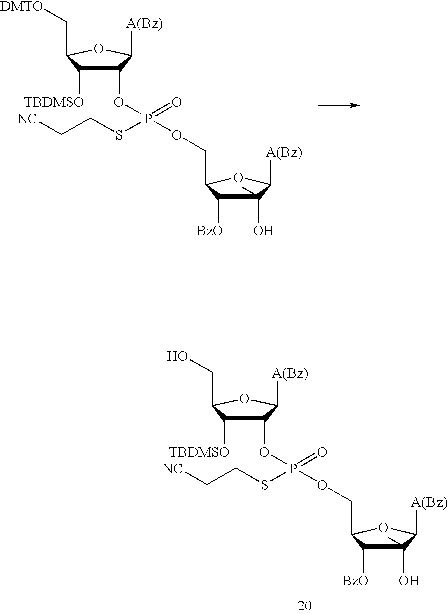

HO-APA[2′-C-methyl] (20). A solution of dichloroacetic acid 0.3 mL) in dichloromethane (4 mL) was added to a cooled (ice-water bath) stirred solution of DMT-ApA[2′-C-methyl] (230 mg) and pyrrole (0.4) in dichloromethane (10 mL). After 10 minutes, the mixture was poured into saturated aqueous sodium hydrogen carbonate (20 mL). The layers were separated, and the aqueous layer was extracted with dichloromethane (3×10 mL). The combined organic layers were dried with MgSO₄ and evaporated under reduced pressure. The residue was fractionated by short column chromatography on silica gel using dichloromethane-methanol (99:1 to 95:5 v/v). The appropriate fractions were evaporated under reduced pressure to give the title compound as a colorless solid (160 mg).

### DMT-APAPA[2′-C-methyl] (21)

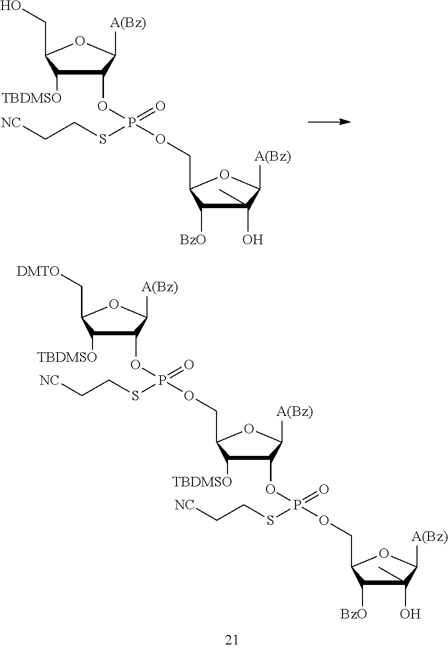

DMT-ApApA[2′-C-methyl] (21). DMT-A-p(H) (220 mg) and HO-ApA[2′-C-methyl] (160 mg) were co-evaporated with anhydrous pyridine (2×2 mL) and then were dissolved in anhydrous pyridine (5 mL). The solution was cooled to −40° C. (acetone-dry ice bath) and bis-(2-chlorophenyl)phosphorochloridate (0.18 mL) in dry dichloromethane (1 mL) was added over 5 minutes, and stirred for an additional 5 minutes. N-[(2-cyanoethyl)sulfanyl]phthalimide (80 mg) was added, and the mixture was stirred for 15 minutes at the same temperature. Water-pyridine (1 mL, 1:1 v/v) was then added. The reaction mixture was then poured into saturated aqueous sodium bicarbonate (20 mL) and extracted with dichloromethane (2×20 mL). The combined organic layers were washed with saturated aqueous sodium bicarbonate (3×20 mL), dried with MgSO₄ and concentrated. The residue was purified by silica gel chromatography using methanol-dichloromethane (0:100-5:95 v/v). The appropriate fractions with were combined and evaporated to give the title compound as a colorless solid (235 mg).

### HO-APAPA[2′-C-methyl] (22)

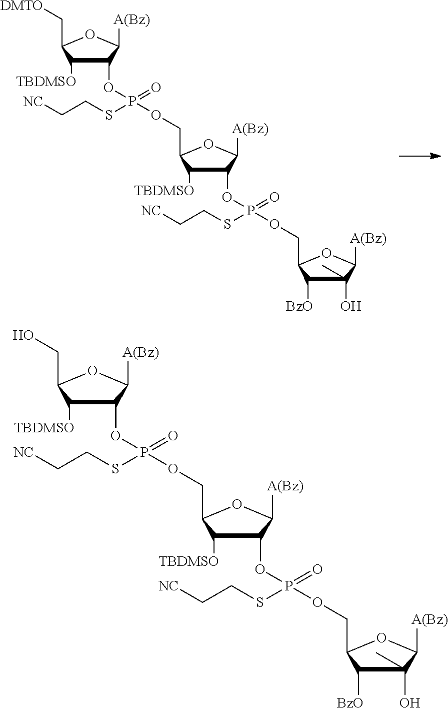

HO-APAPA[2′-C-methyl] (22). A solution of dichloroacetic acid 0.5 mL) in dichloromethane (5 mL) was added to a cooled (ice-water bath) stirred solution of DMT-ApApA[2′-C-methyl] (235 mg) and pyrrole (0.4) in dichloromethane (10 mL). After 10 minutes, the mixture was poured into saturated aqueous sodium hydrogen carbonate (20 mL). The layers were separated, and the aqueous layer was extracted with dichloromethane (3×10 mL). The combined organic layers were dried with MgSO₄ and evaporated under reduced pressure. The residue was fractionated by short column chromatography on silica gel using dichloromethane-methanol (99:1 to 95:5 v/v). The appropriate fractions were evaporated under reduced pressure to give the title compound as a colorless solid (185 mg).

### Trimer Synthesis

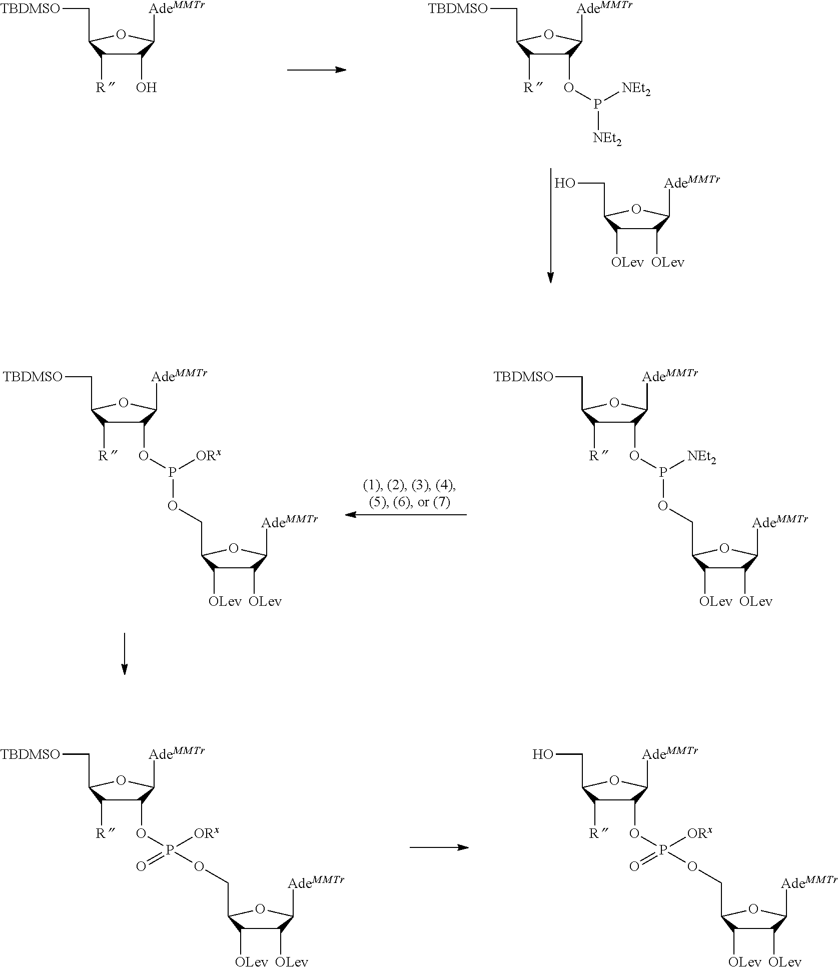

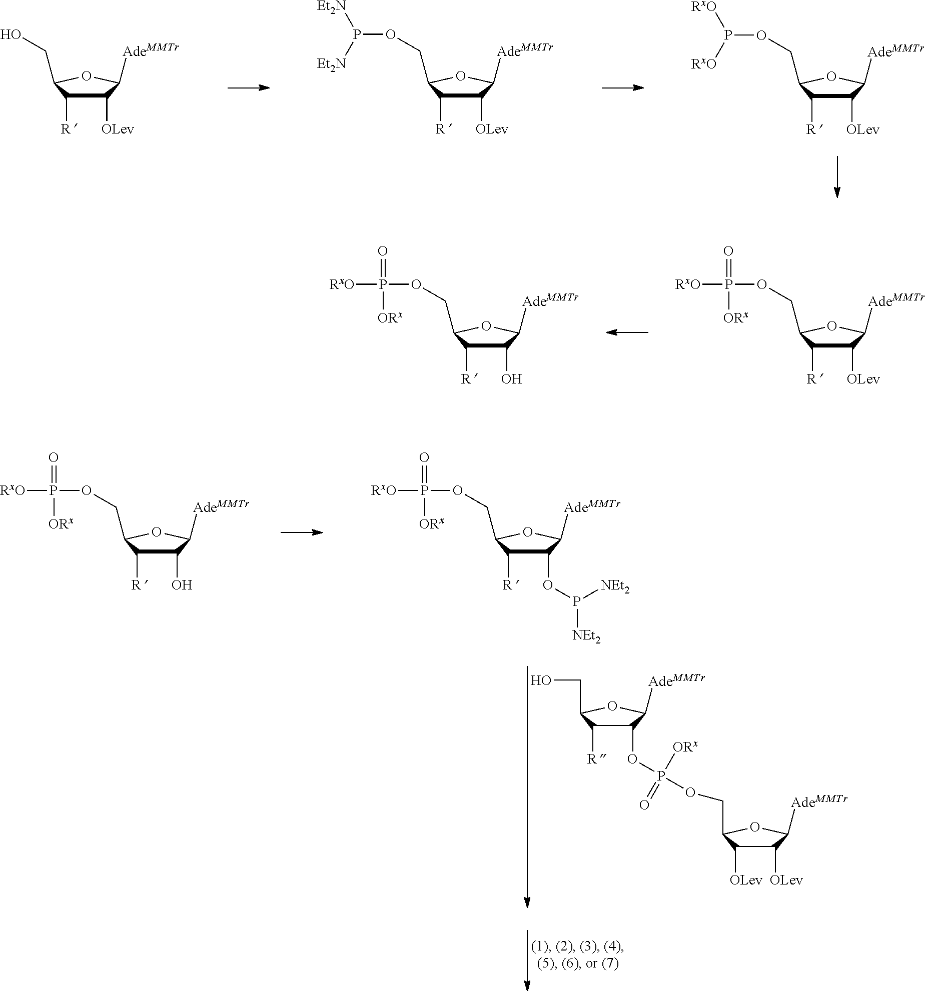

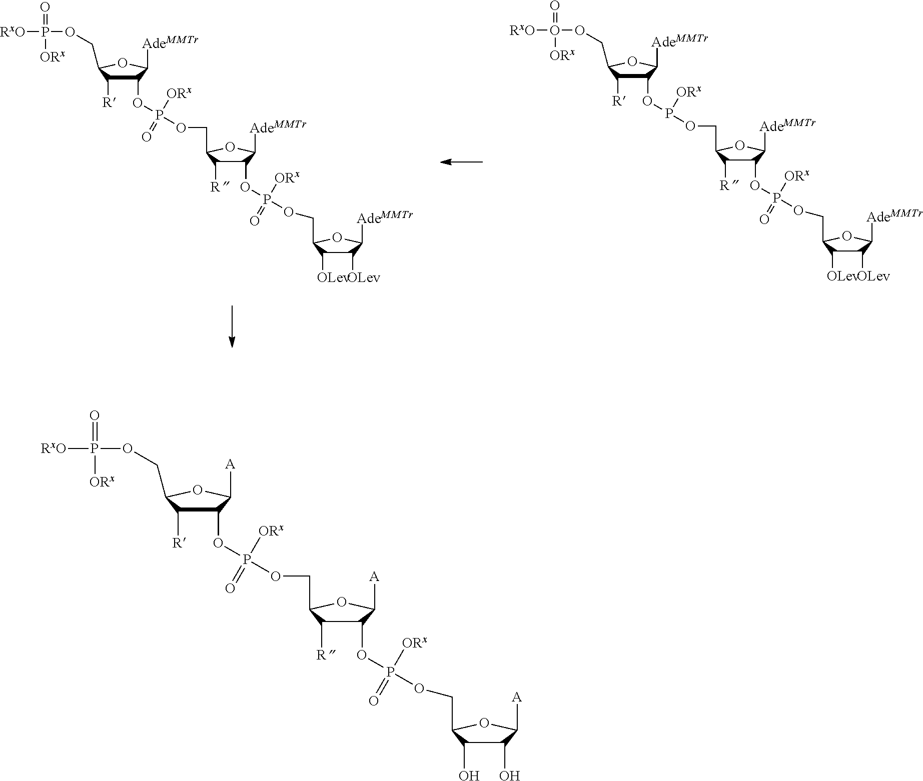

### R″=

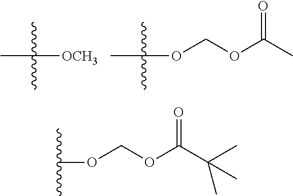

### R^{x}=

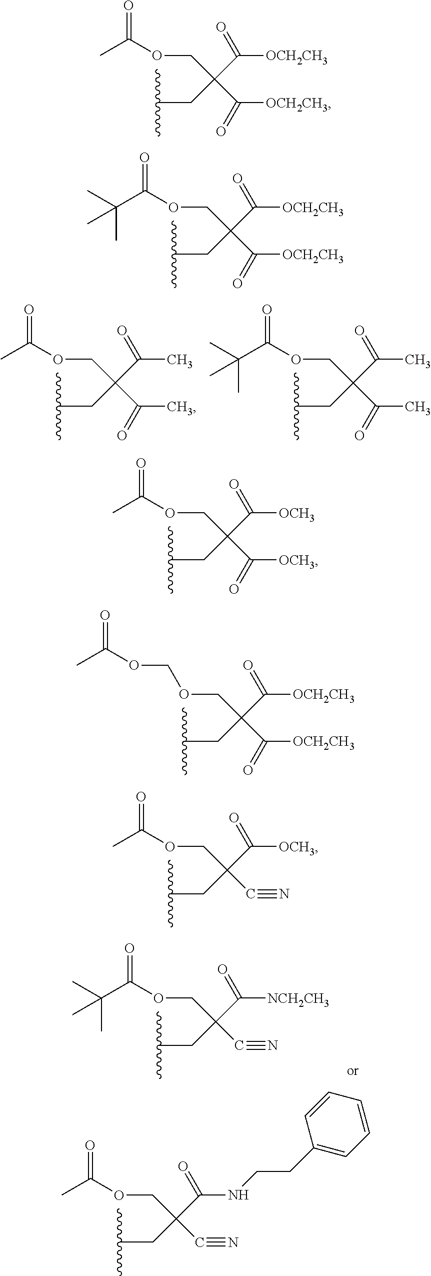

2′-O-levulinoyl-N⁶-(4-methoxytrityl)-3′-O-pivaloyloxymethyladenosine 5′-Bis[3-acetyloxy-2,2-bis(ethoxycarbonyl)propyl]phosphate. Compound 9b (1.5 mmol, 1.10 g, dried over P₂O₅ overnight) was dissolved in dry dichloromethane (7 mL) under nitrogen. Anhydrous triethylamine (7.3 mmol, 1.02 mL) and bis(diethylamino)chlorophosphine (2.1 mmol, 0.43 mL) were added, and the mixture was stirred for 2 hours. The product was isolated by passing the mixture through a short silica gel column with a 7:3 mixture of ethyl acetate and hexane containing 0.5% triethylamine. The solvent was removed under reduced pressure, and the residue was coevaporated from dry acetonitrile to remove the traces of Et₃N. The identity of the phosphitylated product was verified by ³¹P spectroscopy. ³¹P NMR (202 MHz, CD₃CN) δ: 133.3. The phosphitylated nucleoside was dissolved in dry acetonitrile (1 mL) under nitrogen. 2,2-Bis(ethoxycarbonyl)-3-acetyloxymethyl)propanol (4.2 mmol, 1.10 g, coevaporated twice with dry MeCN and dried over P₂O₅ overnight), was dissolved in dry MeCN (2 mL), and tetrazole (4.4 mmol, 9.76 mL of 0.45 mol L⁻¹ solution in MeCN) were added. The progress of the reaction was followed by ³¹P NMR spectroscopy. The spectrum was recorded after half an hour. ³¹P NMR (202 MHz, CD₃CN) δ: 138.8. The phosphite ester formed was oxidized with I₂ (0.1 mol L⁻¹) in a mixture of THF, H₂O and 2,6-lutidine (4:2:1, v/v/v, 10 mL) after a half an hour. The mixture was stirred over night at room temperature. Aqueous 5% NaHCO₃ was added, and the mixture was extracted twice with dichloromethane. The organic phase was dried over Na₂SO₄ and evaporated to dryness. The product was purified by Silica gel chromatography eluting with 5% MeOH in DCM. The purification was repeated eluting with a mixture of dichloromethane and ethyl acetate (1:1) and then changing to 5% MeOH in DCM. The di-protected phosphate-2′-Lev protected nucleoside was obtained as clear oil in 22% yield (0.44 g). ¹H NMR (500 MHz, CDCl₃) δ: 8.03 (s, 1H, H2), 7.95 (s, 1H, H8), 7.23-7.37 (m, 12H, MMTr), 6.94 (s, 1H, N⁶H), 6.80-6.83 (m, 2H, MMTr), 6.09 (d, J=3.5 Hz, 1H, H1′), 5.76 (dd, J=5.5 and 3.5 Hz, 1H, H2′), 5.34 (d, J=6.5 Hz, 1H, OCH₂O), 5.20 (d, J=6.5 Hz, 1H, OCH₂O), 4.97 (m, 1H, H3′), 4.51-4.62 (m, 8H, CH₂OAc and POCH₂C), 4.17-4.33 (m, 11H, H4′, H5′, H5″ and OCH₂CH₃), 3.81 (s, 3H, MeO MMTr), 2.77-2.80 (m, 2H, CH₂CH₂ Lev), 2.66-2.70 (m, 2H, CH₂CH₂ Lev), 2.20 (s, 3H, CH₃ Lev), 2.05 (s, 3H, OAc), 2.01 (s, 3H, Ac), 1.20-1.32 (m, 21H, CH₂CH₃ and CH₃ Piv). ³¹P NMR (202 MHz, CD₃CN) δ: −2.59.

N⁶-(4-methoxytrityl)-3′-O-pivaloyloxymethyladenosine 5′-Bis[3-acetyloxymethyl-2,2-bis(ethoxycarbonyl)propyl]phosphate The di-protected phosphate-2′-Lev protected nucleoside from the previous step (0.3 mmol, 0.44 g) was dissolved in a solution of hydrazine hydrate (3.9 mmol, 0.12 mL) in pyridine (4 mL) and acetic acid (1 mL) on an ice bath. The mixture was stirred for 1.5 hours. The ice bath was removed, and the reaction was allowed to proceed at room temperature for 2 hours. The reaction was quenched with 0.1 M NaH₂PO₃-solution, and the mixture was extracted with dichloromethane. The organic phase was washed with water, dried over Na₂SO₄ and evaporated to dryness. The di-protected phosphate nucleoside was purified by Silica gel chromatography using dichloromethane containing 3-5% methanol as eluent. The di-protected phosphate nucleoside was obtained as clear oil in 88% yield (0.35 g). ¹H NMR (500 MHz, CDCl₃) δ: 8.02 (s, 1H, H2), 7.98 (s, 1H, H8), 7.23-7.37 (m, 12H, MMTr), 6.96 (s, 1H, N⁶H), 6.80-6.83 (m, 2H, MMTr), 5.93 (d, J=5.0 Hz, 1H, H1′), 5.51 (d, J=6.3 Hz, 1H, OCH₂O), 5.42 (d, J=6.3 Hz, 1H, OCH₂O), 4.76 (dd, J=5.5 and 5.0 Hz, 1H, H2′), 4.64 (m, 1H, H3′), 4.50-4.63 (m, 8H, CH₂OAc and POCH₂C), 4.37 (m, 1H, H4′), 4.19-4.31 (m, 10H, OCH₂Me, H5′ and H5″), 3.88 (d, J=5 Hz, 1H, 2′OH) 3.81 (s, 3H, MeO MMTr), 2.05 (s, 3H, OAc), 2.03 (s, 3H, Ac), 1.22-1.32 (m, 21H, CH₂CH₃ and CH₃ Piv). ¹³C NMR (126 MHz, CDCl₃) δ: 178.0 (C═O Piv), 170.1 (C═O Ac), 166.4 (C═OOEt) 158.3 (MMTr), 154.3 (C6), 152.1 (C2), 148.3 (C4), 145.2 (MMTr), 138.8 (C8), 135.9, 130.2, 128.9, 127.9, 126.9 (MMTr), 123.7 (C5), 113.2 (MMTr), 89.4 (C1′) 89.0 (OCH₂O), 81.3 (C4′), 78.7 (C3′), 74.0 (C2′), 71.0 (MMTr), 67.2 (C5′), 65.4 (POCH₂C), 62.3 (CH₂CH₃), 61.2 (CH₂OAc), 58.0 (—C—), 55.2 (OCH₃ MMTr), 38.8 (CMe₃ Piv), 27.0 (CH₃ Piv), 20.6 (Ac), 13.9 (CH₂CH₃). ESI⁺-MS: m/z obsd 1222.4485, calcd (M+H)⁺ 1222.4479.

2′-O-levulinoyl-N⁶-(4-methoxytrityl)-3′-O-methyladenosine 5′-Bis[3-acetyloxymethoxy-2,2-bis(ethoxycarbonyl)propyl]phosphate. Compound 11 (1.5 mmol, 0.99 g, dried over P₂O₅ over 3 nights) was dissolved in dry DCM (3 mL) under nitrogen atmosphere. Anhydrous Et₃N (7.60 mmol, 1.056 mL) and bis(diethylamino)chlorophosphine (2.13 mmol, 447 μL) were added, and the mixture was stirred for 2 hours. The product was isolated by passing the mixture through a short silica gel column eluting with dry ethyl acetate containing 1% Et₃N. The solvent was removed under reduced pressure, and the residue was coevaporated from dry MeCN. The identity of the product was verified by ³¹P spectroscopy. ³¹P NMR (202 MHz, CD₃CN) δ: 133.7. The phosphitylated nucleoside was dissolved in dry MeCN (1 mL) under nitrogen. 2,2-Bis(ethoxycarbonyl)-3-acetyloxymethylenoxy)propanol (3.8 mmol, 0.11 g, coevaporated twice with dry MeCN and dried over P₂O₅ over 3 nights) and tetrazole (3.80 mmol, 8.440 mL of 0.45 mol L⁻¹ solution in MeCN) were added. The progress of the reaction was followed by ³¹P NMR spectroscopy. The spectrum was recorded after 1 hour. ³¹P NMR (202 MHz, CD₃CN) δ: 139.0. The phosphite ester formed was oxidized with 1₂ (0.1 mol L⁻¹) in a mixture of THF, H₂O and 2,6-lutidine (4:2:1, v/v/v, 10 mL) after a half an hour. The mixture was stirred over night at room temperature. Aqueous 5% NaHCO₃ was added, and the mixture was extracted twice with DCM. The organic phase was dried over Na₂SO₄ and evaporated to dryness. The product was purified by Silica gel chromatography eluting with a mixture of DCM and ethyl acetate (1:1). The product was obtained as clear oil in 43% yield (0.85 g). ¹H NMR (400 MHz, CDCl₃) δ: 8.01 (s, 1H, H2), 7.92 (s, 1H, H8), 7.19-7.35 (m, 12H, MMTr), 6.92 (s, 1H, N⁶H), 6.77-6.81 (m, 2H, MMTr), 6.06 (d, J=3.2 Hz, 1H, H1′), 5.76 (dd, J=5.2 and 3.2 Hz, 1H, H2′), 5.20-5.25 (m, 4H, OCH₂O), 4.50-4.53 (m, 4H, POCH₂C), 4.39 (m, 1H, H3′), 4.32 (m, 1H, H5′) 4.09-4.27 (m, 14H, H4′, H5″, OCH₂CH₃ and CH₂O), 3.78 (s, 3H, MeO MMTr), 3.41 (s, 3H, 3′-OMe), 2.63-2.79 (m, 4H, CH₂CH₂ Lev), 2.17 (s, 3H, CH₃ Lev), 2.06 (s, 3H, Ac), 2.04 (s, 3H, Ac), 1.18-1.28 (m, 12H, CH₂CH₃). ¹³C NMR (101 MHz, CDCl₃) δ: 206.0 (C═O Lev), 171.7 (C═O Lev), 170.2 (C═O Ac), 166.6 (C═OOEt) 158.3 (MMTr), 154.2 (C6), 152.4 (C2), 148.3 (C4), 145.2 (MMTr), 139.0 (C8), 137.2, 130.2, 128.9, 127.9, 126.8 (MMTr), 121.5 (C5), 113.2 (MMTr), 89.1 (OCH₂O), 88.8 (OCH₂O), 87.5 (C1′), 80.6 (C4′), 78.1 (C3′), 73.7 (C2′), 71.0 (MMTr), 67.1 (C5′), 65.2 (POCH₂C), 62.1 (CH₂CH₃), 61.8 (CH₂OAc), 59.1 (3′-OMe), 55.2 (OCH₃ MMTr), 53.4 (—C—), 37.8 (CH₂ Lev), 29.7 (CH₃ Lev), 27.8 (CH₂ Lev), 20.9 (Ac), 13.9 (CH₂CH₃). ³¹P NMR (162 MHz, CDCl₃) δ: −2.15: HRMS (ESI) Calcd for C60H75N5O24P, 1280.4534; Found 1280.4538.

N⁶-(4-methoxytrityl)-3′-O-methyladenosine 5′-Bis[3-acetyloxymethoxy-2,2-bis(ethoxycarbonyl)propyl]phosphate. The di-protected phosphate-2′-Lev protected nucleoside (0.66 mmol, 0.85 g, dried over P₂O₅ over three nights) was dissolved in dry DCM (16 mL). Hydrazine acetate (1.19 mmol, 0.11 g) in dry methanol (2 mL) was added. After stiffing the reaction mixture for 3 hours, hydrazine acetate (0.60 mmol, 0.055 g) in dry methanol (1 mL) was added, and the reaction was allowed to proceed for 1 hour. The reaction was quenched with acetone and the mixture was evaporated to dryness. The product was purified by Silica gel chromatography, eluting with a mixture of DCM and ethyl acetate (1:4). The di-protected phosphate protected nucleoside was obtained as clear oil in 89% yield (0.70 g). ¹H NMR (500 MHz, CDCl₃) δ: 8.01 (s, 1H, H2), 7.96 (s, 1H, H8), 7.20-7.35 (m, 12H, MMTr), 6.94 (s, 1H, N⁶H), 6.77-6.81 (m, 2H, MMTr), 5.91 (d, J=5.5 Hz, 1H, H1′), 5.20-5.25 (m, 4H, OCH₂O), 4.80 (m, 1H, H2′), 4.49-4.56 (m, 4H, POCH₂C), 4.33, (m, 1H, H4′), 4.10-4.30 (m, 15H, H5′, H5″, H3′, OCH₂CH₃ and CH₂O), 3.84 (br. s, 1H, 2′OH), 3.78 (s, 3H, MeO MMTr), 3.54 (s, 3H, 3′-OMe), 2.06 (s, 3H, Ac), 2.05 (s, 3H, Ac), 1.20-1.29 (m, 12H, CH₂CH₃). ¹³C NMR (126 MHz, CDCl₃) δ: 170.3 (C═O Ac), 166.6 (C═OOEt) 158.3 (MMTr), 154.2 (C6), 152.2 (C2), 148.5 (C4), 145.2 (MMTr), 138.8 (C8), 137.2, 130.2, 128.9, 127.9, 126.9 (MMTr), 121.5 (C5), 113.2 (MMTr), 89.5 (C1′), 88.7 (OCH₂O), 80.6 (C4′), 79.7 (C3′), 73.3 (C2′), 71.0 (MMTr), 67.1 (C5′), 65.3 (POCH₂C), 62.2 (CH₂CH₃), 61.8 (CH₂OAc), 58.9 (3′-OMe), 55.2 (OCH₃ MMTr), 53.5 (—C—), 20.9 (Ac), 13.9 (CH₂CH₃).: HRMS (ESI) Calcd for C55H69N5O22P, 1182.4166; found 1182.4123.

3′-O-Acetyloxymethyl-5′-O-(tert-butyldimethylsilyl)-N⁶-(4-methoxytrityl)adenosin-2′-yl 2′,3′-di-O-levylinoyl-N⁶-(4-methoxytrityl)adenosin-5′-yl 3-acetyloxy-2,2-bis(ethoxycarbonyl)phosphate. Compound 12 (0.69 mmol, 0.500 g) was dried on P₂O₅ overnight and dissolved in dry DCM (3 mL) under nitrogen. Triethylamine (3.45 mmol; 0.479 mL) and bis(diethylamino)chlorophosphine (0.90 mmol, 0.188 mL) were added, and the mixture was stirred under nitrogen for 2 hours. The product was isolated by passing the mixture through a short silica gel column with a 7:3 mixture of ethyl acetate and hexane containing 0.5% triethylamine. The solvent was removed under reduced pressure, and the residue was coevaporated from dry MeCN and dry DCM to remove the traces of triethylamine. The identity of the product was verified by ³¹P and ¹H NMR spectroscopy. ¹H NMR (500 MHz, CD³CN) δ: 8.14 (s, 1H, H2), 7.88 (s, 1H, H8), 7.25-7.41 (m, 12H, MMTr), 6.93 (s, 1H, N⁶H), 6.86 (d, J=9.0 Hz, 2H, MMTr), 6.05 (d, J=6.5 Hz, 1H, H1′), 5.41 (d, J=6.5 Hz, 1H, OCH₂O), 5.35 (d, J=6.5 Hz, 1H, OCH₂O), 4.96 (ddd, J=11.0, 6.0 and 4.5 Hz, 1H, H2′), 4.51 (dd, J=4.5 and 2.5 Hz, 1H, H3′), 4.12 (m, 1H, H4′), 3.97 (dd, J=11.5 and 4.5 Hz, 1H, H5′), 3.86 (dd, J=11.5 and 3.5 Hz, 1H, H5″), 3.78 (s, 3H, MMTr), 2.85-2.97 (m, 4H, NEt), 2.65-2.72 (m, 4H, NEt), 2.17 (s, 3H, Ac), 1.00 (t, J=7.0 Hz, 6H, NEt), 0.95 (s, 9H, TBDMS), 0.77 (t, J=7.5 Hz, 6H, NEt), 0.12 (s, 3H, TBDMS), 0.11 (s, 3H, TBDMS). ³¹P NMR (202 MHz, CD₃CN) δ: 137.6 ppm.

The phosphitylated nucleoside from the previous step was dissolved in dry MeCN (1.0 mL) under nitrogen, tetrazole (0.68 mmol, 1.51 mL of 0.45 mol L⁻¹ solution in MeCN) and N⁶-(4-methoxytrityl)-2′,3′-di-O-levulinoyladenosine (0.48 mmol, 0.355 g) in MeCN (1.0 mL) was added. The reaction was allowed to proceed for 25 minutes. Tetrazole (0.78 mmol, 1.74 mL of 0.45 mol L⁻¹ solution in MeCN) and diethyl 2-acetyloxymethyl-2-hydroxymethylmalonate (0.69 mmol, 0.180 g) were then added. The course of the reaction was monitored by ³¹P NMR spectroscopy. After an hour, ³¹P NMR signals (202 MHz, CD₃CN) at 140.7 and 140.5 ppm were observed. The phosphite ester that was formed was then oxidized with iodine (0.2 g) in a mixture of THF (4.0 mL), H₂O (2.0 mL) and 2,6-lutidine (1.0 mL). The oxidation was allowed to proceed overnight. The excess of iodine was removed with 5% NaHSO₃. The mixture was extracted twice with DCM. The organic phase was washed with brine, dried on Na₂SO₄ and evaporated to dryness. The crude product was purified on a silica gel column eluting with a 4:1 mixture of ethyl acetate and DCM. The overall yield starting from compound 12 was 40% (0.49 g). ¹H NMR (500 MHz, CDCl)) δ: 8.12, 8.10, 8.08, 8.06, 8.04, 8.01, 8.00, 7.98 (8s, 4H, H2 and H8), 7.22-7.38 (m, 24H, MMTr), 6.95, 6.94, 6.92, 6.91 (4s, 2H, NH), 6.79-6.83 (m, 4H, MMTr), 6.21 (d, J=1.5 Hz, ½H, H1′), 6.19 (d, J=5.5 Hz, ½H, H1′), 6.19 (d, J=2.0 Hz, ½H, H1′), 6.12 (d, J=5.5 Hz, ½H, H1′). 5.83 (dd, J=5.5 and 5.5 Hz. ½H. H2′). 5.80 (dd, J=5.5 and 5.5 Hz, ½H, H2′), 5.70 (m, 1H. H3′), 5.50 (m, 1H, H2′), 5.44 (d, J=6.5 Hz, y, H, OCH₂OAc), 5.43 (d, J=6.5 Hz, ½H, OC₂,OAc), 5.36 (d, J=6.5 Hz, ½H, OCH₂OAc), 5.28 (d, J=6.5 Hz, ½ 2H, OCH₂OAc), 5.39 (m, 1H, H3′), 4.57-4.69 (m, 4H, POCH₂C and CH₂OAc), 4.3 3-4.43 (m, 3H, H4′, H5′ and H5″), 4.12-4.25 (m. 5H, H4′ and OCH₂Me). 4.00 (m, 1H, H5′), 3.83 (m, 1H, H5″), 3.80, 3.80, 3.80, 3.79 (4s, 6H, MeO (MMTr), 2.56-2.83 (m, 8H, CH₂ Lev), 2.21, 2.17, 2.15, 2.13 (4s, 6H, Me Lev), 2.10, 2.08, 2.00, 1.92 (4s, 6H, OAc), 1.15-1.25 (m, 6H, CH₂CH₃), 0.88, 0.87 (2s, 9H, Me₃C—Si), 0.08, 0.06, 0.03, 0.01 (4s, 6H, Me-Si). ³¹P NMR (202 MHz, CD₃CN) δ: −2.4 and −2.5 ppm. (Multiplicity of some signals is due to the presence of Rₚ and Sₚ diastereomers.) ESI-MS: m/z obsd (M+H)⁺ 1768.67, calcd (M+H)⁺ 1768.90; obsd (M+Na)⁺ 1790.66, calcd (M+Na)⁺ 1790.88.

3′-O-Acetyloxymethyl-N⁶-(4-methoxytrityl)adenosin-2′-yl 2′,3′-di-O-levylinoyl-N⁶-(4-methoxytrityl)adenosin-5′-yl 3-acetyloxy-2,2-bis(ethoxycarhonyl) phosphate. 3′-O-Acetyloxymethyl-5′-O-(tert-butyldimethylsilyl)-N⁶-(4-methoxytrityl)adenosin-2′-yl 2′,3′-di-O-levylinoyl-N⁶-(4-methoxytrityl)adenosin-5′-yl 3-acetyloxy-2,2-bis(ethoxycarbonyl)phosphate (0.27 mmol, 0.47 g), dried over P₂O₅; overnight, was dissolved in dry THF (5 mL). Triethylamine trihydrofluoride (1.06 mmol, 173 μL) was added, and the mixture was stirred for three days at room temperature. The mixture was neutralized by adding aqueous triethylammonium acetate (2.0 mol L⁻¹) in small portions. The mixture was evaporated to dryness, and the residue was then dissolved in DCM and washed with water. The organic phase was evaporated to dryness. The product was purified by silica gel chromatography eluting with 3% MeOH in DCM. The yield was 93% (0.41 g). ¹H NMR (500 MHz, CDCl₃) δ: 8.07, 8.04, 8.03, 8.02, 8.00, 7.98, 7.96, 7.95 (8s, 4H, H2 and H8), 7.19-7.35 (m, 24H, MMTr), 7.04 (br. d, J=3.0 Hz, 1H, NH), 6.94 (br. s, 1H, NH), 6.76-6.81 (m, 4H, MMTr), 6.45 (m, 1H. 5′-OH), 6.17 (d, J=5.5 Hz, ½H, H1′), 6.14 (d, J=5.5 Hz, ½H, H1′), 6.06 (d, J=7.0 Hz, ½H, H1′), 5.82 (dd, J=5.5 and 5.5 Hz, ½H, H2′), 5.77 (dd, J=5.5 and 5.5 Hz, ½H, H2′), 5.71 (m. ½H, H2′), 5.66 (m. ½H, H2′), 5.54 (d, J=6.8 Hz, ½H, OCH₂OAc), 5.53 (m. ½H, H3′), 5.43 (m, ½H, H3′), 5.36 (s, 1H, OCH₂OAc), 5.24 (d, J=6.5 Hz, ½, H, OCH₂OAc), 4.77 (dd, J=5.0 and 1.5 Hz, H, H3′), 4.68 (dd, J=5.0 and 1.5 Hz, ½H, H3′), 4.61 (s, ½H, CH₂OAc), 4.02-4.53 (m, 11½H, CH₂OAc, POCH₂C, 2×H4′, H5 and H5″), 3.86-3.95 (m, 1H, H5′), 3.75-3.79 (m, 6H, MeO MMTr), 3.65-3.72 (m, 1H, H5′), 2.51-2.82 (m, 8H, CH₂ Lev), 2.19, 2.18, 2.13, 2.12 (4s, Me Lev), 2.08, 2.06, 1.98, 1.95 (4s, 6H, OAc), 1.13-1.21 (m, 6H, CH₂CH₃). ¹³C NMR (126 MHz, CDCl₃) δ: 206.3, 206.3, 206.1, 206.1 (C═O Lev), 171.8, 171.7, 171.6, 171.5 (C═O Lev), 170.6, 170.4, 170.1, 170.0 (C═O Ac), 166.3, 166.2 (C═OOEt), 158.4, 158.3 (MMTr), 154.6, 154.2 (C6), 152.6, 151.7 (C2), 148.7, 147.3 (C4), 145.1, 145.1, 144.9 (MMTr), 140.5, 138.8 (C8), 137.2, 137.0, 130.2, 128.9, 128.8, 128.0, 127.9, 127.9, 127.0, 126.9, 126.9 (MMTr), 122.5, 121.3 (C5), 113.2, 113.2, 113.2 (MMTr), 89.3 (C1′), 88.9, 88.2 (OCH₂O), 86.5. 86.2 (C4′). 85.9 (C1′), 80.7, 80.1 (C4′), 78.8, 78.3, 77.1-77.3 (C3′, under CDCl₃), 73.3, 73.0 (C2′), 71.1, 71.1, 71.0 (MMTr), 70.5, 70.4 (C2′). 67.5. 67.2 (C5′), 65.7 (POCH₂C), 62.4 (C5′), 62.3 (CH₂CH₃), 62.0, 61.1 (CH₂OAc), 58.0 (—C—), 55.2 (OCH₃ MMTr), 37.7, 37.6 (CH₂C═O Lev), 29.8, 29.7 (CH₃ Lev), 27.6, 27.5, 27.5 (CH₂C═OO Lev, 21.0, 21.0, 20.6, 20.6 (Ac), 13.9 (CH₂CH₃). ³¹P NMR (202 MHz, CDCl₃) δ: −2.3 and −2.7 ppm. (Multiplicity of some signals is due to the presence of Rp and Sp diastereomers.) ESI-MS: m/z obsd (M+H) 1653.5765, calcd (M+H) 1653,5867; obsd (M+Na) 1675.5600, calcd (M+Na) 1675.5687; obsd (M+K)⁺ 1691.5329, calcd (M+K) 1691.5426.

3′-O-Acetyloxymethyl-N⁶-(4-methoxytrityl)adenosin-2′-yl 2′,3′-di-O-levylinoyl-(4-methoxytrityl)adenosin-5′-yl 3-acetyloxy-2,2-bis(ethoxycarbonyl) phosphate. 3′-O-Acetyloxymethyl-N⁶-(4-methoxytrityl)adenosin-2′-yl 2′,3′-di-O-levylinoyl-N⁶-(4-methoxytrityl)adenosin-S′-yl 3-acetyloxy-2,2-bis(ethoxycarhonyl) phosphate (0.35 mmol, 0.58 g) was evaporated once from dry acetonitrile. The residue was dissolved in dry DCM (8 mL). Hydrazine acetate (1.17 mmol, 0.107 g) in dry MeOH (0.9 mL) was added, and the mixture was stirred at room temperature for 2.5 hours. The reaction was quenched with acetone, stirred for 20 minutes and evaporated to dryness. The product was purified on a slilica gel column eluting with DCM containing 5% MeOH. The compound was then subjected to detritylation with 80% (v/v) aq AcOH (10 mL). After stiffing overnight at room temperature, the reaction mixture was evaporated to dryness. The residue was coevaporated twice with water. The product was purified first by Silica gel chromatography eluting with DCM containing 10-20% MeOH and then by HPLC on a Sun Fire™ Prep C18 column (250×10 mm, 5 μm, flow rate 3.0 mL min⁻¹) (150×4.6 mm, 5 μm, flow rate 1.0 mL min⁻¹), using a linear gradient elution from 33% to 100% methanol in 20 minutes. The dimer was obtained in 39% yield (153 mg). ¹H NMR (500 MHz, CD₃CN) δ; 7.99-8.25 (m, 4H, H2 and H8), 6.43, 6.34, 6.27, 6.18 (4 br. s. 3H, NH and 5′OH), 6.09 (m, 1H, H1′), 5.96 (d , J=4.5 Hz, ½H, H1′), 5.91 (br. d, J=4.0 Hz, ½H, H1′). 5.54 (m, 1H, H2′), 5.47 (d, J=6.5 Hz, ½H, OCH₂O), 5.40 (d, J=7.0 Hz, ½H, OCH₂O), 5.30 (m, 1H, OCH₂O), 4.66 (dd , J=5.0 and 2.5 Hz, ½H, H3′), 4.61-4.65 (m, 1H, H3′ and H2′), 4.54 (m, ½H. H2′), 4.46-4.51 (m, 2H. H5⁺ and OCH₂OAc), 4.38-4.44 (m, 1½H, H5″″ and OCH₂OAc), 4.28-4.35 (m, 3H, H3′, OCH₂OAc, POCH₂C and H4′), 4.10-4.25 (m. 5½, H, H3′, CH₂CH₃ and POCH₂C), 4.08 (m, ½H, H4′), 4.03 (m, ½H, H4′), 3.85 (m, 1H, H5′), 3.70 (m, 1H, H5″), 2.10 (s, 1½H, OAc), 2.07 (s, 1½H, OAc), 2.01, 1.97 (2s, 3H, OCH₂OAc), 1.13-1.23 (m, 6H, CH₂CH₃) ¹³C NMR (126 MHz, CD₃CN) δ; 170.4, 170.1 (C═O Ac), 166.4, 166.3, (C═OOEt), 156.5, 156.0 (C6), 152.9, 152.5 (C2), 148.6 (C4), 140.8, 140.6, 139.6, 139.3 (C8), 119.7, 120.6 (C5), 88.7 (C1′), 88.3 (OCH₂O), 87.99 (C1′). 85.8, 85.7, 82.4, 82.0 (C4.). 78.4, 78.1 (C3′), 76.7, 76.5 (C2′), 74.0, 73.9 (C2′). 70.2, 70.0 (C3′), 67.8, 67.6 (POH₂C), 65.5. 65.4 (CS′, POCH₂C), 62.4, 62.3 (CH,CH), 61.8. 61. 7 (C5′), 60.9, 60.8 (CH₂OAc), 57.9 (—C—), 20.3 (Ac) 19.8 (OCH₂OAc), 13.2 (CH₂CH₃). ³¹P NMR (202 MHz, CD₃CN) δ: −2.5 and −2.4. (Multiplicity of some signals is due to the presence of Rp and Sp diastereomers.) ESr-MS: m/z obsd (M+H) 913.2707, calcd 913.2724 (M+H).

5′-O-(tert-butyldimethylsilyl)-N⁶-(4-Methoxytrityl)-3′-O-pivaloyloxymethyladenosin-2′-yl 2′,3′-di-O-levylinoyl-N⁶-(4-methoxytrityl)adenosin-5′-yl 3-acetyloxy-2,2-bis(ethoxycarbonyl) phosphate. Compound 9a (0.73 mmol, 0.600 g) was dried over P₂O₅ overnight and dissolved in dry DCM (4 mL) under nitrogen atmosphere. Triethylamine (3.91 mmol, 0.543 mL) and bis(diethylamino)chlorophosphine (0.94 mmol, 0.197 mL) were added, and the mixture was stirred under nitrogen for 2 hours. The product was isolated by passing the mixture through a short silica gel column with a 7:3 mixture of ethyl acetate and hexane containing 0.5% triethylamine. The solvent was removed under reduced pressure, and the residue was coevaporated from dry MeCN and dry DCM to remove the traces of triethylamine. ³¹P NMR (202 MHz, CD₃CN) 137.2.

The phosphitylated compound from the previous step was dissolved in dry MeCN (1.0 mL) under nitrogen. Tetrazole (0.73 mmol, 1.630 mL of 0.45 mol L⁻¹ solution in MeCN) and N⁶-(4-methoxytrityl)-2′,3′-di-O-levulinoyladenosine (0.55 mmol; 0.400 g) in dry MeCN (1.0 mL) were added. The reaction was allowed to proceed for 15 minutes. Tetrazole (0.73 mmol, 1.630 mL of 0.45 mol L⁻¹ solution in MeCN) and diethyl 2-acetyloxymethyl-2-hydroxymethylmalouate (0.86 mmol, 0.230 g) in dry MeCN were added. The course of the reaction was monitored by ³¹P NMR spectroscopy. After 40 minutes, ³¹P NMR signals (202 MHz, CD₃CN) at 140.6 and 140.5 ppm were observed.

The phosphite ester that was formed was then oxidized with iodine (0.2 g) in a mixture of THF (4.0 mL), H₂O (2.0 mL) and 2,6-lutidine (1.0 mL). The oxidation was allowed to proceed over night. The oxidized product exhibited ³¹P NMR signals (202 MHz, CD₃CN) at −2.4 and −2.6 ppm. The excess of iodine was removed with 5% NaHSO₃. The mixture was extracted three times with DCM. The organic phase was dried on Na₂SO₄ and evaporated to dryness. The crude product was purified by Silica gel chromatography eluting first with a 1:1 mixture of ethyl acetate and DCM, then with ethyl acetate and finally with ethyl acetate containing 5% MeOH. Yield=35% (0.50 g). ¹H NMR (500 MHz, CDCl₃) δ: 7.98-8.07 (m, 4H, H2 and H8), 7.21-7.38 (m, 24H, MMTr), 6.94 (br, s, 1H, NH), 6.90 (br, s, 1H, NH), 6.78-6.82 (m, 4H, MMTr), 6.19 (d, J=2.0 Hz, 1H, H1′), 6.13 (d, J=5.5 Hz, 1H, H1′), 5.78 (dd, J=5.3 and 5.3 Hz, 1H, H2′), 5.68 (dd, J=5.3 and 5.3 Hz, 1H, H2′). 5.58 (m, 1H, H3′), 5.52 (d, J=6.3 Hz, 1H, OCH₂O), 5.32 (d, J=6.3 Hz, 1H, OCH₂O), 5.03 (m, 1H, H3′), 4.54-4.65 (m, 4H, OCH₂OAc and POCH₂C), 4.17-4.38 (m, 8H, 2×H4′, H5′, H5″, 2×CH₂CH)), 3.94 (m, 1H, H5′), 3.78-3.83 (m, 7H, H5″ and MeO MMTr), 2.55-2.78 (m, 8H, CH₂CH₂ Lev), 2.18, 2.14 (2s, 6H, CH₃ Lev), 2.01 (s, 3H, OAc), 1.23 (2s, 9H, Cme₃ Piv), 1.15-1.25 (m, 6H, CH₂CH₃), 0.83 (s, 9H, SiCMe₃), 0.03 (s, 6H, Si-Me). ³¹P NMR (202 MHz, CDCl₃) δ: −2.4 and −2.6 ppm. (Multiplicity of some signals is due to the presence of Rₚ and Sₚ diastereomers.)

N⁶-(4-Methoxytrityl)-3′-O-pivaloyloxymethyladenosin-2′-yl 2′,3′-di-O-levylinoyl-N⁶-(4-methoxytrityl)adenosin-5′-yl 3-acetyloxy-2,2-bis(ethoxycarbonyl) phosphate. The tert-butyldimethylsilyl group on the compound from the previous step was removed by treatment with Bu₄NF in THF under acidic conditions. Bu₄NF (0.8 mmol, 0.22 g) was dissolved in dry THF (6.8 mL) and AcOH (1.2 mL) was added. The compound from the previous step (0.3 mmol, 0.50 g) was added, and the mixture was stirred at room temperature for 2 days. 1% NaHCO₃ solution was added, and the mixture was extracted twice with dichloromethane. The organic phase was dried over Na₂SO₄ and evaporated to dryness. The product was purified by Silica gel chromatography eluting with dichloromethane containing 3-5% MeOH to afford a diastereomeric mixture (1:1) of the product as clear oil. Yield=0.33 g (70%). ¹H NMR (500 MHz, CDCl₃) δ: 8.04. 8.03, 8.03, 8.01 . 8.00. 7.98, 7.95. 7.95 (8s, 4H, H2 and H8), 7.19-7.35 (m, 24H, MMTr), 7.04 (m, 1H, NH), 6.92 (br. s 1H, NH), 6.77-6.81 (m, 4H, MMTr), 6.48 (dd, J=2.0 and 11.5 Hz. ½H, 5′-OH), 6.40 (dd, J=2.5 and 11.5 Hz. ½H. 5′-OH), 6.16 (d, J=5.5 Hz, ½H, HI′). 6.14 (d, J=5.0 Hz. ½H, H1′), 6.04 (d, J=6.5 Hz. ½H. H1′), 6.03 (d, J=7.0 Hz. ½H, H1′). 5.80 (t. J=5.5 Hz. ½H, H2′). 5.77 (t, J=5.5 Hz. ½H, H2′), 5.71 (m, ½H H3′), 5.64-5.67 (m, 1H, H3′ and OCH₂O), 5.50-5.56 (m, 1H, H2′ and OCH₂O), 5.46 (m, ½H H2′), 5.29 (d. J=6.5 Hz, Y, H, OCH₂O), 5.19 (d, J=6.5 Hz, Y, H, OCH₂O), 4.75 (dd, J=1.5 and 5.5 Hz. ½H, H3′), 4.70 (dd. J=1.5 and 5.5 Hz, ½H. H3.), 4.50-4.55 (m, 2H, OCH₂OAc and POCH₂C), 4.08-4.46 (m, 10H, OCH₂OAc, POCH₂C, 2×H4′, H5′, H5″, 2×CH₂CH₃), 3.88 (m, 1H, H5′ and H5″) 3.75-3.78 (m, 6H, MeO MMTr), 3.65-3.72 (m, 1H, H5″ and H5″), 2.55-2.80 (m, 8H, CH₂CH₂ Lev), 2.18, 2.17, 2.13, 2.12 (4s, 6H, CH₃ Lev), 1.99, 1.95 (2s, 3H, OAc), 1.20, 1.19 (2s, 9H, CMe3 Piv), 1.13-1.22 (m, 6H, CH₂CH₃). ¹³C NMR (126 MHz, CDCh) 0:206.3. 206.3, 206.1, 206.1 (C═O Lev), 177.7, 177.9 (C═O Piv), 171.8, 171.7, 171.6, 171.5 (C═O Lev), 170.1, 170.0 (C═O Ac), 166.3, 166.2, 166.1, 166.0 (C═OOEt) 158.4, 158.3 (MMTr), 154.6, 154.2 (C6), 152.6, 151.7 (C2), 148.7, 147.3 (C4), 145.2, 145.1, 145.1, 145.0 (MMTr), 140.5, 140.3, 138.6 (C8), 137.2, 137.0, 130.2, 128.9, 127.9, 127.9, 126.9, 126.9 (MMTr), 122.5, 121.3 (C5), 113.2, 113.2 (MMTr), 89.3 (C1′). 89.1 , 88.9 (OCH₂O), 86.8, 86.6 (C4′), 86.0, 85.9 (C I′), 80.7 (C4′), 78.8. 78.6 (C3′). 73.3 (C2). 71.0 (MMTr), 67.2 (C5′), 65.4 (POCH₂C), 62.3 (CH₂CH₃), 61.2 (CH₂OAc), 58.0 (—C—), 55.2 (OCH₃ MMTr), 38.8 (CMe₃ Piv), 37.8, 37.7, 37.7 (CH₂C═O Lev), 29.8, 29.8 (CH₃ Lev), 27.6, 27.5, 27.5 (CH₂C=00 Lev), 27.0 (CH₃, Piv), 20.6, 20.6 (Ac), 13.9 (CH₂CH₃). ³¹P NMR (202 MHz, CDCl₃) δ: −2.2 and −2.7. (Multiplicity of some signals is due to the presence of Rₚ and Sₚ diastereomers.) ESI-MS: m/z obsd (M+Nar 1717.6164, calcd (M+Nar 1717.6151.

3′-O-pivaloyloxymethyladeno sin-2′-yl adenosinl-5′-yl 3-acetyloxy-2,2-bis(ethoxycarbonyl)phosphate. The compound from the previous step (0.087 mmol, 0.100 g) was dissolved in a solution of hydrazine hydrate (2.50 mmol, 0.078 mL) in a mixture of pyridine (4 mL) and AcOH (1 mL) on an ice bath. The mixture was stirred for 1 hour. The ice bath was removed, and the reaction was allowed to proceed at room temperature for 3 hours. The reaction was quenched with 0.1 mol L⁻¹ NaH₂PO₃ solution (25 mL), and the product was extracted into DCM. The organic phase was washed with water, dried over Na₂SO₄ and evaporated to dryness. The product was purified by Silica gel chromatography eluting with dichloromethane containing 5% methanol. The compound was then subjected to detritylation with 80% (v/v) aq AcOH (10 mL). After stirring overnight at room temperature, the reaction mixture was evaporated to dryness. The residue was coevaporated twice with water. The product was purified first by Silica gel chromatography eluting with DCM containing 10-20% MeOH, and then by HPLC on a Thermo Hypersil Hypurity™ Elite C18 column (150×4.6 mm, 5 μm, flow rate 1.0 mL min⁻¹), using a linear gradient elution from water to MeCN in 30 minutes. The product dimer was obtained in 29% (24 mg) yield. ¹H NMR (500 MHz, CD₃CN) δ: 8.23, 8.20, 8.20, 8.11 (4s, 2H, H2), 8.11, 8.06, 8.06, 8.00 (4s, 2H, H8), 6.49, 6.38, 6.33, 6.22 (4 br. s, 3H, NH and 5′OH), 6.10 (m, 1H, H1′), 5.96 (d, J=4.5 Hz, ½H, H1′).5.91 (br. d, J=4.0 Hz, ½H, H1′), 5.52-5.60 (m. 1½H, H2′ and OCH₂O), 5.50 (d, J=7.0 Hz, Y, H OCH, O), 5.27 (m, 1H, OCH₂O), 4.70 (dd, J=5.0 and 2.5 Hz, ½H, H3′), 4.67 (dd, J=5.0 and 2.0 Hz, ½H, H3′), 4.63 (m, ½H, H2′), 4.53 (m, ½H, H2′), 4.42-4.52 (m, 2H, H5′ and OCH₂OAc), 4.30-4.42 (m, 3½H, OCH₂Oac, H3′ and POCH₂C), 4.29 (m, 1H, H4′), 4.07-4.25 (m, 6H, H3, H5′, H5″, CH₂CH₃ and H4′), 4.03 (m, ½H, H4′), 3.85 (m, 1H, H5′), 3.69 (m, 1H, H5″), 1.96 (s, 3H, OAc), 1.23, 1.21 (2s, 9H, CMe₃ Piv), 1.13-1.23 (m, 6H, CH₂CH₃). ¹³C NMR (126 MHz, CD₃CN) δ: 177.6, 177.6 (C═O Piv), 170.1, 170.0 (C═O Ac), 166.4, 166.3, (C═OOEt), 156.5, 156.0 (C6), 152.9, 152.5 (C2), 149.7, 148.6 (C4), 140.8, 140.5, 139.8, 139.1 (C8), 119.7, 120.6 (C5), 88.8 (OCH₂O), 88.8, 88.0, 88.0 (C1′). 85.9, 85.7, 82.4, 82.3 (C4′), 78.3, 78.0 (C3′), 76.7, 76.5, 74.0, 73.9 (C2′), 70.2. 70.0 (C3′), 67.8, 65.5 (C5′), 65.4, 65.3 (POCH₂C), 62.4. 62.3 (CH₂CH₃), 61.8, 61.7 (C5′), 60.9. 60.8 (CH₂OAc), 57.9, 57.8 (—C—), 38.5, 38.5 (CMe₃ Piv), 26.2, 26.2 (CH₃, Piv), 19.8, 19.8 (Ac), 13.2, 13.2 (CH₂CH₃). ³¹P NMR (202 MHz, CD₃CN) δ: −2.4 and −2.4. (Multiplicity of some signals is due to the presence of Rₚ and Sₚ diastereomers.) ESI-MS: m/z obsd (M+H) ‘955.3215, calcd 955.31 93 (M+H)⁺.

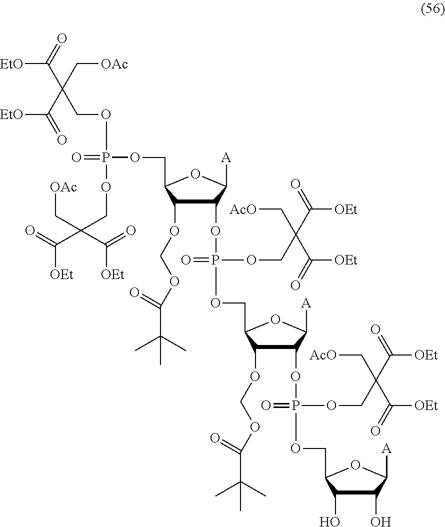

N⁶-(4-methoxytrityl)-3-O-pivaloyloxymethyladenosine 5′-Bis[3-acetyloxymethyl-2,2-bis(ethoxycarbonyl)propyl]phosphate (0.29 mmol, 0.340 g, dried over P₂O₅ over night) was dissolved in dry dichloromethane (2 mL) under nitrogen atmosphere. Anhydrous Et₃N (1.42 mmol, 0.198 mL) and bis(diethylamino)chlorophosphine (0.34 mmol, 0.072 mL) were added. The mixture was stirred for 2.5 hours. The product was isolated by passing the mixture through a short silica gel column with a 7:3 mixture of ethyl acetate and hexane containing 0.5% triethylamine. The solvent was removed under reduced pressure. The product was coevaporated from dry acetonitrile to remove the traces of Et₃N. The identity of the product was verified by ³¹P NMR spectroscopy. ³¹P NMR (202 MHz, CD₃CN) δ: 137.4 and −2.43.

The product from the previous step was dissolved in dry acetonitrile (400 μL) under nitrogen atmosphere. 3′-O-Pivaloyloxymethyl-6-N-(4-methoxytrityl)adenosine-2′ yl 2′,3′-di-O-levulinoyl-6-N-(4-methoxytrityl)adenosine-5′-yl 3-acetyloxy-2,2-bis(ethoxycarbonyl)phosphate (0.16 mmol, 0.270 g, dried over P₂O₅ over night) in dry acetonitrile (600 μL) and tetrazole (0.19 mmol, 0.422 mL of 0.45 mol L⁻¹ solution in MeCN) were added. The reaction was allowed to proceed for 10 minutes. 2,2-Bis(ethoxycarbonyl)-3-acetyloxy)propanol (0.29 mmol, 0.080 g, coevaporated twice with dry MeCN and dried over P₂O₅ over night) in dry acetonitrile (200 μL) and tetrazole (0.22 mmol, 0.487 mL of 0.45 mol L⁻¹ solution in MeCN) were added, and the mixture was stirred for 10 minutes. ³¹P NMR (202 MHz, CD₃CN) δ: 151.6, 151.3, 150.8, 150.1, 140.5, 140.4, −2.1−(−2.6) (m).

The phosphite ester from the previous step was oxidized with I₂ (0.1 mol L⁻¹) in a mixture of THF. After approximately half an hour, H₂O and 2,6-lutidine (4:2:1, v/v/v, 7 ml) was added. The mixture was then stirred over night at room temperature. Aqueous 5% NaHCO₃ was added, and the mixture was extracted twice with dichloromethane. The organic phase was dried over Na₂SO₄ and evaporated to dryness. The product was purified by silica gel chromatography, eluting with a mixture of dichloromethane and ethyl acetate (1:1) and changing the eluent to ethyl acetate and then to dichloromethane containing 10% methanol. The protected trimer (a diastereomeric mixture) was obtained as yellowish oil in 33% yield (0.30 g). The identity was verified by ³¹P NMR spectroscopy. ³¹P NMR (202 MHz, CD₃CN) δ: −1,5−(−3.0) (m).

The levulinoyl groups on the protected trimer (0.100 g) were removed by dissolving the protecting trimer in a solution of hydrazine hydrate (2.50 mmol, 0.078 mL) in pyridine (4 mL) and acetic acid (1 mL) on an ice bath. The mixture was stirred for 1 hour. The ice bath was removed, and the reaction was allowed to proceed at room temperature for 3 hours. The reaction was quenched with 0.1 M NaH₂PO₃-solution (25 mL), and the mixture was extracted with dichloromethane. The organic phase was washed with water, dried over Na₂SO₄ and evaporated to dryness. The protected trimer was purified by Silica gel chromatography eluting with dichloromethane containing 5% methanol.

The trityl groups on the protected trimer were removed by dissolving the product from the previous step in 80% (v/v) aqueous AcOH (10 mL). After stiffing overnight at room temperature, the reaction mixture was evaporated to dryness. The residue was coevaporated twice with water. The trimer was purified first by Silica gel chromatography eluting with dichloromethane containing 10-20% methanol, then by HPLC on a Thermo Hypersil Hypurity™ Elite C18 column (150×4.6 mm, 5 μm, flow rate 1.0 mL min⁻¹), using a linear gradient elution from water to acetonitrile in 30 minutes. Overall yield of the trimer starting from the levulinoyl protected trimer was 39% (27 mg). ¹H NMR (500 MHz, CD₃CN) δ: 7.98-8.25 (m, 6H, H2 and H8), 6.19-6.53 (m, 3H, NH), 5.90-6.18 (d, 3H, H1′), 5.17-5.59 (m, 6H, H2′, H3′ and 2×OCH₂O), 4.85-5.20 (m, 1H, H3′), 4.61 (m, 1H, H2′), 4.09-4.58 (m, 44H, H2′, H3′, 2′OH, 3′OH, OCH₂OAc, POCH₂C, H4′, H5′, H5″, CH₂CH₃), 1.94-2.04 (m, 12H, OAc), 1.13-1.27 (m, 42H, CMe₃ Piv, CH₂CH₃). ³¹P NMR (202 MHz, CDCl₃) δ: −2,7−(−2.0) (m). (Multiplicity of some signals is due to the presence of Rₚ and Sₚ diastereomers.) ESI⁺-MS: m/z obsd 2210.6789, calcd (M+H)⁺ 2210.6903.

To run the kinetic measurements, 2 mg of the slowest migrating diastereomer was separated on a Sun Fire™ Prep C18 column (250×10 mm, 5 μm, flow rate 3.0 mL min⁻¹) eluting from 40% acetonitrile to 80% acetonitrile in 30 minutes.

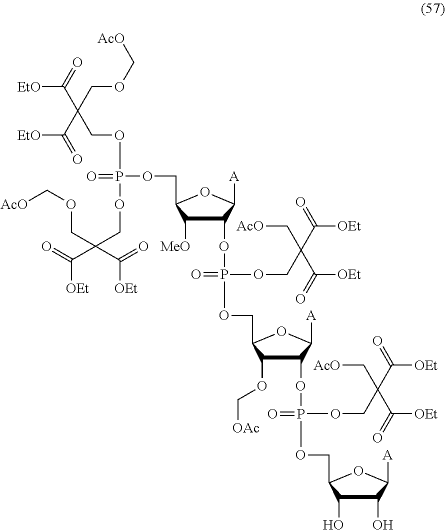

N⁶-(4-methoxytrityl)-3-O-methyladenosine 5′-B is [3-acetyloxymethoxy-2,2-bis(ethoxycarbonyl)propyl]phosphate (0.42 mmol, 0.500 g, dried over P₂O₅ over night) was dissolved in dry DCM (3 mL) under nitrogen atmosphere. Anhydrous Et₃N (2.11 mmol, 294 μL) and bis(diethylamino)chlorophosphine (0.59 mmol, 125 μL) were added. The mixture was stirred for 2.5 hours. The product was isolated by passing the mixture through a short silica gel column eluting with a 9:1 mixture of dry ethyl acetate and hexane containing 1% Et₃N. The solvent was removed under reduced pressure. The identity of the product was verified by ³¹P NMR spectroscopy. ³¹P NMR (202 MHz, CD₃CN) δ: 139.2, −2.3. The compound was coevaporated from dry acetonitrile to remove the traces of Et₃N and dissolved in dry MeCN (0.5 mL) under nitrogen. Dry DCM (0.5 mL) was then added.

3′-O-Acetyloxymethyl-6-N-(4-methoxytrityl)adenosine-2′yl 2′,3′-di-O-levulinoyl-6-N-(4-methoxytrityl)adenosine-5′-yl 3-acetyloxy-2,2-bis(ethoxycarbonyl)phosphate (0.25 mmol, 0.42 g, dried over P₂O₅ over night), dissolved in dry MeCN (2 mL) and dry DCM (0.5 mL), and tetrazole (0.51 mmol, 1.128 mL of 0.45 mol L⁻¹ solution in MeCN) were added. The progress of the reaction was followed by ³¹P NMR spectroscopy. Tetrazole (0.25 mmol, 0.564 mL of 0.45 mol L⁻¹ solution in MeCN) was added after 50 minutes, and the reaction was allowed to proceed for an additional 15 minutes. 2,2-Bis(ethoxycarbonyl)-3-acetyloxy)propanol (0.42 mmol, 0.110 g, coevaporated twice with dry MeCN and dried over P₂O₅ over night) and tetrazole (0.76 mmol, 1.692 mL of 0.45 mol L⁻¹ solution in MeCN) were added, and the mixture was stirred for 30 minutes. ³¹P NMR (202 MHz, CD₃CN) δ: 140.2-140.6 (m), −2,2−(−2.6) (m).

The product from the previous step was oxidized with I₂ (0.1 mol L⁻¹) in a mixture of THF. After 45 minutes, H₂O and 2,6-lutidine (4:2:1, v/v/v, 7 mL) were added. The mixture was stirred overnight at room temperature. Aqueous 5% NaHCO₃ was added, and the mixture was extracted twice with DCM. The organic phase was dried over Na₂SO₄ and evaporated to dryness. The product was purified by Silica gel chromatography eluting with a mixture of DCM and ethyl acetate (3:7) and changing eluent to DCM containing 5% MeOH. The protected trimer (a diastereomeric mixture) was obtained as yellowish oil in 43% yield (0.58 g). The identity was verified by ³¹P NMR spectroscopy. ³¹P NMR (202 MHz, CD₃CN) δ: −1.9−(−2.9) (m).

The protected trimer was evaporated once from dry MeCN and dissolved in dry DCM. The levulinoyl groups were removed using hydrazine acetate (0.74 mmol, 0.070 g) in dry MeOH (0.9 mL). The mixture was stirred at room temperature for 2.5 hours. The reaction was quenched with acetone, stirred for 20 minutes and evaporated to dryness. The product was purified by Silica gel chromatography eluting with DCM containing 5% MeOH. The crude product was obtained in 0.510 g yield.

The trityl groups on the protected trimer were removed by dissolving the product from the previous step (0.280 g) in 80% (v/v) aq AcOH (10 mL). After stirring overnight at room temperature, the reaction mixture was evaporated to dryness. The residue was coevaporated twice with water. The trimer was purified by HPLC on a Sun Fire™ Prep C18 column (250×10 mm, 5 μm, flow rate 3.0 mL min⁻¹) (150×4.6 mm, 5 μm, flow rate 1.0 mL min⁻¹), using a linear gradient elution from 17% to 100% MeOH in 20 minutes and isocratic elution with MeOH for 6 minutes. Overall yield of the trimer starting from starting from the levulinoyl protected trimer was 38% (81 mg). To run the kinetic measurements, the diastereomers were separated, on a Sun Fire™ Prep C18 column (250×10 mm, 5 μm, flow rate 3.0 mL min⁻¹) eluting with 55% MeCN for 30 minutes. ¹H NMR (500 MHz, CD₃OD) δ: 8.07-8.28 (m, 6H, H2 and H8), 6.19-6.26 (m, 1H, H1′), 6.08-6.12 (m, 1H, H1′), 5.90-5.95 (m, 1H, H1′), 5.19-5.69 (m, 7H, H2′ and 3×OCH₂O), 4.80-5.00 (m, 1H, H3′), 4.71-4.75 (m, 1H, H2′), 4.09-4.63 (m, 44H, H2′, 2×H3′, 3×H4′, 3×H5′, 3×H5″, 4×CH₂O, 4×POCH₂C, 8×CH₂CH₃), 3.49-3.58 (m, 3H, 3′-OMe) 1.95-2.17 (m, 15H, OAc), 1.15-1.28 (m, 42H, CH₂CH₃). ³¹P NMR (202 MHz, CDCl₃) δ: −2,6−(−1.4) (m). (Multiplicity of some signals is due to the presence of Rₚ and Sₚ diastereomers.) HRMS (ESI) Calcd for C80H114N15O47P3, 2129.6205; found 2129.6054.

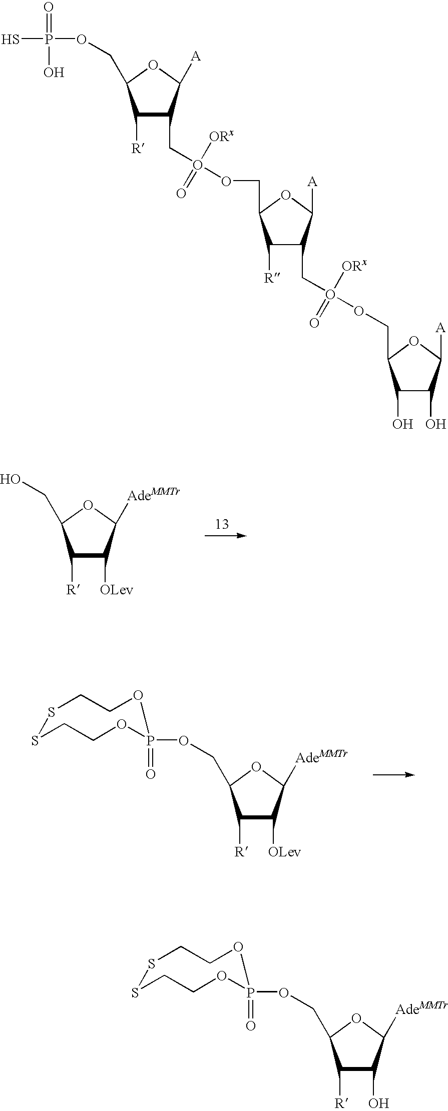

The 5′-phosphorothioate trimer shown above can be synthesized using

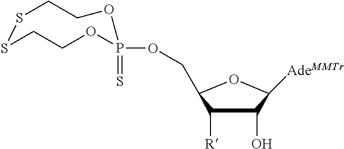

in place of

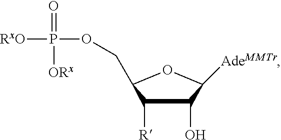

and the procedures and conditions described herein.

The trimers of formula (I) can also be synthesized using the following procedure.

### General Procedure for Synthesis of Phosphoramidite Monomers (27)

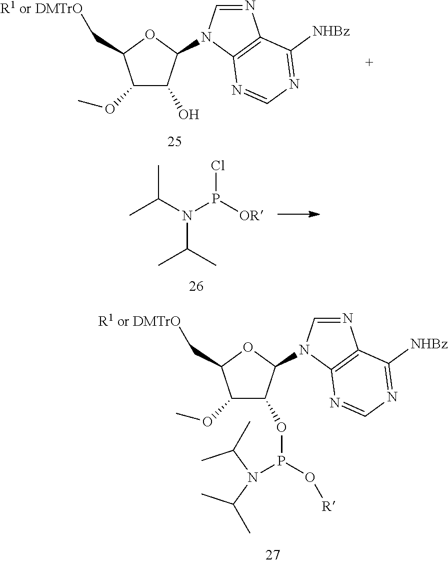

Compound 27 were prepared by reacting a 5′-modified 2′-hydroxy nucleoside 25 with an appropriate substituted phosphoramidic chloride 26 under basic conditions, for example, in the presence of excess diisopropylethyl amine.

### General Procedure for Solution-Phase Synthesis of Trimers

Phosphoramidite (compound 27) was activated in the presence of tetrazole or thioethyl tetrazole, and reacted an appropriate nucleoside (28) to form an intermediate phosphate triester. The trimester was oxidized in situ with DDTT (3-[dimethylaminomethylidene]amino-3H-1,2,4-dithiazole-3-thione) to afford the phosphorothioate dimer (29). The 5′-hydroxy was deprotected under acidic conditions using acetic acid-water or dichloroacetic acid to give the dimer (30).

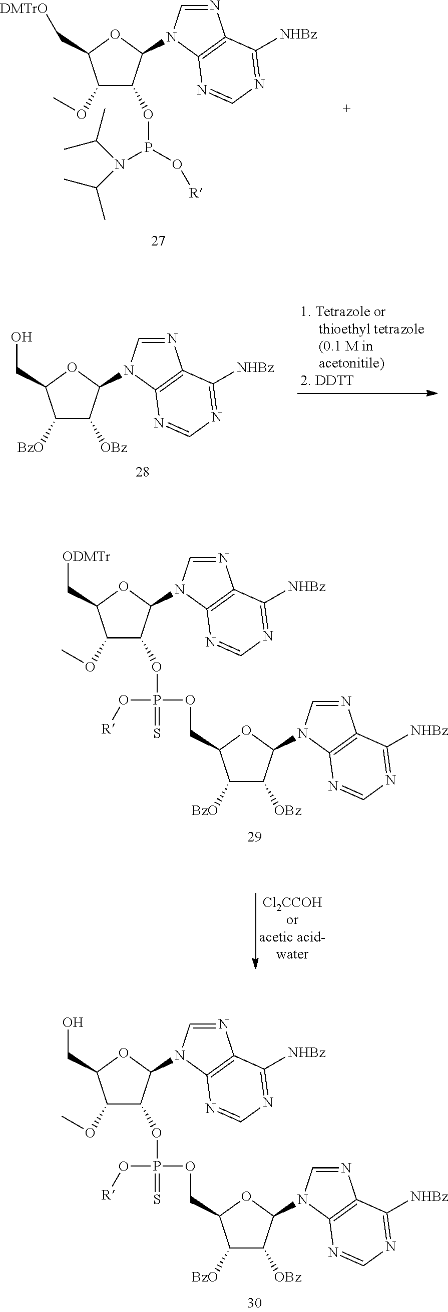

Phosphoramidite (27) was activated in the presence of tetrazole or thioethyl tetrazole, and coupled to dimer (30). The newly formed phosphate triester was oxidized in situ with DDTT (3-[dimethylaminomethylidene]amino-3H-1,2,4-dithiazole-3-thione) to afford the phosphorothioate trimer (31). The benzoyl protecting groups were removed using saturated aqueous NH₄OH or triethyl amine-water at 60° C. for 18 hours to afford compound 32.

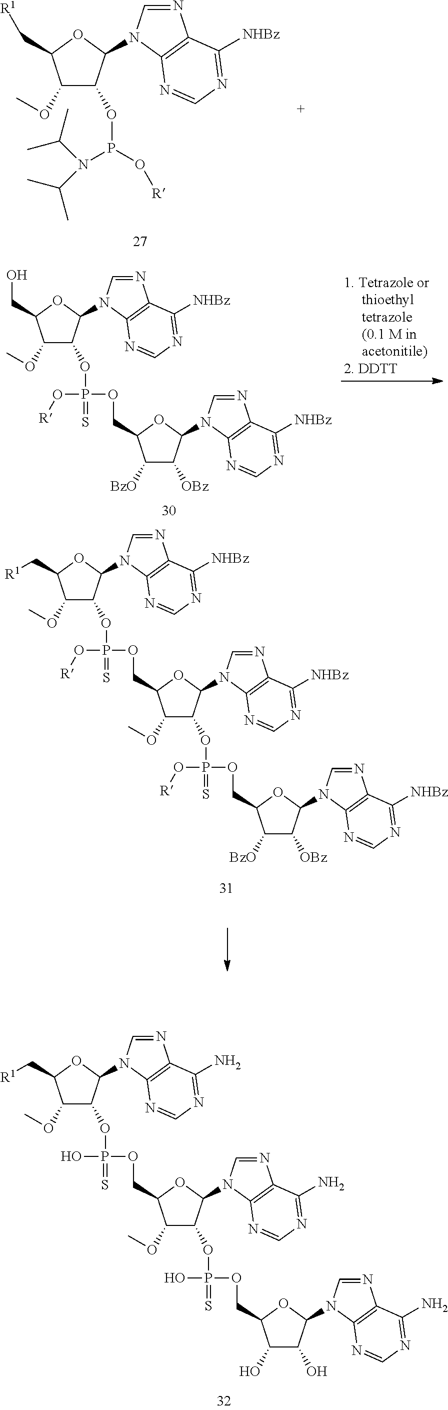

As an example, the trimer shown below was synthesized according to the general procedure for solution-phase synthesis.

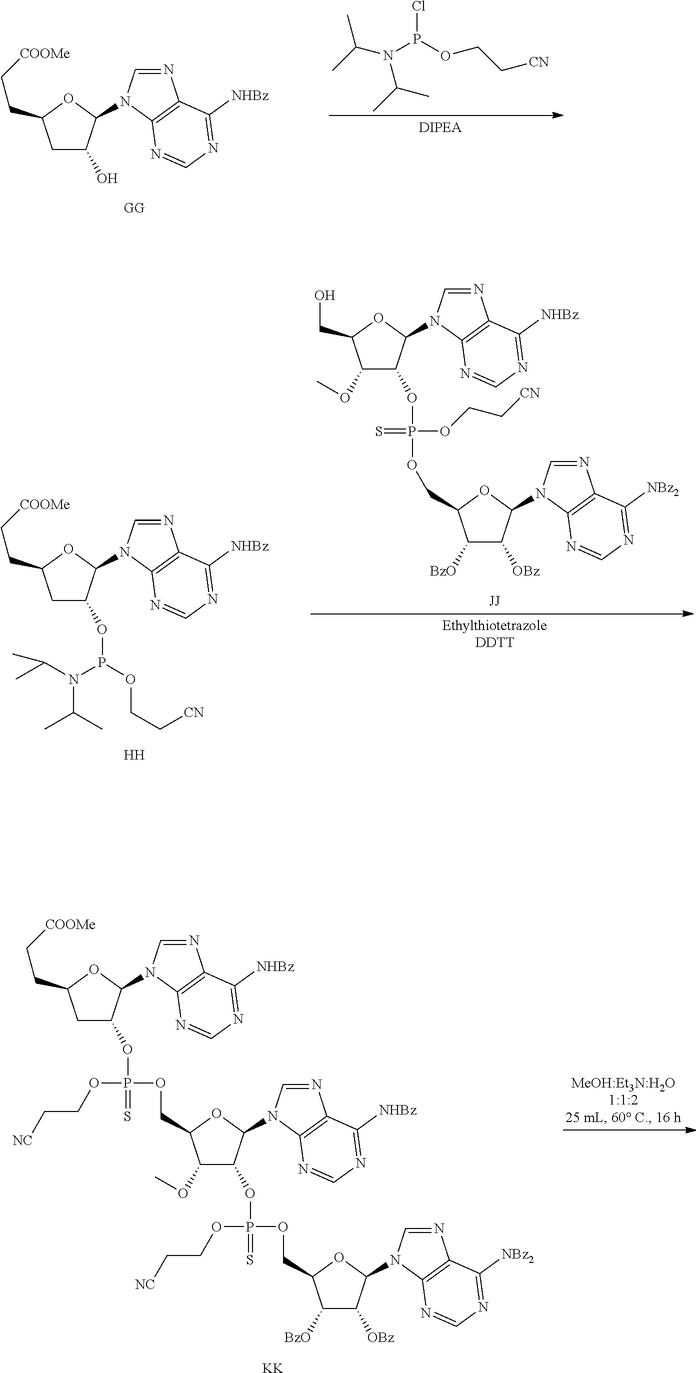

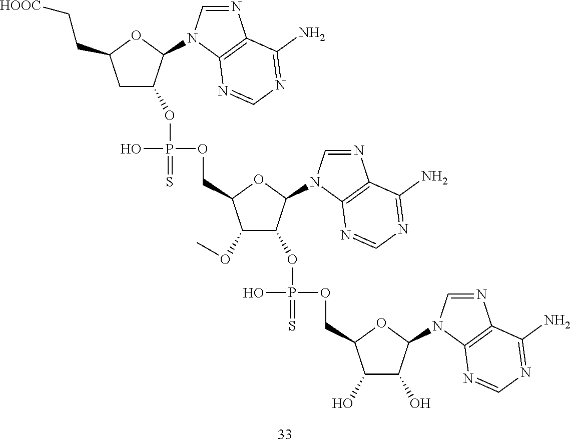

Methyl 3-((2R,4R,5R)-5-(6-benzamido-9H-purin-9-yl)-4-(((2-cyanoethoxy)(diisopropylamino)phosphino)oxy)tetrahydrofuran-2-yl)propanoate (HH). Methyl 3-((2R,4R,5R)-5-(6-benzamido-9H-purin-9-yl)-4-hydroxytetrahydrofuran-2-yl)propanoate (0.32 g, 0.8 mmol) was co-evaporated (2×) with anhydrous DCM (10 mL) and then redissolved in 5.6 mL of DCM. Hunig's base (1.03 mL, 5.6 mmol) was added. The reaction mixture was cooled to 0° C. 2-Cyanoethyl N,N-diisopropylchlorophosphoramidite (0.53 g, 2.24 mmol) was added dropwise, and the reaction mixture stirred at room temperature for 4 hours. The reaction mixture was evaporated and partitioned between ethyl acetate and saturated NaHCO₃. After a normal extractive workup, the organic later was dried to afford the crude product. The product was purified through silica gel chromatography using an ethyl acetate-hexane (containing 10% Et₃N) gradient to afford the desired product as a foam (0.36 g, 76%). ¹H NMR (CD₃CN, 400 MHz) δ 9.25 (br s, 1H), 8.68, 8.67 (2s, 1H), 8.26 (s, 1H), 8.01 (d, 2H, J=8.0 Hz), 7.66 (t, 1H, J=6.8 Hz), 7.56 (t, 2H, J=8.0 Hz), 6.19, 6.12 (2 br s, 1H), 5.02 (m, 1H), 4.44 (m, 1H), 3.85-3.57 (m overlapping with 2s, 7H), 2.64 (m, 2H), 2.16-1.93 (m overlapping with solvent and water peaks), 1.24-1.09 (m, 14H). ³¹P NMR (CD₃CN, 400 MHz) δ 149.84, 149.07.

Compound (KK). Compound 30 (R′═(CH₂)₂CN, 0.21 g, 0.18 mmol) and thioethyl tetrazole (0.06 g, 0.44 mmol) were co-evaporated together (2×) with anhydrous toluene (5 mL), and then anhydrous acetonitrile (3×, 5 mL). The residue was redissolved in anhydrous acetonitrile (0.8 mL) under Ar. Compound HH (0.17 g, 0.27 mmol) was co-evaporated (2×) with 5 mL of anhydrous acetonitrile and redissolved in 0.8 mL of anhydrous acetonitrile. The solution of compound HH was added dropwise to the solution of compound 30. The reaction mixture was stirred for 1 hour. DDTT (3-{dimethylaminomethylidene}amino-3H-1,2,4-dithiazole-3-thione (0.05 M solution in 3:2 pyridine/acetonitrile, 5.3 mL, 0.26 mmol) was added, and the resulting mixture was stirred for 1 hour. The reaction mixture was evaporated to dryness, and then partitioned between water and ethyl acetate. After a normal extractive workup, the organic later was dried to afford the crude product. The product was purified through silica gel chromatography using an ethyl acetate-methanol gradient to afford compound KK as a foam (0.22 g, 73%).

Compound (33). Compound KK (0.22 g) was dissolved in a mixture of MeOH/Et₃N/water (1:1:2 ratio, 25 mL) under Ar. The flask sealed and heated at 60° C. for 18 hours. The reaction mixture was evaporated to dryness, redissolved in water and purified by reverse phase HPLC using 50 mM TEAB in a water/MeOH gradient (20-40%). The desired fractions were co-evaporated multiple times with MeOH (to remove excess TEAB). The fractions were then redissolved in water and lyophilized to afford compound 33 as a triethyl ammonium salt (0.07 g, 56%).

The first eluting diastereomer (33a) was further purified by strong anion exchange chromatography using a GE HiLoad 16/10 Q Sepharose column and TEAB buffer 20 mM (A) and 500 mM (B). A step gradient of 4-40% in 2 column volumes, 40% 2 column volumes, and 80% for two column volumes. The desired fractions were co-evaporated multiple times with MeOH to remove excess TEAB. The fractions were then redissolved in water and lyophilized to give compound 33a (23.0 mg). The second and third diastereomer co-eluted during the first reverse phase separation and were repurified using reverse phase separation with the same column (water and acetonitrile both containing 0.1% acetic acid and a 1-40% gradient). The desired fractions were lyophilized to provide pure 10.5 mg (33b) and 11.2 mg (33c). The fourth diastereomer (33d) was obtained pure from the first reverse phase separation (18.0 mg).

Compound 33a: m/z 996.2; NMR: ³¹P NMR (D₂O, 400 MHz) δ 56.5 and 55.3 ppm; ¹H NMR (D₂O, 400 MHz) δ: 8.40 (s 1H), 8.20 (s 1H), 8.09 (s 1H), 8.01 (s 1H), 7.91 (s 1H), 7.90 (s 1H), 5.99 (s 1H), 5.96 (d 1H), 5.83 (d 1H), 5.46 (m 1H), 5.23 (m 1H), 4.41 (m 3H), 4.0-4.3 (m 6H), 3.64 (s 3H), 2.45 (dd 1H), 2.17 (m 3H), 1.79 (m 2H). Compound 33b: m/z 996.2, ³¹P NMR (D₂O, 400 MHz) δ 55.7 and 55.3 ppm, ¹H NMR (D₂O, 400 MHz) δ: 8.40 (s 1H), 8.22 (s 1H), 8.1 (s 1H), 8.03 (s 1H), 7.98 (s 1H), 7.89 (s 1H), 5.99 (s 1H), 5.95 (d 1H), 5.89 (d 1H), 5.27 (m 2H), 4.5-4.0 (m 9H), 2.45 (dd 1H), 2.19, (m 3H), 1.79 (m 2H). Compound 33c: m/z 996.2; ³¹P NMR (D₂O, 400 MHz) δ 56.6 and 54.1 ppm; ¹H NMR (D₂O, 400 MHz) δ: 8.22 (s 1H), 8.20 (s 1H), 8.10 (s 1H), 8.00 (s 1H), 7.98 (s 1H), 7.91 (s 1H), 6.03 (s 1H), 5.98 (d 1H), 5.85 (d 1H), 5.49 (m 1H), 5.00 (m 1H), 4.5-4.0 (m 9H), 3.65 (s 3H), 2.35 (dd 1H), 2.4-2.0 (m 3H), 1.85 (m 2H). Compound 33d: m/z 996.2; ³¹P NMR (D₂O, 400 MHz) δ 55.6 and 54.1 ppm; ¹H NMR (D₂O, 400 MHz) δ: 8.21 (s, 1H), 8.17 (s 1H), 8.11 (s 1H), 7.99 (s 1H), 7.95 (s 2H), 6.00 (s 1H), 5.95 (d 1H), 5.89 (d 1H), 5.28 (m 1H), 5.05 (m 1H), 4.5-4.0 (m 9H), 3.62 (s 3H), 2.36 (dd 1H), 2.0-2.4 (m 3H), 1.83 (m 2H). Those skilled in the art understand that when L¹ and L² are both chiral, four diastereomers can be present when considering only L¹ and L². In the present application, the stereochemistry shown for L¹ and L² were assigned based on understanding of those skilled in the art, as exemplified by the following literature reference Wang, et al., Nat. Chem. Biol. (2007) 3(11):689-690, and references cited therein.

Other compounds prepared using the procedure for preparing compound 33 include:

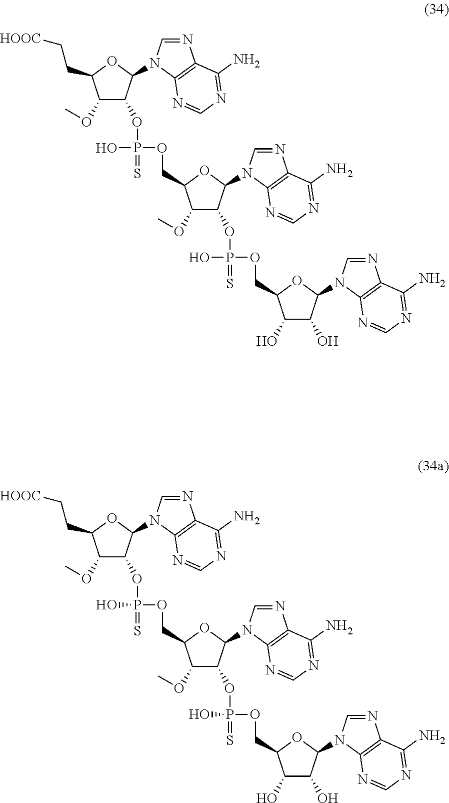

### LC/MS m/z 1026.8 (M−1), ³¹P NMR (D₂O) 57.02 and 56.40.

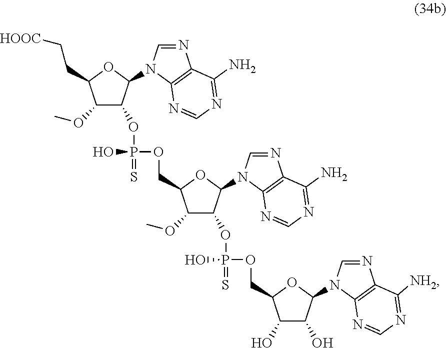

### LC/MS m/z 1026.8 (M−1), ³¹P NMR (D₂O) 56.83 and 55.6.

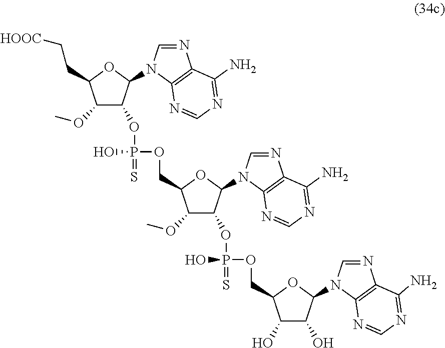

### LC/MS m/z 1026.5 (M−1), ³¹P NMR (D₂O) 56.56 and 55.01.

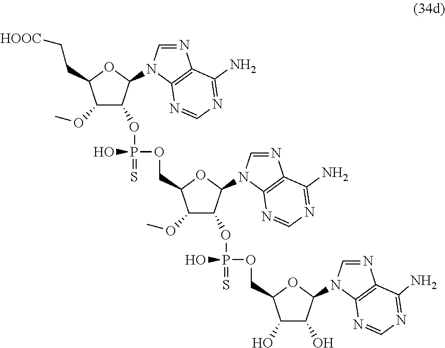

### LC/MS m/z 1026.3 (M−1), ³¹P NMR (D₂O) 55.08 and 55.60.

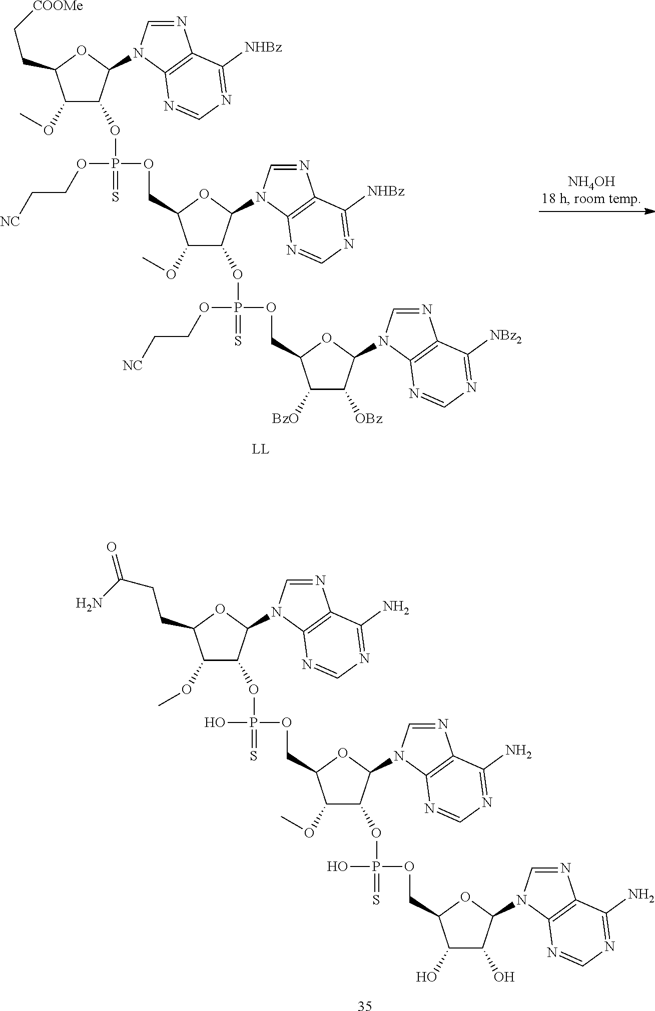

Compound 35 was prepared according to the procedure for obtaining compound 33 except compound LL (0.23 g) in acetonitrile (1 mL), MeOH (1 mL) and NH₄OH (6 mL, 28% aqueous) were combined in a tube. The tube was sealed and left at room temperature for 18 hours. Compound 35 was purified by strong anion exchange chromatography using a GE HiLoad 16/10 Q Sepharose column and TEAB buffer 20 mM (A) and 500 mM (B), using a step gradient. The desired fractions were co-evaporated multiple times with MeOH to remove excess TEAB. The fractions were then redissolved in water and lyophilized to afford pure compound 35 (2.8 mg). LC/MS m/z 1025.8 (M−1).

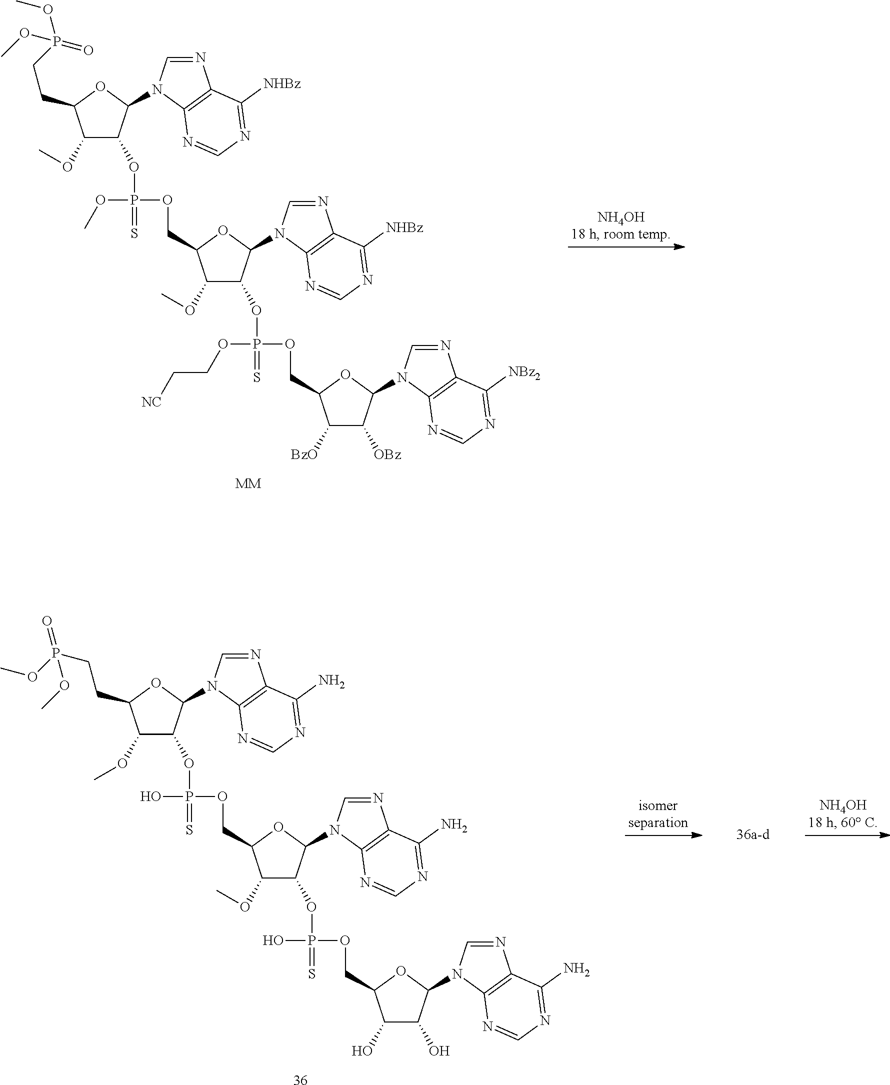

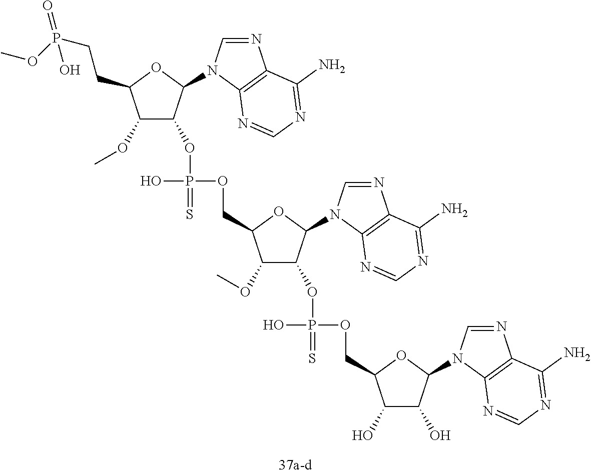

Compounds 36 and 37 were prepared using a similar procedure for preparing compound 33 using dimethyl (2-((2R,3S,4R,5R)-5-(6-benzamido-9H-purin-9-yl)-4-hydroxy-3-methoxytetrahydrofuran-2-yl)ethyl)phosphonate and N,N-diisopropylmethylphosphonamidic chloride as the starting materials. Compound MM was deprotected using the procedure described with respect to compound 35 to afford compound 38 as a mixture of 4 isomers. The isomers were separated according to the procedure described for compound 33. The diasteromers (36a-d) were separately subject to NH₄OH at 60° C. for 18 hours. Compounds 37a-d were obtained as their ammonium salts after drying and lyophilization. For compounds 36a-d, LC/MS m/z 1091.0 (M−1); ³¹P NMR (D₂O) δ7.83, 57.27 and 38.95; 57.68, 56.54 and 38.94; 57.48, 56.12 and 38.95; and 56.45, 56.05 and 38.96. For compounds 37a-d, LC/MS m/z 1076.6 (M−1); 55.72, 55.02 and 26.75; 56.9, 55.7 and 28.1; 56.61, 55.08 and 28.14; and 55.49, 54.96 and 28.06.

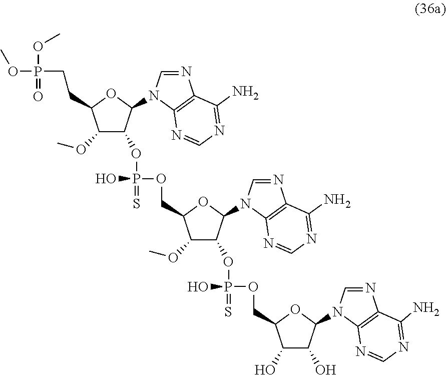

### LC/MS m/z 1091.0 (M−1), ³¹P NMR (D₂O) 57.83, 57.27 and 38.95.

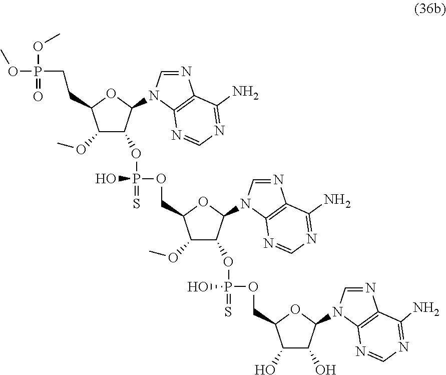

### LC/MS m/z 1091.0 (M−1), ³¹P NMR (D₂O) 57.68, 56.54 and 38.94.

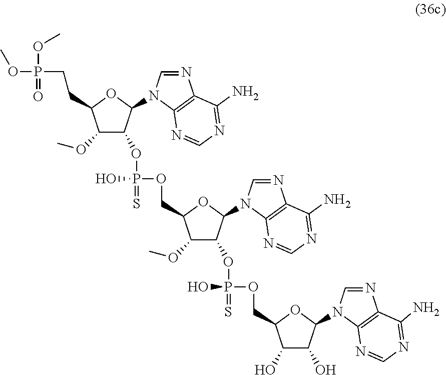

### LC/MS m/z 1091.0 (M−1), ³¹P NMR (D₂O) 57.48, 56.12 and 38.95.

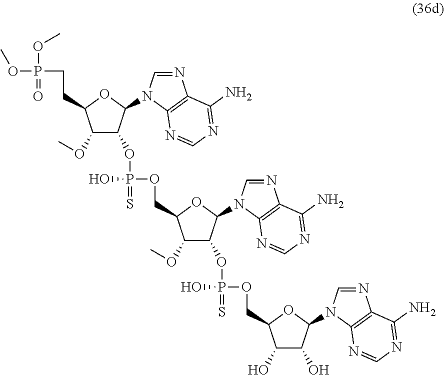

### LC/MS m/z 1091 (M−1), ³¹P NMR (D₂O) 56.45, 56.05 and 38.96.

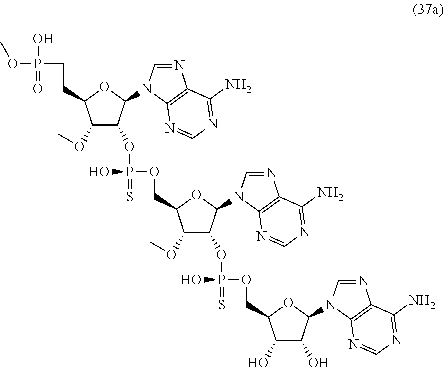

### LC/MS m/z 1076.6 (M−1), ³¹P NMR (D₂O) 55.72, 55.02 and 26.75.

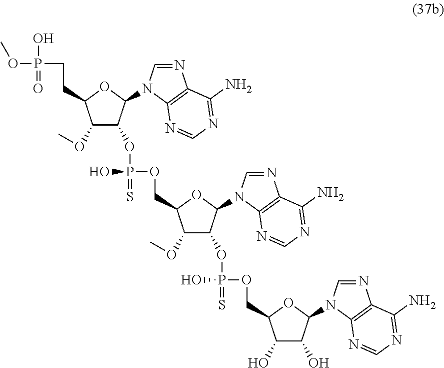

### LC/MS m/z 1076.6 (M−1), ³¹P NMR (D₂O) 56.9, 55.7 and 28.1.

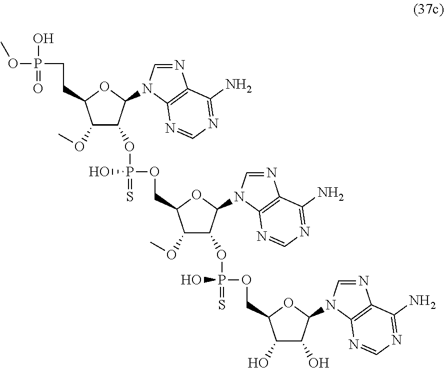

### LC/MS m/z 1076.6 (M−1), ³¹P NMR (D₂O) 56.61, 55.08 and 28.14.

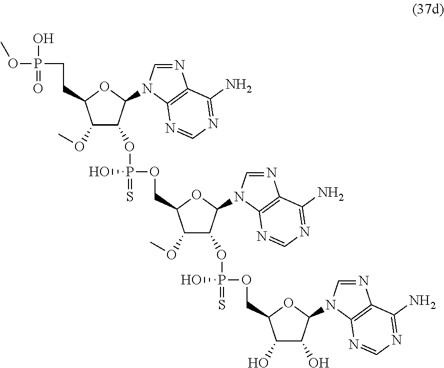

### LC/MS m/z 1076.6 (M−1), ³¹P NMR (D₂O) 55.49, 54.96 and 28.06.

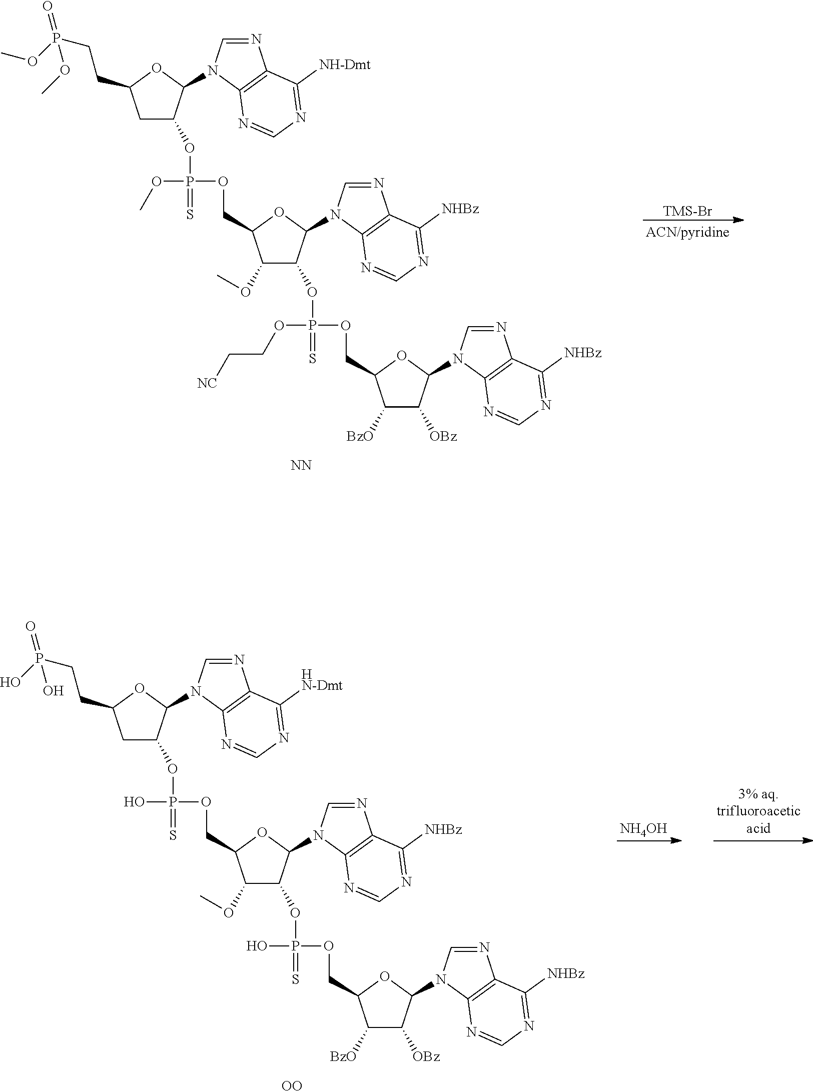

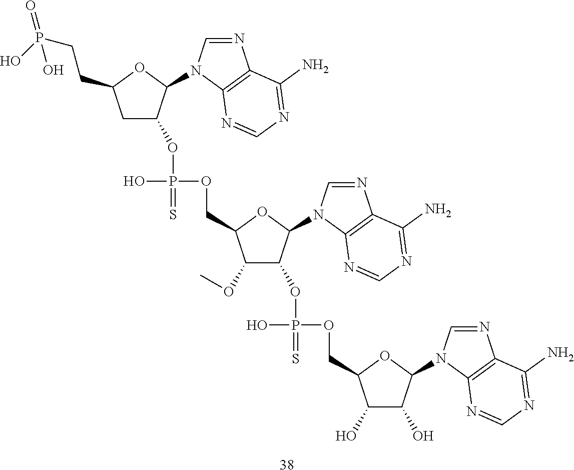

Compound NN was prepared using a similar procedure for preparing compound 33. Compound NN (35 mg; 17 mmole) was dissolved in 5 mL ACN containing 0.4 mL pyridine. TMS-Br (0.2 mL) was added, and the reaction stirred under argon for 1 hour. The solvents were removed, and the residue was co-evaporated with ACN (3×, 1 mL). The residue was dissolved in ACN/water (2:1; 1.5 mL) and left at room temperature for 5 minutes to hydrolyze the trimethylsilyl esters. The solvents were removed in vacuo, and the residue was co-evaporated with ACN (3×, 1 mL) to yield compound OO.

Compound OO was dissolved in methanol (4 mL) and concentrated aqueous ammonium hydroxide (4 mL) was added. The reaction mixture was heated in a sealed vial at 60° C. for 2 hours. After cooling, the solvents were removed in vacuo. The crude material was dissolved in 6 mL water and applied to a Glen ResearchPoly Pak II cartridge previously washed with acetonitrile and then 1M TEAB, in turn washed with 4 mL water, 6 mL of 2% TFA in water (passed through the cartridge over 10 minutes) and 4 mL of water. The product was eluted with 20% acetonitrile in water. The desired fractions were concentrated to dryness to yield compound 38. The crude product was subjected to IE purification using a gradient 20 mM->0.5 M TEAB—peak fractions were pooled and evaporated to dryness followed by co-evaporation with methanol to yield purified compound 38 (6 mg) as a mixture of 4 diastereomers. LC/MS (neg. mode) m/z 1032.5 (M−1).

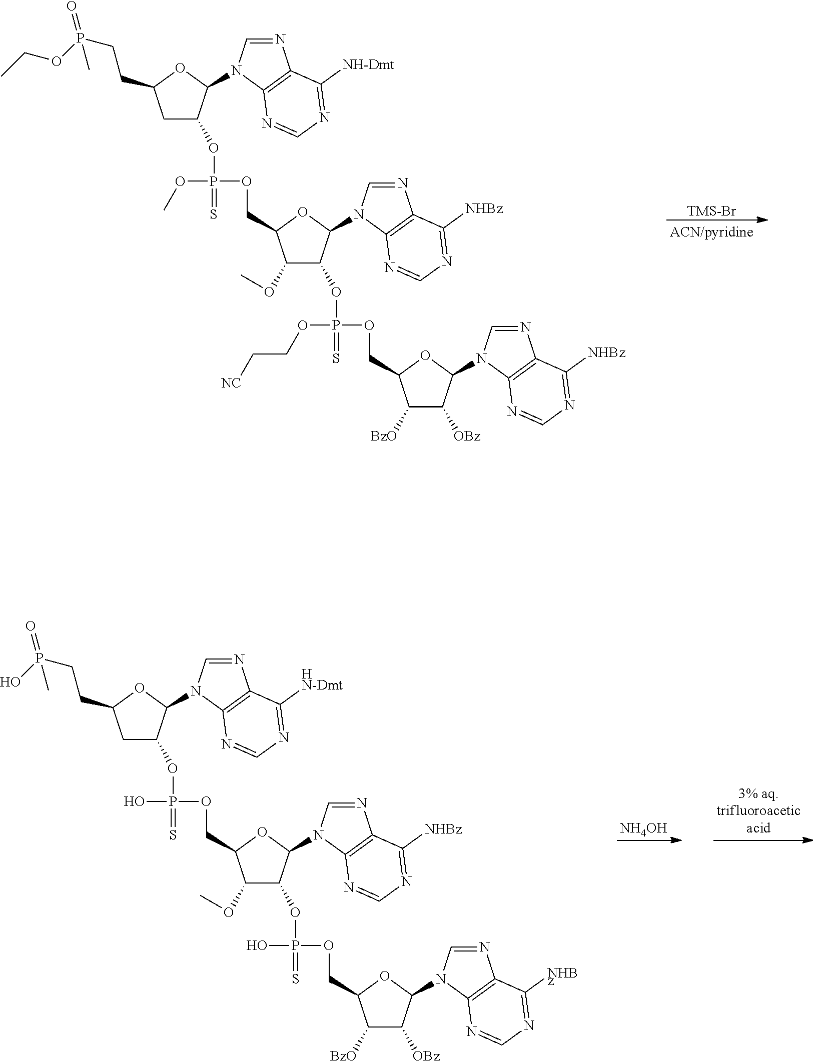

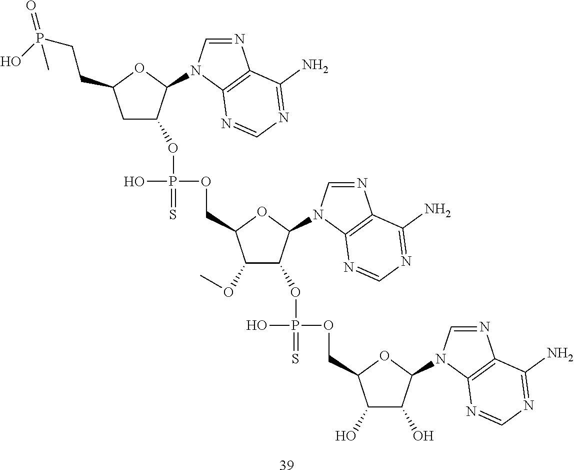

Compound 39 was prepared using procedures similar for preparing Compounds 33 and 38. LCMS m/z 1030.5 (M−1).

Additional compounds that can be obtained using the general procedure for solution-phase synthesis include the following:

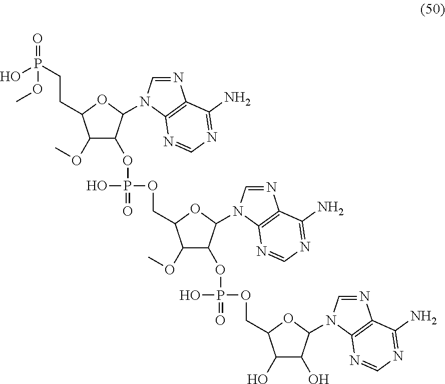

### LC/MS m/z 1044.3 (M−1)

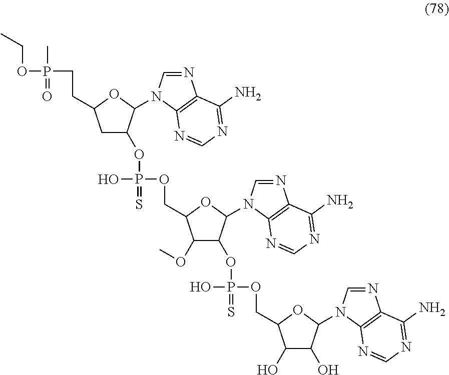

### LC/MS m/z 1058.5 (M−1)

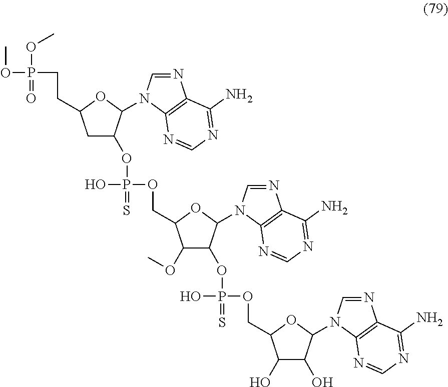

### LC/MS m/z 1060.2 (M−1); ³¹P NMR (D₂O) δ 56.6, 56.5, 55.8, 55.7, 55.5, 55.4, 54.24, 54.19, 37.8 and 37.7.

### General Procedure for Solid-Phase Synthesis of Trimers and Oligomers

The trimers and oligomers were synthesized through the stepwise coupling of monomeric units to a solid-support loaded with 5′-O-DMT-A(NH-Bz)-2′-O-Acetyl-3′-succinyl-CPG (1 micromole scale; Glen Research) or DMT-N⁶-Pac-2′-O-adenosine.

The general steps used in the synthesis:

1. Detritylation with 3% Trichloroacetic acid (TCA) or 5% dichloroacetic acid (DCA) in dichloromethane (DCM); wash with DCM

2. Coupling step—Phosphoramidite 0.1M in acetonitrile (ACN)/0.25M thioethyl tetrazole (ETT) in ACN (1:1 v/v)

3a. Oxidation—20 mM iodine in tetrahydrofuran (THF)/pyridine/water; or

3b. Sulfurization—50 mM 3-[dimethylaminomethylidene]amino-3H-1,2,4-dithiazole-3-thione) (DDTT) in pyridine/ACN (40:60 V/v)

4. Capping

a) Cap Mix A—THF/pyridine/acetic anhydride+

b) Cap Mix B—16% methylimidazole in tetrahydrofuran

The above steps were repeated until the synthesis of the trimer or oligomer was completed. The cycle is shown below.

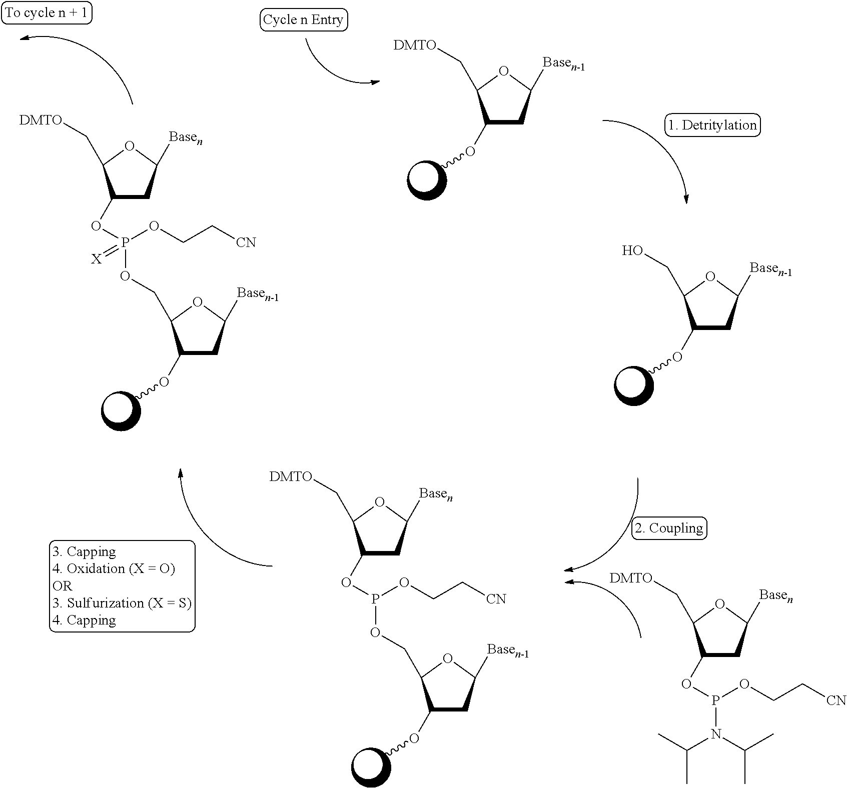

As described herein, ribavirin can be included in a compound of Formulae (I) and (II). One reagent suitable for incorporating ribavirin is 5′-O-DMT-3′-(TBDMS)-ribavirin-2′-(2-cyanoethyl)-N,N-(diisopropyl)phosphoramite.

For compounds with a 5′-methylphosphonate terminal moiety, after the third adenosine were coupled on the solid supports, an additional coupling with 2-cyanoethyl-(N,N-diisopropylamino)methylphosphoramidite, prepared as described herein, was conducted by standard DNA synthesis cycle, followed by the standard cleavage from solid supports and deprotection.

Removal of the support: The compound was deprotected by treating the solid support with 1 mL concentrated aqueous ammonium hydroxide for 30 minutes at room temperature. The support was then filtered off.

Deprotection: After the support was filtered off, the supernatant was heated in a sealed vial at 60° C. for 2-18 hours (depending on the protecting group(s)). The supernatant was then cooled to room temperature, and the ammonium hydroxide solution was removed under vacuum in a SpeedVac.

Deprotection of the trimer with a terminal 5′-CH₂—COOMe-A unit. The compound was cleaved and the protecting groups were removed by heating the solid support with triethylamine-water at 60° C. for 18 hours.

Removal of 3′-O-TBDMS group(s). 3′-O-TBDMS groups were removed by dissolving the compound in 115 μL DMSO. To this solution was added 60 μL triethylamine (TEA) and 75 μL of TEA-3HF (Sigma-Aldrich). The mixture was heated in a sealed vial at 60° C. for 60 minutes. After cooling, the mixture was added to 1M triethylammonium bicarbonate (TEAB) (5 mL).

Purification. Compounds described herein can be purified by ion exchange chromatography (1E) or reverse phase HPLC. For ion exchange chromatography, the compound was diluted to approximately 10 mL with 20 mM TEAB and purified using a GE Akta system equipped with a HiLoad 16/10 Q Sepharose column equilibrated with 20 mM TEAB. A gradient of 4-100% of 0.5M TEAB was used. Fractions having a volume of 2-5 mL were collected. Peak fractions were pooled and evaporated to dryness, and co-evaporation with methanol (3×). The compounds were characterized by HPLC and LC/MS.

Compounds with at least one 3′-hydroxy group can also be purified as follows. After synthesis on DNA synthesizer, the column was treated with aqueous ammonia (29%, 1.0-1.5 mL) by syringe. The resulting ammonia solution stood at room temperature for 2.5-3 days or was heated at 55° C. for 8 hours and then evaporated to dryness. Glen Research RNA purification cartridges were used to purify the trimers: The residue was dissolved in 115 μL of DMSO and 60 μL of triethylamine, and 75 μL of triethylamine trihydrofluoride was added. The resulting solution was heated at 65° C. for 60 minutes, cooled, diluted with 1.75 mL of Glen RNA quenching solution, load onto RNA purification cartridge previously washed with acetonitrile and then 2 M TEAA, wash in turn with 1 mL of acetonitrile-1.0 M ammonium bicarbonate (1:10), 1 mL of water, 2 mL of 2% TFA in water and 2 mL of water. The product was washed down with 30% acetonitrile in 1.0 M ammonium bicarbonate. Collected fractions containing the trimers were diluted with same volume of water and lyophilized. The residue was dissolved in 0.5 mL of water and 0.5 mL of 29% aqueous ammonia, and the resulting solution stood at room temperature for 90 minutes and then was evaporated. The residue was dissolved in water and UV absorbance was measured to quantify the trimers.

Compound 40 was obtained using the general procedure for solid-phase synthesis.

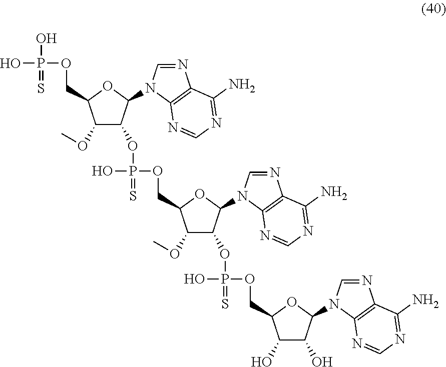

The representative trimer was assembled on 5′-O-DMT-A(NH-Bz)-2′-β-Acetyl-3′-succinyl-CPG (1 micromole scale; Glen Research) by the addition of a). 5′-O -DMT-A(NH-Bz)-3′-O-Methyl-2′-O-(BCE-phosphoramidite); b). 5′-O-DMT-A(NH-Bz)-3′-O-Methyl-2′-O-(BCE-phosphoramidite); and c). [3-(4,4′-Dimethoxytrityloxy)-2,2-dicarboxymethylamido]propyl-(2-cyanoethyl)-(N,N-diisopropyl)-phosphoramidite. Sulfurization was conducted after each of the aforementioned additions. The terminal DMT group was removed from the synthesizer, and the protecting group(s) were removed to give compound 40. LC/MS=1080.6, ³¹P NMR (D₂O) δ 56.7, 55.4 and 44.2. ¹H NMR (D₂O) δ 7.8-8.5 (22s, 6H) 5.8-6.2 (m, 3H) 5.2-5.6 (m, 2H) 3.9-4.5 (m, 13H) and 3.5-3.7 (7s, 6H).

Compounds prepared using the general procedure for solid-phase synthesis include the following:

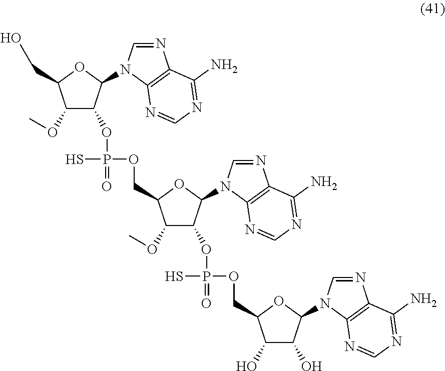

### LC/MS m/z 984.2 (M−1), 1007.0 (M+23),

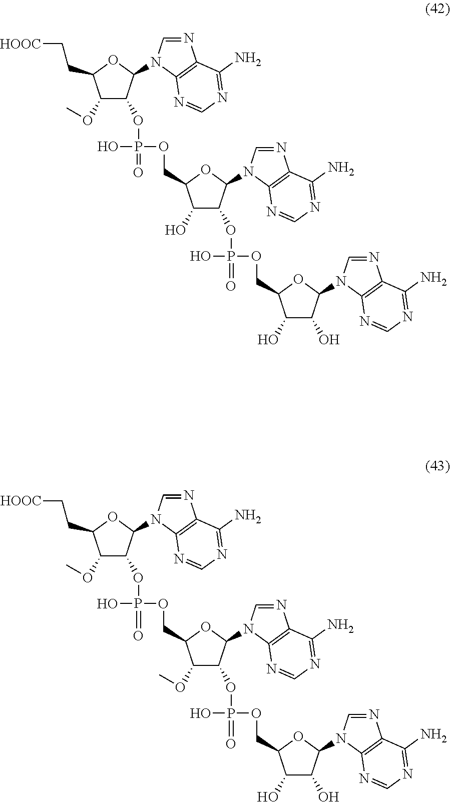

### LC/MS m/z 994.8

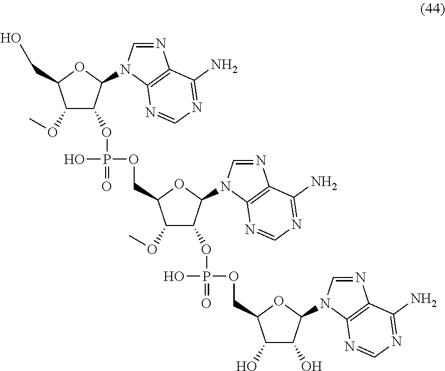

### LC/MS m/z 952.5 (M−1)

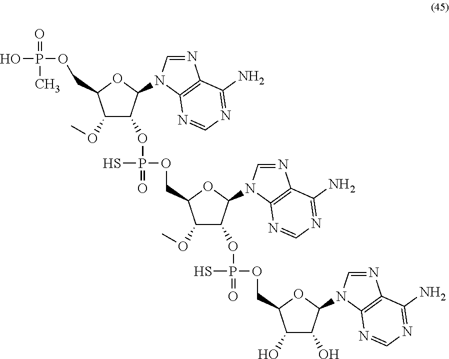

### LC/MS m/z 1062.4 (M−1).

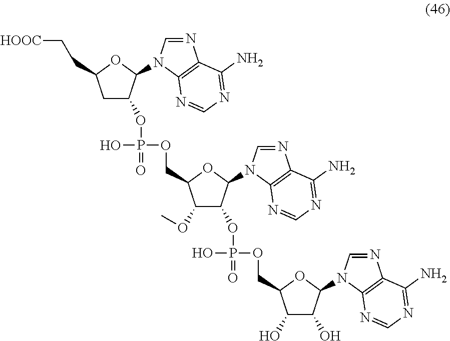

### LC/MS m/z 964.7 (M−1)

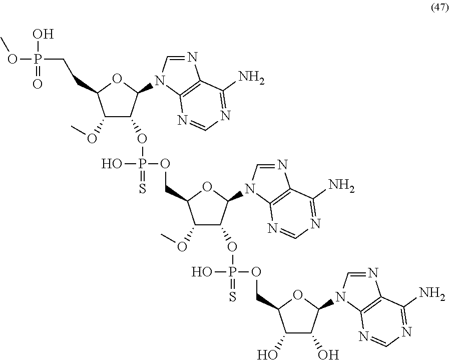

### LC/MS m/z 1076.3 (M−1)

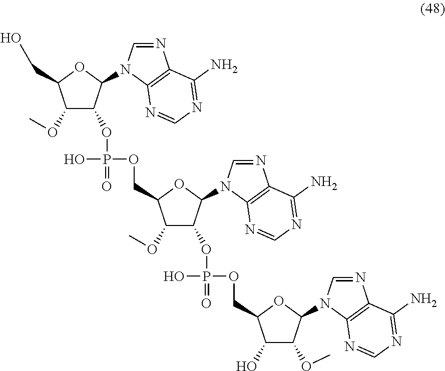

### LC/MS m/z 966.5 (M−1)

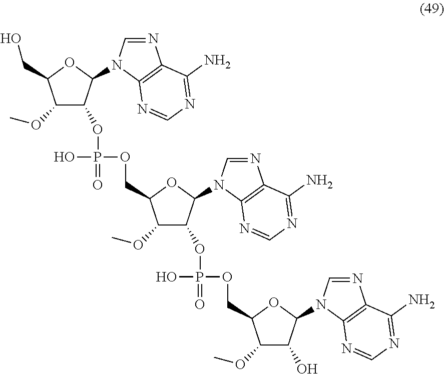

### LC/MS m/z 966.7 (M−1)

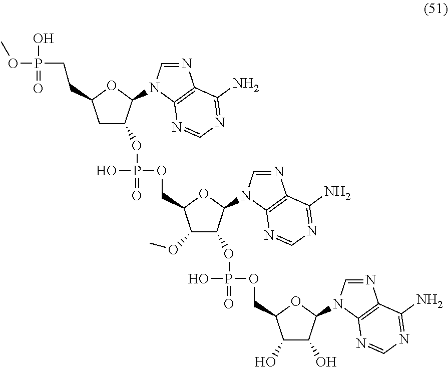

### LC/MS m/z 1014.3 (M−1)

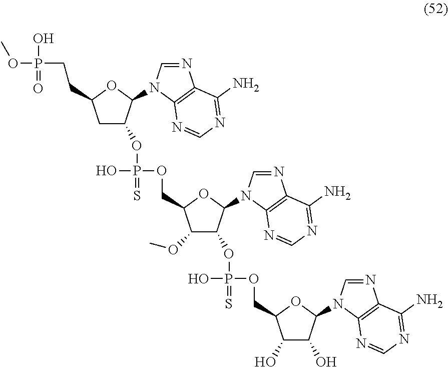

### LC/MS m/z 1046.2 (M−1)

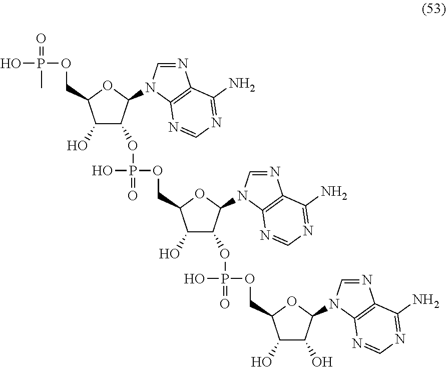

### LS/MS m/z 1002.;3 (M−1)

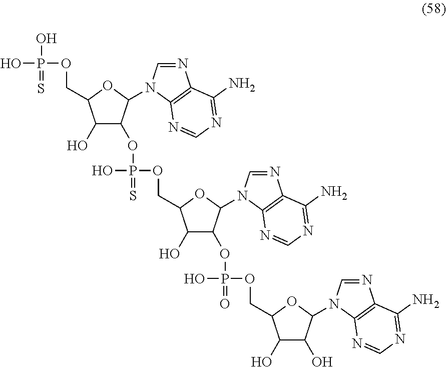

### MALDI 1038.32

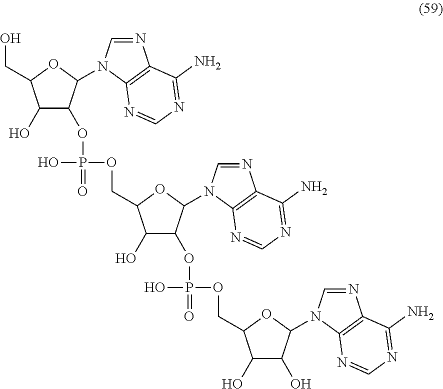

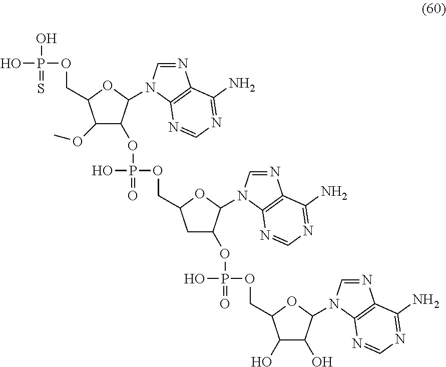

### MALDI 1019.97

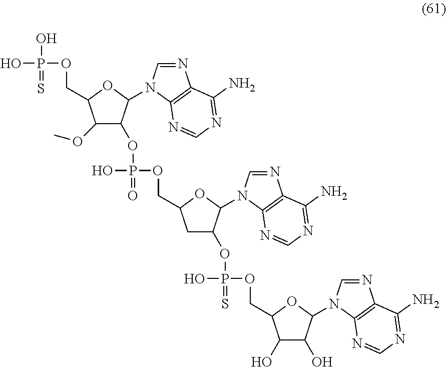

### MALDI 1037.24

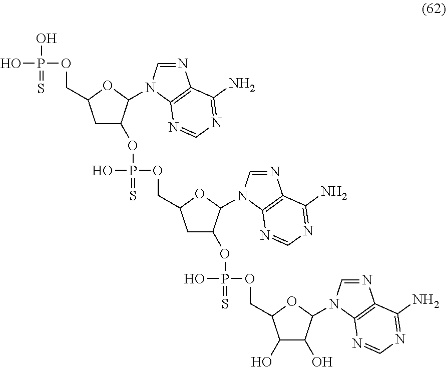

### MALDI 1022.28

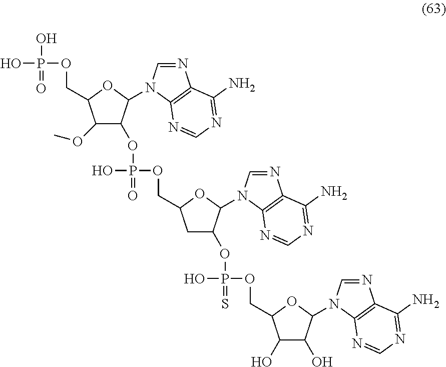

### MALDI 1021.84

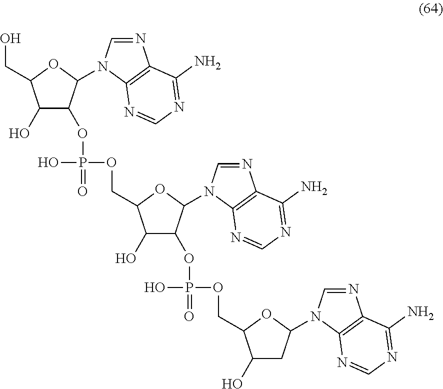

### LC/MS m/z 908.3 (M−1)

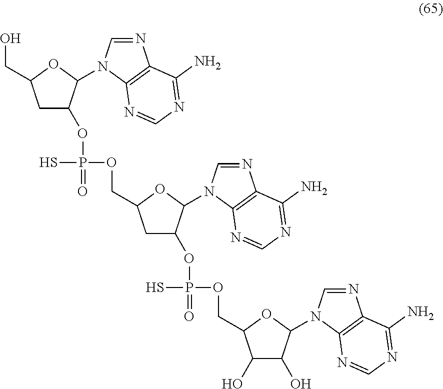

### LC/MS m/z 924.4 (M−1)

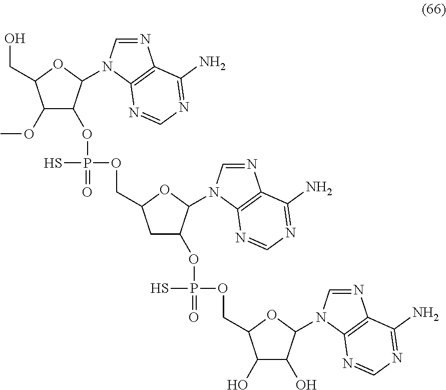

### LC/MS m/z 954.5 (M−1). 976.2 (M+23)

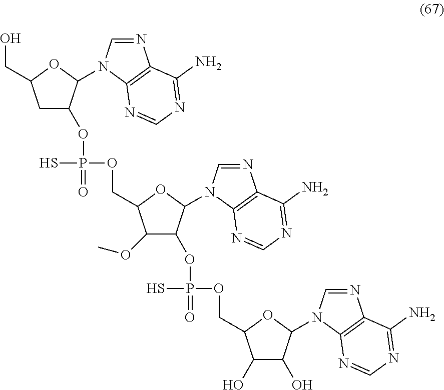

### LC/MS m/z 954.5 (M−1)

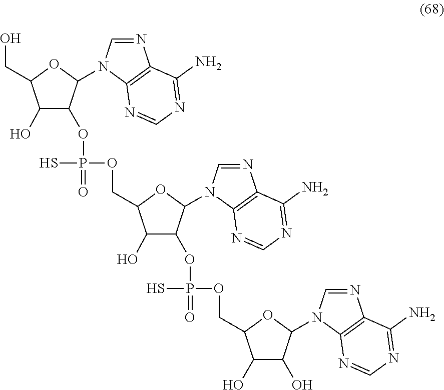

### LC/MS m/z 956.4 (M−1)

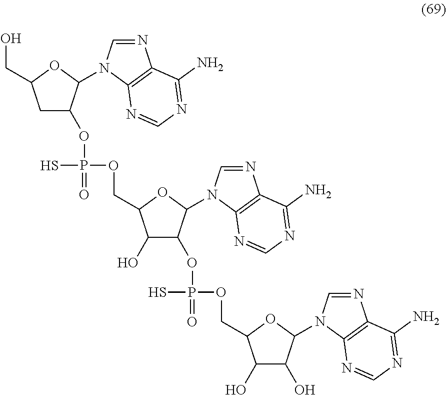

### MALDI 942

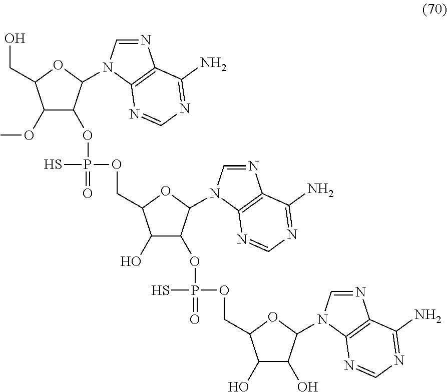

### LC/MS m/z 970.5 (M−1)

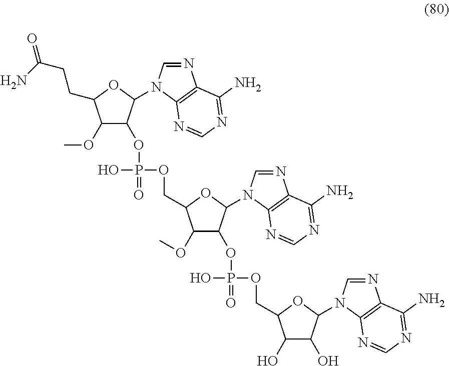

### LC/MS m/z 993.8 (M−1)

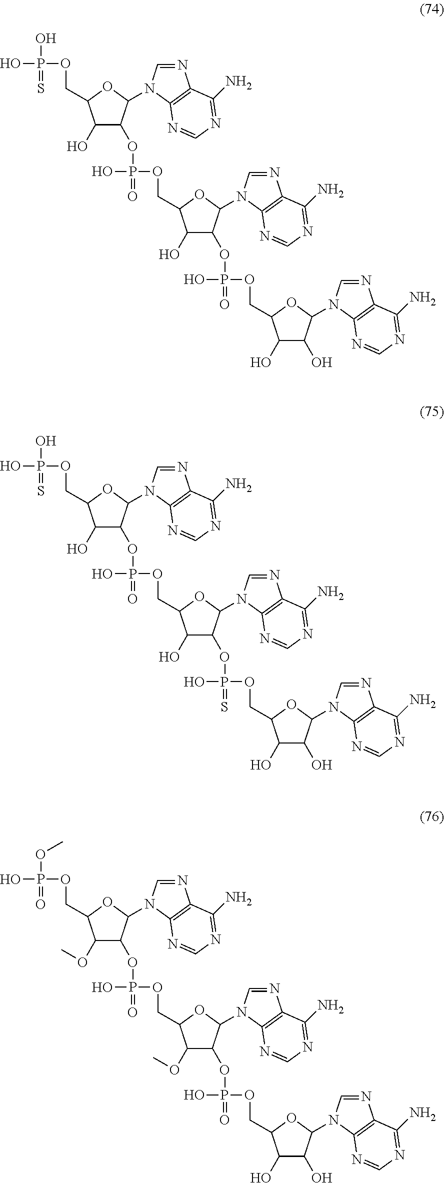

### LC/MS m/z 1044.3 (M−1)

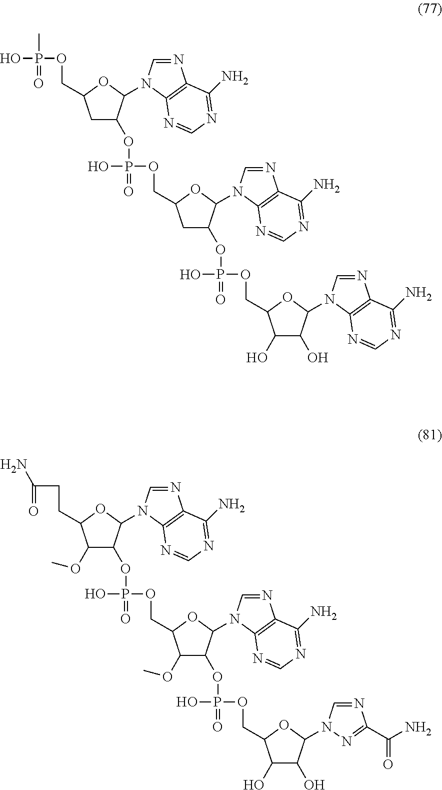

Oligomers of the general formula, ps-[A(3′-OMe)-ps]ₙA where n=3, 4, 5, 6, were assembled on 5′-O-DMT-A(NH-Bz)-2′-O-Acetyl-3′-O-succinyl-CPG (1 micromole scale; Glen Research) utilizing n cycles with the addition of a). 5′-O-DMT-A(NH-Bz)-3′-β-Methyl-2′-O-(BCE-phosphoramidite); and b). [3-(4,4-Dimethoxytrityloxy)-2,2-dicarboxymethylamido]propyl-(2-cyanoethyl)-(N,N-diisopropyl)-phosphoramidite and sulfurization after each addition. Prior to deprotection the terminal DMT group was removed on the synthesizer. Examples of compounds obtained by this method include the following.

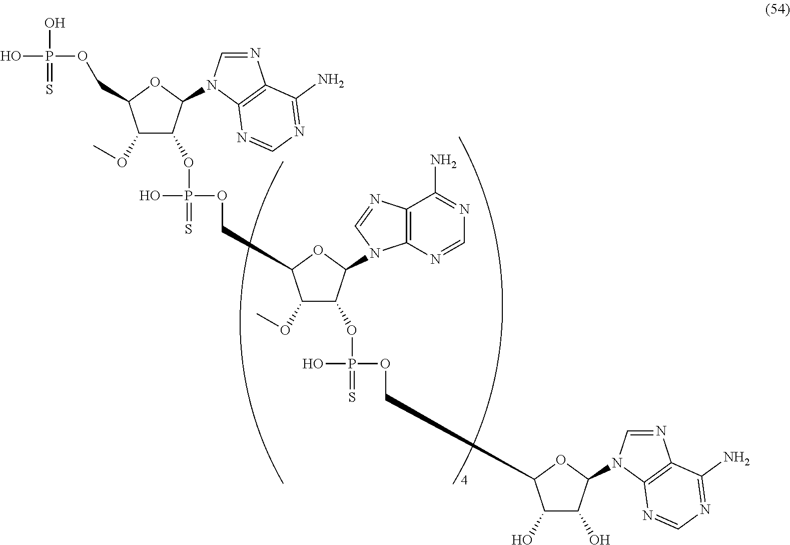

### LC/MS m/z 1079.0 ({M−2H}/2)

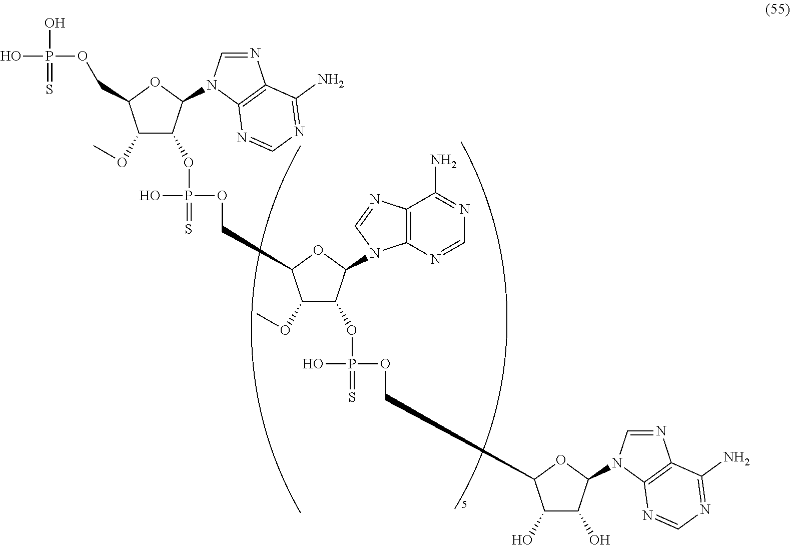

### LC/MS m/z 1258.7 ({M−2H}/2).

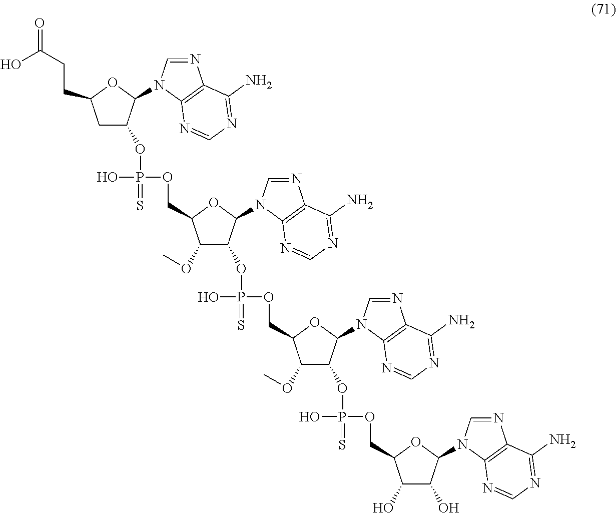

### LC/MS m/z 1355.5 (M−1)

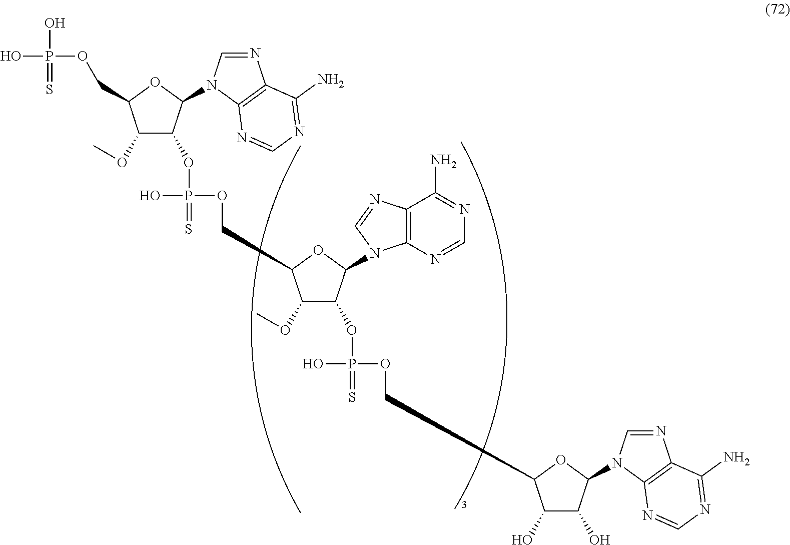

### LC/MS m/z 1799

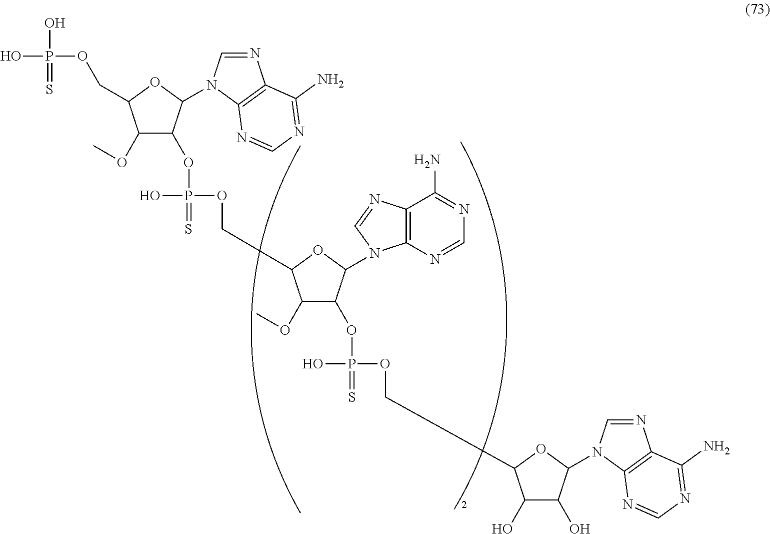

### LC/MS m/z 1440

### Kinetic Studies

### Preparation of the cell extract. 10×10⁶ of human prostate carcinoma cells (PC3) are treated with 10 mL of RIPA-buffer [15 mM Tris-HCl pH 7.5, 120 mM NaCl, 25 mM KCl, 2 mM EDTA, 2 mM EGTA, 0.1% Deoxycholic acid, 0.5% Triton X-100, 0.5% PMSF supplemented with Complete Protease Inhibitor Cocktail (Roche Diagnostics GmBH, Germany)] at 0° C. for 10 minutes. Most of the cells are disrupted by this hypotonic treatment and the remaining ones are disrupted mechanically. The cell extract is centrifuged (900 rpm, 10 minutes), and the pellet is discarded. The extract is stored at −20° C.

Stability of Test Trimers in the cell extract. The cell extract is prepared as described above (1 mL), and is diluted with a 9-fold volume of HEPES buffer (0.02 mol L⁻¹, pH 7.5, I=0.1 mol L⁻¹ with NaCl). A test compound (0.1 mg) is added into 3 mL of the HEPES buffered cell extract, and the mixture is kept at 22±1° C. Aliquots of 150 μL are withdrawn at appropriate intervals, filtered with SPARTAN 13A (0.2 μm) and cooled in an ice bath. The aliquots are analyzed immediately by HPLC-ESI mass spectroscopy (Hypersil RP 18, 4.6×20 cm, 5 μm). For the first 10 minutes, 0.1% aq formic acid containing 4% MeCN is used for elution and then the MeCN content is increased to 50% by a linear gradient for 40 minutes.

Stability of Test Trimers towards Porcine Liver Esterase. The stability of the test trimers was followed by an HPLC and HPLC-MS methods. The reactions were carried out in sealed tubes immersed in a thermostated water bath (37.0±0.1° C.). The oxonium ion concentration of the reaction solutions (3.0 mL) was adjusted with N-[2-hydroxyethyl]piperazine-N-[2-ethanesulfonic acid] (HEPES) buffer (0.040/0.024 mol L⁻¹; pH 7.5). The ionic strength of the solutions was adjusted to 0.1 mol L⁻¹ with sodium chloride. The oxonium ion concentrations of the buffer solutions were calculated with the aid of the known pKa values of the buffer acids under the experimental conditions. The initial concentration of the test trimers was 0.15 mmol L⁻¹ and the hog liver carboxyesterase concentration was 2.6 U mL⁻¹. Samples (200 μL) were withdrawn at appropriate intervals, and made acidic (pH 2) with 1 mol L⁻¹ aqueous hydrogen chloride. The samples were then cooled in an ice-bath and filtered with minisart RC 4 filters (0.45 μm). The composition of the samples was analyzed on an ODS Hypersil C18 column (4×250 mm 5 μm, flow rate 1 mL min⁻¹), using a mixture of acetic acid/sodium acetate buffer (0.045/0.015 mol L⁻¹) and MeCN, containing ammonium chloride (0.1 mol L⁻¹). A separation of the products was obtained on using a 5 min isocratic elution with the buffer containing 2% MeCN, followed by a linear gradient (23 min) up to 40.0% MeCN. The products were identified by the mass spectra (LC/MS) using a mixture of water and MeCN, containing a formic acid (0.1%) as an eluent (Gemini C18 column (2×150 mm 5 μm, flow rate 200 μL min⁻¹). Signals were recorded on a UV-detector at a wavelength of 260 nm. The pseudo first-order rate constants for the disappearance of compounds 56 and 57 were obtained by applying the integrated first-order rate equation to the time-dependent diminution of the concentration of the starting material.

Analysis of the reverse phase-HPLC profiles showed that three of the four phosphate protecting groups were cleaved from compound 56. For compound 57, the fully deprotected 2-5A trimer was obtained.

Stability tests in human serum. Stability tests in human serum are carried out as described for the whole cell extract. The measurements are carried out in serum diluted 1:1 with HEPES buffer (0.02 mol L⁻¹, pH 7.5, I=0.1 mol L⁻¹ with NaCl).

### RNASE L Activation Studies

RNase L activation FRET assay was performed using a 36 nucleotide synthetic oligoribonucleotide substrate: 6-FAM-UUA UCA AAU UCU UAU UUG CCC CAU UUU UUU GGU UUA-BHQ-1 (Integrated DNA Technologies, Inc., Coralville, Iowa). This RNA probe corresponds to a segment of the intergenic region of respiratory syncytial virus (RSV) genomic RNA, and contains several cleavage sites for RNase L (UU or UA). Upon RNA cleavage, the fluorescent FAM group is released from the BHQ quencher. The recombinant human RNase L expressed from a baculovirus vector in insect cells was used in this assay at an effective concentration of 120 nM, together with 200 nM FRET probe with a final volume of 10 μl cleavage buffer [25 mM Tris•HCl (pH 7.4), 100 mM KCl, 10 mM MgCl₂, 50 μM ATP, 7 mM 2-mercaptoethanol, and 0.005% tween 20].

Compounds described herein or the native 2-5A trimer were added with a 384-well black polypropylene plate. Fluorescence was measured in a continuous mode up to 30 minutes with a Wallac 1420 Victor³V multilabel counter (PerkinElmer Life Sciences, Shelton, Conn.) (excitation 485 nm; emission 535 nm). False positives were eliminated by screening the compounds in parallel in the absence of RNase L. Measured EC₅₀ is defined as the concentration at which fluorescence is 50% that of the positive control (native 2-5A). EC₅₀ was calculated by fitting the data to the sigmoidal equation Y=% Min+(% Max−% Min)/(1+X/EC₅₀), where Y corresponds to the percent relative enzyme activity, % Max is the relative activity at saturating compound concentration, % Min is the basal enzyme activity in the absence of compound, and X corresponds to the compound concentration. The EC₅₀ values were derived from the mean of a minimum of two independent experiments.

The EC₅₀ values of the majority of compounds tested were <20 μM, and for a number of compounds the EC₅₀ was <1 μM.

### Bovine Viral Diarrhea Virus (BVDV) Assay

The antiviral activity of test compounds were determined by evaluating the inhibition of virus-induced celling killing, or cytopathic effect (CPE). Madin-Darby bovine kidney (MDBK) cells were incubated in Dulbecco's modified Eagles medium (DMEM) supplemented with 10% heat-inactivated equine serum, 2 μM L-gluamine, 200 U/mL of penicillian and 0.2 mg/mL of streptomycin. Cells were plated in 48-well microtiter plate at a density of 1×10⁴ cells/well. The next day, the cells were infected with BVDV at a low MOI (0.01), selected after a titration experiment, where several MOI ratios were tested. Test compounds were added at various concentrations and incubated in 37° C., 5% CO₂ incubator for 48 hours before staining with crystal violet.

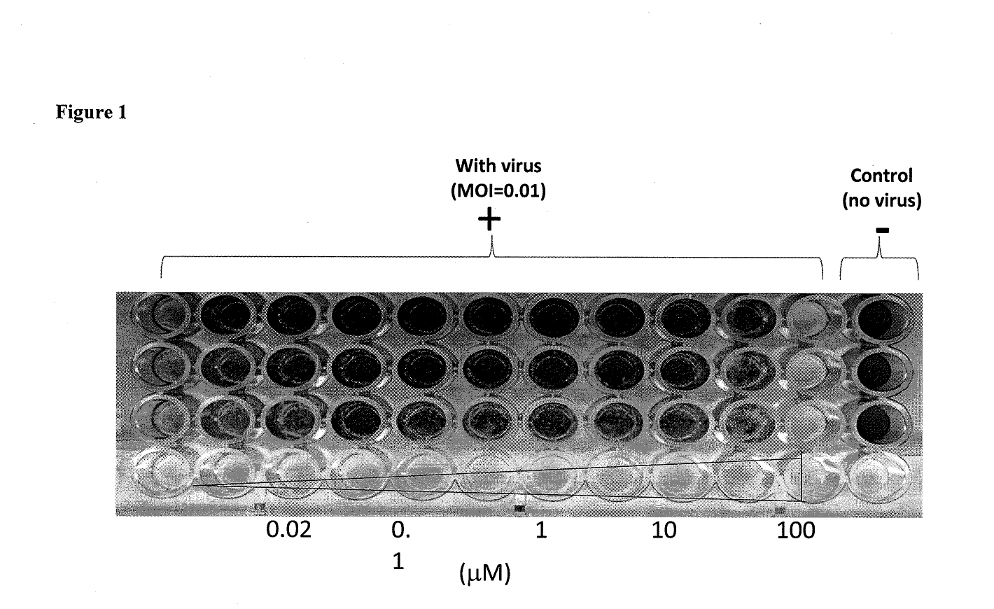

FIG. 1 shows the 48-well plate after staining with crystal violet. The right 3 wells are the positive control (no virus). As shown in FIG. 1, the wells with a compound described herein remained relatively blue in color. This demonstrates that a compound described herein with an EC₅₀ of approximately 8 nM protects against the cytopathic effects of the virus.

### Yellow Fever (YFV) Assay

The antiviral activity of test compounds are determined by evaluating the inhibition of yellow fever virus (YFV)-induced cell killing, or cytopathic effect (CPE). The experiment is performed in Vero cells by CPE inhibition assays, as determined by microscopic examination, increase of neutral red (NR) dye uptake, and virus yield reduction (VYR). Eight concentrations of compound are evaluated against the Jimenez strain of YFV in 96-well flat-bottomed microplates plated with Vero cells (American Type Culture Collection, ATCC). Compounds are added 5-10 min prior to the addition of virus. Virus is added at 2 plaque forming units (PFU) per well. Tests are read after incubation at 37° C. for 6 days. For NR uptake, dye is added (0.034% in medium) to plates after visual examination for 2 hours after which the dye is eluted from the cells and absorbed dye quantified. The Cell-Titer Glo system (Promega, Madison, Wis.) is used to determine cell viability by assaying for the presence of ATP in infected and uninfected cells treated with compounds. Appropriate negative and positive controls are used for comparison. Vero cells are plated in half-growth area 96-well plates and luminescence read on an LB960 Cetro luminometer. Potency is measured as EC₅₀: the concentration of compound at which the viral load in the infected cells is reduced by 50%.

A compound described herein was shown to be active against YFV with an EC₅₀ of 1.4 ug/ml.

### Encephalomyocarditis Virus (EMCV) Assay

The antiviral activity of test compounds were determined by evaluating the inhibition of endomyocarditis virus (EMCV)-induced cell killing, or cytopathic effect (CPE). A549 cells (ATCC, Cat # CCL-185) were incubated in F-12 Ham Media (Sigma-Aldrich Cat # N-4888) supplemented with 10% heat inactivated bovine serum, 2 μM L-glutamine, 200 U/ml of penicillin and 0.2 mg/ml of streptomycin. Cells were plated in 96-well microtiter plates at a density of 6×10⁴ cells/well. The next day the cells were pre-incubated with compounds at various concentrations for 2 hours and then infected with EMCV at an multiplicity of infection (MOI) of 0.0005-0.001, selected after a titration experiment, where several MOI ratios were tested. The cells were then incubated in a 37° C., 5% CO₂ incubator for an additional 48 to 72 hours until complete CPE occurred in the no drug control wells. The plates were immediately fixed and stained with crystal violet. The antiviral activity of a potential therapeutic agent against EMCV was determined by evaluating the inhibition of virus induced cell killing, or cytopathic effect (CPE).

A compound described herein was shown to be active against EMCV with an EC₅₀ of approximately 5 uM.

### Influenza Virus Assay

The antiviral activity of test compounds were determined by evaluating the inhibition of influenza A/Weiss/43 virus induced cell killing, or cytopathic effect (CPE). Test compounds were screened in a 96-well plate format using a standard CPE assay using Madin-Darby canine kidney (MDCK) cells. Briefly, MDCK cells were plated at 1×10³ cells/well in 96-well culture plates and incubated in a 37° C., 5% CO₂ incubator overnight. Each test compound was then diluted into an appropriate volume of dimethyl sulfoxide (DMSO) and an appropriate volume of serum-free medium so that the final DMSO concentration was 1% and then added to the cells in a 50 μL volume. Diluted influenza virus A/Weiss/43 was then added to the cells with medium at a concentration of 100 Tissue Culture Infectious Doses (TCID) 50 in a 50 μL volume. The plates were then incubated at 37° C., 5% CO₂ for 3 days. At the end of the culture period 20 μL of MTT was added into each well and incubated at 37° C. for 4 hours. The amount of reduced MTT (formazan) from the cells was then measured and the data was used to calculate % inhibition of influenza-induced CPE.

A compound described herein was shown to be active against the influenza A/Weiss/43 virus with an EC₅₀ of 8 uM.

### Hepatitis C Virus (HCV) Assay

The HCV sub-genomic replicon (1377/N53-3′, accession No. AJ242652), stably maintained in HuH-7 hepatoma cells, is created by Lohmann et al. Science 285: 110-113 (1999). The replicon-containing cell culture, designated GS4.3, is obtained from Dr. Christoph Seeger of the Institute for Cancer Research, Fox Chase Cancer Center, Philadelphia, Pa.

GS4.3 cells are maintained at 37° C., 5% CO₂, in DMEM (Gibco 11965-092) are supplemented with L-glutamine 200 mM (100×) (Gibco25030-081), non-essential amino acids (NEAA)(Biowhittaker 13-114E), heat-inactivated (HI) Fetal Bovine Serum(FBS)(Hyclone SH3007.03) and 750 μg/ml geneticin (G418)(Gibco 10131-035). Cells are sub-divided 1:3 or 4 every 2-3 days.

24 h prior to the assay, GS4.3 cells are collected, counted, and plated in 96-well plates (Costar 3585) at 7500 cells/well in 100 μl standard maintenance medium (above) and are incubated in the conditions above. To initiate the assay, culture medium is removed, cells are washed once with PBS (Gibco 10010-023) and 90 μl Assay Medium (DMEM, L-glutamine, NEAA, 10% HI FBS, no G418) are added. Inhibitors are made as a 10× stock in Assay Medium, (3-fold dilutions from 10 μM to 56 μM final concentration, final DMSO concentration 1%), 10 μl are added to duplicate wells, plates are rocked to mix, and are incubated as above for 72 h.

An NPTII Elisa kit is obtained from AGDIA, Inc. (Compound direct ELISA test system for Neomycin Phosphotransferase II, PSP 73000/4800). Manufacturer's instructions are followed, with some modifications. 10×PEB-1 lysis buffer is made up to include 500 μM PMSF (Sigma P7626, 50 mM stock in isopropanol). After 72 h incubation, cells are washed once with PBS and 150 μl PEB-1 with PMSF is added per well. Plates are agitated vigorously for 15 minutes, room temperature, then frozen at −70° C. Plates are thawed, lysates are mixed thoroughly, and 100 μl are applied to an NPTII Elisa plate. A standard curve is made. Lysate from DMSO-treated control cells are pooled, serially diluted with PEB-1 with PMSF, and are applied to duplicate wells of the ELISA plate, in a range of initial lysate amount of 150 ul-2.5 ul. In addition, 100 μl buffer alone is applied in duplicate as a blank. Plates are sealed and gently agitated at room temperature for 2 h. Following capture incubation, the plates are washed 5×300 μl with PBS-T (0.5% Tween-20, PBS-T is supplied in the ELISA kit). For detection, a 1× dilution of enzyme conjugate diluent MRS-2 (5×) is made in PBS-T, into which 1:100 dilutions of enzyme conjugates A and B are added, as per instructions. Plates are resealed, and incubated with agitation, covered, room temperature, for 2 h. The washing is then repeated and 100 μl of room temperature TMB substrate is added. After approximately 30 minutes incubation (room temperature, agitation, covered), the reaction is stopped with 50 μl 3M sulfuric acid. Plates are read at 450 nm on a Molecular Devices Versamax plate reader.

Inhibitor effect is expressed as a percentage of DMSO-treated control signal, and inhibition curves are calculated using a 4-parameter equation: y=A+((B−A)/((1+((C/x)^{̂D))), where C is half-maximal activity or EC}₅₀.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

What is claimed is:

1. A compound of Formula (I), or a pharmaceutically acceptable salt thereof: 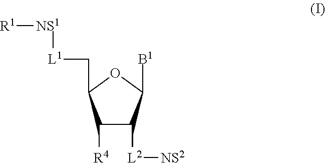
wherein:
R′ is selected from the group consisting of:
—(CH₂)ₐ—OR¹⁶, —O—CH₂—COOR¹⁶, —(CH₂)_{b}—COOR¹⁶, —(CH₂)_{c}—C(═S)OR¹⁶, —(CH₂)_{d}—C(═O)NR¹⁷R¹⁸, —(CH₂)ₑ—NH—SO₂—R¹⁶, —(CH₂)_{f}—NH—SO₂—NR¹⁷R¹⁸, —(CH₂)_{g}—NH—CO₂—R¹⁶, —(CH₂)ₕ—NH—C(═O)—R¹⁶, —(CH₂)ᵢ—NH—C(═O)—NR¹⁷R¹⁸, —CH₂—C(R¹⁹), —CH₂—C(R¹⁹)₂—CH₂—OH 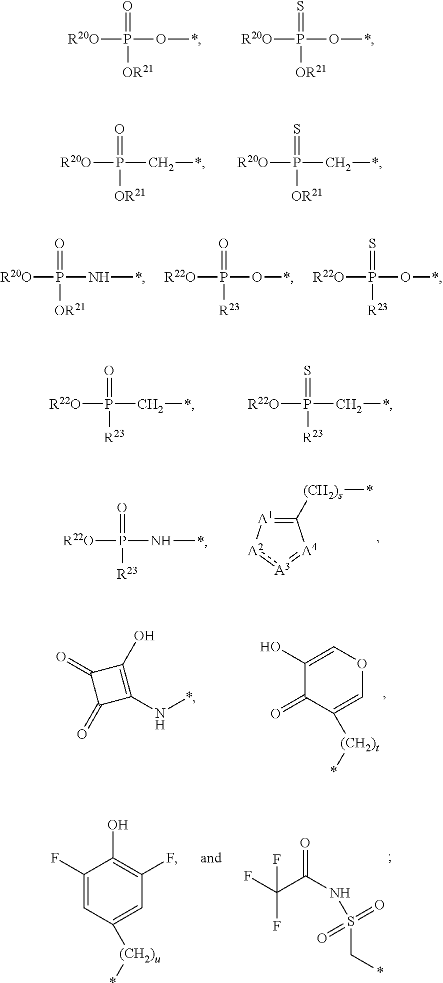
L¹ is 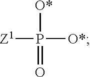
L² is 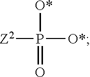
Z¹ is selected from the group consisting of —OR², S⁻ and —SH;
Z² is selected from the group consisting of —OR³, S⁻ and —SH;
R² is selected from the group consisting of absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl and 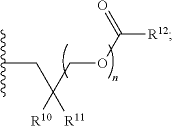
R³ is selected from the group consisting of absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl and 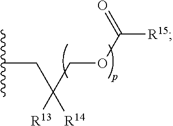
R⁴ is selected from the group consisting of hydrogen, hydroxy, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R⁵)₂—O—C(═O)R⁶;
B¹ is an optionally substituted heterocyclic base;
each R¹⁹ is independently hydrogen or halogen;
R²⁰, R²¹ and R²² are each independently selected from the group consisting of absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, pivaloyloxymethoxy, isopropyloxycarbonyloxymethoxy and 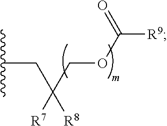
R²³ is independently selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, and NR²⁴R²⁵;
A¹ is CR²⁶ or N;
A² is C(OH), NH, or O (oxygen);
A³ is C(OH) or N (nitrogen);
A⁴ is C(OH), N (nitrogen), or O (oxygen);
R⁷, R⁸, R¹⁰, R¹¹, R¹³ and R¹⁴ are each independently —C≡N or an optionally substituted substituent selected from the group consisting of C₁₋₈ organylcarbonyl, C₁₋₈ alkoxycarbonyl and C₁₋₈ organylaminocarbonyl;
R⁵, R⁶, R⁹, R¹², R¹⁵, R¹⁶, R¹⁷, R⁸, R²⁴, R²⁵ and R²⁶ are each independently selected from the group consisting of hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl;
b, c and d are each independently selected from the group consisting of 1, 2 and 3;
a, e, f, g, h, i, s, t and u are each independently 0 or 1;
m, n and p are each independently 1 or 2;
NS¹ and NS² are each independently selected from the group consisting of a nucleoside, and a protected nucleoside;
each [symbol] is independently a single or double bond, provided that both [symbol] cannot be double bonds;
each * represents a point of attachment; and
provided that when R¹ is 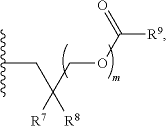
and at least one of R²⁰ and R²¹ is not 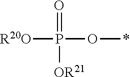
then at least one of Z¹ and Z² is S⁻ or —SH; and
provided that if R⁴ is hydroxy, and Z¹ and Z² are both S⁻ or —SH then R¹ cannot be 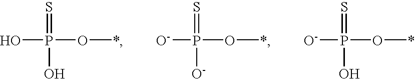
or

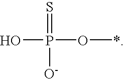

2. The compound of claim 1, wherein L¹ is 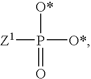
and Z¹ is selected from the group consisting of S⁻ and —SH.

3. The compound of claim 1, wherein L² is 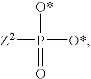
and Z² is selected from the group consisting of S⁻ and —SH.

4. The compound of claim 1, wherein R¹ is selected fro the group consisting of: —(CH₂)ₐ—OR¹⁶, wherein R¹⁶ is hydrogen, and a is 1, —(CH₂)_{b}—COOR¹⁶, wherein R¹⁶ is hydrogen or an optionally substituted C₁₋₆ alkyl, and b is 1, —(CH₂)_{c}—C(═S)OR¹⁶, wherein R¹⁶ is hydrogen or an optionally substituted C₁₋₆ alkyl, and c is 1, —(CH₂)_{c}—C(═O)NR¹⁷R¹⁸, wherein R¹⁷ and R¹⁸ are both hydrogen or an optionally substituted C₁₋₆ alkyl, and c is 1.

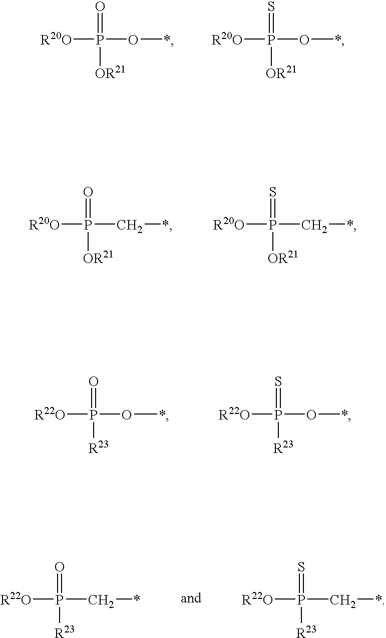

5. The compound of claim 4, wherein R²⁰ and R²¹ are both hydrogen.

6. The compound of claim 4, wherein one of R²⁰ and R²¹ is hydrogen, and the other of R²⁰ and R²¹ is selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl.

7. The compound of claim 4, wherein both R²⁰ and R²¹ are independently selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl.

8. The compound of claim 4, wherein both R²⁰ and R²¹ are independently selected from the group consisting of pivaloyloxymethoxy, isopropyloxycarbonyloxymethoxy and

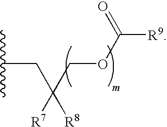

9. The compound of claim 4, wherein R²² is selected from the group consisting of absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl; and R²³ is independently selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl.

10. The compound of claim 4, wherein R²² is hydrogen, and R²³ is NR²⁴R²⁵, wherein R²⁴ and R²⁵ are each independently selected from the group consisting of hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl.

11. The compound of claim 1, wherein R⁴ is selected from the group consisting of hydroxy, hydrogen, an optionally substituted —O—C₁₋₆ alkyl and —OC(R⁵)₂—O—C(═O)R⁶.

12. The compound of claim 11, wherein the optionally substituted —O—C₁₋₆ alkyl is an unsubstituted methoxy.

13. The compound of claim 1, wherein NS¹ has the structure: 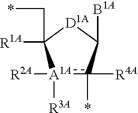
wherein: [symbol]
is a single or a double bond;
A^{1A} is selected from the group consisting of C, O and S;
B^{1A} is an optionally substituted heterocyclic base;
D^{1A} is C═CH₂ or O;
R^{1A} is selected from the group consisting of hydrogen, azido, —CN, an optionally substituted C₁₋₄ alkyl and an optionally substituted C₁₋₄ alkoxy;
R^{2A} is absent or selected from the group consisting of hydrogen, halogen, hydroxy and an optionally substituted C₁₋₄ alkyl;
R^{3A} is absent or selected from the group consisting of hydrogen, halogen, azido, amino, hydroxy, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R^{5A})₂—O—C(═O)R^{6A}; R^{4A} is absent or selected from the group consisting of hydrogen, halogen, hydroxy, —CN, —NC, an optionally substituted C₁₋₄ alkyl, an optionally substituted haloalkyl and an optionally substituted hydroxyalkyl;
each R^{5A} and R^{6A} are independently hydrogen or an optionally substituted C₁₋₄-alkyl; and
* represents a point of attachment.

14. The compound of claim 13, wherein:
wherein: [symbol] is a single bond; A^{1A} is C; B^{1A} is an optionally substituted heterocyclic base; D^{1A} is O; R^{1A} is hydrogen; R^{2A} is hydrogen; R^{3A} is selected from the group consisting of hydrogen, hydroxy, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R^{5A})₂—O—C(═O)R^{6A}; each R^{5A} and R^{6A} are independently hydrogen or an optionally substituted C₁₋₄-alkyl; and * represents a point of attachment.

15. The compound of claim 1, wherein NS¹ is selected from the group consisting of: 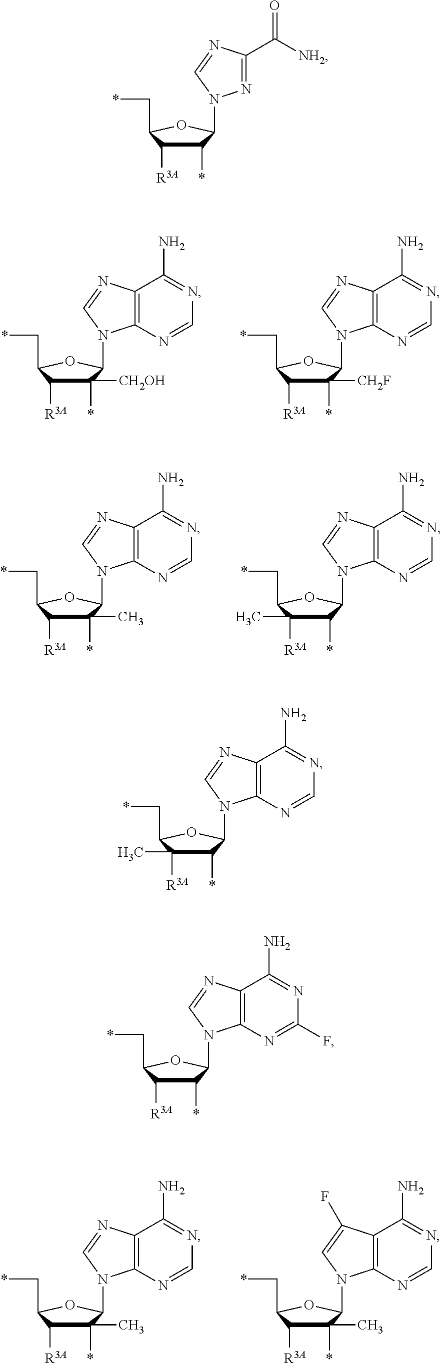 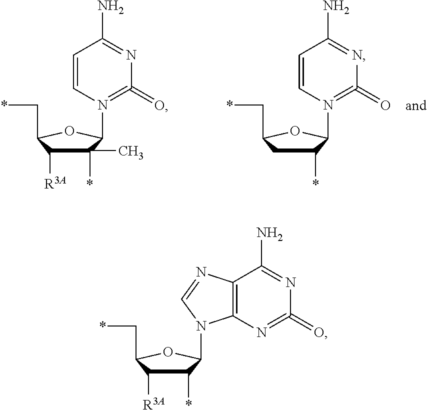
wherein:
* represents a point of attachment.

16. The compound of claim 1, wherein NS² has the structure: 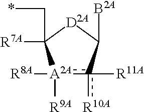
wherein:
each [symbol] is independently a single or a double bond, provided that both [symbol] cannot be double bonds;
A^{2A} is selected from the group consisting of C, O and S;
B^{2A} is an optionally substituted heterocyclic base;
D^{2A} is C═CH₂ or O;
R^{7A} is selected from the group consisting of hydrogen, azido, —CN, an optionally substituted C₁₋₄ alkyl and an optionally substituted C₁₋₄ alkoxy;
R^{8A} is absent or selected from the group consisting of hydrogen, halogen, hydroxy and an optionally substituted C₁₋₄ alkyl;
R^{9A} is absent or selected from the group consisting of hydrogen, halogen, azido, amino and hydroxy;
R^{10A} is absent or selected from the group consisting of hydrogen, halogen, hydroxy, —CN, —NC, an optionally substituted C₁₋₄ alkyl and an optionally substituted C₁₋₄ alkoxy;
R^{11A} is absent or selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R^{10A} indicated by [symbol] is a double bond, then R^{10A} is a C₂₋₄ alkenyl and R^{11A} is absent; and
* represents a point of attachment.

17. The compound of claim 1, wherein NS² is selected from the group consisting of: 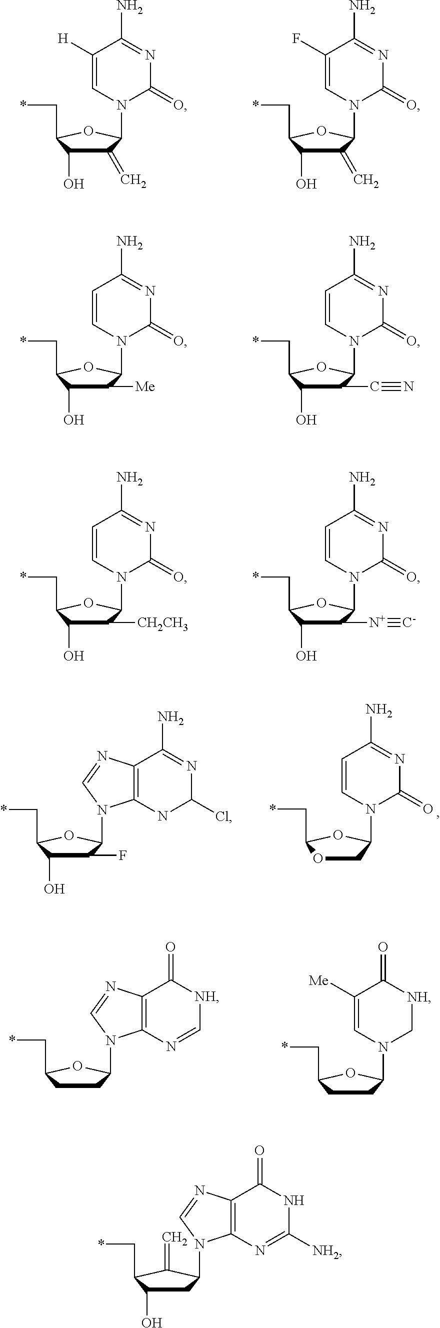 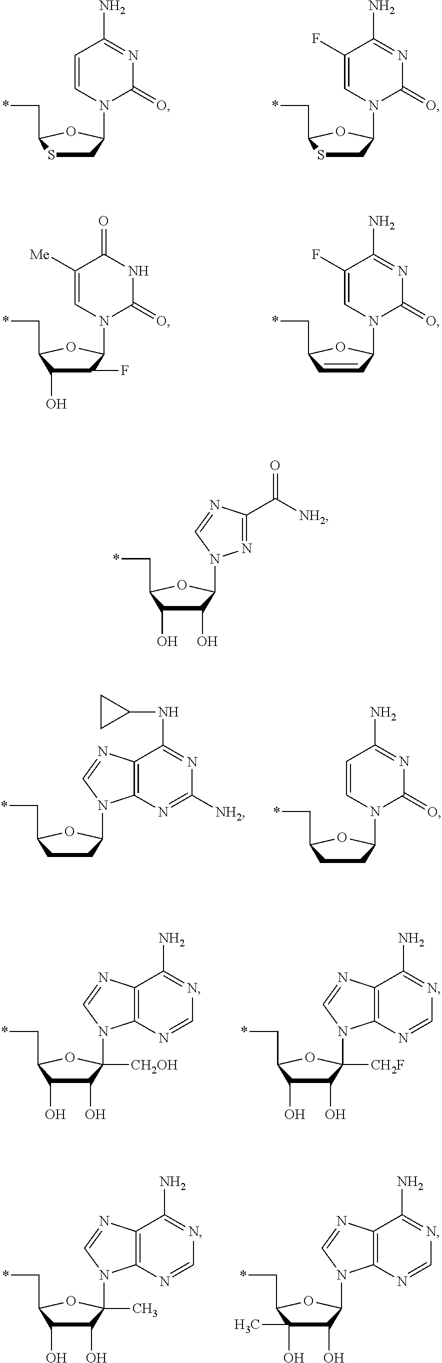 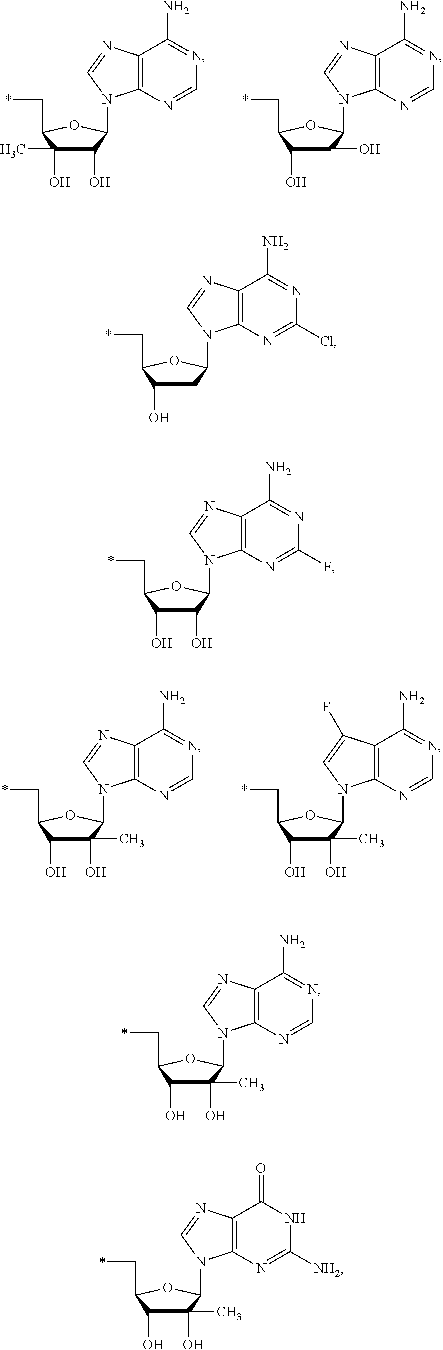 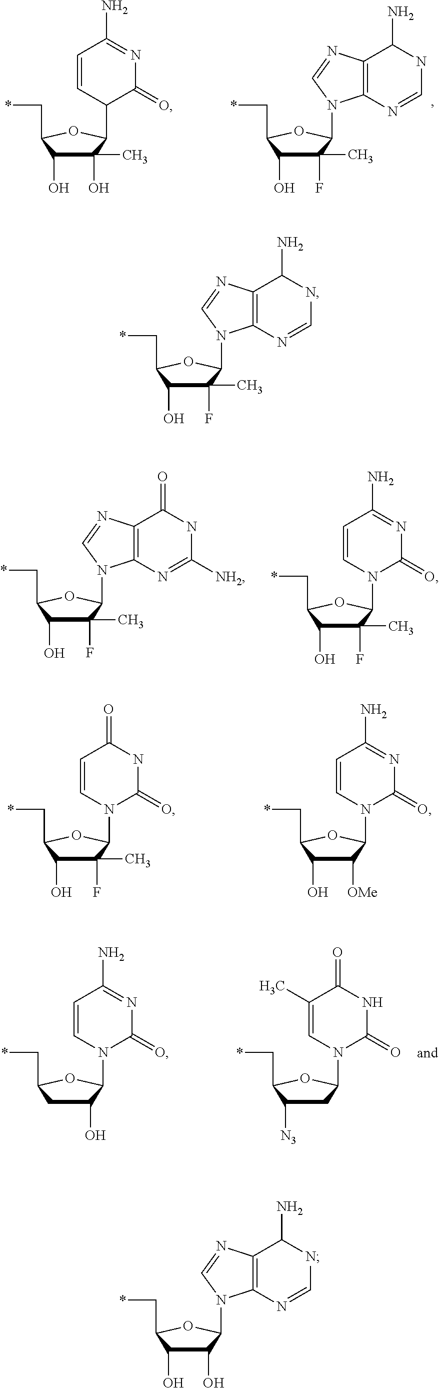
wherein * represents a point of attachment.

18. The compound of claim 1, wherein
NS¹ is 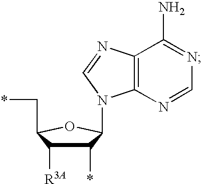
and NS² is 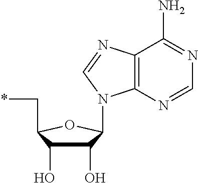
wherein * represents a point of attachment.

19. The compound of claim 1 having the structure:

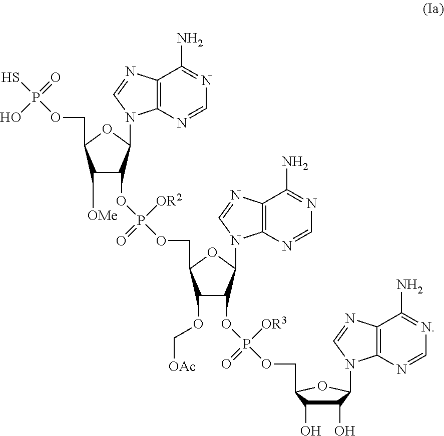

20. The compound of claim 1, wherein the compound of formula (I) is selected from the group consisting of:

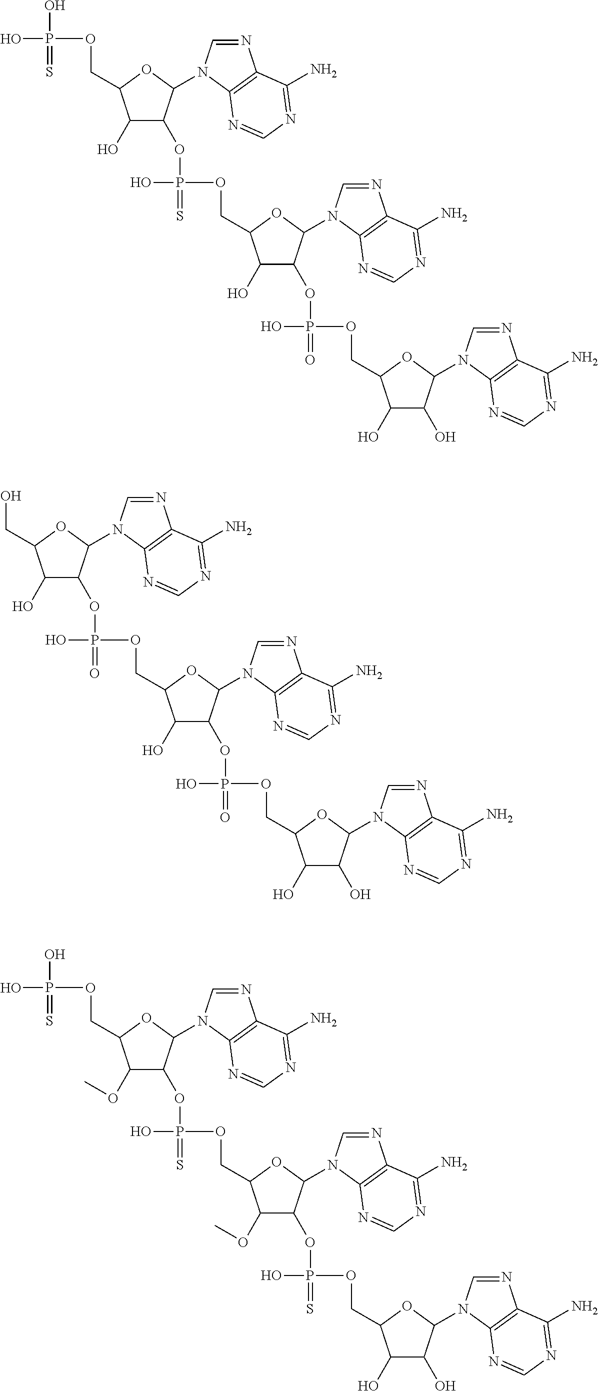

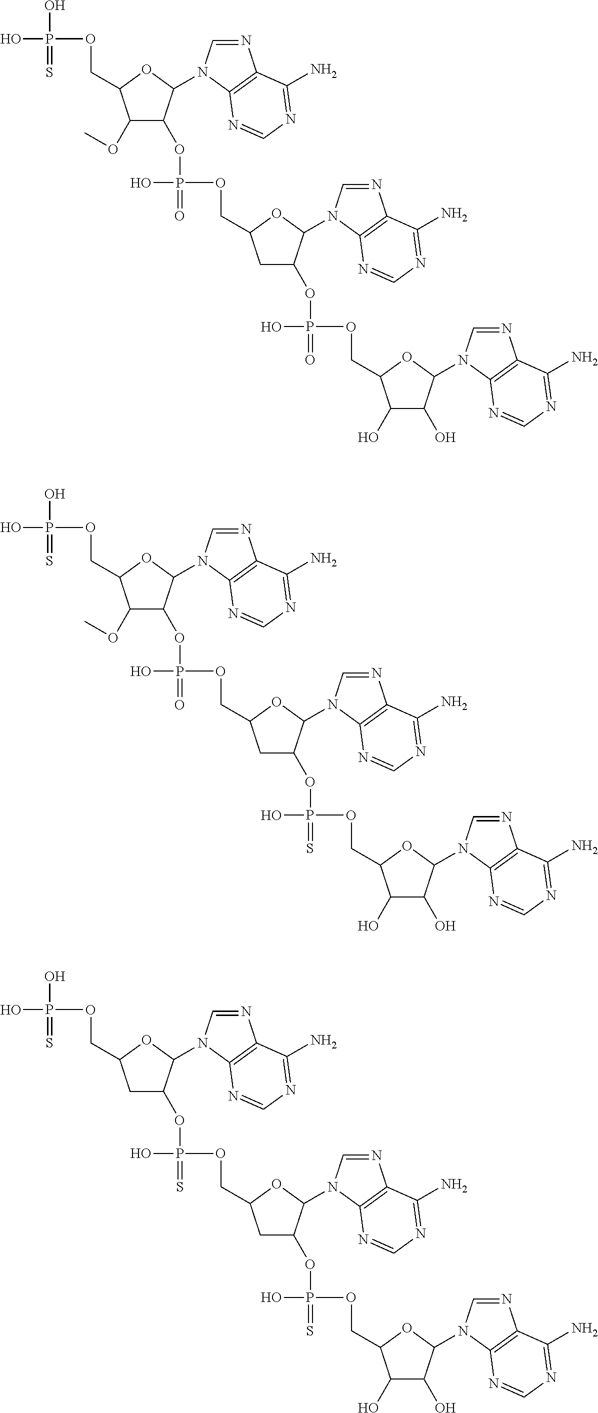

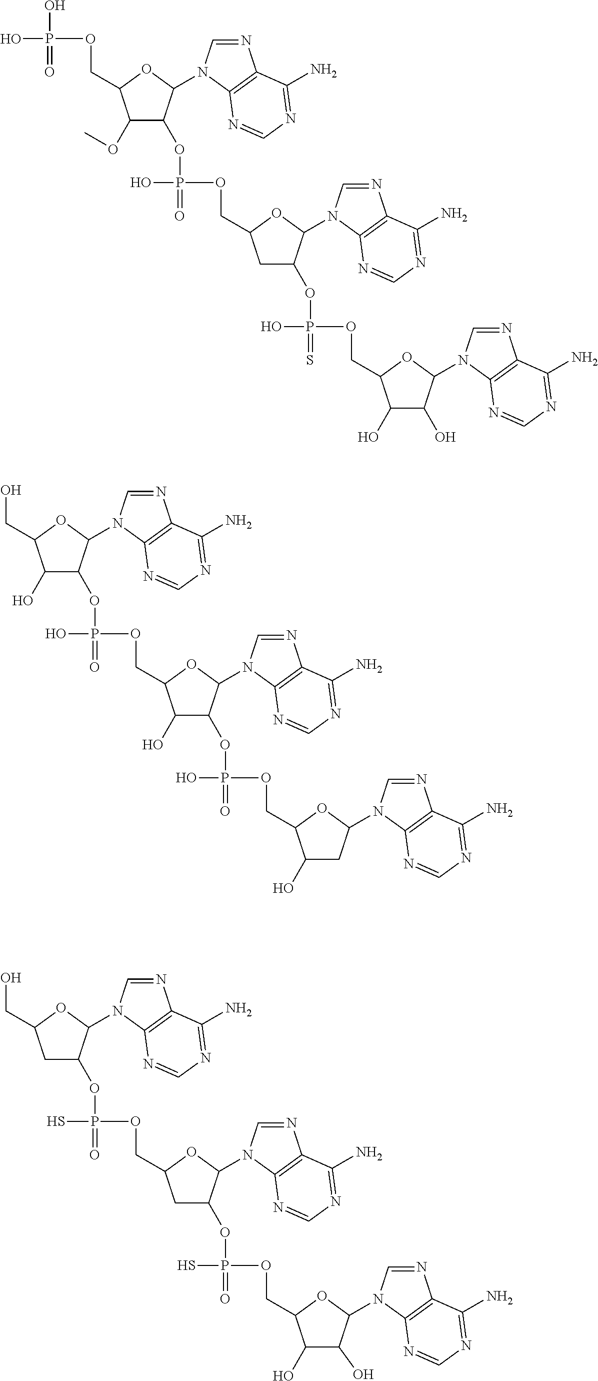

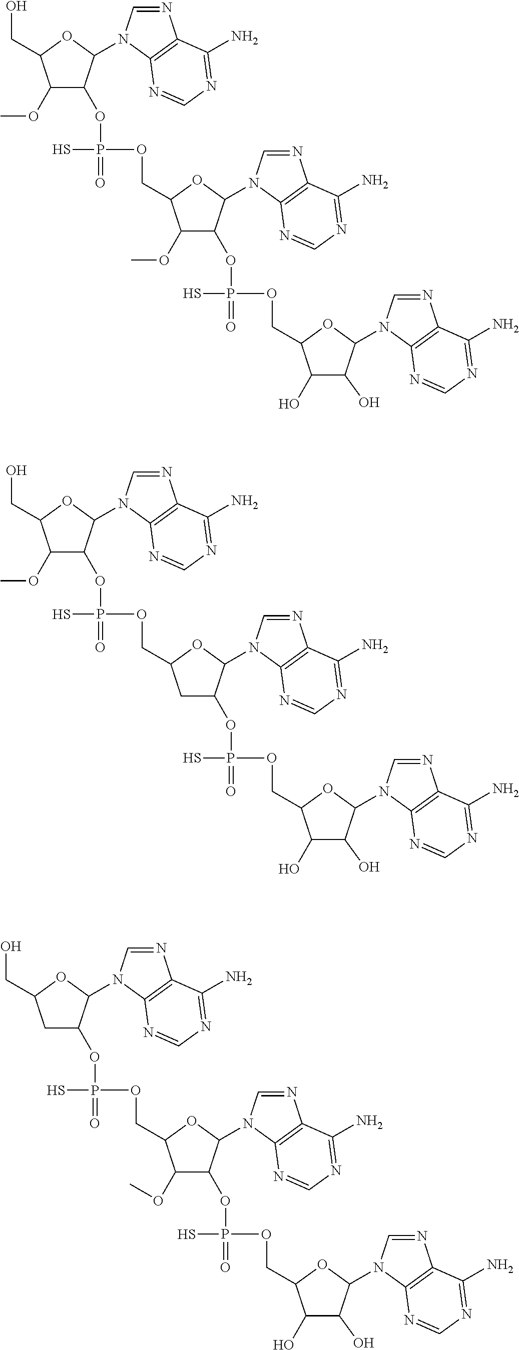

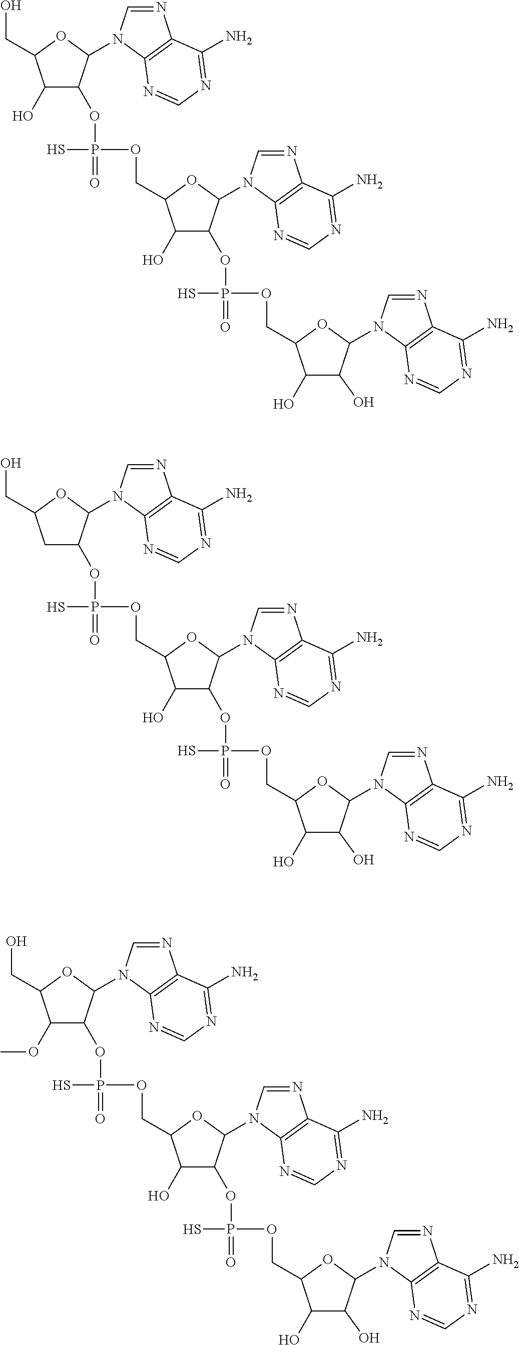

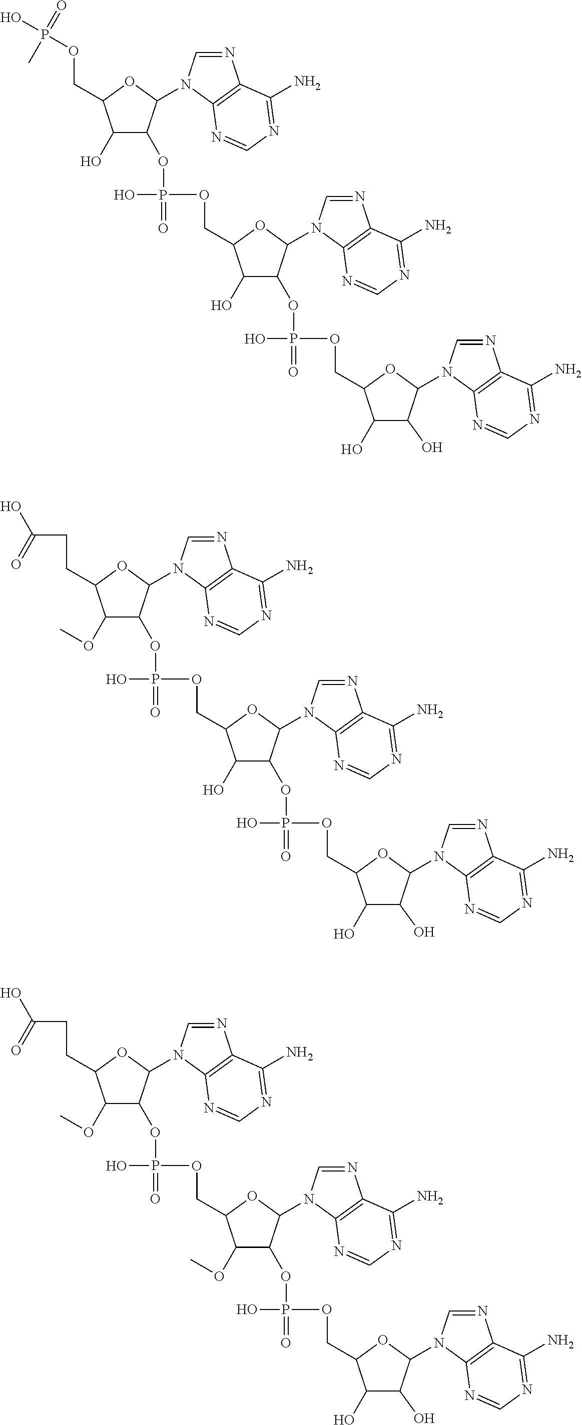

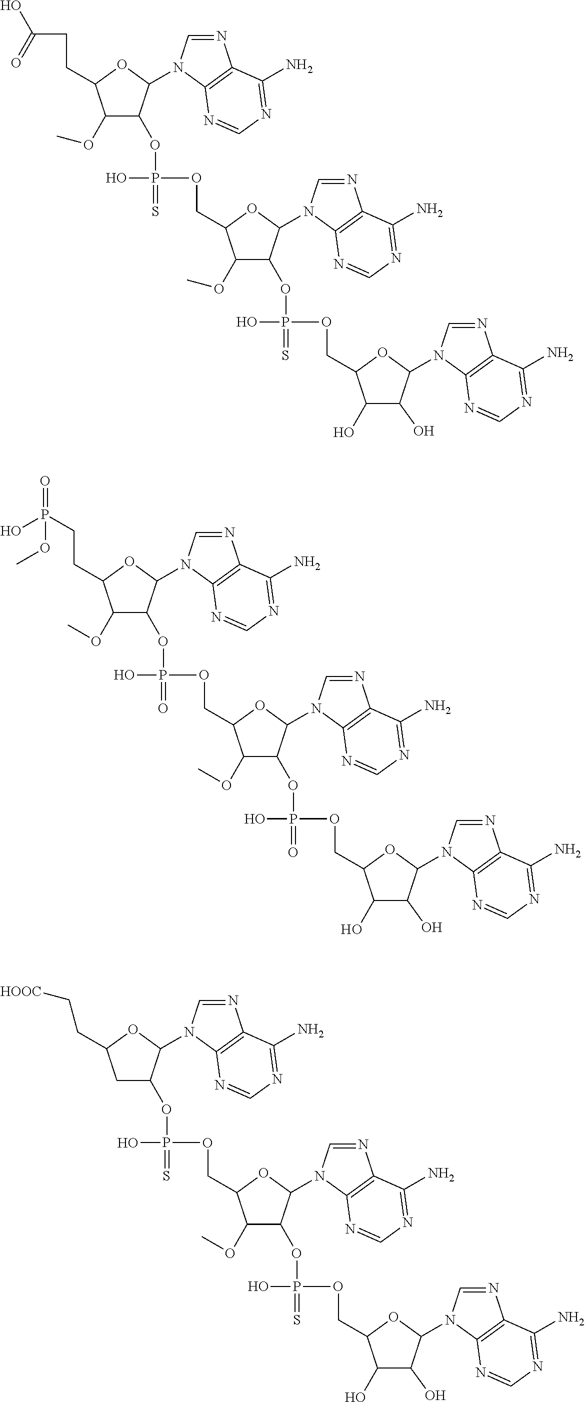

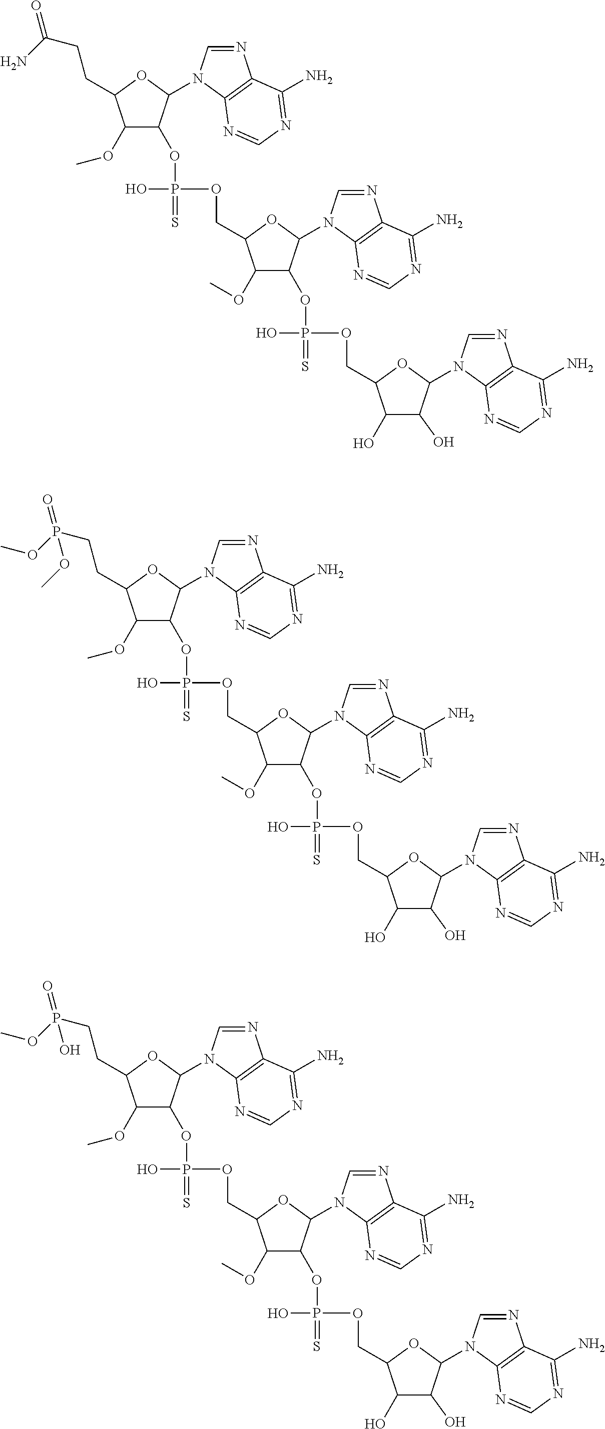

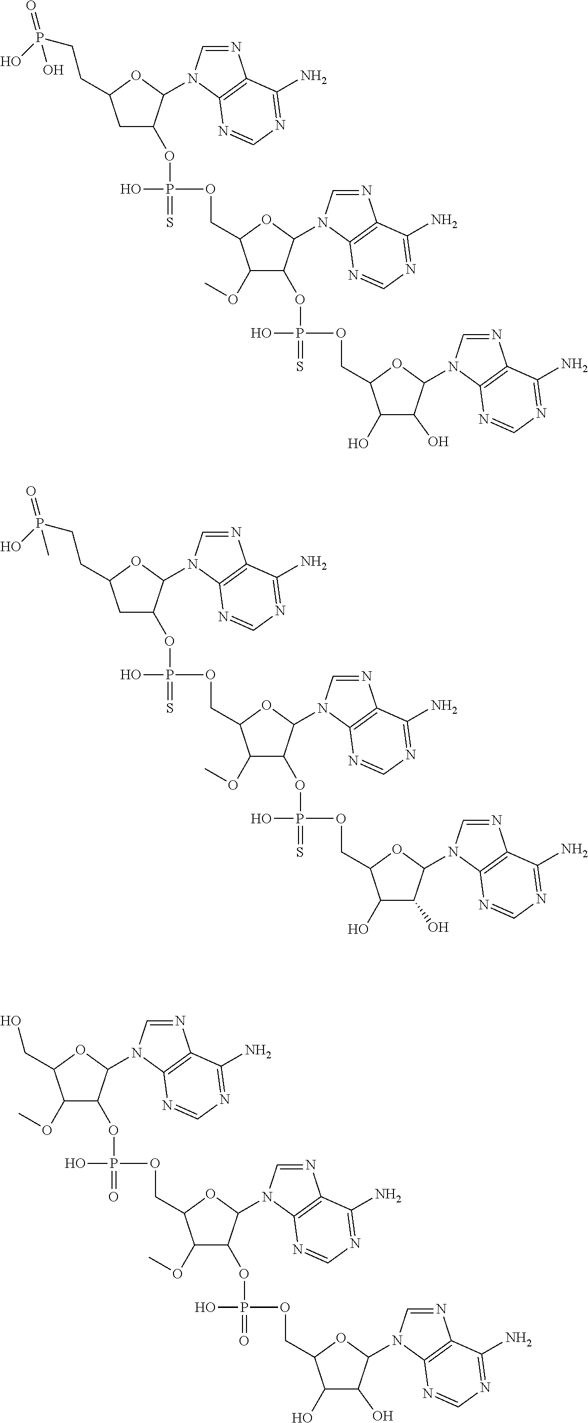

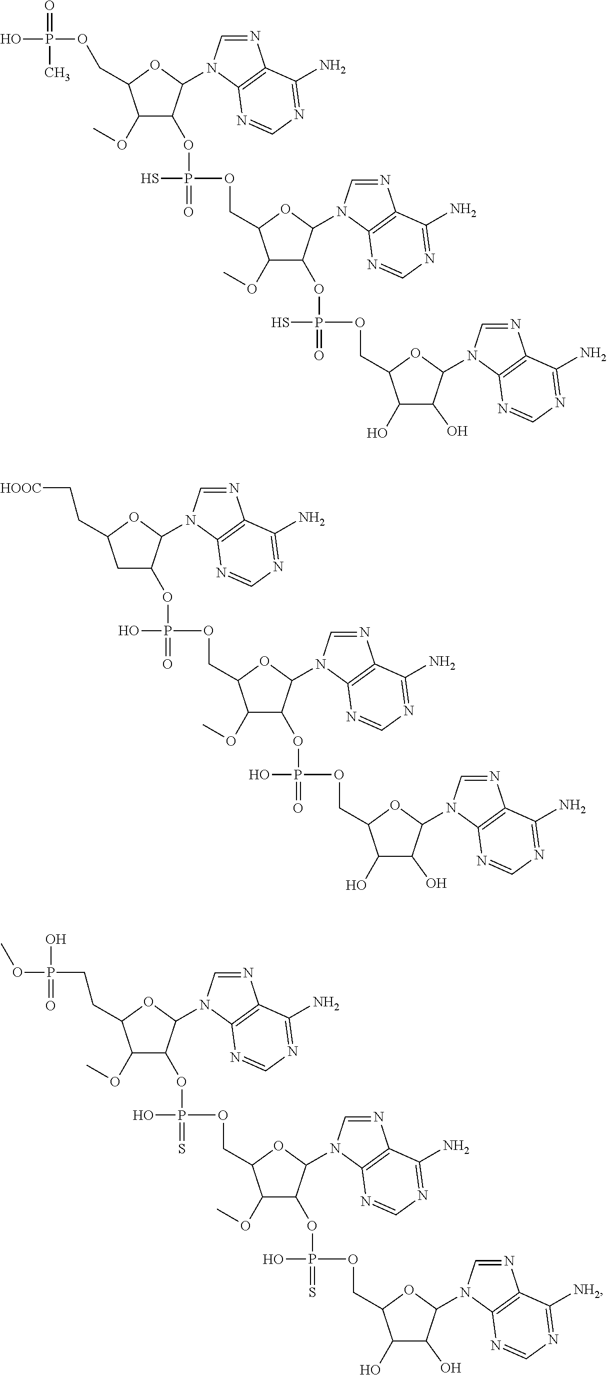

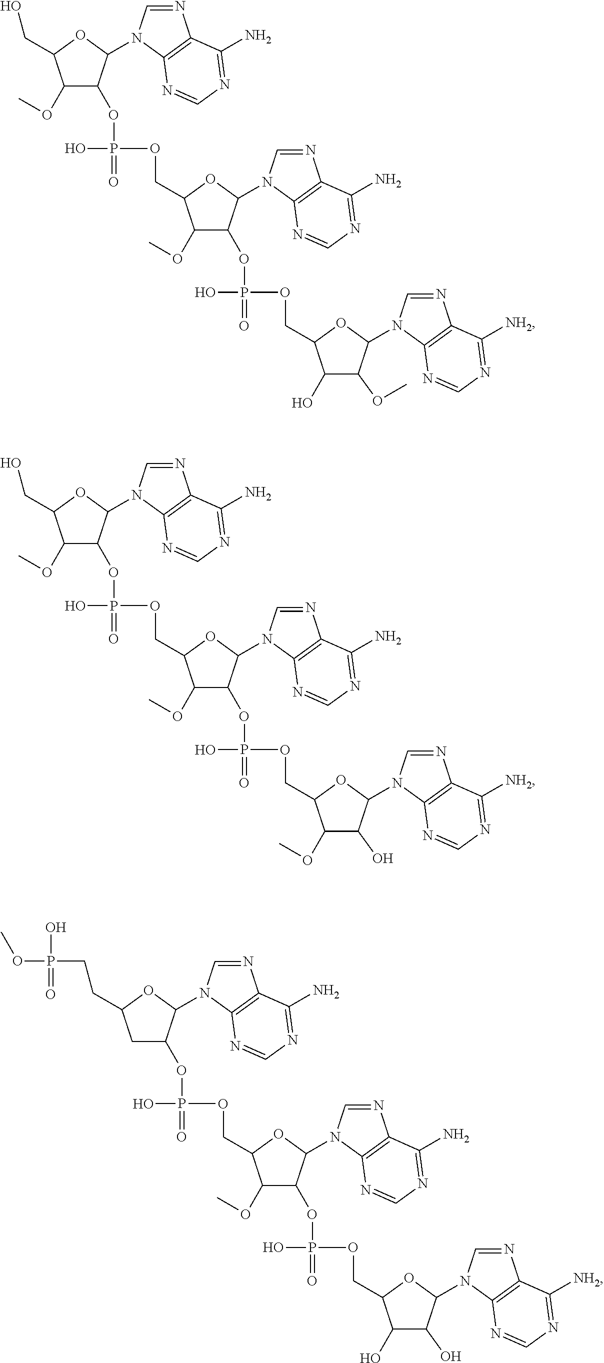

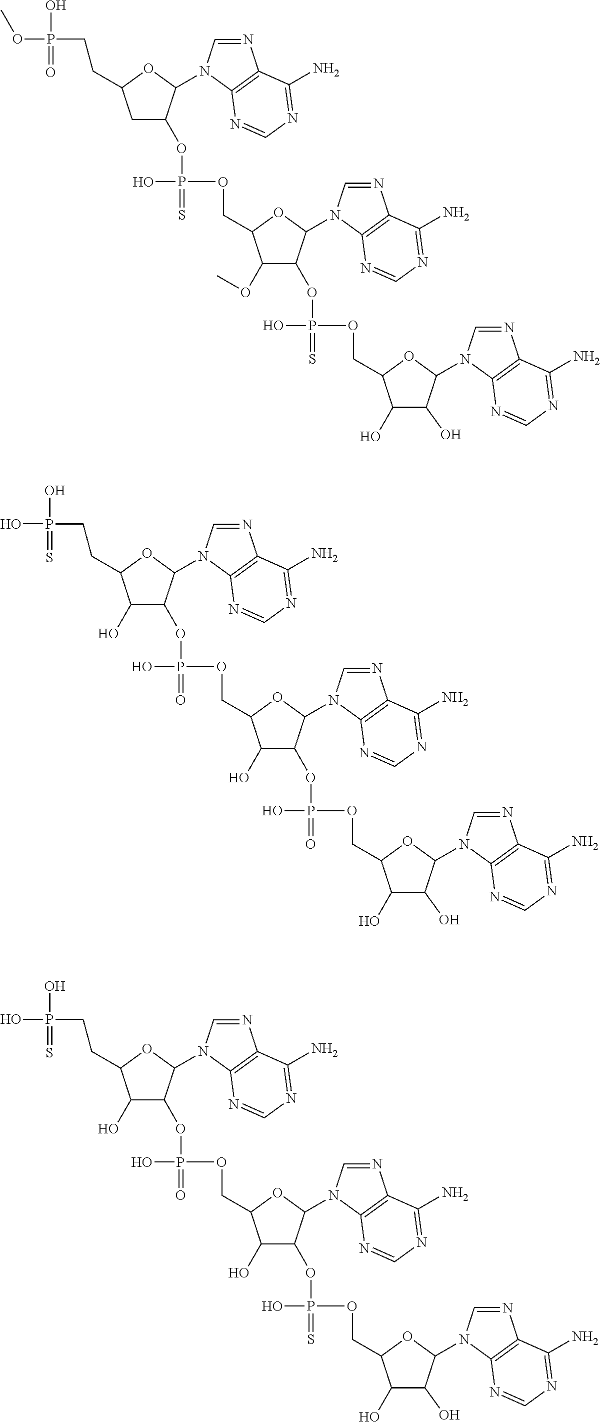

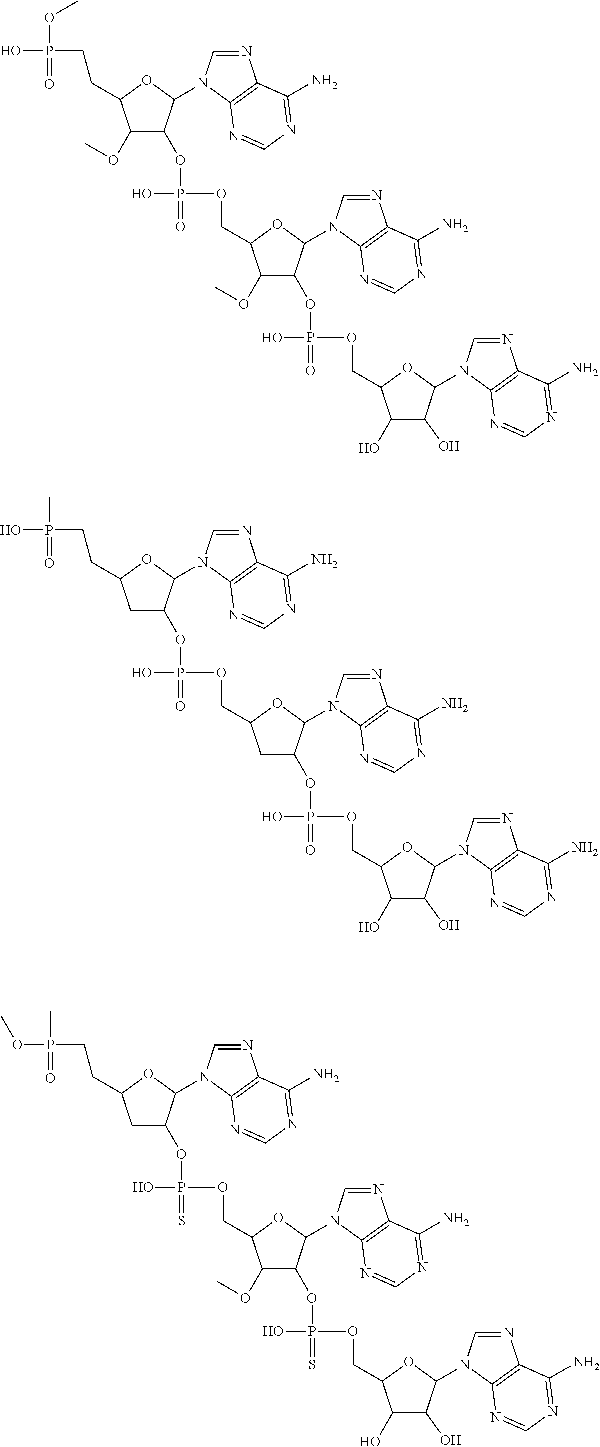

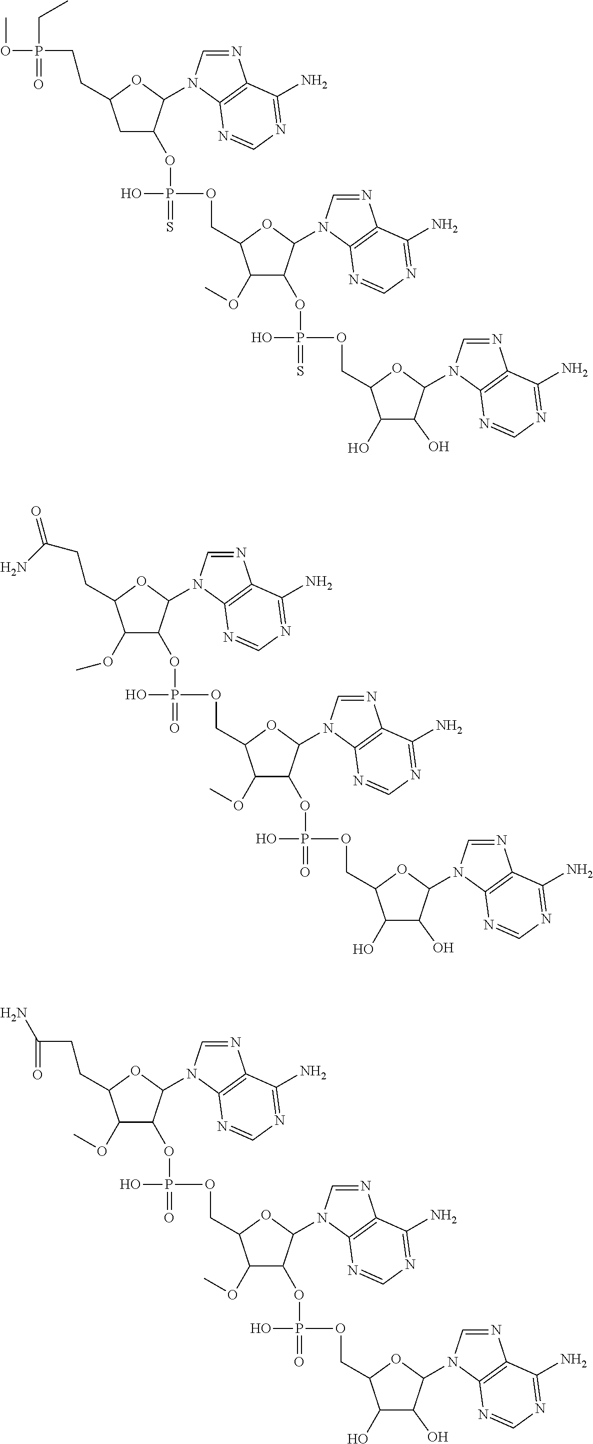

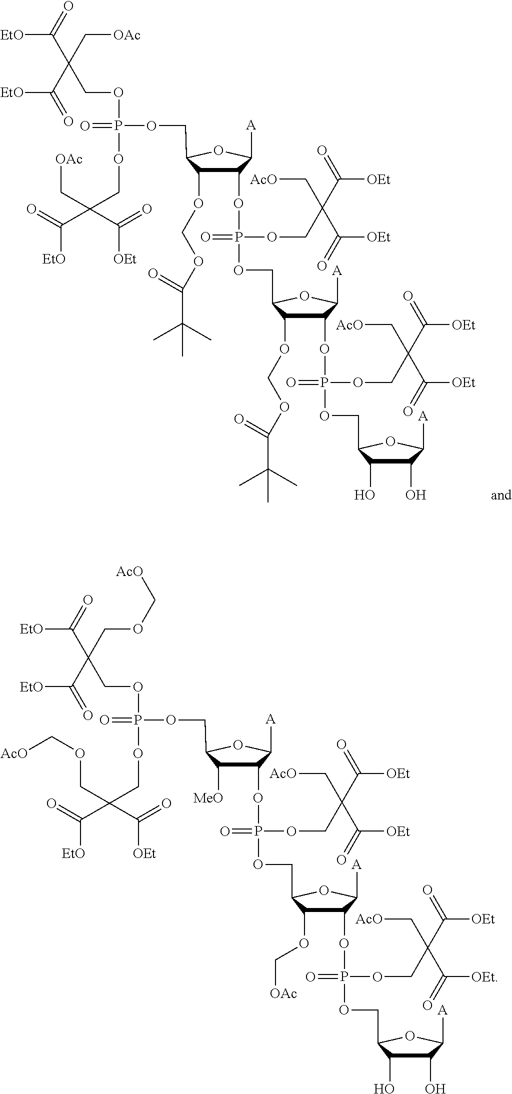

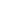

21. A compound of Formula (II), or a pharmaceutically acceptable salt thereof: 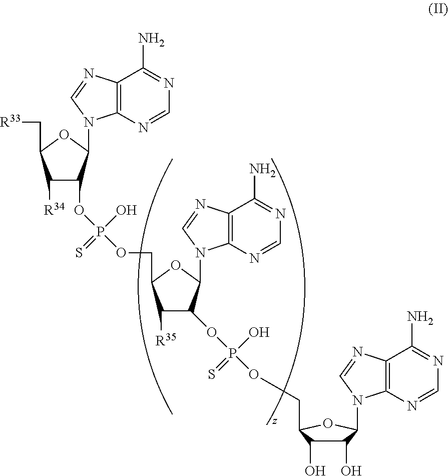
wherein:
R³³ is selected from the group consisting of:
—(CH₂)_{A}—OR³⁶, —O—CH₂—COOR³⁶, —(CH₂)_{B}—COOR³⁶, —(CH₂)_{C}—C(═S)OR³⁶, —(CH₂)_{D}—C(═O)NR³⁷R³⁸, —(CH₂)_{E}—NH—SO₂—R³⁶, —(CH₂)_{F}—NH—SO₂—NR³⁷R³⁸, —(CH₂)_{G}—NH—CO₂—R³⁶, —(CH₂)_{H}—NR—C(═O)—R³⁶, —(CH₂)_{I}—NH—C(═O)—NR³⁷R³⁸, —CH₂—C(R³⁹)₂—CH₂—OH, 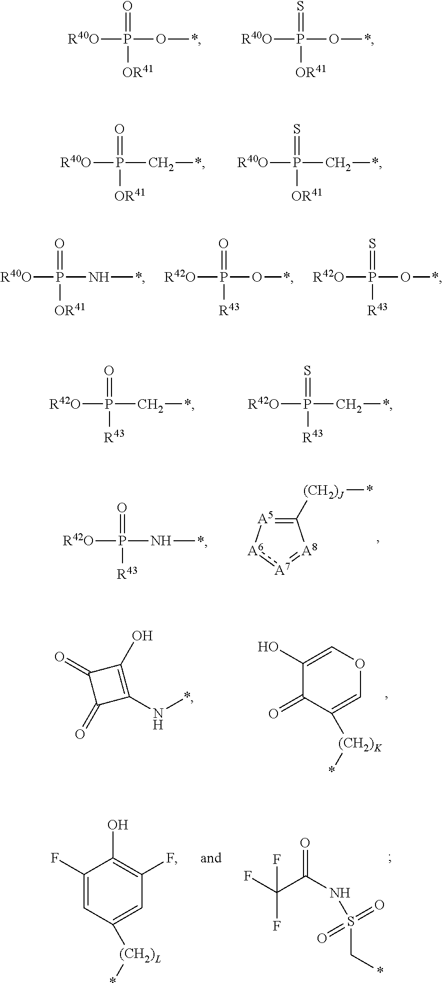
R³⁴ and each R³⁵ are each independently selected from the group consisting of hydrogen, hydroxy, an optionally substituted —O—C₁₋₆ alkyl, an optionally substituted —O—C₂₋₆ alkenyl, an optionally substituted —O—C₂₋₆ alkynyl, an optionally substituted —O—C₃₋₆ cycloalkyl, an optionally substituted —O—C₃₋₆ cycloalkenyl, an optionally substituted —O—C₃₋₆ cycloalkynyl and —OC(R⁵⁰)₂—O—C(═O)R⁵¹;
R³⁶, R³⁷, R³⁸, R⁵⁰ and R⁵¹ are each independently selected from the group consisting of hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl;
each R³⁹ is independently hydrogen or halogen;
R⁴⁰, R⁴¹ and R⁴² are each independently selected from the group consisting of absent, hydrogen, an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, pivaloyloxymethoxy, isopropyloxycarbonyloxymethoxy and 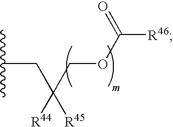
R⁴³ is independently selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl, an optionally substituted C₃₋₆ cycloalkynyl, and NR⁴⁷R⁴⁸;
R⁴⁴ and R⁴⁵ are each independently —C≡N or an optionally substituted substituent selected from the group consisting of C₁₋₈ organylcarbonyl, C₁₋₈ alkoxycarbonyl and C₁₋₈ organylaminocarbonyl;
R⁴⁶, R⁴⁷, R⁴⁸ and R⁴⁹ are each independently selected from the group consisting of hydrogen, an optionally substituted C₁₋₆-alkyl, an optionally substituted C₂₋₆ alkenyl, an optionally substituted C₂₋₆ alkynyl, an optionally substituted C₃₋₆ cycloalkyl, an optionally substituted C₃₋₆ cycloalkenyl and an optionally substituted C₃₋₆ cycloalkynyl;
A⁵ is CR⁴⁹ or N;
A⁶ is C(OH), NH, or O (oxygen);
A⁷ is C(OH) or N (nitrogen);
A⁸ is C(OH), N (nitrogen), or O (oxygen);
B, C and D are each independently selected from the group consisting of 1, 2 and 3;
A, E, F, G, H and I are each independently 0 or 1;
J, K and L are each independently 0 or 1;
M is 1 or 2;
each [symbol] is a single or double bond; and
Z is an integer in the range of 2-10.

22. The compound of claim 21, wherein the compound of formula (II) is selected from the group consisting of:

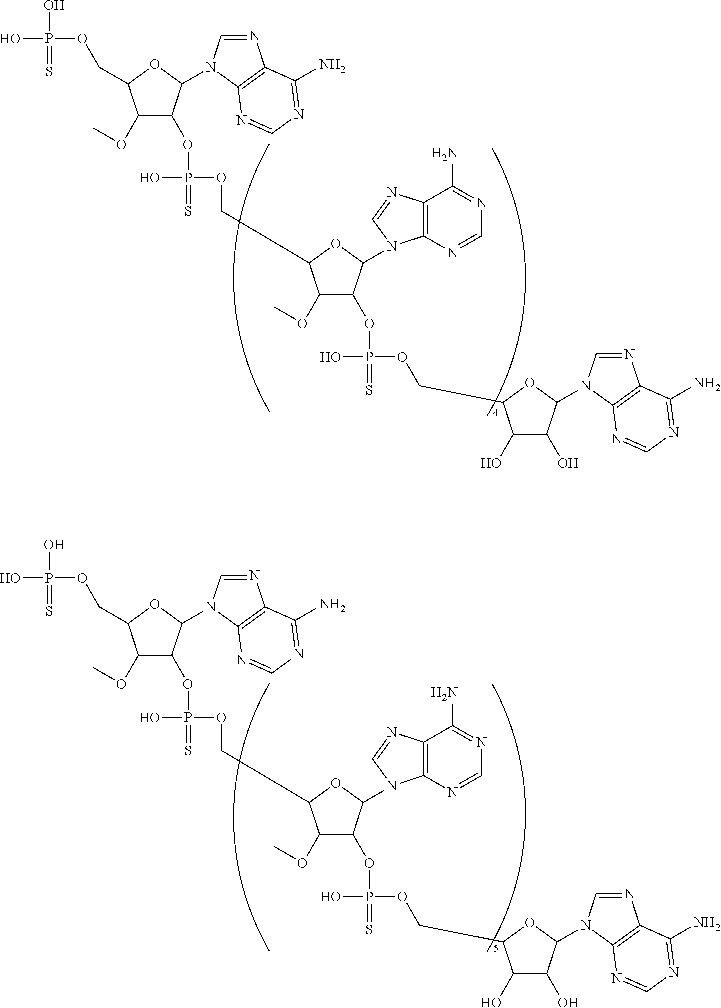

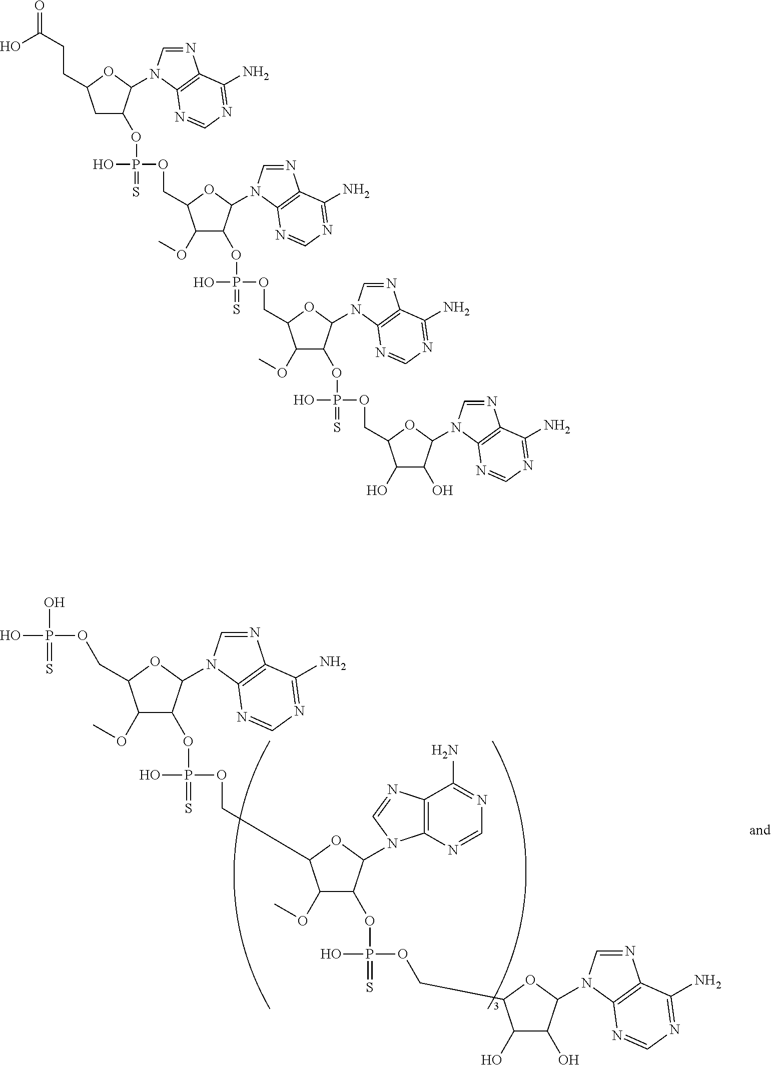

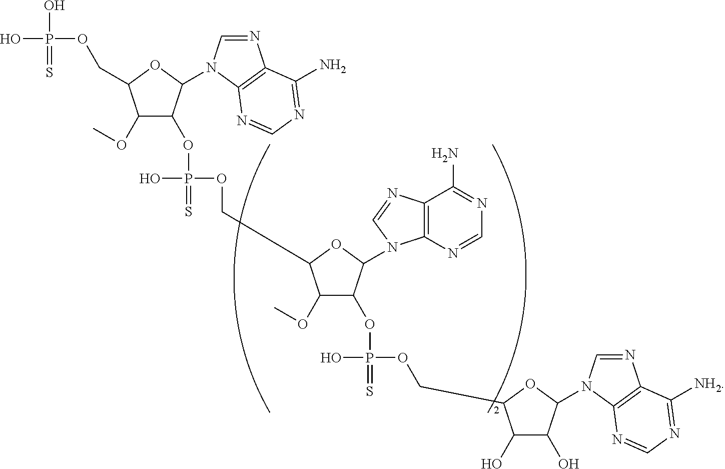

23. A pharmaceutical composition comprising a compound of claim 1, or a pharmaceutically acceptable salt thereof, and a pharmaceutically acceptable carrier, diluent, excipient or combination thereof.

24. A method of ameliorating or treating a neoplastic disease comprising administering to a subject suffering from a neoplastic disease a therapeutically effective amount of a compound of claim 1, or a pharmaceutically acceptable salt thereof.

25. A method of ameliorating or treating a viral infection comprising administering to a subject suffering from a viral infection a therapeutically effective amount of a compound of claim 1, or a pharmaceutically acceptable salt thereof.

26. The method of claim 25, wherein the viral infection is caused by a virus selected from the group consisting of an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae, a Togaviridae, an Arenaviridae and a Bornaviridae.

27. A method of ameliorating or treating a bacterial infection comprising administering to a subject suffering from a bacterial infection a therapeutically effective amount of a compound of claim 1, or a pharmaceutically acceptable salt thereof.

28. A method of ameliorating or treating a parasitic disease comprising administering to a subject suffering from a parasitic disease a therapeutically effective amount of a compound of claim 1, or a pharmaceutically acceptable salt thereof.

29. A pharmaceutical composition comprising a compound of claim 21, or a pharmaceutically acceptable salt, and a pharmaceutically acceptable carrier, diluent, excipient or combination thereof.

30. A method of ameliorating or treating a neoplastic disease comprising administering to a subject suffering from a neoplastic disease a therapeutically effective amount of a compound of claim 21, or a pharmaceutically acceptable salt thereof.

31. A method of ameliorating or treating a viral infection comprising administering to a subject suffering from a viral infection a therapeutically effective amount of a compound of claim 21, or a pharmaceutically acceptable salt thereof.

32. The method of claim 31, wherein the viral infection is caused by a virus selected from the group consisting of an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae, a Togaviridae, an Arenaviridae and a Bornaviridae.

33. A method of ameliorating or treating a bacterial infection comprising administering to a subject suffering from a bacterial infection a therapeutically effective amount of a compound of claim 21, or a pharmaceutically acceptable salt thereof.

34. A method of ameliorating or treating a parasitic disease comprising administering to a subject suffering from a parasitic disease a therapeutically effective amount of a compound of claim 21, or a pharmaceutically acceptable salt.
